(19) United States
(12) Patent Application Publication
(10) Pub. No.: US 2024/0262879 A1
(43) Pub. Date: Aug. 8, 2024

(54) IL-12 Fc FUSION PROTEINS

(71) Applicant: Boehringer Ingelheim International GmbH, Ingelheim am Rhein (DE)

(72) Inventors: Stephen R. COMEAU, Avon, NY (US); Phillip KIM, San Diego, CA (US); Aleksandra Katarzyna KOWALCZYK, Stuttgart (DE); Randal Scott KUDRA, Danbury, CT (US); Emma LANGLEY, San Diego, CA (US); Chen LI, Irvine, CA (US); Philipp MUELLER, Mittelbiberach (DE); Andrew K. URICK, Newtown, CT (US)

(21) Appl. No.: 18/417,047

(22) Filed: Jan. 19, 2024

(30) Foreign Application Priority Data
Jan. 20, 2023 (EP) 23152699.7

(51) Int. Cl. C07K 14/54; A61K 38/00; A61P 35/00; C07K 16/18

(52) U.S. Cl. CPC C07K 14/5434; A61P 35/00; C07K 16/18; A61K 38/00; C07K 2317/569; C07K 2319/30

(57) ABSTRACT

This invention relates to IL-12 Fc fusion proteins and their use in medicine, pharmaceutical compositions comprising the same, and methods of using the same as agents for treatment and/or prevention of cancer.

### RELATED APPLICATION DISCLOSURE

This application claims the benefit European Patent Application No. 23152699.7, filed Jan. 20, 2023, which is hereby incorporated by reference herein in its entirety.

### SEQUENCE DISCLOSURE

This application includes, as part of its disclosure, a “Sequence Listing XML” pursuant to 37 C.F.R. § 1.831(a) which is submitted in XML file format via the USPTO patent electronic filing system in a file named “01-3544-US-1-2024-01-19_Sequence_Listing.xml”, created on Dec. 15, 2023, and having a size of 511,806 bytes, which is hereby incorporated by reference herein in its entirety.

### FIELD OF THE INVENTION

This invention relates to IL-12 Fc fusion proteins and their use in medicine, pharmaceutical compositions comprising the same, and methods of using the same as agents for treatment and/or prevention of cancer.

### BACKGROUND OF THE INVENTION

Interleukin-12 (IL-12) is a cytokine with proven anti-tumor potential showing promising preclinical efficacy in mouse tumor models. However, drug related toxicities were observed in clinical trials, resulting in suboptimal IL-12 dosing regimen and lack of efficacy in patients.

To overcome drug related toxicities masking of IL-12 was proposed to prevent systemic activity & toxicity to create a therapeutic window. Masking of the IL-12 activity can be done by e.g. fusing a domain of the IL-12 receptor to IL-12 via a protease-cleavable linker and subsequent local activation by protease-mediated removal of the IL-12 receptor at the tumor site in cancer patients.

Currently, there are no approved therapies based on IL-12. Hence, there is still a high unmet need for providing new therapeutic IL-12 based biological molecules which may be used for the treatment of cancer.

### SUMMARY OF THE INVENTION

In a first aspect, the present invention relates to an Interleukin-12 (IL-12) Fc fusion protein comprising a first polypeptide chain and a second polypeptide chain, wherein a) the first polypeptide chain comprises a first Fc domain and an IL-12p35 subunit and an IL-12p40 subunit of IL-12, and b) the second polypeptide chain comprises a second Fc domain and a masking moiety that binds to the IL-12p35 and/or IL-12p40 subunit in the first polypeptide chain; wherein the first and second polypeptide chain are linked via the first Fc domain and the second Fc domain, wherein the IL-12p35 subunit or the IL-12p40 subunit is linked to the C-terminus of the first Fc domain via a first peptide linker, which first peptide linker is protease-cleavable, wherein the masking moiety is linked to the C-terminus of the second Fc domain via a second linker, preferably a peptide linker, and wherein the first or the second polypeptide chain further comprises a binding moiety selected from the group consisting of: a collagen binding moiety, a heparin binding moiety, and a fibronectin binding moiety.

In a further embodiment relating to the IL-12 Fc fusion protein according to the first aspect or any of its embodiments the binding moiety is linked to the C-terminus of the IL-12p35 subunit or to the C-terminus of the IL-12p40 subunit, or the binding moiety is linked to the C-terminus of the masking moiety, and in each case optionally via a third polypeptide linker.

In a further embodiment relating to the IL-12 Fc fusion protein according to the first aspect or any of its embodiments the binding moiety is located between the IL-12p35 subunit and the IL-12p40 subunit, or the binding moiety is located between the C-terminus of the first Fc domain and the N-terminus of the IL-12p35subunit or the N-terminus of the IL-12p40 subunit, and in either case the binding moiety may be optionally flanked on one or both sides by a linker or linkers, preferably a peptide linker.

In a further embodiment relating to the IL-12 Fc fusion protein according to the first aspect or any of its embodiments the binding moiety is a collagen binding moiety.

In a further embodiment relating to the IL-12 Fc fusion protein according to the first aspect or any of its embodiments the collagen binding moiety binds to collagen I.

In a further embodiment relating to the IL-12 Fc fusion protein according to the first aspect or any of its embodiments the collagen binding moiety binds to collagen I and has the sequence LxxLxLxxN (SEQ ID NO:41), wherein L is Leucine and N is Asparagine and x is any amino acid.

In a further embodiment relating to the IL-12 Fc fusion protein according to the first aspect or any of its embodiments the collagen binding moiety has a length of 20 amino acids (aa), 19aa, 18aa, 17aa, 16aa, 15aa, 14aa, 13aa, 12aa, 11aa, 10a, or 9aa.

In a further embodiment relating to the IL-12 Fc fusion protein according to the first aspect or any of its embodiments the collagen binding moiety comprises or consists of any one of the amino acid sequences of SEQ ID NOs:40-47.

In a further embodiment relating to the IL-12 Fc fusion protein according to the first aspect or any of its embodiments the binding moiety is a heparin binding moiety.

In a further embodiment relating to the IL-12 Fc fusion protein according to the first aspect or any of its embodiments the heparin binding moiety has the sequence VRIQRKKEKMKET (SEQ ID NO:50).

In a further embodiment relating to the IL-12 Fc fusion protein according to the first aspect or any of its embodiments the collagen binding moiety binds to collagen IV.

In a further embodiment relating to the IL-12 Fc fusion protein according to the first aspect or any of its embodiments the collagen binding moiety has the sequence KLWVLPK (SEQ ID NO:40).

In a further embodiment relating to the IL-12 Fc fusion protein according to the first aspect or any of its embodiments the binding moiety is a fibronectin binding moiety.

In a further embodiment relating to the IL-12 Fc fusion protein according to the first aspect or any of its embodiments the fibronectin binding moiety has the sequence GGWSHW (SEQ ID NO:49).

In a further embodiment relating to the IL-12 Fc fusion protein according to the first aspect or any of its embodiments the IL-12p35 subunit and the IL-12p40 subunit are human.

In a further embodiment relating to the IL-12 Fc fusion protein according to the first aspect or any of its embodiments the IL-12p35 subunit comprises a polypeptide having at least 95% identity to SEQ ID NO:1 and the IL-12p40 subunit comprises a polypeptide having at least 95% identity to SEQ ID NO:2, preferably the IL-12p35 subunit comprises the polypeptide of SEQ ID NO:1 and the IL-12p40 subunit comprises the polypeptide of SEQ ID NO:2.

In a further embodiment relating to the IL-12 Fc fusion protein according to the first aspect or any of its embodiments the IL-12p40 subunit and the IL-12p35 subunit are linked in a single-chain having the configuration (written from N-terminus to C-terminus) IL-12p40-IL-12p35 or IL-12p35-IL-12p40.

In a further embodiment relating to the IL-12 Fc fusion protein according to the first aspect or any of its embodiments the single-chain IL-12p40-IL-12p35 is linked via its IL-12p40 subunit to the C-terminus of the first Fc domain, or the single-chain IL-12p35-IL-12p40 is linked via its IL-12p35 subunit to the first Fc domain, and in both cases via the first peptide linker, which first peptide linker is protease-cleavable.

In a further embodiment relating to the IL-12 Fc fusion protein according to the first aspect or any of its embodiments the IL-12p40 subunit and the IL-12p35 subunit are linked to each other via a linker that is rich in amino acid residues glycine and serine, preferably having a length of 5 to 20 amino acids and only including the amino acids glycine and serine, more preferably a glycine and serine linker having the amino acid sequence of SEQ ID NO:22.

In a further embodiment relating to the IL-12 Fc fusion protein according to the first aspect or any of its embodiments the single-chain IL-12p40-IL-12p35 comprises a polypeptide having at least 95% identity to SEQ ID NO:8, or the single-chain IL-12p35-IL-12p40 comprises a polypeptide having at least 95% identity to SEQ ID NO:9.

In a further embodiment relating to the IL-12 Fc fusion protein according to the first aspect or any of its embodiments the second peptide linker is not protease-cleavable.

In a further embodiment relating to the IL-12 Fc fusion protein according to the first aspect or any of its embodiments the masking moiety binds to the IL-12p40 subunit and is selected from the group consisting of: an IL-12 receptor or an IL-12p40 binding fragment thereof, an scFv, or an immunoglobulin single variable domain, preferably a VHH.

In a further embodiment relating to the IL-12 Fc fusion protein according to the first aspect or any of its embodiments the first and the second Fc domain each comprise one or more mutations that promote heterodimerization of the Fc domains.

In a further embodiment relating to the IL-12 Fc fusion protein according to the first aspect or any of its embodiments the (a) first Fc domain is a human IgG1 Fc domain comprising the mutation T366W and the second Fc domain is a human IgG1 Fc domain comprising the mutations T366S, L368A and Y407V, or the (b) first Fc domain is a human IgG1 Fc domain comprising the mutations T366S, L368A and Y407V and the second Fc domain is a human IgG1 Fc domain comprising the mutation T366W.

In a further embodiment relating to the IL-12 Fc fusion protein according to the first aspect or any of its embodiments the first and the second Fc domain are human IgG1 Fc domains and one of the first or the second Fc domain comprises the mutations H435R and Y436F.

In a further embodiment relating to the IL-12 Fc fusion protein according to the first aspect or any of its embodiments the first and the second Fc domain are human IgG1 Fc domains and either the first Fc domain, or the second Fc domain, or both Fc domains comprise the mutations L234A and L235A.

In a further embodiment relating to the IL-12 Fc fusion protein according to the first aspect or any of its embodiments the first Fc domain comprises the amino acid sequence of SEQ ID NO:15 and the second Fc domain comprises the amino acid sequence of SEQ ID NO:16, OR the first Fc domain comprises the amino acid sequence of SEQ ID NO:17 and the second Fc domain comprises the amino acid sequence of SEQ ID NO:18, OR the first Fc domain comprises the amino acid sequence of SEQ ID NO:16 and the second Fc domain comprises the amino acid sequence of SEQ ID NO:15, OR the first Fc domain comprises the amino acid sequence of SEQ ID NO:18 and the second Fc domain comprises the amino acid sequence of SEQ ID NO:17.

In a further embodiment relating to the IL-12 Fc fusion protein according to the first aspect or any of its embodiments the protease-cleavable linker is cleavable by a matrix metalloproteinase (MMP), preferably an MMP-2, MMP-9, or MMP-13.

In a further embodiment relating to the IL-12 Fc fusion protein according to the first aspect or any of its embodiments the protease-cleavable linker comprises or consists of any one of the amino acid sequences of SEQ ID NOs:232-241.

In a second aspect the invention relates to an IL-12 Fc fusion protein comprising a first polypeptide chain and a second polypeptide chain, wherein a) the first polypeptide chain comprises or consists of the amino acid sequence of SEQ ID NO:208 and the second polypeptide chain comprises or consists of the amino acid sequence of SEQ ID NO:209, b) the first polypeptide chain comprises or consists of the amino acid sequence of SEQ ID NO:210 and the second polypeptide chain comprises or consists of the amino acid sequence of SEQ ID NO:211, c) the first polypeptide chain comprises or consists of the amino acid sequence of SEQ ID NO:212 and the second polypeptide chain comprises or consists of the amino acid sequence of SEQ ID NO:213, d) the first polypeptide chain comprises or consists of the amino acid sequence of SEQ ID NO:214 and the second polypeptide chain comprises or consists of the amino acid sequence of SEQ ID NO:215, e) the first polypeptide chain comprises or consists of the amino acid sequence of SEQ ID NO:216 and the second polypeptide chain comprises or consists of the amino acid sequence of SEQ ID NO:217, f) the first polypeptide chain comprises or consists of the amino acid sequence of SEQ ID NO:218 and the second polypeptide chain comprises or consists of the amino acid sequence of SEQ ID NO:219, g) the first polypeptide chain comprises or consists of the amino acid sequence of SEQ ID NO:220 and the second polypeptide chain comprises or consists of the amino acid sequence of SEQ ID NO:221, h) the first polypeptide chain comprises or consists of the amino acid sequence of SEQ ID NO:222 and the second polypeptide chain comprises or consists of the amino acid sequence of SEQ ID NO:223, i) the first polypeptide chain comprises or consists of the amino acid sequence of SEQ ID NO:224 and the second polypeptide chain comprises or consists of the amino acid sequence of SEQ ID NO:225, j) the first polypeptide chain comprises or consists of the amino acid sequence of SEQ ID NO:226 and the second polypeptide chain comprises or consists of the amino acid sequence of SEQ ID NO:227, k) the first polypeptide chain comprises or consists of the amino acid sequence of SEQ ID NO:228 and the second polypeptide chain comprises or consists of the amino acid sequence of SEQ ID NO:229, l) the first polypeptide chain comprises or consists of the amino acid sequence of SEQ ID NO:230 and the second polypeptide chain comprises or consists of the amino acid sequence of SEQ ID NO:231, OR m) the first polypeptide chain comprises or consists of the amino acid sequence of SEQ ID NO:242 and the second polypeptide chain comprises or consists of the amino acid sequence of SEQ ID NO:243.

In a further embodiment relating to the IL-12 Fc fusion protein according to the first aspect or any of its embodiments the masking moiety comprises an IL-12 binding immunoglobulin single variable domain comprising the three CDRs contained within any one of the sequences of SEQ ID NOs:61-109.

In a further embodiment relating to the IL-12 Fc fusion protein according to the first aspect or any of its embodiments the masking moiety comprises an IL-12 binding immunoglobulin single variable domain comprising any one of the amino acid sequences of SEQ ID NOs:61-109.

In a third aspect the invention relates to a cleavage product capable of binding to a human IL-12 receptor comprising the IL-12 cytokine after proteolytic cleavage of the cleavable linker as defined in any one of the IL-12 Fc fusion proteins of the aforementioned aspects and the embodiments relating thereto.

In a further embodiment relating to the cleavage product according to the third aspect or any of its embodiments the cleavage product comprises the IL-12 cytokine and the binding moiety.

In a further embodiment relating to the cleavage product according to the third aspect or any of its embodiments the cleavage product comprises or consists of the amino acid sequence of any one of SEQ ID NOs:208, 210, 212, 214, 216, 218, 220, 222, 224, 226, 228, 230 or 242 after proteolytic cleavage of the cleavable linker.

In a fourth aspect the invention relates to an IL-12 binding immunoglobulin single variable domain comprising the three CDRs contained within any one of the sequences of SEQ ID NOs:61-109.

In a further embodiment relating to the IL-12 binding immunoglobulin single variable domain according to the fourth aspect or any of its embodiments said immunoglobulin single variable domain is a VHH.

In a further embodiment relating to the IL-12 binding immunoglobulin single variable domain according to the fourth aspect or any of its embodiments said immunoglobulin single variable domain comprises the amino acid sequence of any one of SEQ ID NOs:61-109.

In a fifth aspect the invention relates to a nucleic acid encoding at least one polypeptide of the IL-12 Fc fusion proteins of the aforementioned aspects or any embodiments related thereto, or a nucleic acid encoding one of the polypeptide chains of an IL-12 Fc fusion protein of the aforementioned aspects or any embodiments related thereto, or a nucleic acid encoding an IL-12 binding immunoglobulin single variable domain of the aforementioned aspects or any embodiments related thereto.

In a sixth aspect the invention relates to a vector comprising the nucleic acid of the fifth aspect, optionally wherein the vector comprises nucleic acids encoding both chains of the IL-12 Fc fusion protein.

In a seventh aspect the invention relates to a host cell comprising the nucleic acid of the fifth aspect or the vector of the sixth aspect, optionally wherein the cell comprises one or more nucleic acids encoding both chains of the IL-12 Fc fusion protein.

In an eight aspect the invention relates to a method of producing an IL-12 Fc fusion protein comprising culturing the host cell of the seventh aspect under a condition that produces the fusion protein and optionally purifying said IL-12 Fc fusion protein.

In a ninth aspect the invention relates to a composition comprising the IL-12 Fc fusion protein of any of the aforementioned aspects or the embodiments relating thereto.

In a tenth aspect the invention relates to a pharmaceutical composition comprising the IL-12 Fc fusion protein of any of the aforementioned aspects or the embodiments relating thereto and a pharmaceutically acceptable carrier.

In an eleventh aspect the invention relates to a kit comprising the IL-12 Fc fusion protein of any of the aforementioned aspects or the embodiments relating thereto, or the composition of the ninth aspect, or the pharmaceutical composition of the tenth aspect.

In a twelfth aspect the invention relates to an IL-12 Fc fusion protein as defined in any of the aforementioned aspects or the embodiments relating thereto for use in medicine.

In a thirteenth aspect the invention relates to a cleavage product as defined in the third aspect or any embodiment relating thereto for use in medicine.

In a fourteenth aspect the invention relates to a method of treating or reducing the incidence of cancer in a subject, the method comprising administering to the subject an effective amount of an IL-12 Fc fusion protein according to any of the aforementioned aspects or the embodiments relating thereto.

In a fifteenth aspect the invention relates to an IL-12 Fc fusion protein according to any of the aforementioned aspects or the embodiments relating thereto for use in treating or preventing cancer.

In a sixteenth aspect the invention relates to the use of an IL-12 Fc fusion protein according to any of the aforementioned aspects or the embodiments relating thereto for the manufacture of a medicament.

In a seventeenth aspect the invention relates to the use of an IL-12 Fc fusion protein according to any of the aforementioned aspects or the embodiments relating thereto for the manufacture of a medicament for reduction of the incidence of or treatment of cancer.

In any of the aforementioned aspects and embodiments, the IL-12 Fc fusion protein, the cleavage product, the IL-12 binding immunoglobulin single variable domain, the nucleic acid, the vector or the host cell may be isolated, i.e. an isolated IL-12 Fc fusion protein, an isolated cleavage product, an isolated IL-12 binding immunoglobulin single variable domain, an isolated nucleic acid, an isolated vector or an isolated host cell.

### BRIEF DESCRIPTION OF THE FIGURES

FIGS. 1A-1H: Exemplary formats scouted for assembly of IL-12 Fc fusion proteins, with a cleavable IL-12 (p35 as kidney shape, p40 as three individual spheres), an antibody fragment masking domain, cleavable linker (light grey with star) connecting the IL-12 to the Fc, and either Knob-in-holes or wild type Fc.

FIG. 2: Functional characterization of 47 VHH-Fc that were discovered via llama immunizations, followed by subsequent phage panning. Only one of the constructs shows inhibition of IL12 >90% with respect to the control molecule in the Promega Bioassay.

FIGS. 3A-3B: Single-chain chimeric IL-12 (BI-066) (FIG. 3A) or MMP9-cleaved chimeric IL-12 Fc fusion protein (BI-057) (FIG. 3B) was serially diluted and added to the Promega IL-12 bioassay cells. After incubation Bio-Glo™ reagent was added and luminescence was measured. Data were analyzed in the GraphPad Prims and EC₅₀ was calculated.

FIG. 4: Chimeric IL-12 Fc fusion protein (BI-057) was proteolytically cleaved with activated MMP9 (cleaved) or incubated without the enzyme (uncleaved) for 24 h. Next, both samples were serially diluted and added to the Promega IL-12 bioassay cells. After incubation, Bio-Glo™ reagent was added and luminescence was measured. Data were analyzed in the GraphPad Prims and EC₅₀ value was calculated.

FIGS. 5A-5D: C57BI/6 mice were injected with B16.F10 melanoma cells. Treatment started when the tumor reached volume of 70-100 mm³. Animals were treated with vehicle or chimeric IL-12 Fc fusion protein at the doses indicated in the figure legends. Treatment schedule is depicted by dotted lines. Tumor growth (FIG. 5A and FIG. 5C) and body weight changes (FIG. 5B and FIG. 5D) were monitored twice weekly and are presented as spaghetti plots depicting individual mice.

FIGS. 6A-6B: C57BI/6 mice were injected with B16.F10 melanoma cells. Treatment started when the tumor reached volume of 70-100 mm³. Animals were treated with vehicle or unmasked chimeric IL-12 Fc fusion protein at the doses indicated in the figure legends (FIG. 6A: 0.08 mg/kg; FIG. 6B: 1.6 mg/kg). Treatment schedule is depicted by dotted lines. Body weight changes were monitored twice weekly and are presented as spaghetti plots depicting individual mice.

FIGS. 7A-7B: C57BI/6 mice were injected with MC38 colon carcinoma cells. Treatment started when the tumor reached volume of 70-100 mm³. Animals were treated with vehicle or chimeric IL-12 Fc fusion protein at the doses indicated in the figure legends. Treatment schedule is depicted by dotted lines. Tumor growth (FIG. 7A) and body weight changes (FIG. 7B) were monitored twice weekly and are presented as means of the group.

FIGS. 8A-8B: C57BI/6 mice were injected with B16.F10 melanoma cells. Treatment started when the tumor reached volume of 70-100 mm³. Animals were treated with vehicle or chimeric IL-12 Fc fusion protein at the dose indicated in the figure legends. Treatment schedule is depicted by dotted lines. Tumor growth (FIG. 8A) and body weight changes (FIG. 8B) were monitored twice weekly and are presented as spaghetti plots depicting individual mice.

FIGS. 9A-9C: Cynomolgus monkeys were injected with three different doses of human IL-12 Fc fusion protein on day 1. Blood was collected at day 1 (pre-dose), 8 and 15. Values of ALT (FIG. 9A), Bilirubin (FIG. 9B) and Creatinine (FIG. 9C) for each animal are shown on the graphs. Doted line depicts reference value.

FIGS. 10A-10F: Gene expression of MMP2 (FIG. 10A), MMP9 (FIG. 10B), MMP13 (FIG. 10C), TIMP1 (FIG. 10D), TIMP2 (FIG. 10E), TIMP3 (FIG. 10F) in normal and cancer tissue. White fill, fine line, GTEX normal tissue corresponding to TCGA cancer tissues, white fill, bold line, TCGA adjacent normal tissue, grey fill, bold line, TCGA cancer tissue.

FIG. 11: 5 Variants show effective cleavage of the parental molecule into the component IL-12 and Fc-mask domains with the addition of MMP9.

FIG. 12: Treatment with MMP9 using anti-p40 Western antibody, show release of single-chain IL12 from the full prodrug, with released IL-12 migrating to approximately the 62 kD band in a reducing environment. Detection antibodies used in the assay are: Anti IL12 p40 (R&D Systems, AF309) 1:2500; Anti goat IgG HRP (R&D Systems, HAF017) 1:1000.

FIGS. 13A-13E: Human IL-12 Fc fusion proteins BI-050 (FIG. 13A), BI-051 (FIG. 13B), BI-052 (FIG. 13C), BI-054 (FIG. 13D), and BI-055 (FIG. 13E) were proteolytically cleaved with activated MMP9 (cleaved) or incubated without the enzyme (uncleaved) for 24 h. Next, all samples were serially diluted and added to the Promega IL-12 bioassay cells. After incubation, Bio-Glo™ reagent was added and luminescence was measured. Data were analyzed in the GraphPad Prims and EC50 value were calculated (see Table 15).

FIGS. 14A-14B: BI-059 at 2.5 UM was incubated with either buffer control, 6.5 nM (0.025 μg) activated recombinant human MMP9, or 5 g of human colorectal cancer tumor lysate for 2 hours at 37 ºC. Tumor lysate was also incubated with buffer alone as a control. SDS-PAGE and Western blotting was performed with an antibody against human Fc (FIG. 14A) and human IL12p40 (FIG. 14B), which show a size shift following cleavage of full length IL-12 Fc fusion protein by MMPs that corresponds to the released masking domain/Fc fragment and free IL-12, respectively.

FIG. 15: C57BI/6 mice were injected with MC38 colon carcinoma cells. Treatment started when the tumor reached volume of 70-100 mm³. Animals were treated twice on day 1 and 4 with vehicle or chimeric IL-12 Fc fusion at the dose indicated in the figure legend. Animals were sacrificed at day 5 and tumors were collected. Tissue was digested followed by IFNγ evaluation.

FIGS. 16A-16F: C57BI/6 mice were injected with MC38 colon carcinoma cells. Treatment started when the tumor reached volume of 70-100 mm³. Animals were treated twice on day 1 and 4 with vehicle or chimeric IL-12 Fc fusion at the doses indicated in the figure legend. Animals were sacrificed at day 5 and tumors were collected. Tissue was digested followed by flow cytometry evaluation of tumor infiltrating leukocytes for marker expression as indicated.

FIGS. 17A-17I: Gene expression of Collagen I A1 (FIG. 17A), Collagen I A2 (FIG. 17B), Fibronectin (FIG. 17C), Collagen IV A1 (FIG. 17D), Collagen IV A2 (FIG. 17E), Collagen IV A3 (FIG. 17F), Collagen IV A4 (FIG. 17G), Collagen IV A5 (FIG. 17H), Collagen IV A6 (FIG. 17I) in normal and cancer tissue. White fill, fine line, GTEX normal tissue corresponding to TCGA cancer tissues, white fill, bold line, TCGA adjacent normal tissue, grey fill, bold line, TCGA cancer tissue.

FIGS. 18A-18B: Human IL-12 Fc fusion protein BI-051 was serially diluted and added to collagen I-coated plates for 10 min. After washing step, biotinylated anti-human Fc Ab was used to detect bound protein. SA-HRP followed by the substrate were added to wells and OD was measured in a Tecan plate reader (FIG. 18A). OD values are presented. The interaction analysis was performed using Biacore T200 (FIG. 18B) equipped with a CM5 chip in which onto active surface, human collagen type 1 (Merck, CC050) was amine-coupled (3000 RU) and reference cell was amine-coupled without any ligand. BI-051 and mAb IgG1 (negative control) were diluted to a final concentration of 5 μM in Biacore running buffer (phosphate buffered saline, pH 7.4 containing 0.05% Tween-20). The Biacore measurements were carried out using reference subtraction. The interaction occurred solely on the active surface that immobilized human collagen type 1.

FIG. 19: Separate chains comprising human IL-12 Fc fusion protein BI-051 (knob chain and hole chain) were serially diluted and added to collagen I-coated plates for 10 min. After washing step, biotinylated anti-human Fc Ab was used to detect bound protein. SA-HRP followed by the substrate were added to wells and OD was measured in a Tecan plate reader. OD values are presented.

FIGS. 20A-20D: Plates coated with fibroblast-produced collagen I were prepared as described in the Material and Methods section. Upon decellularization process, 5 μg of fluorescently labeled human IL-12 Fc fusion protein (BI-051) was added to the wells (FIG. 20B). Wells incubated with PBS (FIG. 20D) served as control. After washing steps, wells were stained with anti-collagen I antibody (FIG. 20A and FIG. 20C) and visualized in Opera Phenix system.

FIGS. 21A-21B: C57BI/6 mice were injected with MC38 colon carcinoma cells. Treatment started when the tumor reached volume of 70-100 mm³. Animals were treated with vehicle, chimeric IL-12 Fc fusion protein (BI-065) or chimeric IL-12 Fc fusion protein containing fibronectin TME linker (BI-059) at the dose of 0.5 mg/kg. Treatment schedule is depicted by doted lines. Tumor growth was monitored twice weekly and is presented as spaghetti plots depicting individual mice (FIG. 21A). Percentage of surviving mice is presented in (FIG. 21B).

FIG. 22: C57BI/6 mice were injected with B16.F10 melanoma cells. Treatment started when the tumor reached volume of 70-100 mm³. Animals were treated with vehicle, chimeric IL-12 Fc fusion protein (BI-065) or chimeric IL-12 Fc fusion protein containing fibronectin TME linker (BI-059) at the dose of 1.5 mg/kg. Treatment schedule is depicted by dotted lines. Tumor growth was monitored twice weekly and is presented as spaghetti plots depicting individual mice.

FIGS. 23A-23B: C57BI/6 mice were injected with MC38 colon carcinoma cells. Treatment started when the tumor reached volume of 70-100 mm³. Animals were treated with vehicle, chimeric IL-12 Fc fusion protein (BI-065) or chimeric IL-12 Fc fusion protein containing collagen I TME linker (BI-057) at the dose of 0.5 mg/kg. Treatment schedule is depicted by dotted lines. Tumor growth (FIG. 23A) and changes in body weight (FIG. 23B) were monitored twice weekly and are presented as spaghetti plots depicting individual mice.

FIGS. 24A-24B: C57BI/6 mice were injected with MC38 colon carcinoma cells. Treatment started when the tumor reached volume of 70-100 mm³. Animals were treated with vehicle, chimeric IL-12 Fc fusion protein (BI-065) or chimeric IL-12 Fc fusion protein containing collagen I TME linker (BI-057) at the dose of 1.5 mg/kg. Treatment schedule is depicted by dotted lines. Tumor growth (FIG. 24A) and changes in body weight (FIG. 24B) were monitored twice weekly and are presented as spaghetti plots depicting individual mice.

FIGS. 25A-25B: Chimeric IL-12 Fc fusion protein BI-057 or BI-065 were serially diluted and added to collagen I-coated plates for 120 min. Rat collagen (Corning) (FIG. 25A) or human collagen (Millipore/R&D) (FIG. 25B) were tested. After washing step, biotinylated anti-human Fc Ab was used to detect bound protein. SA-HRP followed by the substrate were added to wells and OD was measured in a Tecan plate reader. OD values are presented.

FIGS. 26A-26B: Precision cut liver slices were prepared from fibrotic rat livers. IL-12 Fc fusion proteins (BI-057 or BI-065; in amounts indicated in figure legends) were added to the slices and incubated for 24 hours (FIG. 26A) or 2 hours followed by 22 h recovery period (FIG. 26B). After 2 h incubation, fusion proteins were thoroughly washed and incubated for another 22 h in medium before harvest. After 24 h, slices were collected, lysed and homogenized. The amount of IL-12 Fc fusion proteins recovered from the slices was determined with the MSD U-PLEX Biomarker Assay using IL-12 Fc fusion proteins as standards.

FIGS. 27A-27D: C57BI/6 albino mice were injected with KPCY pancreatic carcinoma cells s.c. Animals were injected intravenously with Dylight 650-labelled chimeric IL-12 Fc fusion protein (BI-201) or chimeric IL-12 Fc fusion protein containing collagen I TME linker (BI-202) at the dose of 30 μg. To assess kinetics of protein retention, mice were imaged with IVIS under auto-exposure epi-illumination fluorescence settings at indicated time points (FIG. 27A). Image analysis to determine total radiant efficiency was performed using Living Image. Statistical analysis at the final day of experiment was performed using Mann-Whitney test (FIG. 27B). Using fluorescence data half life (FIG. 27C) and clearance (FIG. 27D) were calculated.

FIGS. 28A-28B: Balb/c mice were injected with EMT6 mammary carcinoma cells s.c. Animals were injected intravenously with Dylight 650-labelled chimeric IL-12 Fc fusion protein (BI-200) or chimeric IL-12 Fc fusion protein containing collagen I TME linker (BI-051) at the dose of 50 μg. To assess kinetics of protein retention, mice were imaged with IVIS under auto-exposure epi-illumination fluorescence settings at indicated time points (FIG. 28A). Image analysis to determine total radiant efficiency was performed using Living Image. Statistical analysis at 30 h was performed using Mann-Whitney test (FIG. 27B).

FIGS. 29A-29B: C57BI/6 mice were injected with PDA30364 pancreatic carcinoma cells s.c. Animals were injected intratumorally with chimeric IL-12 Fc fusion protein (BI-065) or chimeric IL-12 Fc fusion protein containing collagen I TME linker (BI-057) at the dose of 150 pmol. Blood was collected 48 h after the injection and levels of IFNγ (FIG. 29A) and CXCL10 (FIG. 29B) were determined by LegendPlex. Statistical analysis was performed using Mann-Whitney test.

FIGS. 30A-30B: C57BI/6 mice were injected orthotopic with EMT6 mammary carcinoma cells. Treatment started when the tumor reached volume of 50-120 mm³. Animals were treated with vehicle or chimeric IL-12 Fc fusion protein (BI-065) or chimeric IL-12 Fc fusion protein containing collagen I TME linker (BI-057) at the dose indicated in the figure legends. Blood was collected 24 h (FIG. 30A) or 72 h (FIG. 30B) after the injection and levels of IFNγ were determined by LegendPlex. Statistical analysis was performed using Mann-Whitney test.

### DETAILED DESCRIPTION OF THE INVENTION

The inventors set out to design conditionally active IL-12 fusion proteins that would allow systemic administration of IL-12 (known to be toxic) to patients for the treatment of tumors. On the way, many challenges needed to be overcome, including the choice of an appropriate molecule design, finding the proper ways to block the activity of IL-12 to allow for systemic administration, aligning chemistry, manufacturing and controls (CMC) properties with molecule function and ensuring that the IL-12 reaches the tumor and then regains its activity within the tumor or nearby the tumor.

The present invention is based on the concept of providing an Interleukin-12 (IL-12) Fc fusion protein comprising a first polypeptide chain and a second polypeptide chain, wherein a) the first polypeptide chain comprises a first Fc domain and an IL-12p35 subunit and an IL-12p40 subunit of IL-12, and b) the second polypeptide chain comprises a second Fc domain and a masking moiety that binds to the IL-12p35 and/or IL-12p40 subunit in the first polypeptide chain; wherein the first and second polypeptide chain are linked via the first Fc domain and the second Fc domain, wherein the IL-12p35 subunit or the IL-12p40 subunit is linked to the C-terminus of the first Fc domain via a first peptide linker, which first peptide linker is protease-cleavable, wherein the masking moiety is linked to the C-terminus of the second Fc domain via a second peptide linker, and wherein the first or the second polypeptide chain further comprises a binding moiety selected from the group consisting of: a collagen binding moiety, a heparin binding moiety, and a fibronectin binding moiety.

An Fc based fusion protein approach was chosen to increase the half-life of the fusion protein after systemic administration. This Fc fusion protein comprises two polypeptide chains. The first polypeptide chain comprises a first Fc domain and both the subunits IL-12p35 and IL-12p40 of IL-12 that form together the active IL-12 cytokine and is linked via a protease-cleavable linker to the C-terminus of the first Fc domain. The second polypeptide chain comprises a second Fc domain and a masking moiety which is linked to the C-terminus of the second Fc domain. Both polypeptide chains together, dimerize via their respective Fc domains to form a dimeric Fc fusion protein, i.e. the polypeptide chains are linked via the binding of the two Fc domains that form together the Fc part of the fusion protein. In this dimeric Fc fusion protein the masking moiety on the second chain binds to the IL-12 cytokine on the first chain and thereby blocks, inhibits or attenuates the activity of the IL-12 cytokine. Additionally, the Fc based fusion protein comprises a binding moiety selected from the group consisting of: a collagen binding moiety, a heparin binding moiety, and a fibronectin binding moiety.

After systemic administration of the IL-12 Fc fusion protein the activity of the IL-12 on the first polypeptide chain is still blocked, inhibited or attenuated by the masking moiety. It is proposed that tumor specific activation is then achieved via a dual-fold mechanism involving the protease-cleavable linker and the binding moiety. The protease-cleavable linker is preferably cleaved by proteases that are tumor-specific or upregulated in the tumor micro environment (TME). Additionally, the binding moiety binds to its respective structures in the TME, such as the extracellular matrix (ECM) and together with the protease-cleavable linker provides for a therapeutic window to allow for optimal biological activity within the TME without dose-limiting systemic toxicity.

Retaining the IL-12 Fc fusion protein within the TME may have further advantages, such as enabling a longer exposure of the fusion protein to the environment of upregulated protease activity in the TME, which could increase cleavage efficiency and ultimately the level of cleavage product to be released. Also, once cleaved the cleavage product may be retained within the tumor, which could increase the potency of the response as well as reduce systemic toxicity, by preventing, reducing or slowing down the cleavage product from entering the circulation.

In one aspect, the unmasked IL-12 provides potent, Th1-polarizing stimuli to T-cells at the tumor site to improve their effector function.

In another aspect, the IL-12 Fc fusion protein has improved pharmacokinetic and/or toxicologic properties compared to unmasked IL-12. In another aspect, the IL-12 Fc fusion protein has improved pharmacokinetic and/or toxicologic properties compared to other masked IL-12 fusion proteins.

In another aspect, the IL-12 Fc fusion protein can be produced as a stable molecule with high process performance and productivity.

In another aspect, the IL-12 Fc fusion may have tumor agnostic properties, i.e. may be used for the treatment of several cancers. In a related aspect the IL-12 Fc fusion protein may be useful for immune modulated treatment of cancers or tumors.

In one aspect, the cleavage product of the IL-12 Fc fusion protein is prevented, reduced or slowed down from entering the circulation after cleavage in the TME.

In another aspect, the cleavage product of the IL-12 Fc fusion protein shows increased retention within the tumor or the TME. In a related aspect, the cleavage product of the IL-12 Fc fusion protein shows increased potency response. In a related aspect, the cleavage product of the IL-12 Fc fusion protein shows reduced systemic toxicity.

In another aspect, the IL-12 activity of the uncleaved IL-12 Fc fusion protein is at least 50-fold, 75-fold, 100-fold, 125-fold, 150-fold, 175-fold, 200-fold, 225-fold, 250-fold, 275-fold, 300-fold, 325-fold, 350-fold, 375-fold, 400-fold, 425-fold, 450-fold, 475-fold, 500-fold, 525-fold, 550-fold, 575-fold, or 600-fold lower compared to the IL-12 activity of the IL-12 Fc fusion protein after cleavage of the cleavable linker. In other words, the delta EC₅₀ of the uncleaved IL-12 Fc fusion protein and the cleaved IL-12 Fc fusion protein as measured in an IL-12 bioassay (EC₅₀ uncleaved IL-12 Fc fusion protein: EC₅₀ cleaved IL-12 Fc fusion protein), e.g. as in the Promega IL-12 Bioassay described in the examples, is at least 50, 75, 100, 125, 150, 175, 200, 225, 250, 275, 300, 325, 350, 400, 425, 450, 475, 500, 525, 550, 575, or 600.

### Definitions

Terms not specifically defined herein should be given the meanings that would be given to them by one of skill in the art in light of the disclosure and the context. As used in the specification, however, unless specified to the contrary, the following terms have the meaning indicated and the following conventions are adhered to.

When used herein the term “comprising” and variations thereof such as “comprises” and “comprise” can be substituted with the term “containing” or “including” or “having”.

The term “sequence” as used herein (for example in terms like “heavy/light chain sequence”, “antibody sequence”, “variable domain sequence”, “constant domain sequence” or “protein sequence”), should generally be understood to include both the relevant amino acid sequence as well as nucleic acid sequences or nucleotide sequences encoding the same, unless the context requires a more limited interpretation.

The “Fc domain” of an antibody is not involved directly in binding of an antibody to an antigen, but exhibits various effector functions. An “Fc domain of an antibody” is a term well known to the skilled artisan and defined on the basis of papain cleavage of antibodies. Depending on the amino acid sequence of the constant region of their heavy chains, antibodies or immunoglobulins are divided in the classes: IgA, IgD, IgE, IgG and IgM. According to the heavy chain constant regions the different classes of immunoglobulins are called a, 0, E, Y, and u respectively. Several of these may be further divided into subclasses (isotypes), e.g. lgG1, IgG2, IgG3, and IgG4, IgA1, and IgA2. The Fc part of an antibody is directly involved in ADCC (antibody dependent cell-mediated cytotoxicity) and CDC (complement-dependent cytotoxicity) based on complement activation, Clq binding and Fc receptor binding. Complement activation (CDC) is initiated by binding of complement factor Clq to the Fc part of most IgG antibody subclasses. While the influence of an antibody on the complement system is dependent on certain conditions, binding to Clq is caused by defined binding sites in the Fc part. Such binding sites are e.g. L234, L235, D270, N297, E318, K320, K322, P331 and P329 (numbering according to EU numbering (Edelman et al, Proc Natl Acad Sci USA. 1969 May; 63(1):78-85)). Most crucial among these residues in mediating Clq and Fcgamma receptor binding in IgG1 are L234 and L235 (Hezareh et al., J. Virology 75 (2001) 12161-12168, Shields et al (2001) JBC, 276 (9): 6591-6604). Antibodies of subclass IgG1 and IgG3 usually show complement activation and Clq and C3 binding, whereas IgG2 and IgG4 do not activate the complement system and do not bind Clq and C3.

A “single-chain Fv” or “scFv” antibody fragment is a single chain Fv variant comprising the VH and VL domains of an antibody where the domains are present in a single polypeptide chain. The single chain Fv is capable of recognizing and binding an antigen. The scFv polypeptide may optionally also contain a polypeptide linker positioned between the VH and VL domains in order to facilitate formation of a desired three-dimensional structure for antigen binding by the scFv (see, e.g., Pluckthun, 1994, In The Pharmacology of monoclonal Antibodies, Vol. 113, Rosenburg and Moore eds., Springer-Verlag, New York, pp. 269-315).

A “non-cleavable linker” or “not protease-cleavable linker” as used herein refers to a peptide linker that does not contain a peptide sequence or a mimic of a peptide sequence, which is the target for a protease. Exemplary non-cleavable linkers are described in the Linkers section.

An antigen binding molecule/protein (such as an immunoglobulin, an antibody, an antigen binding unit, or a fragment of such antigen binding molecule/protein) that can “bind”, “bind to”, “specifically bind”, or “specifically bind to”, that “has affinity for”, “is specific for” and/or that “has specificity for” a certain epitope, antigen or protein (or for at least one part, fragment or epitope thereof) is said to be “against” or “directed against” said epitope, antigen or protein or is a “binding” molecule/protein with respect to such epitope, antigen or protein.

As used herein, the terms “binding” and “specific binding” refer to the binding of the antibody or antigen binding moiety (such as an immunoglobulin, an antibody, an antigen binding unit, or a fragment of such antigen binding molecule/protein) to an epitope of the antigen in an in vitro assay, preferably in a plasmon resonance assay ((Malmqvist M., “Surface plasmon resonance for detection and measurement of antibody-antigen affinity and kinetics.”, Curr Opin Immunol. 1993 April; 5(2):282-6.)) with purified wild-type antigen. Antibody affinity can also be measured using kinetic exclusion assay (KinExA) technology (Darling, R. J., and Brault P-A., “Kinetic exclusion assay technology: Characterization of Molecular Interactions.” ASSAY and Drug Development Technologies. 2004, Dec. 2(6): 647-657).

Generally, the term “specificity” refers to the number of different types of antigens or epitopes to which a particular antigen binding molecule/protein (such as an immunoglobulin, an antibody, an antigen binding unit, or a fragment of such antigen binding molecule/protein) can bind. The specificity of an antigen-binding molecule/protein can be determined based on its affinity and/or avidity. The affinity, represented by the equilibrium constant for the dissociation of an antigen with an antigen-binding protein (K_{D}), is a measure for the binding strength between an epitope and an antigen-binding site on the antigen-binding molecule/protein: the lesser the value of the K_{D}, the stronger the binding strength between an epitope and the antigen-binding molecule/protein (alternatively, the affinity can also be expressed as the affinity constant (K_{A}), which is 1/K_{D}). As will be clear to the skilled person (for example on the basis of the further disclosure herein), affinity can be determined in a manner known per se, depending on the specific antigen of interest. Avidity is the measure of the strength of binding between an antigen-binding molecule/protein (such as an immunoglobulin, an antibody, an antigen binding unit, or fragment of such antigen binding molecule/protein) and the pertinent antigen. Avidity is related to both the affinity between an epitope and its antigen binding site on the antigen-binding molecule/protein and the number of pertinent binding sites present on the antigen-binding molecule/protein.

For application in man, it is often desirable to reduce immunogenicity of therapeutic molecules, such as antibodies or binding proteins comprising an antigen binding unit as described herein, originally derived from other species, like mouse. This can be done by construction of chimeric antibodies/binding proteins, or by a process called “humanization”. In this context, a “chimeric antibody”; or “chimeric antigen binding unit” is understood to be an antibody or an antigen binding unit comprising a sequence part (e.g. a variable domain) derived from one species (e.g. mouse) fused to a sequence part (e.g. the constant domains) derived from a different species (e.g. human). In this context, a “humanized antibody”, “a humanized binding protein” or a “humanized antigen binding unit” is an antibody, a protein or antigen binding unit comprising a variable domain originally derived from a non-human species, wherein certain amino acids have been mutated to make the overall sequence of that variable domain more closely resemble a sequence of a human variable domain. Methods of humanization of antibodies are well-known in the art (Billetta R, Lobuglio A F. “Chimeric antibodies”. Int Rev Immunol. 1993; 10 (2-3):165-76; Riechmann L, Clark M, Waldmann H, Winter G (1988). “Reshaping human antibodies for therapy”. Nature: 332:323).

An “optimized antibody” or an “optimized antigen binding unit or protein” is a specific type of humanized antibody or humanized antigen binding unit/protein which includes an immunoglobulin amino acid sequence variant, or fragment thereof, which is capable of binding to a predetermined antigen and which comprises one or more FRs having substantially the amino acid sequence of a human immunoglobulin and one or more CDRs having substantially the amino acid sequence of a non-human immunoglobulin. This non-human amino acid sequence often referred to as an “import” sequence is typically taken from an “import” antibody domain, particularly a variable domain. In general, an optimized antibody includes at least the CDRs (or HVLs) of a non-human antibody or derived from a non-human antibody, inserted between the FRs of a human heavy or light chain variable domain. It will be understood that certain mouse FR residues may be important to the function of the optimized antibodies and therefore certain of the human germline sequence heavy and light chain variable domains residues are modified to be the same as those of the corresponding mouse sequence. During this process undesired amino acids may also be removed or changed, for example to avoid deamidation, undesirable charges or lipophilicity or non-specific binding. An “optimized antibody”, an “optimized antibody fragment” or “optimized” may sometimes be referred to as “humanized antibody”, “humanized antibody fragment” or “humanized”, or as “sequence-optimized”.

Furthermore, technologies have been developed for creating antibodies or VH/VL domains based on sequences derived from the human genome, for example by phage display or use of transgenic animals (WWW. Ablexis.com/technology-alivamab.php; WO 90/05144; D. Marks, H. R. Hoogenboom, T. P. Bonnert, J. McCafferty, A. D. Griffiths and G. Winter (1991) “By-passing immunisation. Human antibodies from V-gene libraries displayed on phage.” J.Mol.Biol., 222, 581-597; Knappik et al., J. Mol. Biol. 296: 57-86, 2000; S. Carmen and L. Jermutus, “Concepts in antibody phage display”. Briefings in Functional Genomics and Proteomics 2002 1(2):189-203; Lonberg N, Huszar D. “Human antibodies from transgenic mice”. Int Rev Immunol. 1995; 13(1):65-93; Brüggemann M, Taussig M J. “Production of human antibody repertoires in transgenic mice”. Curr Opin Biotechnol. 1997 August; 8(4):455-8). Such antibodies or antigen binding units or VH/VL domains are “human antibodies,” “human antigen binding units,” or “human VH/VL domains” in the context of the present invention.

As used herein, the terms “identical” or “percent identity”, in the context of two or more nucleic acids or polypeptide sequences, refer to two or more sequences or subsequences that are the same or have a specified percentage of nucleotides or amino acid residues that are the same, when compared and aligned for maximum correspondence. To determine the percent identity, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In some embodiments, the two sequences that are compared are the same length after gaps are introduced within the sequences, as appropriate (e.g., excluding additional sequence extending beyond the sequences being compared). For example, when variable region sequences are compared, the leader and/or constant domain sequences are not considered. For sequence comparisons between two sequences, a “corresponding” CDR refers to a CDR in the same location in both sequences (e.g., CDR-H1 of each sequence).

The determination of percent identity or percent similarity between two sequences can be accomplished using a mathematical algorithm. A preferred, non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul et al., 1990, J. Mol. Biol. 215:403-410. BLAST nucleotide searches can be performed with the NBLAST program, score=100, wordlength=12, to obtain nucleotide sequences homologous to a nucleic acid encoding a protein of interest. BLAST protein searches can be performed with the XBLAST program, score=50, wordlength=3, to obtain amino acid sequences homologous to a protein of interest. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti, 1994, Comput. Appl. Biosci. 10:3-5; and FASTA described in Pearson and Lipman, 1988, Proc. Natl. Acad. Sci. USA 85:2444-8. Within FASTA, ktup is a control option that sets the sensitivity and speed of the search. If ktup=2, similar regions in the two sequences being compared are found by looking at pairs of aligned residues; if ktup=1, single aligned amino acids are examined. ktup can be set to 2 or 1 for protein sequences, or from 1 to 6 for DNA sequences. The default if ktup is not specified is 2 for proteins and 6 for DNA. Alternatively, protein sequence alignment may be carried out using the CLUSTAL W algorithm, as described by Higgins et al., 1996, Methods Enzymol. 266:383-402.

The term “linked” as used herein refers to the coupling of the different components of the IL-12 Fc fusion protein, and includes (i) via a means, such as a linker, capable of linking the different components, or (ii) any chemical association to link the different components of the IL-12 Fc fusion protein, including both covalent and non-covalent interactions, preferably covalent interactions. The covalent interactions may be e.g a direct covalent bond between residues, such as a peptide bond or a disulfide bond. The linker may be a peptide linker or a non-peptide linker, preferably a peptide linker. If the linker is a peptide linker, it may be composed of one or more amino acids.

An “immunoglobulin single variable domain” (ISVD) is an antibody fragment consisting of a single variable antibody domain. Like a whole antibody, it is able to bind selectively to a specific antigen. With a molecular weight of only 12-18 kDa, they are much smaller than conventional antibodies (150-160 kDa) which are composed of two heavy and two light protein chains, and even smaller than Fab fragments (˜50 kDa, one light chain and half a heavy chain) and single chain variable fragments (˜25 kDa, two variable domains, one from a light and one from a heavy chain). Generally, an immunoglobulin single variable domain will have an amino acid sequence comprising 4 framework regions (FR1 to FR4) and 3 complementarity determining regions (CDR1 to CDR3), preferably according to the following formula: FR1-CDR1-FR2-CDR2-FR3-CDR3-FR4. The term immunoglobulin single variable domain, as used herein, includes—but is not limited to—variable domains of camelid heavy chain antibodies (VHHs), also referred to as Nanobodies™, domain antibodies (dAbs), and immunoglobulin single variable domain derived from shark (IgNAR domains).

“VHH domains”, also known as VHHs, VHH domains, VHH antibody fragments, and VHH antibodies, have originally been described as the antigen binding immunoglobulin (variable) domain of “heavy chain antibodies” (i.e. of “antibodies devoid of light chains”; Hamers-Casterman C, Atarhouch T, Muyldermans S, Robinson G, Hamers C, Songa E B, Bendahman N, Hamers R.: “Naturally occurring antibodies devoid of light chains”; Nature 363, 446-448 (1993)). The term “VHH domain” has been chosen in order to distinguish these variable domains from the heavy chain variable domains that are present in conventional 4-chain antibodies (which are referred to herein as “VH domains” or “VH domains”) and from the light chain variable domains that are present in conventional 4-chain antibodies (which are referred to herein as “VL domains” or “VL domains”). VHH domains can specifically bind to an epitope without an additional antigen binding domain (as opposed to VH or VL domains in a conventional 4-chain antibody, in which case the epitope is recognized by a VL domain together with a VH domain). VHH domains are small, robust and efficient antigen recognition units formed by a single immunoglobulin domain.

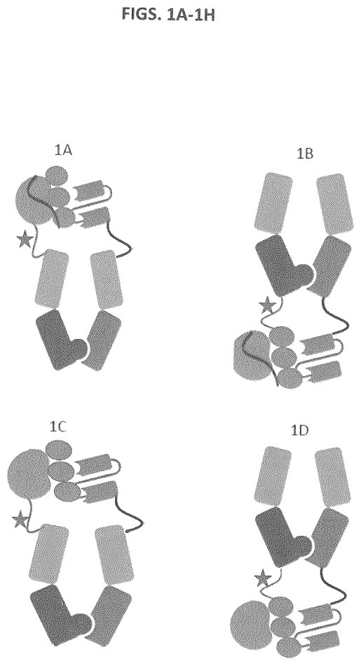

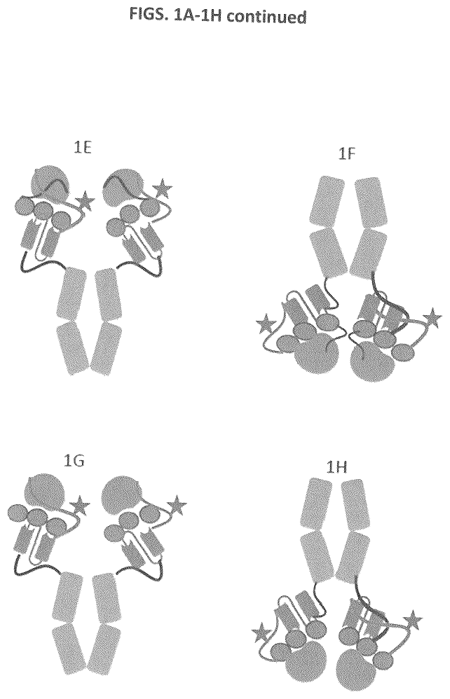

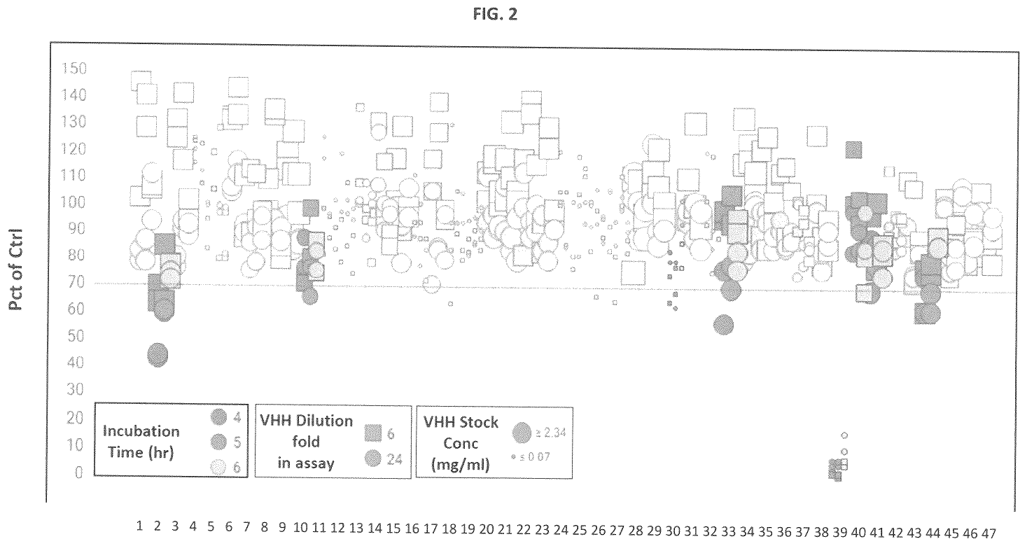

In the context of the present invention, the terms VHH domain, VHH, VHH domain, VHH antibody fragment, VHH antibody, as well as “Nanobody®” and “Nanobody® domain” (“Nanobody” being a trademark of the company Ablynx N.V.; Ghent; Belgium) are used interchangeably and are representatives of ISVDs (having the structure: FR1-CDR1-FR2-CDR2-FR3-CDR3-FR4 and specifically binding to an epitope without requiring the presence of a second immunoglobulin variable domain), and which can also be distinguished from VH domains by the so-called “hallmark residues”, as defined in e.g. WO2009/109635, FIG. 1.

Methods of obtaining VHH domains binding to a specific antigen or epitope have been described earlier, e.g. in WO2006/040153 and WO2006/122786. VHH domains derived from camelids can be “humanized” by replacing one or more amino acid residues in the amino acid sequence of the original VHH sequence by one or more of the amino acid residues that occur at the corresponding position(s) in a VH domain from a conventional 4-chain antibody from a human being. A humanized VHH domain can contain one or more fully human framework region sequences, and, in an even more specific embodiment, can contain human framework region sequences derived from DP-29, DP-47, DP-51, or parts thereof, optionally combined with JH sequences, such as JH5.

### IL-12

Interleukin-12 (IL12) is a heterodimeric molecule composed of an alpha chain (the IL-12p35 subunit) and a beta chain (the IL-12p40 subunit) covalently linked by a disulfide bridge to form the biologically active 70 kDa dimer. It is produced by antigen-presenting cells, such as dendritic cells and macrophages, and is crucial for the recruitment and effector functions of CD8+T and NK cells. Therefore, IL-12 is a major contributor to effective anti-tumor immune responses. IL-12 signals through IL-12Rβ1 and IL-12Rβ2 receptors expressed on target cells, which allow downstream Jak2 and Tyk2 to promote the phosphorylation of and homo-dimerization of STAT4. Further studies demonstrated that IL-12 is not only required for the activation of effector anti-tumor immune responses but can also directly inhibit immune suppression. Thus, the use of IL-12 as a cancer immunotherapy could be beneficial in controlling tumor growth by activating anti-tumor cytotoxic immune responses. Overall, IL-12 targets and modulates T cells, NK cells and antigen-presenting cells (APCs) that regulate the fate of the anti-tumor immune response against the cancer cells.

In certain embodiments, the IL-12 cytokine comprises an IL-12p35 amino acid sequence as set forth in SEQ ID NO:1. In certain embodiments, the IL-12 cytokine comprises an IL-12p40 amino acid sequence as set forth in SEQ ID NO:2. In certain embodiments, the IL-12 cytokine comprises an IL-12p35 amino acid sequence as set forth in SEQ ID NO:1 and comprises an IL-12p40 amino acid sequence as set forth in SEQ ID NO:2.

In another embodiment, as opposed to keeping IL-12 as a native heterodimer, the IL-12 cytokine is composed of a single-chain IL-12 having the configuration (written from N-terminus to C-terminus) IL-12p40-IL-12p35 or IL-12p35-IL-12p40. In certain embodiments, the IL-12p40-IL-12p35 comprises an amino acid sequence as set forth in SEQ ID NO:3. In certain embodiments, the IL-12p35-IL-12p40 comprises an amino acid sequence as set forth in SEQ ID NO:4.

In another embodiment, the IL-12 cytokine may include subunits from different species, i.e. a chimeric IL-12 cytokine. In a related embodiment, the IL-12p35 subunit is derived from mouse and the IL-12p40 subunit is derived from human. In certain embodiments, the IL-12 cytokine comprises an IL-12p35 amino acid sequence as set forth in SEQ ID NO:5. In certain embodiments, the IL-12 cytokine comprises an IL-12p35 amino acid sequence as set forth in SEQ ID NO:5 and comprises an IL-12p40 amino acid sequence as set forth in SEQ ID NO:2.

It has been found that such a chimeric molecule is invaluable to generate in vitro as well in vivo data in mouse, without the need for a further surrogate masking moeity. The masking moiety will be specific for the human IL-12p40 subunit and thereby blocks the activity of the IL-12 cytokine. The IL-12p35 subunit derived from mouse nonetheless forms a functional IL-12 cytokine with the human IL-12p40 subunit and is active in mouse models. For use in humans, the IL-12p35 subunit from mouse will be replaced with the IL-12p35 subunit from human, however, the masking moiety and all other components of the IL-12 Fc fusion protein remain the same.

In certain embodiments, the chimeric IL-12p40-IL-12p35 comprises an amino acid sequence as set forth in SEQ ID NO:6. In certain embodiments, the chimeric IL-12p35-IL-12p40 comprises an amino acid sequence as set forth in SEQ ID NO:7.

In a related embodiment, the subunits within the single-chain IL-12 cytokine may be linked to each other via a linker, e.g. IL-12p40(linker) IL-12p35 or IL-12p35(linker)IL-12p40. The linker may be a peptide linker and especially any peptide linker as disclosed herein and preferably a GS linker. Hence, in a related embodiment the subunits in the single-chain IL-12 cytokine comprising the amino acid sequence as set forth in any one of SEQ ID NOs:3, 4, 6 or 7 are linked to each other via a linker as disclosed herein and preferably a GS linker. In a related embodiment, the GS linker has the following amino acid sequence GGGGSGGGGSGGGGS (SEQ ID NO:22). In a preferred embodiment, the single-chain IL-12 cytokine is provided in the configuration IL-12p40-15GS-IL-12p35 (SEQ ID NO:8). In another embodiment, the single-chain IL-12 cytokine is provided in the configuration IL-12p35-15GS-IL-12p40 (SEQ ID NO:9).

In a related embodiment, the single-chain IL-12 cytokine is provided in the configuration IL-12p40-15GS-IL-12p35 (SEQ ID NO:10). In another embodiment, the single-chain IL-12 cytokine is provided in the configuration IL-12p35-15GS-IL-12p40 (SEQ ID NO:11).

In another embodiment, the IL-12p35 subunit of the IL-12 Fc fusion protein comprises a polypeptide having at least 90%, 91%, 92%, 93%, 94%, 95%, 96%, 97%, 98%, or 99% identity to SEQ ID NO:1 and the IL-12p40 subunit of the IL-12 Fc fusion protein comprises a polypeptide having at least 90%, 91%, 92%, 93%, 94%, 95%, 96%, 97%, 98%, or 99% identity to SEQ ID NO:2, preferably the IL-12p35 subunit comprises or consists of the polypeptide of SEQ ID NO:1 and the IL-12p40 subunit comprises or consists of the polypeptide of SEQ ID NO:2.

In another embodiment, the single-chain IL-12p40-IL-12p35 is linked via its IL-12p40 subunit to the C-terminus of the first Fc domain. In another embodiment, the single-chain IL-12p35-IL-12p40 is linked via its IL-12p35 subunit to the first Fc domain. In both cases the single-chain IL-12p40-IL-12p35 or IL-12p35-IL-12p40 is linked via the first peptide linker to the C-terminus, which first peptide linker is protease-cleavable.

In another embodiment, the IL-12p40 subunit and the IL-12p35 subunit are linked to each other via a linker that is rich in amino acid residues glycine and serine. In a related embodiment, the linker has a length of 5 to 20 amino acids and only includes the amino acids glycine and serine. In a preferred embodiment, the linker has the amino acid sequence of SEQ ID NO:22.

In a preferred embodiment, the IL-12 Fc fusion protein comprises a single-chain IL-12p40-IL-12p35 polypeptide having at least 90%, 91%, 92%, 93%, 94%, 95%, 96%, 97%, 98%, or 99% identity to SEQ ID NO:8. In another embodiment, the IL-12 Fc fusion protein comprises a single-chain IL-12p35-IL-12p40 polypeptide having at least 90%, 91%, 92%, 93%, 94%, 95%, 96%, 97%, 98%, or 99% identity to SEQ ID NO:9.

The IL-12 cytokine may comprise a variant of the IL-12p35 and/or IL-12p40 sequence. The variant encodes for a protein that retains IL-12 functional activity as compared to the wild type IL-12. The variant may encode for an IL-12 subunit or any single chain IL-12 as disclosed herein. In one embodiment, the variant encodes for an IL-12 subunit or any single-chain IL-12 as show in any of SEQ ID NOs:1-11, additionally having at least 1, 2, 3, 4, 5, 6, 7, 8, 9, 10, 11, 12, 13, 14, 15, 16, 17, 18, 19 or 20 amino acid mutation, deletion, substitution and/or addition compared to the amino acid sequence shown in any of SEQ ID NOs:1-11.

Functional activity of IL-12 can be measured in an assay as shown in Example 5.

TABLE 1

| | | SEQ ID |
|---|---|---|
| Identifier | Sequence | NO: |
| IL-12p35 (human) | RNLPVATPDPGMFPCLHHSQNLLRAVSNMLQKARQTLEFYPCTSE | 1 |
| | EIDHEDITKDKTSTVEACLPLELTKNESCLNSRETSFITNGSCLA | |
| | SRKTSFMMALCLSSIYEDLKMYQVEFKTMNAKLLMDPKRQIFLDQ | |
| | NMLAVIDELMQALNFNSETVPQKSSLEEPDFYKTKIKLCILLHAF | |
| | RIRAVTIDRVMSYLNAS | |
| IL-12p40 (human) | IWELKKDVYVVELDWYPDAPGEMVVLTCDTPEEDGITWTLDQSSE | 2 |
| | VLGSGKTLTIQVKEFGDAGQYTCHKGGEVLSHSLLLLHKKEDGIW | |
| | STDILKDQKEPKNKTFLRCEAKNYSGRFTCWWLTTISTDLTFSVK | |
| | SSRGSSDPQGVTCGAATLSAERVRGDNKEYEYSVECQEDSACPAA | |
| | EESLPIEVMVDAVHKLKYENYTSSFFIRDIIKPDPPKNLQLKPLK | |
| | NSRQVEVSWEYPDTWSTPHSYFSLTFCVQVQGKSKREKKDRVFTD | |
| | KTSATVICRKNASISVRAQDRYYSSSWSEWASVPCS | |
| IL-12p40-IL-12p35 | IWELKKDVYVVELDWYPDAPGEMVVLTCDTPEEDGITWTLDOSSE | 3 |
| (human) | VLGSGKTLTIQVKEFGDAGQYTCHKGGEVLSHSLLLLHKKEDGIW | |
| | STDILKDQKEPKNKTFLRCEAKNYSGRFTCWWLTTISTDLTFSVK | |
| | SSRGSSDPQGVTCGAATLSAERVRGDNKEYEYSVECQEDSACPAA | |
| | EESLPIEVMVDAVHKLKYENYTSSFFIRDIIKPDPPKNLQLKPLK | |
| | NSRQVEVSWEYPDTWSTPHSYFSLTFCVQVQGKSKREKKDRVFTD | |
| | KTSATVICRKNASISVRAQDRYYSSSWSEWASVPCSRNLPVATPD | |
| | PGMFPCLHHSQNLLRAVSNMLQKARQTLEFYPCTSEEIDHEDITK | |
| | DKTSTVEACLPLELTKNESCLNSRETSFITNGSCLASRKTSFMMA | |
| | LCLSSIYEDLKMYQVEFKTMNAKLLMDPKRQIFLDONMLAVIDEL | |
| | MQALNFNSETVPQKSSLEEPDFYKTKIKLCILLHAFRIRAVTIDR | |
| | VMSYLNAS | |
| IL-12p35-IL-12p40 | RNLPVATPDPGMFPCLHHSONLLRAVSNMLQKARQTLEFYPCTSE | 4 |
| (human) | EIDHEDITKDKTSTVEACLPLELTKNESCLNSRETSFITNGSCLA | |
| | SRKTSFMMALCLSSIYEDLKMYQVEFKTMNAKLLMDPKRQIFLDQ | |
| | NMLAVIDELMQALNFNSETVPQKSSLEEPDFYKTKIKLCILLHAF | |
| | RIRAVTIDRVMSYLNASIWELKKDVYVVELDWYPDAPGEMVVLTC | |
| | DTPEEDGITWTLDQSSEVLGSGKTLTIQVKEFGDAGQYTCHKGGE | |
| | VLSHSLLLLHKKEDGIWSTDILKDQKEPKNKTFLRCEAKNYSGRF | |
| | TCWWLTTISTDLTFSVKSSRGSSDPQGVTCGAATLSAERVRGDNK | |
| | EYEYSVECQEDSACPAAEESLPIEVMVDAVHKLKYENYTSSFFIR | |
| | DIIKPDPPKNLQLKPLKNSRQVEVSWEYPDTWSTPHSYFSLTFCV | |
| | QVQGKSKREKKDRVFTDKTSATVICRKNASISVRAQDRYYSSSWS | |
| | EWASVPCS | |
| IL-12p35 (mouse) | RVIPVSGPARCLSQSRNLLKTTDDMVKTAREKLKHYSCTAEDIDH | 5 |
| | EDITRDQTSTLKTCLPLELHKNESCLATRETSSTTRGSCLPPQKT | |
| | SLMMTLCLGSIYEDLKMYQTEFQAINAALQNHNHQQIILDKGMLV | |
| | AIDELMQSLNHNGETLROKPPVGEADPYRVKMKLCILLHAFSTRV | |
| | VTINRVMGYLSSA | |
| IL-12p40-IL-12p35 | IWELKKDVYVVELDWYPDAPGEMVVLTCDTPEEDGITWTLDQSSE | 6 |
| (chimeric) | VLGSGKTLTIQVKEFGDAGQYTCHKGGEVLSHSLLLLHKKEDGIW | |
| | STDILKDQKEPKNKTFLRCEAKNYSGRFTCWWLTTISTDLTFSVK | |
| | SSRGSSDPQGVTCGAATLSAERVRGDNKEYEYSVECQEDSACPAA | |
| | EESLPIEVMVDAVHKLKYENYTSSFFIRDIIKPDPPKNLQLKPLK | |
| | NSRQVEVSWEYPDTWSTPHSYFSLTFCVQVQGKSKREKKDRVFTD | |
| | KTSATVICRKNASISVRAQDRYYSSSWSEWASVPCSRVIPVSGPA | |
| | RCLSQSRNLLKTTDDMVKTAREKLKHYSCTAEDIDHEDITRDQTS | |
| | TLKTCLPLELHKNESCLATRETSSTTRGSCLPPQKTSLMMTLCLG | |
| | SIYEDLKMYQTEFQAINAALQNHNHQQIILDKGMLVAIDELMQSL | |
| | NHNGETLRQKPPVGEADPYRVKMKLCILLHAFSTRVVTINRVMGY | |
| | LSSA | |
| IL-12p35-IL-12p40 | RVIPVSGPARCLSQSRNLLKTTDDMVKTAREKLKHYSCTAEDIDH | 7 |
| (chimeric) | EDITRDQTSTLKTCLPLELHKNESCLATRETSSTTRGSCLPPQKT | |
| | SLMMTLCLGSIYEDLKMYQTEFQAINAALQNHNHQQIILDKGMLV | |
| | AIDELMQSLNHNGETLROKPPVGEADPYRVKMKLCILLHAFSTRV | |
| | VTINRVMGYLSSAIWELKKDVYVVELDWYPDAPGEMVVLTCDTPE | |
| | EDGITWTLDQSSEVLGSGKTLTIQVKEFGDAGQYTCHKGGEVLSH | |
| | SLLLLHKKEDGIWSTDILKDQKEPKNKTFLRCEAKNYSGRFTCWW | |
| | LTTISTDLTFSVKSSRGSSDPQGVTCGAATLSAERVRGDNKEYEY | |
| | SVECQEDSACPAAEESLPIEVMVDAVHKLKYENYTSSFFIRDIIK | |
| | PDPPKNLQLKPLKNSRQVEVSWEYPDTWSTPHSYFSLTFCVQVQG | |
| | KSKREKKDRVFTDKTSATVICRKNASISVRAQDRYYSSSWSEWAS | |
| | VPCS | |
| IL-12p40- 15GS-IL- | IWELKKDVYVVELDWYPDAPGEMVVLTCDTPEEDGITWTLDOSSE | 8 |
| 12p35 (human) | VLGSGKTLTIQVKEFGDAGQYTCHKGGEVLSHSLLLLHKKEDGIW | |
| | STDILKDQKEPKNKTFLRCEAKNYSGRFTCWWLTTISTDLTFSVK | |
| | SSRGSSDPQGVTCGAATLSAERVRGDNKEYEYSVECQEDSACPAA | |
| | EESLPIEVMVDAVHKLKYENYTSSFFIRDIIKPDPPKNLQLKPLK | |
| | NSRQVEVSWEYPDTWSTPHSYFSLTFCVQVQGKSKREKKDRVFTD | |
| | KTSATVICRKNASISVRAQDRYYSSSWSEWASVPCSGGGGSGGGG | |
| | SGGGGSRNLPVATPDPGMFPCLHHSQNLLRAVSNMLQKAROTLEF | |
| | YPCTSEEIDHEDITKDKTSTVEACLPLELTKNESCLNSRETSFIT | |
| | NGSCLASRKTSFMMALCLSSIYEDLKMYQVEFKTMNAKLLMDPKR | |
| | QIFLDQNMLAVIDELMQALNENSETVPQKSSLEEPDFYKTKIKLC | |
| | ILLHAFRIRAVTIDRVMSYLNAS | |
| IL-12p35-15GS-IL- | RNLPVATPDPGMFPCLHHSQNLLRAVSNMLQKARQTLEFYPCTSE | 9 |
| 12p40 (human) | EIDHEDITKDKTSTVEACLPLELTKNESCLNSRETSFITNGSCLA | |
| | SRKTSFMMALCLSSIYEDLKMYQVEFKTMNAKLLMDPKRQIFLDQ | |
| | NMLAVIDELMQALNENSETVPQKSSLEEPDFYKTKIKLCILLHAF | |
| | RIRAVTIDRVMSYLNASGGGGSGGGGSGGGGSIWELKKDVYVVEL | |
| | DWYPDAPGEMVVLTCDTPEEDGITWTLDQSSEVLGSGKTLTIQVK | |
| | EFGDAGQYTCHKGGEVLSHSLLLLHKKEDGIWSTDILKDQKEPKN | |
| | KTFLRCEAKNYSGRFTCWWLTTISTDLTFSVKSSRGSSDPQGVTC | |
| | GAATLSAERVRGDNKEYEYSVECQEDSACPAAEESLPIEVMVDAV | |
| | HKLKYENYTSSFFIRDIIKPDPPKNLQLKPLKNSRQVEVSWEYPD | |
| | TWSTPHSYFSLTFCVQVQGKSKREKKDRVFTDKTSATVICRKNAS | |
| | ISVRAQDRYYSSSWSEWASVPCS | |
| IL-12p40-15GS-IL- | IWELKKDVYVVELDWYPDAPGEMVVLTCDTPEEDGITWTLDQSSE | 10 |
| 12p35 (chimeric) | VLGSGKTLTIQVKEFGDAGQYTCHKGGEVLSHSLLLLHKKEDGIW | |
| | STDILKDQKEPKNKTFLRCEAKNYSGRFTCWWLTTISTDLTFSVK | |
| | SSRGSSDPQGVTCGAATLSAERVRGDNKEYEYSVECQEDSACPAA | |
| | EESLPIEVMVDAVHKLKYENYTSSFFIRDIIKPDPPKNLQLKPLK | |
| | NSRQVEVSWEYPDTWSTPHSYFSLTFCVQVQGKSKREKKDRVFTD | |
| | KTSATVICRKNASISVRAQDRYYSSSWSEWASVPCSGGGGSGGGG | |
| | SGGGGSRVIPVSGPARCLSQSRNLLKTTDDMVKTAREKLKHYSCT | |
| | AEDIDHEDITRDQTSTLKTCLPLELHKNESCLATRETSSTTRGSC | |
| | LPPQKTSLMMTLCLGSIYEDLKMYQTEFQAINAALQNHNHQQIIL | |
| | DKGMLVAIDELMQSLNHNGETLRQKPPVGEADPYRVKMKLCILLH | |
| | AFSTRVVTINRVMGYLSSA | |
| IL-12p35-15GS-IL- | RVIPVSGPARCLSQSRNLLKTTDDMVKTAREKLKHYSCTAEDIDH | 11 |
| 12p40 (chimeric) | EDITRDQTSTLKTCLPLELHKNESCLATRETSSTTRGSCLPPQKT | |
| | SLMMTLCLGSIYEDLKMYQTEFQAINAALQNHNHQQIILDKGMLV | |
| | AIDELMQSLNHNGETLRQKPPVGEADPYRVKMKLCILLHAFSTRV | |
| | VTINRVMGYLSSAGGGGSGGGGSGGGGSIWELKKDVYVVELDWYP | |
| | DAPGEMVVLTCDTPEEDGITWTLDQSSEVLGSGKTLTIQVKEFGD | |
| | AGQYTCHKGGEVLSHSLLLLHKKEDGIWSTDILKDQKEPKNKTFL | |
| | RCEAKNYSGRFTCWWLTTISTDLTFSVKSSRGSSDPQGVTCGAAT | |
| | LSAERVRGDNKEYEYSVECQEDSACPAAEESLPIEVMVDAVHKLK | |
| | YENYTSSFFIRDIIKPDPPKNLQLKPLKNSRQVEVSWEYPDTWST | |
| | PHSYFSLTFCVQVQGKSKREKKDRVFTDKTSATVICRKNASISVR | |
| | AQDRYYSSSWSEWASVPCS | |
| IL-12Rß1 (human) | MEPLVTWVVPLLFLFLLSRQGAACRTSECCFQDPPYPDADSGSAS | 12 |
| P42701 | GPRDLRCYRISSDRYECSWQYEGPTAGVSHFLRCCLSSGRCCYFA | |
| | AGSATRLQFSDQAGVSVLYTVTLWVESWARNQTEKSPEVTLQLYN | |
| | SVKYEPPLGDIKVSKLAGOLRMEWETPDNQVGAEVQFRHRTPSSP | |
| | WKLGDCGPQDDDTESCLCPLEMNVAQEFQLRRROLGSQGSSWSKW | |
| | SSPVCVPPENPPQPQVRESVEQLGODGRRRLTLKEQPTQLELPEG | |
| | CQGLAPGTEVTYRLQLHMLSCPCKAKATRTLHLGKMPYLSGAAYN | |
| | VAVISSNQFGPGLNQTWHIPADTHTEPVALNISVGTNGTTMYWPA | |
| | RAQSMTYCIEWQPVGODGGLATCSLTAPQDPDPAGMATYSWSRES | |
| | GAMGQEKCYYITIFASAHPEKLTLWSTVLSTYHFGGNASAAGTPH | |
| | HVSVKNHSLDSVSVDWAPSLLSTCPGVLKEYVVRCRDEDSKQVSE | |
| | HPVQPTETQVTLSGLRAGVAYTVQVRADTAWLRGVWSQPQRFSIE | |
| | VOVSDWLIFFASLGSFLSILLVGVLGYLGLNRAARHLCPPLPTPC | |
| | ASSAIEFPGGKETWQWINPVDFQEEASLQEALVVEMSWDKGERTE | |
| | PLEKTELPEGAPELALDTELSLEDGDRCKAKM | |
| IL-12Rß2 (human) | MAHTFRGCSLAFMFIITWLLIKAKIDACKRGDVTVKPSHVILLGS | 13 |
| Q99665 | TVNITCSLKPRQGCFHYSRRNKLILYKFDRRINFHHGHSLNSQVT | |
| | GLPLGTTLFVCKLACINSDEIQICGAEIFVGVAPEQPQNLSCIQK | |
| | GEQGTVACTWERGRDTHLYTEYTLQLSGPKNLTWQKOCKDIYCDY | |
| | LDFGINLTPESPESNFTAKVTAVNSLGSSSSLPSTFTFLDIVRPL | |
| | PPWDIRIKFQKASVSRCTLYWRDEGLVLLNRLRYRPSNSRLWNMV | |
| | NVTKAKGRHDLLDLKPFTEYEFQISSKLHLYKGSWSDWSESLRAQ | |
| | TPEEEPTGMLDVWYMKRHIDYSRQQISLFWKNLSVSEARGKILHY | |
| | QVTLQELTGGKAMTQNITGHTSWTTVIPRTGNWAVAVSAANSKGS | |
| | SLPTRINIMNLCEAGLLAPROVSANSEGMDNILVTWQPPRKDPSA | |
| | VQEYVVEWRELHPGGDTQVPLNWLRSRPYNVSALISENIKSYICY | |
| | EIRVYALSGDQGGCSSILGNSKHKAPLSGPHINAITEEKGSILIS | |
| | WNSIPVQEQMGCLLHYRIYWKERDSNSQPOLCEIPYRVSQNSHPI | |
| | NSLQPRVTYVLWMTALTAAGESSHGNEREFCLQGKANWMAFVAPS | |
| | ICIAIIMVGIFSTHYFQQKVFVLLAALRPQWCSREIPDPANSTCA | |
| | KKYPIAEEKTOLPLDRLLIDWPTPEDPEPLVISEVLHQVTPVERH | |
| | PPCSNWPOREKGIQGHQASEKDMMHSASSPPPPRALQAESROLVD | |
| | LYKVLESRGSDPKPENPACPWTVLPAGDLPTHDGYLPSNIDDLPS | |
| | HEAPLADSLEELEPQHISLSVFPSSSLHPLTFSCGDKLTLDQLKM | |
| | RCDSLML | |

### Fc

According to the invention, the first and the second polypeptide chains of the IL-12 Fc fusion protein are linked to each other via their respective Fc domains, i.e. both polypeptide chains dimerize via their Fc domains.

In the context of the present invention, an Fc domain is for example derived from the heavy chain of an IgG, for example an IgG1, IgG2 or IgG4. For example, an Fc domain of the present invention is a Fc domain of a heavy chain of an IgG₁ and comprises a hinge region and two constant domains (CH2 and CH3). Examples of Fc domains (including a hinge region) are shown in SEQ ID NO:14.

For all constant region (CL, CH1, hinge, CH2, and CH3) positions discussed in the present invention, numbering is according to the EU numbering scheme (Kabat et al., 1991, Sequences of Proteins of Immunological Interest, 5th Ed., United States Public Health Service, National Institutes of Health, Bethesda), which refers to the numbering of the EU antibody (Edelman et al., 1969, Proc Natl Acad Sci USA 63:78-85), unless otherwise specified. This means that the amino acid numbers indicated herein correspond to the positions in a heavy chain of the corresponding sub-type (e.g. IgG₁ or IgG₄), according to the EU numbering system, unless otherwise specified. For all variable region (VL and VH) and J segment (JH and JL) positions discussed in the present invention, numbering is according to the Kabat numbering scheme (Kabat et al., 1991, Sequences of Proteins of Immunological Interest, 5th Ed., United States Public Health Service, National Institutes of Health, Bethesda). Exceptions to these numbering schemes are noted where they occur. Those skilled in the art of antibodies will appreciate that these conventions consist of nonsequential numbering in specific regions of an immunoglobulin sequence, enabling a normalized reference to conserved positions in immunoglobulin families. Accordingly, the positions of any given immunoglobulin as defined by EU numbering or Kabat numbering will not necessarily correspond to its sequential sequence.

In some embodiments, the first Fc domain and the second Fc domain in a fusion protein of the present invention each comprise one or more amino acid changes which reduce the formation of homodimers of the first or second polypeptide chains instead of heterodimers of a first and a second polypeptide chain. Through these changes, a “protrusion” is generated in one of the Fc domains by replacing one or more, small amino acid side chains from the interface of one of the heavy chains with larger side chains (e.g. tyrosine or tryptophan). Compensatory “cavities” of identical or similar size are created on the interface of the other Fc domain by replacing large amino acid side chains with smaller ones (e.g. alanine or threonine). This provides a mechanism for increasing the yield of the heterodimer over other unwanted end-products such as homodimers, in particular homodimers of the Fc domain with the “protrusion” (see for example Ridgway et al. Protein Eng, 1996. 9 (7): p. 617-21; Atwell et al, JMB, 1997, 270, 26-35).

In some embodiments, such amino acid changes are a tyrosine (Y) at position 366 [T366Y] of the first Fc domain and a threonine (T) at position 407 [Y407T] of the second Fc domain. In some embodiments, the first Fc domain comprises a serine (S) at position 366 [T366S] and the second Fc domain comprises a tryptophan (W) at position 366 [T366W], an alanine (A) at position 368 [L368A] and a valine (V) at position 407 [Y407V]. In preferred embodiments, the first Fc domain comprises a tryptophan (W) at position 366 [T366W] and the second Fc domain comprises a serine (S) at position 366 [T366S], an alanine (A) at position 368 [L368A] and a valine (V) at position 407 [Y407V]. For example, position 366 of the Fc domain according to EU numbering, corresponding to the amino acid position 151 in the human IgG₁ Fc sequence of SEQ ID NO:14, is changed from T at position 151 in SEQ ID NO:14 to W at position 151 in SEQ ID NO:15; and positions 366, 368 and 407 according to EU numbering, corresponding to the amino acid positions 151, 153 and 192, respectively, in SEQ ID NO:14, are changed from T, L and Y at these positions in SEQ ID NO:14 to S, A and V at these positions in SEQ ID NO:16. In any of these embodiments, the amino acid changes described for the first Fc domain may be located in the second Fc domain and the respective amino acid changes for the second Fc domain may be located in the first Fc domain. In other words, the term “first” and “second” can be exchanged in these embodiments. In some embodiments, such a Fc domain is an Fc domain derived from the heavy chain of an IgG₁.

In some embodiments, the first Fc domain comprises a cysteine (C) at position 354 [S354C] in addition to the tryptophan (W) at position 366 [T366W] and the second Fc domain comprises a cysteine (C) at position 349 [Y349C] in addition to the serine (S) at position 366 [T366S], the alanine (A) at position 368 [L368A] and the valine (V) at position 407 [Y407V]. In one aspect, such Fc domain is an Fc domain derived from the heavy chain of an IgG₁.

In some embodiments, the first and/or the second Fc domain of the present invention derived from an IgG1 also includes the “KO” mutations (L234A, L235A).

In some embodiments, the first Fc domain or the second Fc domain in an Fc fusion protein of the present invention further comprises one or more amino acid changes which reduce the binding of the Fc domain to protein A. In some embodiments, such amino acid changes are an arginine at position 435 [H435R] and a phenylalanine at position 436 [Y436F] of one of the Fc domains. Both changes are derived from the sequence of human IgG3 (lgG3 does not bind to protein A). These two mutations are located in the CH3 domain and are incorporated in one of the Fc domains to reduce binding to Protein A (see for example Jendeberg et al. J Immunol Methods, 1997. 201 (1): p. 25-34). These two changes facilitate the removal of homodimers of heavy chains comprising these changes during protein purification.

In some embodiments, in a fusion protein of the present invention, the Fc domain, which comprises a threonine (T) at position 407 [Y407T], further comprises an arginine at position 435 [H435R] and a phenylalanine at position 436 [Y436F]. In this case, the other heavy chain comprises a tyrosine (Y) at position 366 [T366Y], but does not include the two changes at positions 435 and 436. Alternatively, in some embodiments, in a fusion protein of the present invention, the Fc domain, which comprises a serine (S) at position 366 [T366S], an alanine (A) at position 368 [L368A] and a valine (V) at position 407 [Y407V], further comprises an arginine at position 435 [H435R] and a phenylalanine at position 436 [Y436F]. In this case, the other Fc domain comprises a tryptophan (W) at position 366 [T366W], but does not include the two changes at positions 435 and 436. Thus, the Fc domain comprising the amino acid change resulting in a “cavity” as described above also comprises the amino acid changes, which reduce binding to Protein A. Homodimers comprising this Fc domain are removed through reduced binding to Protein A. The production of homodimers of the other Fc domain, which comprises the “protrusion”, is reduced by the presence of the “protrusion”.

In a preferred embodiment, the first Fc domain comprises or consists of an amino acid sequence as shown in SEQ ID NO:14. In a preferred embodiment, the first Fc domain comprises or consists of an amino acid sequence as shown in SEQ ID NO:15. In a preferred embodiment, the first Fc domain comprises or consists of an amino acid sequence as shown in SEQ ID NO:16. In a preferred embodiment, the first Fc domain comprises or consists of an amino acid sequence as shown in SEQ ID NO:17. In another preferred embodiment, the second Fc domain comprises or consists of an amino acid sequence as shown in SEQ ID NO:18. In a related preferred embodiment, the IL-12 Fc fusion protein comprises a first Fc domain comprising or consisting of an amino acid sequence as shown in SEQ ID NO:17 and a second Fc domain comprising or consisting of an amino acid sequence as shown in SEQ ID NO:18.

In any of the aforementioned embodiments the serine at position 5 in the amino acid sequence of SEQ ID NOs:14-18 may be replaced by a cysteine.

In some embodiments, the Fc domain of a fusion protein of the present invention may or may not further comprise YTE mutations (M252Y/S254T/T256E, EU numbering (Dall'Acqua, Kiener et al. 2006)). These mutations have been shown to improve the pharmacokinetic properties of Fc domains through preferential enhancement of binding affinity for neonatal FcRn receptor at pH 6.0.

TABLE 2

| | | SEQ ID |
|---|---|---|
| Identifier | Sequence | NO: |
| IgG1 Fc domain | EPKSSDKTHTCPPCPAPELLGGPSVFLFPPKPKDTLMISRTPEVT | 14 |
| (human) | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLTCLVKGFYPSDIAVEWESNGQPENNYKTTP | |
| | PVLDSDGSFFLYSKLTVDKSRWQQGNVFSCSVMHEALHNHYTQKS | |
| | LSLSPG | |
| IgG1 Fc domain, | EPKSSDKTHTCPPCPAPELLGGPSVFLFPPKPKDTLMISRTPEVT | 15 |
| T366W Knob | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPG | |
| IgG1 Fc domain, | EPKSSDKTHTCPPCPAPELLGGPSVFLFPPKPKDTLMISRTPEVT | 16 |
| T366S/L368A/ | CVVVDVSHEDPEVKENWYVDGVEVHNAKTKPREEQYNSTYRVVSV | |
| Y407V Hole | LTVLHQDWLNGKEYKCKVSNKALPAPIEKTISKAKGQPREPQVYT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNHYTQKS | |
| | LSLSPG | |
| IgG1 Fc domain, | EPKSSDKTHTCPPCPAPEAAGGPSVFLFPPKPKDTLMISRTPEVT | 17 |
| L234A/L235A, | CVVVDVSHEDPEVKENWYVDGVEVHNAKTKPREEQYNSTYRVVSV | |
| T366W Knob | LTVLHQDWLNGKEYKCKVSNKALPAPIEKTISKAKGQPREPQVYT | |
| | LPPSREEMTKNQVSLWCLVKGFYPSDIAVEWESNGQPENNYKTTP | |
| | PVLDSDGSFFLYSKLTVDKSRWQQGNVFSCSVMHEALHNHYTOKS | |
| | LSLSPG | |
| IgG1 Fc domain, | EPKSSDKTHTCPPCPAPEAAGGPSVFLFPPKPKDTLMISRTPEVT | 18 |
| L234A/L235A, | CVVVDVSHEDPEVKFNWYVDGVEVHNAKTKPREEQYNSTYRVVSV | |
| T366S/L368A/ | LTVLHQDWLNGKEYKCKVSNKALPAPIEKTISKAKGQPREPQVYT | |
| Y407V Hole, | LPPSREEMTKNQVSLSCAVKGFYPSDIAVEWESNGQPENNYKTTP | |
| H435R/Y436F | PVLDSDGSFFLVSKLTVDKSRWQQGNVFSCSVMHEALHNRFTQKS | |
| | LSLSPG | |

### Protease-Cleavable Linker

The protease-cleavable linker links the Fc domain to the IL-12 cytokine, i.e. the protease-cleavable linker is positioned between the C-terminus of the Fc domain and the IL-12 cytokine. Once the protease-cleavable linker is cleaved by its respective protease the IL-12 cytokine is set free and is no longer attached to the Fc fusion protein.

On the Fc domain side, the protease-cleavable linker may be linked directly to the C-terminus of the Fc domain (i.e. without a linker) or it may be linked to the C-terminus of the Fc domain via a linker, such as any linker as described in the linker section below, and preferably a peptide linker e.g. having a length of about 4 to 20 amino acids. In a preferred embodiment, the protease-cleavable linker is linked to the C-terminus of the Fc domain via a linker having the sequence GGGGSGGGG (SEQ ID NO:24).

On the IL-12 cytokine side, the protease-cleavable linker may be linked directly to the IL-12 cytokine or it may be linked to the IL-12 cytokine via a linker, such as any linker as described in the linker section below, and preferably a peptide linker e.g. having a length of about 4 to 20 amino acids. Depending on the configuration of the IL-12 cytokine, the protease-cleavable linker is linked to the IL-12p40 subunit or to the IL-12p35 subunit. In a preferred embodiment, the IL-12 cytokine is provided in a single-chain configuration and the protease-cleavable linker is linked to (a) the IL-12p40 subunit of the single-chain IL-12p40-IL-12p35, or (b) the IL-12p35 subunit of the single-chain IL-12p35-IL-12p40. In a related embodiment, the protease-cleavable linker is linked to the IL-12p35 subunit or the IL-12p40 subunit via a linker having the sequence GGGGS (SEQ ID NO:27).

The protease-cleavable linker usually has a short amino acid (aa) sequence from 2 aa to 20 aa, 4 aa to 15 aa, 4 aa to 12 aa, or 2 aa to 10 aa. In certain embodiments the protease-cleavable linker may have a length of 2, 3, 4, 5, 6, 7, 8, 9, 10, 11, 12, 13, 14, 15, 16, 17, 18, 19, or 20 aa.

Preferably, the desired protease is enriched, selectively expressed and/or more active at the desired site of the cytokine activity, e.g. the TME. Thus, the IL-12 Fc fusion protein is preferentially or selectively cleaved at the site of desired cytokine activity.

Proteases known to be associated with diseased cells or tissues include but are not limited to serine proteases, cysteine proteases, aspartate proteases, threonine proteases, glutamic acid proteases, metalloproteases, asparagine peptide lyases, serum proteases, cathepsins, Cathepsin B, Cathepsin C, Cathepsin D, Cathepsin E, Cathepsin K, Cathepsin L, kallikreins, hKI, hK10, hK15, plasmin, collagenase, Type IV collagenase, stromelysin, Factor Xa, chymotrypsin-like protease, trypsin-like protease, elastase-like protease, subtilisin-like protease, actinidain, bromelain, calpain, caspases, caspase-3, Mirl-CP, papain, HIV-1 protease, HSV protease, CMV protease, chymosin, renin, pepsin, matriptase, legumain, plasmepsin, nepenthesin, metalloexopeptidases, metalloendopeptidases, matrix metalloproteases (MMP), MMP1, MMP2, MMP3, MMP8, MMP9, MMP13, MMP11, MMP14, urokinase plasminogen activator (uPA), enterokinase, prostate-specific antigen (PSA, hK3), interleukin-1b converting enzyme, thrombin, FAP (FAP-a), dipeptidyl peptidase, meprins, granzymes and dipeptidyl peptidase IV (DPPIV/CD26).

Proteases capable of cleaving amino acid sequences encoded by the protease-cleavable linker sequences provided herein can, for example, be selected from the group consisting of a prostate specific antigen (PSA), a matrix metalloproteinase (MMP), an A Disintigrin and a Metalloproteinase (ADAM), a plasminogen activator, a cathepsin, a caspase, a tumor cell surface protease, and an elastase. The MMP can, for example, be matrix metalloproteinase 2 (MMP2) or matrix metalloproteinase 9 (MMP9). Preferably, the protease-cleavable linker is cleaved by MMP2, MMP9 or MMP13.

TABLE 3

| Cleavable | SEQ ID |
|---|---|
| MMP Linkers | NO: |
| GPLGVRG | 232 |
| GPLGLRG | 233 |
| GPLGLAR | 234 |
| GPAALVGA | 235 |
| GPAALIGG | 236 |
| GPLNLVGR | 237 |
| GPAGLVAD | 238 |
| GPANLVAP | 239 |
| VPLSLYSG | 240 |
| SGESPAYYTA | 241 |

In a preferred embodiment the protease-cleavable linker sequence is GPLGVRG (SEQ ID NO:232).

Cleavage of the protease-cleavable linker can be easily determined as shown in Example 7-8.

### Masking Moiety

The masking moiety as used herein refers to a moiety that binds to the IL-12p35 and/or IL-12p40 subunit of the IL-12 cytokine. In one embodiment, by binding of the masking moeity to the IL-12p35 and/or IL-12p40 subunit the affinity of the IL-12 cytokine for its cognate receptor is decreased. In another embodiment, by binding of the masking moeity to the IL-12p35 and/or IL-12p40 subunit the functional activity of the IL-12 cytokine is blocked, inhibited or attenuated.

Binding of the masking moiety to the IL-12p35 and/or IL-12p40 subunit of the IL-12 cytokine can be easily measured by methods well known in the art e.g. see Example 2. The strength or affinity of specific binding can be expressed in terms of dissociation constant (K_{D}) of the interaction, wherein a smaller K_{D} represents greater affinity and a larger K_{D} represents lower affinity. Binding properties can be determined by methods such as bio-layer interferometry and surface plasmon resonance based methods, including Biacore and Octet methodologies. One such method entails measuring the rates of antigen-binding site/antigen or receptor/ligand complex association and dissociation, wherein rates depend on the concentration of the complex partners, the affinity of the interaction, and geometric parameters that equally influence the rate in both directions. Thus, both the association rate (kₐ) and the dissociation rate (k_{d}) can be determined, and the ratio of kₐ/k_{d}; is equal to the dissociation constant K_{D}.

Specific binding to the IL-12p35 and/or IL-12p40 subunit can be exhibited, for example, by a masking moiety having a K_{D} of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater.

In one embodiment, the masking moiety may be an IL-12 receptor or an IL-12p35 or IL-12p40 binding fragment thereof. In one embodiment, the masking moiety may be an IL-12p40 binding fragment of the IL-12 receptor. In one embodiment, the masking moiety may be an IL-12p35 binding fragment of the IL-12 receptor.

The IL-12 receptor is a type I cytokine receptor that binds IL-12. It consists of the beta 1 and beta 2 subunits. The IL-12 receptor, beta 1, or IL-12Rβ1 in short, is a subunit of the interleukin 12 receptor. IL12RB1, is the name of its human gene. IL-12Rβ1 is also known as CD212 (cluster of differentiation 212). The human IL-12Rβ1 has the amino acid sequence as shown in SEQ ID NO:12. The IL-12 receptor, beta 2 subunit is a subunit of the interleukin 12 receptor. IL12RB2 is its human gene. The human IL-12Rβ2 has the amino acid sequence as shown in SEQ ID NO:13. In some embodiments, the masking moiety comprises an amino acid sequence having 90%, 91%, 92%, 93%, 94%, 95%, 96%, 97%, 98%, or 99% identity to human IL-12Rβ1 having the amino acid sequence as shown in SEQ ID NO: 12. In some embodiments, the masking moiety comprises an amino acid sequence having 90%, 91%, 92%, 93%, 94%, 95%, 96%, 97%, 98%, or 99% identity to human IL-12RB2 having the amino acid sequence as shown in SEQ ID NO: 13.

In some embodiments, the masking moiety comprises the extracellular domain of IL-12Rβ1 or IL-12R32 or a fragment, portion, or variant thereof that retains affinity to IL-12. The extracellular domain is underlined in the SEQ ID NOs:12 and 13 in TABLE 1. In some embodiments, the masking moiety comprises an amino acid sequence having 90%, 91%, 92%, 93%, 94%, 95%, 96%, 97%, 98%, or 99% identity to the extracellular domain of human IL-12Rβ1 having the underlined amino acid sequence as shown in SEQ ID NO:12. In some embodiments, the masking moiety comprises an amino acid sequence having 90%, 91%, 92%, 93%, 94%, 95%, 96%, 97%, 98%, or 99% identity to the extracellular domain of human IL-12Rβ2 having the amino acid sequence as shown in SEQ ID NO:13.

The masking moiety may also be an scFv or an immunoglobulin single variable domain. Preferably, the masking moiety is a VHH and more preferably a humanized VHH.

In some embodiments, the scFv comprises the same light chain CDRs and heavy chain CDRs or the same light chain variable region (VL) and heavy chain variable region (VH) as the IL-12 antibody briakinumab or ustekinumab.

The CDRs disclosed herein and depicted in SEQ ID NOs:61-109 are presented according to the Kabat nomenclature. The underlined sequence corresponds to the respective CDR-1, CDR-2 and CDR-3 according to Kabat nomenclature. The CDR's are identified again individually in Kabat nomenclature in SEQ ID NOs:333-479.

As additional nomenclatures are known in the art, the CDR sequences based on the most commonly used of these nomenclatures are shown as well, but only for those instances in which the application of these alternative nomenclatures resulted in different amino acid sequences. These numbering systems are based on (i) CCG (Chemical Computing Group as illustrated in Almagro et al., Proteins 2011; 79:3050-3066 and Maier et al, Proteins 2014; 82:1599-1610), (ii) Chothia (Chothia and Lesk, 1987, J. Mol. Biol. 196: 901-917), (iii) IMGT (Lefranc M P, Dev Comp Immunol. 2003 January; 27(1):55-77) and (iv) North (North B, J Mol Biol. (2011) 406:228-56).

The amino acid residues of a VHH domain are numbered according to the general numbering for VH domains given by Kabat et al. (“Sequence of proteins of immunological interest”, US Public Health Services, NIH Bethesda, MD, Publication No. 91), as applied to VHH domains from Camelids, as shown e.g. in FIG. 2 of Riechmann and Muyldermans, J. Immunol. Methods 231, 25-38 (1999).

According to this numbering,
FR1 comprises the amino acid residues at positions 1-30,
CDR1 comprises the amino acid residues at positions 31-35,
FR2 comprises the amino acids at positions 36-49,
CDR2 comprises the amino acid residues at positions 50-65,
FR3 comprises the amino acid residues at positions 66-94,
CDR3 comprises the amino acid residues at positions 95-102, and
FR4 comprises the amino acid residues at positions 103-113.

The total number of amino acid residues in a VHH domain will usually be in the range of from 110 to 120, often between 112 and 115. It should however be noted that smaller and longer sequences may also be suitable for the purposes described herein.

However, it should be noted that—as is well known in the art for VH domains and for VHH domains—the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence.

Alternative methods for numbering the amino acid residues of VH domains, which methods can also be applied in an analogous manner to VHH domains, are known in the art. However, in the present description, claims and figures, the numbering according to Kabat and applied to VHH domains as described above will be followed, unless indicated otherwise.

In some embodiments the masking moiety comprises or consists of an amino acid sequence selected from the group consisting of any one of SEQ ID NOs:61-109.

In some embodiments the masking moiety is an IL-12 binding immunoglobulin single variable domain comprising the three CDRs contained within any one of the sequences of SEQ ID NOs:61-109.

In some embodiments the masking moiety is an IL-12 binding VHH comprising the three CDRs contained within any one of the sequences of SEQ ID NOs:61-109.

In some embodiments the masking moiety is a VHH and comprises the three CDRs contained within any one of the sequences of SEQ ID NOs:61-109 and further comprises framework regions (FR1, FR2, FR3, FR4) having at least 90%, 91%, 92%, 93%, 94%, 95%, 96%, 97%, 98%, or 99% identity to the framework regions as shown in any one of the sequences of SEQ ID NOs:61-109.

In some embodiments the masking moiety is a VHH and comprises the three CDRs contained within any one of the sequences of SEQ ID NOs:61-109 and further comprises framework regions (FR1, FR2, FR3, FR4) having 1, 2, 3, 4, 5, 6, 7, 8, 9, 10, 11, 12, 13, 14, 15, 16, 17, 18, 19, or up to 20 amino acid differences, such as substitutions, deletions or additions in the framework region as shown in any one of the sequences of SEQ ID NOs:61-109.

In some embodiments the masking moiety is a VHH and the VHH further comprises an additional alanine at its C-terminus. In some embodiments the masking moiety is a VHH having an amino acid sequence as shown in any one of SEQ ID NOs:61-109 and further comprising an additional alanine attached to its C-terminus.

It will be understood that any of the aforementioned (and below listed) VHH sequences and the CDRs contained in the VHH sequences may found utility beyond their use as a masking moiety in the IL-12 Fc fusion protein. Therefore, the present disclosure is not to be understood to limit the VHH sequences to their use as masking moieties but provides the sequences per se, i.e. for any and all purposes.

TABLE 4

| | | SEQ ID |
|---|---|---|
| Identifier | Sequence | NO: |
| BI-001 | QVQLVESGGGLVQPGGSLRLSCAASGREFTSVSMAWFRQRPGKER | 61 |
| | EFVAFSARISETTEYADFVKGRFTIWRDNANAKVTVYLQMNNLKP | |
| | EDTGVYYCAASEGPATIRQNTYPDWGQGTQVTVSS | |
| BI-001 | SVSMA | 333 |
| CDR-1 (Kabat) | | |
| BI-001 | FSARISETTEYADFVKG | 334 |
| CDR-2 (Kabat) | | |
| BI-001 | SEGPATIRQNTYPD | 335 |
| CDR-3 (Kabat) | | |
| BI-002 | EVQLVESGGGLVQPGGSLRLSCAASGFTFSSYYMYWIRQAPGKGL | 62 |
| | EWVSTIKPNGSGIYGNSVAGRFTISRDNAKNMLYLQMNMLRPEDT | |
| | ALYYCARDVRGTVRGQGTQVTVSS | |
| BI-002 | SYYMY | 336 |
| CDR-1 (Kabat) | | |
| BI-002 | TIKPNGSGIYGNSVAG | 337 |
| CDR-2 (Kabat) | | |
| BI-002 | DVRGTV | 338 |
| CDR-3 (Kabat) | | |
| BI-003 | QVQLVESGGGLVQPGGSLRLSCAASGNQLSLYNMGWYRQAPGKQR | 63 |
| | ELVASISRAGRSSYGDSVKGRFTISRDNAKNTVYLQMSSLKPEDT | |
| | AVYYCKASFLDDYWGQGTQVTVSS | |
| BI-003 | LYNMG | 339 |
| CDR-1 (Kabat) | | |
| BI-003 | SISRAGRSSYGDSVKG | 340 |
| CDR-2 (Kabat) | | |
| BI-003 | SFLDDY | 341 |
| CDR-3 (Kabat) | | |
| BI-004 | EVQLVESGGGLVQPGGSLRLSCAASGFSFSSSWMFWVRQPPGKGL | 64 |
| | EWVSTISPSGDYSRYADSVQGRFTISRDNTKNTLSLQMNSLKPED | |
| | TALYYCARDLRGTMRGQGTQVTVSS | |
| BI-004 | SSWMF | 342 |
| CDR-1 (Kabat) | | |
| BI-004 | TISPSGDYSRYADSVQG | 343 |
| CDR-2 (Kabat) | | |
| BI-004 | DLRGTM | 344 |
| CDR-3 (Kabat) | | |
| BI-005 | QVQLVESGGGLVQPGGSLRLSCAASGFTFSNFVMKWYHQAPGKER | 65 |
| | DLVASIDTTHFTNYADSVKGRFTISRDNSKNTVYLQMNSLKSEDT | |
| | AVYYCKVRRRDYEDYWGQGTQVTVSS | |
| BI-005 | NFVMK | 345 |
| CDR-1 (Kabat) | | |
| BI-005 | SIDTTHFTNYADSVKG | 346 |
| CDR-2 (Kabat) | | |
| BI-005 | RRRDYEDY | 347 |
| CDR-3 (Kabat) | | |
| BI-006 | EVQLVESGGGLVQAGGSLRLSCAASGRTFSRYNMGWFRQAPGKER | 66 |
| | EFVAAIIWSGGVINYADSVKGRFTISRDNAKNTVYLQMNSLKPED | |
| | TARYYCAADDKYYDRIVRGTADYWGQGTQVTVSS | |
| BI-006 | RYNMG | 348 |
| CDR-1 (Kabat) | | |
| BI-006 | AIIWSGGVINYADSVKG | 349 |
| CDR-2 (Kabat) | | |
| BI-006 | DDKYYDRIVRGTADY | 350 |
| CDR-3 (Kabat) | | |
| BI-007 | EVQLVESGGGLVQPGGSLRLSCVASGSIGSVVSWGWYRQAPGLER | 67 |
| | ELVASDASGGRPNYQDSVKGRFTISRDSAKNTVYLQMNSLKPEDT | |
| | AVYYCNLRGLQLDMGLYDSWGQGTQVTVSS | |
| BI-007 | VVSWG | 351 |
| CDR-1 (Kabat) | | |
| BI-007 | SDASGGRPNYQDSVKG | 352 |
| CDR-2 (Kabat) | | |
| BI-007 | RGLQLDMGLYDS | 353 |
| CDR-3 (Kabat) | | |
| BI-008 | EVQLVESGGGLVQAGDSLKLSCATSGRPSRDYAMGWFRQAPGKKR | 68 |
| | DFVAGISSGGGFTNYADSVKARFTISKDNAKNTVYLQMNSLKPED | |
| | TAVYYCAAQSGTNYISRTSPPYWGQGTQVTVSS | |
| BI-008 | DYAMG | 354 |
| CDR-1 (Kabat) | | |
| BI-008 | GISSGGGFTNYADSVKA | 355 |
| CDR-2 (Kabat) | | |
| BI-008 | QSGTNYISRTSPPY | 356 |
| CDR-3 (Kabat) | | |
| BI-009 | EVQLVESGGGLVHPGGSLRLSCVASGFRFTPYTMGWYRQAPGKQR | 69 |
| | ELVASISSVYSTNYADSVKGRFTVSRDNVKGTVSLQMNSLKPEDT | |
| | AVYYCNAPGLLHEEGTDYWGQGTQVTVSS | |
| BI-009 | PYTMG | 357 |
| CDR-1 (Kabat) | | |
| BI-009 | SISSVYSTNYADSVKG | 358 |
| CDR-2 (Kabat) | | |
| BI-009 | PGLLHEEGTDY | 359 |
| CDR-3 (Kabat) | | |
| BI-010 | QVQLVESGGGSVQVGGSLRLSCVGSGRTLNMYNMGWFRQAPGKER | 70 |
| | EFVAAISGKGLISDYRDSVKGRFTISRDNARNTMYLQMNSLKPED | |
| | TAVYHCAAGQWSAGPFTRERSYEYWGQGTQVTVSS | |
| BI-010 | MYNMG | 360 |
| CDR-1 (Kabat) | | |
| BI-010 | AISGKGLISDYRDSVKG | 361 |
| CDR-2 (Kabat) | | |
| BI-010 | GQWSAGPFTRERSYEY | 362 |
| CDR-3 (Kabat) | | |
| BI-011 | EVQLVESGGGLVQAGGSLRLSCAASGRTFSRWTMAWFRQAPGKER | 71 |
| | DFVAAVGWWNDSTYYADSVKGRFTISRDNNENTLYLQMNSLKPED | |
| | TAVYICASTEKYGLGQPNPRRYDYWGQGTQVTVSS | |
| BI-011 | RWTMA | 363 |
| CDR-1 (Kabat) | | |
| BI-011 | AVGWWNDSTYYADSVKG | 364 |
| CDR-2 (Kabat) | | |
| BI-011 | TEKYGLGQPNPRRYDY | 365 |
| CDR-3 (Kabat) | | |
| BI-012 | QVQLVESGGGLVQPGGSLRLSCAASGRTLSSYTMAWFRQAPGKER | 72 |
| | EFVATISPVGFIMDYADSVKGRFTISRDNTKNTVYLQMNSLKHED | |
| | TALYYCATDLGRKLGTQSREYGYWGQGTQVTVSS | |
| BI-012 | SYTMA | 366 |
| CDR-1 (Kabat) | | |
| BI-012 | TISPVGFIMDYADSVKG | 367 |
| CDR-2 (Kabat) | | |
| BI-012 | DLGRKLGTQSREYGY | 368 |
| CDR-3 (Kabat) | | |
| BI-013 | EVQLVESGGGLVQAGGSLRLSCAASGRTFSTYAVGWFRQAPGKER | 73 |
| | EFVAAISWGGGTVRYADSVKGRSTISRDDAKNTVYLQMNSLKPED | |
| | TAIYYCAARVLHIATKAVDFGSWGQGTQVTVSS | |
| BI-013 | TYAVG | 369 |
| CDR-1 (Kabat) | | |
| BI-013 | AISWGGGTVRYADSVKG | 370 |
| CDR-2 (Kabat) | | |
| BI-013 | RVLHIATKAVDFGS | 371 |
| CDR-3 (Kabat) | | |
| BI-014 | EVQLVESGGGSVQVGGSLRLSCVASGRTFNMYVMGWFRQAPGKER | 74 |
| | EFVAAISGEGLISDYRDSVKGRFTISRDNAKNTMYLQMNSLKPED | |
| | TAVYHCAAGQWTNGPFTRERTYEYWGQGTQVTVSS | |
| BI-014 | MYVMG | 372 |
| CDR-1 (Kabat) | | |
| BI-014 | AISGEGLISDYRDSVKG | 373 |
| CDR-2 (Kabat) | | |
| BI-014 | GQWTNGPFTRERTYEY | 374 |
| CDR-3 (Kabat) | | |
| BI-015 | EVQLVESGGGLVQPGGSLRLSCAASGRDFDRSTMAWYRQAPGKQR | 75 |
| | ELVASIPSDIGTKYADSVKGRFFISRVYAKNTNTVYLQMNSLKPE | |
| | DTAVYYCYAHIDSDYWGQGTQVTVSS | |
| BI-015 | RSTMA | 375 |
| CDR-1 (Kabat) | | |
| BI-015 | SIPSDIGTKYADSVKG | 376 |
| CDR-2 (Kabat) | | |
| BI-015 | HIDSDY | 377 |
| CDR-3 (Kabat) | | |
| BI-016 | EVQLVESGGGLVQPGGSLRLSCAASGRTFSSRAMGWFRQAPGKER | 76 |
| | EFVAAISFGGGTIRYADSVKGRFTISRDDAKNTVYLQMNSLKPED | |
| | TAVYYCAARRLHIATLAADFDSWGQGTQVTVSS | |
| BI-016 | SRAMG | 378 |
| CDR-1 (Kabat) | | |
| BI-016 | AISFGGGTIRYADSVKG | 379 |
| CDR-2 (Kabat) | | |
| BI-016 | RRLHIATLAADFDS | 380 |
| CDR-3 (Kabat) | | |
| BI-017 | EVQLVESGGGLVQAGGSLRLSCSASGRSLNDYIVGWFRQAPGKER | 77 |
| | ELVAAISSGGYIQHYIDSVKGRFTISRDNAKNTVYLQMNGLKPED | |
| | TAVYYCAANQLNGVARKKIESDDYDYWGQGTQVTVSS | |
| BI-017 | DYIVG | 381 |
| CDR-1 (Kabat) | | |
| BI-017 | AISSGGYIQHYIDSVKG | 382 |
| CDR-2 (Kabat) | | |
| BI-017 | NQLNGVARKKIESDDYDY | 383 |
| CDR-3 (Kabat) | | |
| BI-018 | EVQLVESGGGLVEAGGSLRVSCAASGGTFSEYAMGWIRQAPGKER | 78 |
| | EFVAGISRGAGRTVYADSVKGRFTISRDNHKNTVYLQMNSLKPED | |
| | TAVYYCGADDVSYNRVTTAPGEYDYWGQGIQVTVSS | |
| BI-018 | EYAMG | 384 |
| CDR-1 (Kabat) | | |
| BI-018 | GISRGAGRTVYADSVKG | 385 |
| CDR-2 (Kabat) | | |
| BI-018 | DDVSYNRVTTAPGEYDY | 386 |
| CDR-3 (Kabat) | | |
| BI-019 | EVQLVESGGGAVQAGGALKLSCAYSGRAFSRSLMGWFRQAPGKER | 79 |
| | EFVAAISWVSVTPDYGDSVKGRFTISRDNAKSTVTLQMNSLKPED | |
| | TAVYYCAASERYGTPRRRPNDYDYWGQGTQVTVSS | |
| BI-019 | RSLMG | 387 |
| CDR-1 (Kabat) | | |
| BI-019 | AISWVSVTPDYGDSVKG | 388 |
| CDR-2 (Kabat) | | |
| BI-019 | SERYGTPRRRPNDYDY | 389 |
| CDR-3 (Kabat) | | |
| BI-020 | EVQLVESGGGLVQPGGSLRLSCVASGSIGSITSMAWYRQGTGKQR | 80 |
| | ELVASISSGGRPSYQDSVKGRFTISRDNAENTVYLQMNSLKPEDT | |
| | AVYHCNVRGLHLDTGLYESWGQGTQVTVSS | |
| BI-020 | ITSMA | 390 |
| CDR-1 (Kabat) | | |
| BI-020 | SISSGGRPSYQDSVKG | 391 |
| CDR-2 (Kabat) | | |
| BI-020 | RGLHLDTGLYES | 392 |
| CDR-3 (Kabat) | | |
| BI-021 | EVQLVESGGGLVQPGGSLRLSCASPGSISTLYVMGWYRQAPGKQR | 81 |
| | DLVARITRGGSTSYANSVKGRFTISRDNVNNTINLQMNSLKPEDT | |
| | AVYYCYAQTAVGPDYWGQGTQVTVSS | |
| BI-021 | LYVMG | 393 |
| CDR-1 (Kabat) | | |
| BI-021 | RITRGGSTSYANSVKG | 394 |
| CDR-2 (Kabat) | | |
| BI-021 | QTAVGPDY | 395 |
| CDR-3 (Kabat) | | |
| BI-022 | QVQLVESGGGLVQAGGSLRLSCAASGSIFSTLNAIGWYRQAPGKQ | 82 |
| | AELVARITHDGRIVYGDSVKGRFTISRDNAKNTAYLQMNSLNPED | |
| | TAVYFCVAPGMVRGQGTQVTVSS | |
| BI-022 | TLNAIG | 396 |
| CDR-1 (Kabat) | | |
| BI-022 | RITHDGRIVYGDSVKG | 397 |
| CDR-2 (Kabat) | | |
| BI-022 | PGMV | 398 |
| CDR-3 (Kabat) | | |
| BI-023 | QVQLVESGGGLVQAGDSLRLSCTASGRTLTLSMVTVGWFRQGSGK | 83 |
| | EREFVAAISWRGGRSYVADDVKGRFTISRDNARNTVYLQMNSVKP | |
| | EDTAVYYCAAREAIQDLAWTANDFTYWGQGTQVTVSS | |
| BI-023 | LSMVTVG | 399 |
| CDR-1 (Kabat) | | |
| BI-023 | AISWRGGRSYVADDVKG | 400 |
| CDR-2 (Kabat) | | |
| BI-023 | REAIQDLAWTANDFTY | 401 |
| CDR-3 (Kabat) | | |
| BI-024 | QVQLVESGGGSVQAGGSLRLSCAASGRTENTKAIGWFRQAPGKER | 84 |
| | EFVAAISWGGGTIRYADSVKGRVTISRDDAKNTVYLQMNSLKPED | |
| | TAVYYFATRQLHIATLAADFDSRRQGTQVTVSS | |
| BI-024 | TKAIG | 402 |
| CDR-1 (Kabat) | | |
| BI-024 | AISWGGGTIRYADSVKG | 403 |
| CDR-2 (Kabat) | | |
| BI-024 | RQLHIATLAADFDS | 404 |
| CDR-3 (Kabat) | | |
| BI-025 | EVQLVESGGGLVQPGGSLRLSCAASGRTLSSYTMAWFRQAPGKER | 85 |
| | EFVATISPVGFIMDYADSVKGRFTISRDNTKNTVYLQMNSLKHED | |
| | TALYYCATDLGRKLGTQSCEYGYWGQGTQVTVSS | |
| BI-025 | SYTMA | 405 |
| CDR-1 (Kabat) | | |
| BI-025 | TISPVGFIMDYADSVKG | 406 |
| CDR-2 (Kabat) | | |
| BI-025 | DLGRKLGTQSCEYGY | 407 |
| CDR-3 (Kabat) | | |
| BI-026 | EVQLVESGGGLVQPGGSLRLSCVASGSTGSITSMAWYRQAPGKQR | 86 |
| | ELVASINSGGRPNYQESVKGRFTISRDNAENTLYLQMNSLSPEDT | |
| | AVYLCNLRGLRLDTGLYESWGQGTQVTVSS | |
| BI-026 | ITSMA | 408 |
| CDR-1 (Kabat) | | |
| BI-026 | SINSGGRPNYQESVKG | 409 |
| CDR-2 (Kabat) | | |
| BI-026 | RGLRLDTGLYES | 410 |
| CDR-3 (Kabat) | | |
| BI-027 | QVQLVESGGGSVQAGGSLRLSCAASGRTFSSRAMGWFRQAPGKER | 87 |
| | EFVAAISFGGGTIRYADSVKGRFTISRDDAKNTVYLQMNSLKPED | |
| | TAVYYCAARRLHIATLAADFDSWGQGTQVTVSS | |
| BI-027 | SRAMG | 411 |
| CDR-1 (Kabat) | | |
| BI-027 | AISFGGGTIRYADSVKG | 412 |
| CDR-2 (Kabat) | | |
| BI-027 | RRLHIATLAADFDS | 413 |
| CDR-3 (Kabat) | | |
| BI-028 | EVQLVESGGGLVQTGGSLRLSCAASGLINGYVMAWFRQAPGKERE | 88 |
| | FVSGIGWGSSRTYYADSVKGRFTISRDNAINTVALQMNSLKPEDT | |
| | AVYYCAAQGRISPIYTRANEYPYWGQGTQVTVSS | |
| BI-028 | YVMA | 414 |
| CDR-1 (Kabat) | | |
| BI-028 | GIGWGSSRTYYADSVKG | 415 |
| CDR-2 (Kabat) | | |
| BI-028 | QGRISPIYTRANEYPY | 416 |
| CDR-3 (Kabat) | | |
| BI-029 | QVQLVESGGGLVQPGGSLRLSCAASGFTFSSYGMSWYRQAPGKEH | 89 |
| | ELVAGISAGSTKYADSVKGRFTISRDNAKKTVYLQMNSLKPEDTA | |
| | VYYCSRWPRLFEDWGQGTQVTVSS | |
| BI-029 | SYGMS | 417 |
| CDR-1 (Kabat) | | |
| BI-029 | GISAGSTKYADSVKG | 418 |
| CDR-2 (Kabat) | | |
| BI-029 | WPRLFED | 419 |
| CDR-3 (Kabat) | | |
| BI-030 | EVQLVESGGGLVQPGGSLRLSCVASGDIGSMTSTGWYRQAPGKQR | 90 |
| | DLVASINSGGRPNYQDSVKGRFTISRDSAKNTVYLQMNSLKPEDT | |
| | AVYYCNLRGLVLSTGLYESWGQGTQVTVSS | |
| BI-030 | MTSTG | 420 |
| CDR-1 (Kabat) | | |
| BI-030 | SINSGGRPNYQDSVKG | 421 |
| CDR-2 (Kabat) | | |
| BI-030 | RGLVLSTGLYES | 422 |
| CDR-3 (Kabat) | | |
| BI-031 | QVQLVESGGGLVQPGGSLRLSCAAPGSIATLYVMGWYRQAPGKQR | 91 |
| | ELVARITRGGSTSYANAVKGRFTISRDNAKNTVNLQMNSLKPEDT | |
| | AIYYCNAQTAVGPDYWGQGTQVTVSS | |
| BI-031 | LYVMG | 423 |
| CDR-1 (Kabat) | | |
| BI-031 | RITRGGSTSYANAVKG | 424 |
| CDR-2 (Kabat) | | |
| BI-031 | QTAVGPDY | 425 |
| CDR-3 (Kabat) | | |
| BI-032 | EVQLVESGGGLVQAGGSLRPSCAASGRTFSNYNMGWFRQAPGKER | 92 |
| | ESVATISRSGVITDYADSVKGRFTISRDNAKNTVYLQMDSLKPED | |
| | TAVYYCAAARSPVWGRGPDEYDTWGQGTQVTVSS | |
| BI-032 | NYNMG | 426 |
| CDR-1 (Kabat) | | |
| BI-032 | TISRSGVITDYADSVKG | 427 |
| CDR-2 (Kabat) | | |
| BI-032 | ARSPVWGRGPDEYDT | 428 |
| CDR-3 (Kabat) | | |
| BI-033 | EVQLVESGGGLVQAGGSLRLSCAASGRTFSAYVMGWFRQTPGKGR | 93 |
| | EFVAAISTGGQISDYANSVKGRFTISRDNAKNTAYLQMNNLKPED | |
| | TAVYYCAANRENFLNRGAGDYEYWGQGTQVTVSS | |
| BI-033 | AYVMG | 429 |
| CDR-1 (Kabat) | | |
| BI-033 | AISTGGQISDYANSVKG | 430 |
| CDR-2 (Kabat) | | |
| BI-033 | NRENFLNRGAGDYEY | 431 |
| CDR-3 (Kabat) | | |
| BI-034 | QVQLVESGGGLVQPGGSLRLSCVASGSTGSITSMAWYRQAPGKQR | 94 |
| | ELVASINSGGRPNYQESVKGRFTISRDNAENTLYLQMNSLSPEDT | |
| | AVYLCNLRGLRLDTGLYESWGQGTQVTVSS | |
| BI-034 | ITSMA | 432 |
| CDR-1 (Kabat) | | |
| BI-034 | SINSGGRPNYQESVKG | 433 |
| CDR-2 (Kabat) | | |
| BI-034 | RGLRLDTGLYES | 434 |
| CDR-3 (Kabat) | | |
| BI-035 | QVQLVESGGGLVQPGGSLRLSCAASGSIAEIYVMGWYRQAPGKQR | 95 |
| | EIVATTPSSGRTNIADSVKGRFIISRDFVKNTVALQMNSLKPEDT | |
| | AVYYCYARLTPTSVASWGPGTQVTVSS | |
| BI-035 | IYVMG | 435 |
| CDR-1 (Kabat) | | |
| BI-035 | TTPSSGRTNIADSVKG | 436 |
| CDR-2 (Kabat) | | |
| BI-035 | RLTPTSVAS | 437 |
| CDR-3 (Kabat) | | |
| BI-036 | EVQLVESGGGSVQAGGSLRLSCATSGRGESTYAMGWFRQAPGKER | 96 |
| | EFVAAISFGGGTVRYVDSVKGRFTISRDDAKGTVYLQMNSLKPED | |
| | TAVYYCAARRLHIATLAADFGSWGQGTQVTVSS | |
| BI-036 | TYAMG | 438 |
| CDR-1 (Kabat) | | |
| BI-036 | AISFGGGTVRYVDSVKG | 439 |
| CDR-2 (Kabat) | | |
| BI-036 | RRLHIATLAADFGS | 440 |
| CDR-3 (Kabat) | | |
| BI-037 | EVQLVESGGGLVQAGDSLRLSCAASGRTFSTYVMGWFRQAPGKER | 97 |
| | EFVAYISTGGLISDHADSVKGRFTISRDNAKNTVYLQMNSLRPED | |
| | TAVYYCAAASRTRRPIATIKDEYDYWGQGTQVTVSS | |
| BI-037 | TYVMG | 441 |
| CDR-1 (Kabat) | | |
| BI-037 | YISTGGLISDHADSVKG | 442 |
| CDR-2 (Kabat) | | |
| BI-037 | ASRTRRPIATIKDEYDY | 443 |
| CDR-3 (Kabat) | | |
| BI-038 | EVQLVESGGGLVQAGDSLRLSCTASGRTLTLSMVTVGWFRQGSGK | 98 |
| | EREFVAAISWRGGRSSVADDVKGRFTISRDNARNTVYLQMNSVKP | |
| | EDTAVYYCAARTAIQDLAWTANDFTYWGQGTQVTVSS | |
| BI-038 | LSMVTVG | 444 |
| CDR-1 (Kabat) | | |
| BI-038 | AISWRGGRSSVADDVKG | 445 |
| CDR-2 (Kabat) | | |
| BI-038 | RTAIQDLAWTANDFTY | 446 |
| CDR-3 (Kabat) | | |
| BI-039 | QVQLVESGGGLVQAGGSLRLSCAASGRTFETIAMGWFRQVPGKER | 99 |
| | EFVAVIRGSGVATYYPDSVKGRFTISKDSAKNTVYLQMNNLKPED | |
| | TAVYYCAATTNRFKNTDYTTYDYWGQGTQVTVSS | |
| BI-039 | TIAMG | 447 |
| CDR-1 (Kabat) | | |
| BI-039 | VIRGSGVATYYPDSVKG | 448 |
| CDR-2 (Kabat) | | |
| BI-039 | TTNRFKNTDYTTYDY | 449 |
| CDR-3 (Kabat) | | |
| BI-040 | EVQLVESGGGSVQVGGFLRLSCVGSGRTLNMYNMGWFRQAPGKER | 100 |
| | EFVAAISGKGLISDYRDSVKGRFTISRDNARNTMYLQMNSLKPED | |
| | TAVYHCAAGQWSAGPFTRERSYEYWGQGTQVTVSS | |
| BI-040 | MYNMG | 450 |
| CDR-1 (Kabat) | | |
| BI-040 | AISGKGLISDYRDSVKG | 451 |
| CDR-2 (Kabat) | | |
| BI-040 | GQWSAGPFTRERSYEY | 452 |
| CDR-3 (Kabat) | | |
| BI-041 | EVQLVESGGGLVQAGGSLRLSCASSGRTFSNYNMGWFRQAPGKER | 101 |
| | ESVATISRSGVITDYADYVKGRFTISRDNAKNTVYLQMDSLKPED | |
| | TAVYYCAAARSPVWGRGPDEYDTWGQGTQVTVSS | |
| BI-041 | NYNMG | 453 |
| CDR-1 (Kabat) | | |
| BI-041 | TISRSGVITDYADYVKG | 454 |
| CDR-2 (Kabat) | | |
| BI-041 | ARSPVWGRGPDEYDT | 455 |
| CDR-3 (Kabat) | | |
| BI-042 | EVQLVESGGGSVQAGGSLRLSCAASGRTENTKAIGWFRQAPGKER | 102 |
| | EFVAAISWGGGTIRYADSVKGRVTISRDDAKNTVYLQMNSLKPED | |
| | TAVYYCAARQLHIATLAADFDSWGQGTQVTVSS | |
| BI-042 | TKAIG | 456 |
| CDR-1 (Kabat) | | |
| BI-042 | AISWGGGTIRYADSVKG | 457 |
| CDR-2 (Kabat) | | |
| BI-042 | RQLHIATLAADFDS | 458 |
| CDR-3 (Kabat) | | |
| BI-043 | EVQLVESGGGLVQAGGSLRLSCAASGRTYSTVAMGWFRQAPGKER | 103 |
| | EFVGAITWSVGNTAVADSVKGRFAISRDSAKNTVYLQMNSLKVED | |
| | TAVYYCASRTNIGAFNLFRENHYNYWGQGTQVTVSS | |
| BI-043 | TVAMG | 459 |
| CDR-1 (Kabat) | | |
| BI-043 | AITWSVGNTAVADSVKG | 460 |
| CDR-2 (Kabat) | | |
| BI-043 | RTNIGAFNLFRENHYNY | 461 |
| CDR-3 (Kabat) | | |
| BI-044 | EVQLVESGGGLVQPGGSLRLSCKASGSIGSVTSMGWYRQAPGKQR | 104 |
| | DLVASADSNGRTTFQDFVQGRFTISRDSAKNTWYLQMNSLKPEDT | |
| | AVYYCHLRGLQLTMGLYESWGQGTQVTVSS | |
| BI-044 | VTSMG | 462 |
| CDR-1 (Kabat) | | |
| BI-044 | SADSNGRTTFQDFVQG | 463 |
| CDR-2 (Kabat) | | |
| BI-044 | RGLQLTMGLYES | 464 |
| CDR-3 (Kabat) | | |
| BI-045 | EVQLVESGGGAVQAGGALNVSCAASGRAFSRTLMGWFRQAPGKER | 105 |
| | EFVAGISWVSVTPDYGNSVKGRFTISRDNAKSTVYLQMNSLKPED | |
| | TAVYYCAASQRYGTPRRRPNDYEYWGQGIQVTVSS | |
| BI-045 | RTLMG | 465 |
| CDR-1 (Kabat) | | |
| BI-045 | GISWVSVTPDYGNSVKG | 466 |
| CDR-2 (Kabat) | | |
| BI-045 | SQRYGTPRRRPNDYEY | 467 |
| CDR-3 (Kabat) | | |
| BI-046 | EVQLVESGGGSVQTGGSLRLSCKVSEGSFMRYNMGWFRQAPGKER | 106 |
| | DFVAAMSGALALIRYADSVKGRFTISRDNSKNTVYLDMNSLKPED | |
| | TAVYYCAADLEPQYWTKAGQRDTYDVWGQGTQVTVSS | |
| BI-046 | RYNMG | 468 |
| CDR-1 (Kabat) | | |
| BI-046 | AMSGALALIRYADSVKG | 469 |
| CDR-2 (Kabat) | | |
| BI-046 | DLEPQYWTKAGQRDTYDV | 470 |
| CDR-3 (Kabat) | | |
| BI-047 | EVQLVESGGGLVQAGGSLRLSCAASGSISSNIMGWFRQAPGKERE | 107 |
| | FVAVISRRGLILDYGDSVKGRFTMSRDNAKKAVYLQMNSLKPEDT | |
| | AVYYCAVGKTTDRFSEIPSDYDYWGQGTQVTVSS | |
| BI-047 | NIMG | 471 |
| CDR-1 (Kabat) | | |
| BI-047 | VISRRGLILDYGDSVKG | 472 |
| CDR-2 (Kabat) | | |
| BI-047 | GKTTDRFSEIPSDYDY | 473 |
| CDR-3 (Kabat) | | |
| BI-048 | EVQLVESGGGLVQPGGSLRLSCAASGRTFETIAMGWFRQAPGKGL | 108 |
| | EFVAVIRGSGVATYYAESVKGRFTISKDSSKNTVYLQMNSLRAED | |
| | TAVYYCAATTNRFKNTDYTTYDYWGQGTQVTVSS | |
| BI-048 | TIAMG | 474 |
| CDR-1 (Kabat) | | |
| BI-048 | VIRGSGVATYYAESVKG | 475 |
| CDR-2 (Kabat) | | |
| BI-048 | TTNRFKNTDYTTYDY | 476 |
| CDR-3 (Kabat) | | |
| BI-049 | EVQLVESGGGLVQPGGSLRLSCAASGRTFETIAMGWFRQAPGKGL | 109 |
| | EFVAVIRGSGVATYYAESVKGRFTISKDSSKNTVYLQMNSLRAED | |
| | TAVYYCAATTNRFKNREYTTYDYWGQGTQVTVSS | |
| BI-049 | TIAMG | 477 |
| CDR-1 (Kabat) | | |
| BI-049 | VIRGSGVATYYAESVKG | 478 |
| CDR-2 (Kabat) | | |
| BI-049 | TTNRFKNREYTTYDY | 479 |
| CDR-3 (Kabat) | | |
| BI-001 | GREFTSVSMA | 110 |
| CDR-1 (CCG & | | |
| Chothia) | | |
| BI-001 | ARISET | 111 |
| CDR-2 (Chothia) | | |
| BI-002 | GFTFSSYYMY | 112 |
| CDR-1 (CCG & | | |
| Chothia) | | |
| BI-002 | KPNGS | 113 |
| CDR-2 (Chothia) | | |
| BI-003 | GNQLSLYNMG | 114 |
| CDR-1 (CCG & | | |
| Chothia) | | |
| BI-003 | SRAGR | 115 |
| CDR-2 (Chothia) | | |
| BI-004 | GFSFSSSWMF | 116 |
| CDR-1 (CCG & | | |
| Chothia) | | |
| BI-004 | SPSGDY | 117 |
| CDR-2 (Chothia) | | |
| BI-005 | GFTFSNFVMK | 118 |
| CDR-1 (CCG & | | |
| Chothia) | | |
| BI-005 | DTTHE | 119 |
| CDR-2 (Chothia) | | |
| BI-006 | GRTFSRYNMG | 120 |
| CDR-1 (CCG & | | |
| Chothia) | | |
| BI-006 | IWSGGV | 121 |
| CDR-2 (Chothia) | | |
| BI-007 | GSIGSVVSWG | 122 |
| CDR-1 (CCG & | | |
| Chothia) | | |
| BI-007 | ASGGR | 123 |
| CDR-2 (Chothia) | | |
| BI-008 | GRPSRDYAMG | 124 |
| CDR-1 (CCG & | | |
| Chothia) | | |
| BI-008 | SSGGGF | 125 |
| CDR-2 (Chothia) | | |
| BI-009 | GFRFTPYTMG | 126 |
| CDR-1 (CCG & | | |
| Chothia) | | |
| BI-009 | SSVYS | 127 |
| CDR-2 (Chothia) | | |
| BI-010 | GRTLNMYNMG | 128 |
| CDR-1 (CCG & | | |
| Chothia) | | |
| BI-010 | SGKGLI | 129 |
| CDR-2 (Chothia) | | |
| BI-011 | GRTFSRWTMA | 130 |
| CDR-1 (CCG & | | |
| Chothia) | | |
| BI-011 | GWWNDS | 131 |
| CDR-2 (Chothia) | | |
| BI-012 | GRTLSSYTMA | 132 |
| CDR-1 (CCG & | | |
| Chothia) | | |
| BI-012 | SPVGFI | 133 |
| CDR-2 (Chothia) | | |
| BI-013 | GRTFSTYAVG | 134 |
| CDR-1 (CCG & | | |
| Chothia) | | |
| BI-013 | SWGGGT | 135 |
| CDR-2 (Chothia) | | |
| BI-014 | GRTFNMYVMG | 136 |
| CDR-1 (CCG & | | |
| Chothia) | | |
| BI-014 | SGEGLI | 137 |
| CDR-2 (Chothia) | | |
| BI-015 | GRDFDRSTMA | 138 |
| CDR-1 (CCG & | | |
| Chothia) | | |
| BI-015 | PSDIG | 139 |
| CDR-2 (Chothia) | | |
| BI-016 | GRTFSSRAMG | 140 |
| CDR-1 (CCG & | | |
| Chothia) | | |
| BI-016 | SFGGGT | 141 |
| CDR-2 (Chothia) | | |
| BI-017 | GRSLNDYIVG | 142 |
| CDR-1 (CCG & | | |
| Chothia) | | |
| BI-017 | SSGGYI | 143 |
| CDR-2 (Chothia) | | |
| BI-018 | GGTFSEYAMG | 144 |
| CDR-1 (CCG & | | |
| Chothia) | | |
| BI-018 | SRGAGR | 145 |
| CDR-2 (Chothia) | | |
| BI-019 | GRAFSRSLMG | 146 |
| CDR-1 (CCG & | | |
| Chothia) | | |
| BI-019 | SWVSVT | 147 |
| CDR-2 (Chothia) | | |
| BI-020 | GSIGSITSMA | 148 |
| CDR-1 (CCG & | | |
| Chothia) | | |
| BI-020 | SSGGR | 149 |
| CDR-2 (Chothia) | | |
| BI-021 | GSISTLYVMG | 150 |
| CDR-1 (CCG & | | |
| Chothia) | | |
| BI-021 | TRGGS | 151 |
| CDR-2 (Chothia) | | |
| BI-022 | GSIFSTLNAIG | 152 |
| CDR-1 (CCG & | | |
| Chothia) | | |
| BI-022 | THDGR | 153 |
| CDR-2 (Chothia) | | |
| BI-023 | GRTLTLSMVTVG | 154 |
| CDR-1 (CCG & | | |
| Chothia) | | |
| BI-023 | SWRGGR | 155 |
| CDR-2 (Chothia) | | |
| BI-024 | GRTENTKAIG | 156 |
| CDR-1 (CCG & | | |
| Chothia) | | |
| BI-024 | SWGGGT | 157 |
| CDR-2 (Chothia) | | |
| BI-025 | GRTLSSYTMA | 158 |
| CDR-1 (CCG & | | |
| Chothia) | | |
| BI-025 | SPVGFI | 159 |
| CDR-2 (Chothia) | | |
| BI-026 | GSTGSITSMA | 160 |
| CDR-1 (CCG & | | |
| Chothia) | | |
| BI-026 | NSGGR | 161 |
| CDR-2 (Chothia) | | |
| BI-027 | GRTFSSRAMG | 162 |
| CDR-1 (CCG & | | |
| Chothia) | | |
| BI-027 | SFGGGT | 163 |
| CDR-2 (Chothia) | | |
| BI-028 | GLTNGYVMA | 164 |
| CDR-1 (CCG & | | |
| Chothia) | | |
| BI-028 | GWGSSR | 165 |
| CDR-2 (Chothia) | | |
| BI-029 | GFTFSSYGMS | 166 |
| CDR-1 (CCG & | | |
| Chothia) | | |
| BI-029 | SAGS | 167 |
| CDR-2 (Chothia) | | |
| BI-030 | GDIGSMTSTG | 168 |
| CDR-1 (CCG & | | |
| Chothia) | | |
| BI-030 | NSGGR | 169 |
| CDR-2 (Chothia) | | |
| BI-031 | GSIATLYVMG | 170 |
| CDR-1 (CCG & | | |
| Chothia) | | |
| BI-031 | TRGGS | 171 |
| CDR-2 (Chothia) | | |
| BI-032 | GRTFSNYNMG | 172 |
| CDR-1 (CCG & | | |
| Chothia) | | |
| BI-032 | SRSGVI | 173 |
| CDR-2 (Chothia) | | |
| BI-033 | GRTFSAYVMG | 174 |
| CDR-1 (CCG & | | |
| Chothia) | | |
| BI-033 | STGGQI | 175 |
| CDR-2 (Chothia) | | |
| BI-034 | GSTGSITSMA | 176 |
| CDR-1 (CCG & | | |
| Chothia) | | |
| BI-034 | NSGGR | 177 |
| CDR-2 (Chothia) | | |
| BI-035 | GSIAEIYVMG | 178 |
| CDR-1 (CCG & | | |
| Chothia) | | |
| BI-035 | PSSGR | 179 |
| CDR-2 (Chothia) | | |
| BI-036 | GRGFSTYAMG | 180 |
| CDR-1 (CCG & | | |
| Chothia) | | |
| BI-036 | SFGGGT | 181 |
| CDR-2 (Chothia) | | |
| BI-037 | GRTFSTYVMG | 182 |
| CDR-1 (CCG & | | |
| Chothia) | | |
| BI-037 | STGGLI | 183 |
| CDR-2 (Chothia) | | |
| BI-038 | GRTLTLSMVTVG | 184 |
| CDR-1 (CCG & | | |
| Chothia) | | |
| BI-038 | SWRGGR | 185 |
| CDR-2 (Chothia) | | |
| BI-039 | GRTFETIAMG | 186 |
| CDR-1 (CCG & | | |
| Chothia) | | |
| BI-039 | RGSGVA | 187 |
| CDR-2 (Chothia) | | |
| BI-040 | GRTLNMYNMG | 188 |
| CDR-1 (CCG & | | |
| Chothia) | | |
| BI-040 | SGKGLI | 189 |
| CDR-2 (Chothia) | | |
| BI-041 | GRTFSNYNMG | 190 |
| CDR-1 (CCG & | | |
| Chothia) | | |
| BI-041 | SRSGVI | 191 |
| CDR-2 (Chothia) | | |
| BI-042 | GRTENTKAIG | 192 |
| CDR-1 (CCG & | | |
| Chothia) | | |
| BI-042 | SWGGGT | 193 |
| CDR-2 (Chothia) | | |
| BI-043 | GRTYSTVAMG | 194 |
| CDR-1 (CCG & | | |
| Chothia) | | |
| BI-043 | TWSVGN | 195 |
| CDR-2 (Chothia) | | |
| BI-044 | GSIGSVTSMG | 196 |
| CDR-1 (CCG & | | |
| Chothia) | | |
| BI-044 | DSNGR | 197 |
| CDR-2 (Chothia) | | |
| BI-045 | GRAFSRTLMG | 198 |
| CDR-1 (CCG & | | |
| Chothia) | | |
| BI-045 | SWVSVT | 199 |
| CDR-2 (Chothia) | | |
| BI-046 | EGSFMRYNMG | 200 |
| CDR-1 (CCG & | | |
| Chothia) | | |
| BI-046 | SGALAL | 20 |
| CDR-2 (Chothia) | | |
| BI-047 | GSISSNIMG | 202 |
| CDR-1 (CCG & | | |
| Chothia) | | |
| BI-047 | SRRGLI | 203 |
| CDR-2 (Chothia) | | |
| BI-048 | GRTFETIAMG | 204 |
| CDR-1 (CCG & | | |
| Chothia) | | |
| BI-048 | RGSGVA | 205 |
| CDR-2 (Chothia) | | |
| BI-049 | GRTFETIAMG | 206 |
| CDR-1 (CCG & | | |
| Chothia) | | |
| BI-049 | RGSGVA | 207 |
| CDR-2 (Chothia) | | |

### Linkers

Methods of linking molecules are well known in the art. The linker may be a peptide linker or a non-peptide linker. If the linker is a peptide linker, it may be composed of one or more amino acids. For peptide linkers, typically a small linker sequence of glycine and serine (termed a GS mini-linker) amino acids are used. The number of amino acids in the linker can vary, from 4 (GGGS) (SEQ ID NO:19), 6 (GGSGGS) (SEQ ID NO:20), 10 (GGGGSGGGGS) (SEQ ID NO:21), 15 (GGGGSGGGGSGGGGS) (SEQ ID NO:22), 20 (GGGGSGGGGSGGGGSGGGGS) (SEQ ID NO:23) or more.

In some embodiments, the linker is between 5 and 20 amino acids in length. In other embodiments, the linker is rich in amino acid residues G and S. In another embodiment, the linker is between 5 and 20 amino acids in length and is rich in amino acid residues G and S. In another embodiment, the linker only includes the amino acid residues G and S. In another embodiment, the linker is between 2 and 20 amino acids in length and only includes the amino acid residues G and S.

Peptide linkers, as envisaged herein, are (poly)peptide linkers of at least 1 amino acid in length. Preferably, the linkers are 1 to 100 amino acids in length. More preferably, the linkers are 5 to 50 amino acids in length, more preferably 10 to 40 amino acids in length, and even more preferably, the linkers are 15 to 30 amino acids in length. Non-limiting examples of often used small linkers include sequences of glycine and serine amino acids, termed GS mini-linker. Preferred examples of linker sequences are Gly/Ser linkers of different length such as (glyₓser_{y})_{z} linkers, including (gly₄ser)₃, (gly₄ser)₄, (gly₄ser), (gly₃ser), gly₃, and (gly₃ser₂)₃. The number of amino acids in these linkers can vary, for example, they can be 4 (e.g., GGGS) (SEQ ID NO:19), 6 (e.g., GGSGGS) (SEQ ID NO:20), 7 (e.g., GGGSGGS), or multiples thereof, such as e.g. two or three or more repeats of these four/six amino acids. Most preferably, such GS mini-linkers have 20 amino acids and the sequence GGGGSGGGGSGGGGSGGGGS (SEQ ID NO:23). Further examples of such linkers include GGGGSGGGG (SEQ ID NO:24), GSGG (SEQ ID NO:25), or GGGGSGGGGSGGGGSGGGGSGGGGS (SEQ ID NO:26).

Further examples of linkers include the following:

5GS linker:
(SEQ ID NO: 27)
GGGGS
7GS linker:
(SEQ ID NO: 28)
SGGSGGS
8GS linker:
(SEQ ID NO: 29)
GGGSGGGS
9GS linker:
(SEQ ID NO: 30)
GGGGSGGGS
10GS linker:
(SEQ ID NO: 31)
GGGGSGGGGS
15GS linker:
(SEQ ID NO: 32)
GGGGSGGGGSGGGGS
18GS linker:
(SEQ ID NO: 33)
GGGGSGGGGSGGGGGGGS
20GS linker:
(SEQ ID NO: 34)
GGGGSGGGGSGGGGSGGGGS
25GS linker:
(SEQ ID NO: 35)
GGGGSGGGGSGGGGSGGGGSGGGGS
30GS linker:
(SEQ ID NO: 36)
GGGGSGGGGSGGGGSGGGGSGGGGSGGGGS
35GS linker:
(SEQ ID NO: 37)
GGGGSGGGGSGGGGSGGGGSGGGGSGGGGSGGGGS

Said linker can be also a variant as described in Holliger et al. (1993), Proc. Natl. Acad. Sci. USA 90:6444-6448. Other linkers that can be used for the present invention are described by Alfthan et al. (1995), Protein Eng. 8:725-731, Choi et al. (2001), Eur. J. Immunol. 31:94-106, Hu et al. (1996), Cancer Res. 56:3055-3061, Kipriyanov et al. (1999), J. Mol. Biol. 293:41-56 and Roovers et al. (2001), Cancer Immunol. Immunother. 50:51-59.

In a preferred embodiment, the non-cleavable linker is selected from any of the aforementioned GS linkers. In a further preferred embodiment, the non-cleavable linker is between 2 and 20 amino acids in length and only includes the amino acid residues G and S.

### Binding Moiety

The binding moiety may serve to promote the accumulation or retainment of the IL-12 Fc fusion protein and/or the cleavage product in the TME, preferably the ECM and more preferably within the vicinity of the tumor. The binding moiety may be placed either on the first or the second polypeptide chain of the IL-12 Fc fusion protein. Hence, in a preferred embodiment the binding moiety is an extracellular matrix binding moiety.

In some instances it may be desirable to place the binding moiety on the first polypeptide chain. If the binding moiety is placed on the first polypeptide chain this may additionally promote the accumulation and/or retainment of the cleavage product in the TME. In those instances when the binding moiety is placed on the first polypeptide chain, the binding moiety may be linked to the C-terminus of the IL-12p35 subunit or to the C-terminus of the IL-12p40 subunit. In both cases the binding moeity may be linked directly to the C-terminus or optionally via a polypeptide linker, such as any of those polypeptide linkers as disclosed herein. The binding moiety may also be placed between the IL-12p35 subunit and the IL-12p40 subunit. In this case the binding moiety may be optionally flanked on one or both sides by a polypeptide linker or linkers. This may result in different configurations, such as IL-12p35(binding moiety) IL-12p40 or IL-12p40(binding moiety)IL-12p35, wherein the binding moiety is linked directly at its N-terminus and C-terminus to the respective IL-12 subunit. Another configuration may include one or more linkers, such as IL-12p35(linker)(binding moiety)IL-12p40, IL-12p35(linker)(binding moiety)(linker) IL-12p40, IL-12p35(binding moiety)(linker)IL-12p40, IL-12p35(linker)(binding moiety)(linker) IL-12p40, IL-12p40(linker)(binding moiety)IL-12p35, IL-12p40(linker)(binding moiety)(linker)IL-12p35, IL-12p40(binding moiety)(linker)IL-12p35, IL-12p40(linker)(binding moiety)(linker)IL-12p35. In any of those configurations the linker may be a polypeptide linker, such as any of those polypeptide linkers as disclosed herein. In these configurations the binding moiety will expressed together with the IL-12 subunits in a single-chain and will remain with the IL-12 cytokine after cleavage in the TME.

In some other instances it may be desirable to place the binding moiety on the second polypeptide chain. This way the properties of the binding moiety will readily apply for the IL-12 Fc fusion protein but after cleavage the cleavage product is not additionally accumulated and/or retained in the TME. In some instances the binding moiety may be linked to the C-terminus of the masking moiety. The binding moeity may be linked directly to the C-terminus or optionally via a polypeptide linker, such as any of those polypeptide linkers as disclosed herein.

In some instances the binding moiety may be placed on the N-terminus of the first or the second Fc domain. The binding moeity may be linked directly to the N-terminus or optionally via a polypeptide linker, such as any of those polypeptide linkers as disclosed herein.

The binding moieties are selected from the list consisting of a collagen binding moiety, a heparin binding moiety and a fibronectin binding moiety. The binding moieties as disclosed herein have binding specificity for collagen, heparin, or fibronectin. In relation to the present invention, the binding moieties are derived from polypeptides or portions thereof that bind to respectively collagen, heparin, or fibronectin.

In a preferred embodiment the binding moiety is a collagen binding moiety, and more preferably a collagen I binding moiety. In a further preferred embodiment the collagen I binding moiety is placed at the C-terminus of the IL-12p35 subunit and/or the IL-12p40, e.g. IL-12p35-IL-12p40(binding moiety), IL-12p35(linker)IL-12p40(binding moiety), IL-12p35(linker) IL-12p40(linker) (binding moiety), IL-12p35-IL-12p40(linker)(binding moiety), IL-12p40-IL-12p35(binding moiety), IL-12p40(linker)IL-12p35(binding moiety), IL-12p40(linker)IL-12p35(linker)(binding moiety), or IL-12p40-IL-12p35(linker)(binding moiety).

### Collagen Binding Moiety

Collagen is the major component of the tumor microenvironment and participates in cancer fibrosis. Collagen biosynthesis can be regulated by cancer cells through mutated genes, transcription factors, signaling pathways and receptors; furthermore, collagen can influence tumor cell behavior through integrins, discoidin domain receptors, tyrosine kinase receptors, and some signaling pathways. Cancer associated fibroblasts produce high level of extra cellular matrix proteins (ECM) in the TME leading to hyper-expression of various types of collagen in many tumor types. The role of collagen in cancer has been extensively reviewed, including the relationship of collagens and proteases, such as MMP's that work together to modulate the TME (Xu, S., Xu, H., Wang, W. et al. The role of collagen in cancer: from bench to bedside. J Transl Med 17, 309 (2019).

The collagen superfamily comprises 28 members numbered with Roman numerals in vertebrates (I-XXVIII). The common structural feature of collagens is the presence of a triple helix that can range from most of their structure (96% for collagen I) to less than 10% (collagen XII). The diversity of the collagen family is further increased by the existence of several a chains, several molecular isoforms and supramolecular structures for a single collagen type, and the use of alternative promoters and alternative splicing.

Amongst the different collagens, the type I collagen is the most abundant protein in mammals. The fundamental structural unit of type I collagen is a long (300 nm), thin (1.5 nm-diameter) protein that consists of three coiled subunits: two alpha1 (I) chains and one alpha2 (I). Each chain contains 1050 amino acids wound around one another in a characteristic right-handed triple helix. In humans type I collagen is encoded by the COL1A1 and COL1A2 genes. The COL1A1 gene encodes the pro-alpha1 chain of type I collagen. The COL1A2 gene pro-alpha2 chain of type I collagen, whose triple helix comprises two alpha1 chains and one alpha2 chain. Type I is a fibril-forming collagen found in most connective tissues and is abundant in bone, cornea, dermis and tendon.

An exemplary amino acid sequence for the human alpha 1 chain precursor of type I collagen is set forth in SEQ ID NO:38 (NCBI Reference Sequence: NP 000079.2). An exemplary amino acid sequence for the human alpha2 chain precursor of type I collagen is set forth in SEQ ID NO:39 (NCBI Reference Sequence: NP 000000.2)

In some embodiments, the collagen binding moiety comprises one or more (e.g., two, three, four, five, six, seven, eight, nine, ten or more) leucine-rich repeats which bind collagen. In some embodiments, the collagen-binding moeity comprises a proteoglycan. In some embodiments, the collagen-binding moeity comprises a proteoglycan, wherein the proteoglycan is selected from the group consisting of: decorin, biglycan, testican, bikunin, fibromodulin, lumican, chondroadherin, keratin, ECM2, epiphycan, asporin, PRELP, keratocan, osteoadherin, opticin, osteoglycan, nyctalopin, Tsukushi, podocan, podocan-like protein 1 versican, perlecan, nidogen, neurocan, aggrecan, and brevican.

In some embodiments, the collagen-binding moeity comprises a class I small leucine-rich proteoglycan (SLRP). In some embodiments, the collagen-binding domain comprises a class II SLRP. In some embodiments, the collagen-binding domain comprises a class III SLRP. In some embodiments, the collagen-binding domain comprises a class IV SLRP. In some embodiments, the collagen-binding domain comprises a class V SLRP. In some embodiments, the collagen-binding domain comprises one or more leucine-rich repeats from a human proteoglycan Class II member of the small leucine-rich proteoglycan (SLRP) family. In some embodiments, the SLRP is selected from lumican, decorin, biglycan, fibromodulin, keratin, epiphycan, asporin and osteoglycin. In some embodiments, the SLRP is lumican.

It is also hypothesized that collagen rich tumor tissues will show higher activity of MMPs such as MMP2 and MMP9, which are collagenases, which may further contribute to faster cleavage of the IL-12 Fc fusion protein.

In some embodiments, the IL-12 Fc fusion proteins comprises a collagen binding moeity that specifically binds collagen. In some embodiments, the collagen binding moeity specifically binds human type I collagen and/or human type IV collagen. In some embodiments, the collagen binding moeity binds human type I collagen. In some embodiments, the collagen binding moeity binds human type IV collagen. In some embodiments, the collagen binding moeity specifically binds human type I collagen and human type IV collagen. In some embodiments, the collagen binding moeity specifically binds human type I collagen or human type IV collagen.

In a further embodiment, the disclosure provides IL-12 Fc fusion proteins, wherein the collagen binding moiety binds to collagen IV and has the amino acid sequence KLWVLPK (SEQ ID NO:40).

The binding of a collagen binding moeity to collagen can be determined by methods known in the art. In some embodiments, a collagen binding moiety is determined by its ability to compete with a known or reference collagen binding protein for binding to collagen. In some embodiments, a collagen binding moiety is derived from a naturally occurring collagen binding protein or collagen receptor.

In some embodiments, the IL-12 Fc fusion proteins specifically bind collagen with an affinity (KD) of less than about 500 M as determined by a collagen-binding assay. In some embodiments, the IL-12 Fc fusion proteins comprise a collagen binding moeity that specifically binds collagen with an affinity (KD) of less than about 100 μM as determined by a collagen binding assay. In some embodiments, the IL-12 Fc fusion protein comprises a collagen binding moiety that specifically binds collagen with an affinity (KD) of less than about 1 μM as determined by a collagen binding assay. In some embodiments, the IL-12 Fc fusion proteins comprises a collagen binding moiety that specifically binds collagen with an affinity (KD) of less than about 500 nM as determined by a collagen binding assay. In some embodiments, the collagen binding moiety specifically binds collagen with an affinity (KD) of about 0.1-500 UM, 0.1-100 UM, or 0.1-1 μM as determined by a collagen binding assay. In some embodiments, the collagen binding moiety specifically binds collagen with an affinity (KD) of about 100-1000 nM, 100-1000 nM, 100-800 nM, 100-600 nM, or 100-500 nM as determined by a collagen binding assay.

In some embodiments, the collagen binding assay determines a binding affinity of the collagen binding moeity for collagen. In some embodiments, the collagen binding assay determines a binding affinity of the collagen binding moiety for type I collagen. In some embodiments, the collagen binding assay determines a binding affinity for type IV collagen.

In some embodiments, the collagen binding assay is an ELISA. Methods and techniques to perform a collagen-binding ELISA are known in the art (see e.g., Smith et al., (2000) J Biol Chem 275:4205-4209). Accordingly, in some embodiments, the IL-12 Fc fusion proteins comprise a collagen binding moeity that specifically binds collagen with an affinity (KD) of less than about 500 μM as determined by an ELISA. In some embodiments, the IL-12 Fc fusion proteins comprise a collagen binding moeity that specifically binds collagen with an affinity (KD) of less than about 100 UM as determined by an ELISA. In some embodiments, the IL-12 Fc fusion protein comprise a collagen binding moiety that specifically binds collagen with an affinity (KD) of less than about 1 μM as determined by an ELISA. In some embodiments, the IL-12 Fc fusion proteins comprise a collagen binding moiety that specifically binds collagen with an affinity (KD) of less than about 500 nM as determined by an ELISA. In some embodiments, the collagen binding moiety specifically binds collagen with an affinity (KD) of about 0.1-500 UM, 0.1-100 UM, or 0.1-1 μM as determined by an ELISA. In some embodiments, the collagen binding moiety specifically binds collagen with an affinity (KD) of about 100-1000 nM, 100-1000 nM, 100-800 nM, 100-600 nM, or 100-500 nM as determined by an ELISA.

In some embodiments, the collagen binding assay is a surface plasmon resonance (SPR) assay. Methods and techniques to perform a collagen binding SPR assay are known in the art (see e.g., Saenko et al., (2002) Anal Biochem 302(2):252-262). Accordingly, in some embodiments, the IL-12 Fc fusion proteins comprise a collagen binding moeity that specifically binds collagen with an affinity (KD) of less than about 500 μM as determined by an SPR assay. In some embodiments, the IL-12 Fc fusion proteins comprise a collagen binding moeity that specifically binds collagen with an affinity (KD) of less than about 100 μM as determined by an SPR assay. In some embodiments, the IL-12 Fc fusion protein comprise a collagen binding moiety that specifically binds collagen with an affinity (KD) of less than about 1 μM as determined by an SPR assay. In some embodiments, the IL-12 Fc fusion proteins comprise a collagen binding moiety that specifically binds collagen with an affinity (KD) of less than about 500 nM as determined by an SPR assay. In some embodiments, the collagen binding moiety specifically binds collagen with an affinity (KD) of about 0.1-500 UM, 0.1-100 μM, or 0.1-1 μM as determined by an SPR assay. In some embodiments, the collagen binding moiety specifically binds collagen with an affinity (KD) of about 100-1000 nM, 100-1000 nM, 100-800 nM, 100-600 nM, or 100-500 nM as determined by an SPR assay.

The phrase “surface plasmon resonance” includes an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, NI). For further descriptions, see Jonsson, U., et al. (1993) Ann. Biol. Clin. 51: 19-26; Jonsson, U., et al. (1991) Biotechniques 11:620-627; Johnsson, B., et al. (1995) J. Mol. Recognit. 8: 125-131; and Johnnson, B., et al. (1991) Anal. Biochem. 198:268-277.

In some embodiments, the IL-12 Fc fusion proteins comprise a collagen binding moiety that specifically binds collagen and does not specifically bind to one or more non-collagen extracellular matrix (ECM) components including, but not limited to, fibronectin, heparin, vitronectin, tenascin C, osteopontin and fibrinogen. In some embodiments, the collagen binding moiety binds to collagen with a lower KD than to one or more non-collagen ECM components. In some embodiments, the KD of the collagen binding moiety for type I collagen is less than the KD of the collagen binding moiety for an extracellular matrix component selected from fibronectin, heparin, vitronectin, osteopontin, tenascin C, or fibrinogen. In some embodiments, the KD of the collagen binding moiety for type I collagen is less than the KD of the collagen binding moiety for any other type of collagen. In some embodiments, the collagen binding moiety binds to collagen with about 10 percent, about 20 percent, about 30 percent, about 40 percent, about 50 percent, about 60 percent, about 70 percent, about 80 percent, about 90 percent, about 99 percent lower KD than to one or more non-collagen ECM components. In some embodiments, the collagen binding moeity binds to collagen with about 2-fold, about 3-fold, about 4-fold, about 5-fold, about 10-fold lower KD than to one or more non-collagen ECM components.

In some embodiments, the collagen binding moiety binds to type I collagen with a lower KD than to type IV collagen. In some embodiments, the collagen binding moiety competes with a reference collagen binding moiety for binding to collagen. In some embodiments, the collagen binding moiety competes with a reference collagen binding moiety for binding to type I collagen.

In some embodiments, the reference collagen binding moiety comprises one or more (e.g., two, three, four, five, six, seven, eight, nine, ten or more) leucine-rich repeats which bind collagen. In some embodiments, the reference collagen-binding domain comprises a proteoglycan. In some embodiments, the reference collagen binding moiety comprises a proteoglycan, wherein the proteoglycan is selected from the group consisting of: decorin, biglycan, fibromodulin, lumican, chondroadherin, asporin, PRELP, osteoadherin/osteomodulin, opticin, osteoglycin/mimecan, podocan, perlecan, nidogen. In some embodiments, the reference collagen binding moiety is lumican. In some embodiments, the reference collagen binding moiety comprises a class I small leucine-rich proteoglycan (SLRP). SLRPs are known to bind collagen (Chen and Birk (2013) FEBS Journal 2120-2137). In some embodiments, the reference collagen binding moiety comprises a class II SLRP. In some embodiments, the reference collagen binding moiety comprises a class III SLRP. In some embodiments, the reference collagen binding moiety comprises a class IV SLRP. In some embodiments, the reference collagen binding moiety comprises a class V SLRP

In some embodiments, the reference collagen binding moiety comprises the leukocyte associated immunoglobulin-like receptor 1 (LAIR-1) protein.

In some embodiments, the reference collagen binding moiety comprises the leukocyte associated immunoglobulin-like receptor 2 (LAIR-2) protein. In some embodiments, the reference collagen binding moiety comprises Glycoprotein IV.

In its broadest form, the disclosure provides for an IL-12 Fc fusion protein, wherein the collagen binding moiety binds to collagen. Preferably, the collagen binding moiety binds specifically to type I collagen.

In a further preferred embodiment, the disclosure provides IL-12 Fc fusion proteins, wherein the collagen binding moiety binds to collagen I and has the sequence LxxLxLxxN (SEQ ID NO:41), wherein L is Leucine and N is Asparagine and x is any amino acid.

In a further embodiment, the disclosure provides an IL-12 Fc fusion protein, wherein the collagen binding moiety comprises or consists of any of the following sequences: LSELRLHEN (SEQ ID NO:42), LTELHLDNN (SEQ ID NO:43), LSELRLHNN (SEQ ID NO:44), LSELRLHAN (SEQ ID NO:45), LRELHLNNN (SEQ ID NO:46), or LRELHLDNN (SEQ ID NO:47). In a related most preferred embodiment the collagen binding moiety comprises or consists of the sequence LRELHLDNN (SEQ ID NO:47).

In another embodiment, the disclosure provides an IL-12 Fc fusion protein, wherein the collagen binding moiety has a length of 20 amino acids (aa), 19aa, 18aa, 17aa, 16aa, 15aa, 14aa, 13aa, 12aa, 11aa, 10a, or 9aa and comprises the sequence LxxLxLxxN (SEQ ID NO:41), wherein L is Leucine and N is Asparagine and x is any amino acid.

### Fibronectin Binding Moiety

Fibronectin is a high-molecular weight (˜500-˜ 600 kDa) glycoprotein of the extracellular matrix that binds to membrane-spanning receptor proteins called integrins. Fibronectin also binds to other extracellular matrix proteins such as collagen, fibrin, and heparan sulfate proteoglycans (e.g. syndecans). Fibronectin exists as a protein dimer, consisting of two nearly identical monomers linked by a pair of disulfide bonds. The fibronectin protein is produced from a single gene, but alternative splicing of its pre-mRNA leads to the creation of several isoforms. Two types of fibronectin are present in vertebrates: soluble plasma fibronectin (formerly called “cold-insoluble globulin”, or CIg) is a major protein component of blood plasma (300 μg/ml) and is produced in the liver by hepatocytes insoluble cellular fibronectin is a major component of the extracellular matrix. It is secreted by various cells, primarily fibroblasts, as a soluble protein dimer and is then assembled into an insoluble matrix in a complex cell-mediated process. Fibronectin plays a major role in cell adhesion, growth, migration, and differentiation, and it is important for processes such as wound healing and embryonic development. Altered fibronectin expression, degradation, and organization has been associated with a number of pathologies, including cancer, arthritis, and fibrosis. It has been proposed hat fibronectin expression may be upregulated in cancer tissues.

An exemplary amino acid sequence for fibronectin is set forth in SEQ ID NO:48 (UNIPROT Reference Sequence: P02751).

Accordingly, in some embodiments, the IL-12 Fc fusion proteins comprise a fibronectin binding moeity that specifically binds fibronectin. In some embodiments, the fibronectin binding moeity specifically binds human fibronectin.

The binding of a fibronectin binding moeity to fibronectin can be determined by methods known in the art. In some embodiments, a fibronectin binding moiety is determined by its ability to compete with a known or reference fibronectin binding protein for binding to fibronectin. In some embodiments, a fibronectin binding moiety is derived from a naturally occurring fibronectin binding protein or fibronectin receptor.

In some embodiments, the IL-12 Fc fusion proteins specifically bind fibronectin with an affinity (KD) of less than about 500 μM as determined by a fibronectin-binding assay. In some embodiments, the IL-12 Fc fusion proteins comprise a fibronectin binding moeity that specifically binds fibronectin with an affinity (KD) of less than about 100 UM as determined by a fibronectin binding assay. In some embodiments, the IL-12 Fc fusion protein comprise a fibronectin binding moiety that specifically binds fibronectin with an affinity (KD) of less than about 1 μM as determined by a fibronectin binding assay. In some embodiments, the IL-12 Fc fusion proteins comprise a fibronectin binding moiety that specifically binds fibronectin with an affinity (KD) of less than about 500 nM as determined by a fibronectin binding assay. In some embodiments, the fibronectin binding moiety specifically binds fibronectin with an affinity (KD) of about 0.1-500 UM, 0.1-100 UM, or 0.1-1 μM as determined by a fibronectin binding assay. In some embodiments, the fibronectin binding moiety specifically binds fibronectin with an affinity (KD) of about 100-1000 nM, 100-1000 nM, 100-800 nM, 100-600 nM, or 100-500 nM as determined by a fibronectin binding assay.

In some embodiments, the fibronectin binding assay determines a binding affinity of the fibronectin binding moeity for fibronectin.

In some embodiments, the fibronectin binding assay is an ELISA. Methods and techniques to perform a fibronectin-binding ELISA are known in the art (see e.g., Gao et al., (1998) European Journal of Pharmaceutics and Biopharmaceutics, Volume 45, Issue 3, Pages 275-284). Accordingly, in some embodiments, the IL-12 Fc fusion proteins comprises a fibronectin binding moeity that specifically binds fibronectin with an affinity (KD) of less than about 500 μM as determined by an ELISA. In some embodiments, the IL-12 Fc fusion proteins comprises a fibronectin binding moeity that specifically binds fibronectin with an affinity (KD) of less than about 100 μM as determined by an ELISA. In some embodiments, the IL-12 Fc fusion protein comprises a fibronectin binding moiety that specifically binds fibronectin with an affinity (KD) of less than about 1 μM as determined by an ELISA. In some embodiments, the IL-12 Fc fusion proteins comprises a fibronectin binding moiety that specifically binds fibronectin with an affinity (KD) of less than about 500 nM as determined by an ELISA. In some embodiments, the fibronectin binding moiety specifically binds fibronectin with an affinity (KD) of about 0.1-500 UM, 0.1-100 UM, or 0.1-1 μM as determined by an ELISA. In some embodiments, the fibronectin binding moiety specifically binds fibronectin with an affinity (KD) of about 100-1000 nM, 100-1000 nM, 100-800 nM, 100-600 nM, or 100-500 nM as determined by an ELISA.

In some embodiments, the fibronectin binding assay is a surface plasmon resonance (SPR) assay. Methods and techniques to perform a fibronectin binding SPR assay are known in the art (see e.g., Makogonenko et al, (2002) Biochemistry, 41, 25, 7907-7913). Accordingly, in some embodiments, the IL-12 Fc fusion proteins comprises a fibronectin binding moeity that specifically binds fibronectin with an affinity (KD) of less than about 500 UM as determined by an SPR assay. In some embodiments, the IL-12 Fc fusion proteins comprises a fibronectin binding moeity that specifically binds fibronectin with an affinity (KD) of less than about 100 μM as determined by an SPR assay. In some embodiments, the IL-12 Fc fusion protein comprises a fibronectin binding moiety that specifically binds fibronectin with an affinity (KD) of less than about 1 μM as determined by an SPR assay. In some embodiments, the IL-12 Fc fusion proteins comprises a fibronectin binding moiety that specifically binds fibronectin with an affinity (KD) of less than about 500 nM as determined by an SPR assay. In some embodiments, the fibronectin binding moiety specifically binds fibronectin with an affinity (KD) of about 0.1-500 UM, 0.1-100 UM, or 0.1-1 μM as determined by an SPR assay. In some embodiments, the fibronectin binding moiety specifically binds fibronectin with an affinity (KD) of about 100-1000 nM, 100-1000 nM, 100-800 nM, 100-600 nM, or 100-500 nM as determined by an SPR assay.

In some embodiments, the IL-12 Fc fusion proteins comprises a fibronectin binding moiety that specifically binds fibronectin and does not specifically bind to one or more non-fibronectin extracellular matrix (ECM) components including, but not limited to, collagen, heparin, vitronectin, tenascin C, osteopontin and fibrinogen. In some embodiments, the fibronectin binding moiety binds to fibronectin with a lower KD than to one or more non-collagen ECM components. In some embodiments, the KD of the fibronectin binding moiety for fibronectin is less than the KD of the fibronectin binding moiety for an extracellular matrix component selected from collagen, heparin, vitronectin, osteopontin, tenascin C, or fibrinogen. In some embodiments, the fibronectin binding moiety binds to fibronectin with about 10 percent, about 20 percent, about 30 percent, about 40 percent, about 50 percent, about 60 percent, about 70 percent, about 80 percent, about 90 percent, about 99 percent lower KD than to one or more non-collagen ECM components. In some embodiments, the fibronectin binding moeity binds to fibronectin with about 2-fold, about 3-fold, about 4-fold, about 5-fold, about 10-fold lower KD than to one or more non-collagen ECM components.

In some embodiments, the fibronectin binding moiety competes with a reference fibronectin binding moiety for binding to fibronectin. In some embodiments, the reference fibronectin binding moiety comprises a peptide as disclosed e.g. by Sipes et al., (1993) Journal Cell Biol., 121(2):469-77.

In its broadest form, the disclosure provides for an IL-12 Fc fusion protein, wherein the fibronectin binding moiety binds to fibronectin.

In a further preferred embodiment, the disclosure provides IL-12 Fc fusion proteins, wherein the fibronectin binding moiety binds to fibronectin and has the sequence GGWSHW (SEQ ID NO:49).

In another embodiment, the disclosure provides an IL-12 Fc fusion protein, wherein the fibronectin binding moiety has a length of 20 amino acids (aa), 19aa, 18aa, 17aa, 16aa, 15aa, 14aa, 13aa, 12aa, 11aa, 10a, or 9aa and comprises the sequence GGWSHW (SEQ ID NO:49).

### Heparin Binding Moiety

Heparin, also known as unfractionated heparin (UFH), is a medication and naturally occurring glycosaminoglycan. Native heparin is a polymer with a molecular weight ranging from 3 to 30 kDa, although the average molecular weight of most commercial heparin preparations is in the range of 12 to 15 kDa. Heparin is a member of the glycosaminoglycan family of carbohydrates (which includes the closely related molecule heparan sulfate) and consists of a variably sulfated repeating disaccharide unit.

Accordingly, in some embodiments, the IL-12 Fc fusion proteins comprise a heparin binding moeity that specifically binds heparin. In some embodiments, the heparin binding moeity specifically binds human heparin.

The binding of a heparin binding moeity to heparin can be determined by methods known in the art. In some embodiments, a heparin binding moiety is determined by its ability to compete with a known or reference heparin binding protein for binding to heparin. In some embodiments, a heparin binding moiety is derived from a naturally occurring heparin binding protein or heparin receptor.

In some embodiments, the IL-12 Fc fusion proteins specifically binds heparin with an affinity (KD) of less than about 500 UM as determined by a heparin-binding assay. In some embodiments, the IL-12 Fc fusion proteins comprise a heparin binding moeity that specifically binds heparin with an affinity (KD) of less than about 100 μM as determined by a heparin binding assay. In some embodiments, the IL-12 Fc fusion protein comprise a heparin binding moiety that specifically binds heparin with an affinity (KD) of less than about 1 μM as determined by a heparin binding assay. In some embodiments, the IL-12 Fc fusion proteins comprise a heparin binding moiety that specifically binds heparin with an affinity (KD) of less than about 500 nM as determined by a heparin binding assay. In some embodiments, the heparin binding moiety specifically binds heparin with an affinity (KD) of about 0.1-500 μM, 0.1-100 μM, or 0.1-1 μM as determined by a heparin binding assay. In some embodiments, the heparin binding moiety specifically binds heparin with an affinity (KD) of about 100-1000 nM, 100-1000 nM, 100-800 nM, 100-600 nM, or 100-500 nM as determined by a heparin binding assay.

In some embodiments, the heparin binding assay determines a binding affinity of the heparin binding moeity for heparin.

In some embodiments, the heparin binding assay is an ELISA. Methods and techniques to perform a heparin-binding ELISA are known in the art. Accordingly, in some embodiments, the IL-12 Fc fusion proteins comprise a heparin binding moeity that specifically binds fibronectin with an affinity (KD) of less than about 500 μM as determined by an ELISA. In some embodiments, the IL-12 Fc fusion proteins comprise a heparin binding moeity that specifically binds heparin with an affinity (KD) of less than about 100 UM as determined by an ELISA. In some embodiments, the IL-12 Fc fusion proteins comprise a heparin binding moiety that specifically binds heparin with an affinity (KD) of less than about 1 μM as determined by an ELISA. In some embodiments, the IL-12 Fc fusion proteins comprise a heparin binding moiety that specifically binds heparin with an affinity (KD) of less than about 500 nM as determined by an ELISA. In some embodiments, the heparin binding moiety specifically binds heparin with an affinity (KD) of about 0.1-500 UM, 0.1-100 μM, or 0.1-1 μM as determined by an ELISA. In some embodiments, the heparin binding moiety specifically binds heparin with an affinity (KD) of about 100-1000 nM, 100-1000 nM, 100-800 nM, 100-600 nM, or 100-500 nM as determined by an ELISA.

In some embodiments, the heparin binding assay is a surface plasmon resonance (SPR) assay. Methods and techniques to perform a heparin binding SPR assay are known in the art (see e.g., Rusnati et al., (2016) Methods Mol Biol., 1464:73-84). Accordingly, in some embodiments, the IL-12 Fc fusion proteins comprise a heparin binding moeity that specifically binds heparin with an affinity (KD) of less than about 500 UM as determined by an SPR assay. In some embodiments, the IL-12 Fc fusion proteins comprise a heparin binding moeity that specifically binds heparin with an affinity (KD) of less than about 100 μM as determined by an SPR assay. In some embodiments, the IL-12 Fc fusion proteins comprise a heparin binding moiety that specifically binds heparin with an affinity (KD) of less than about 1 μM as determined by an SPR assay. In some embodiments, the IL-12 Fc fusion proteins comprise a heparin binding moiety that specifically binds heparin with an affinity (KD) of less than about 500 nM as determined by an SPR assay. In some embodiments, the heparin binding moiety specifically binds heparin with an affinity (KD) of about 0.1-500 UM, 0.1-100 UM, or 0.1-1 μM as determined by an SPR assay. In some embodiments, the heparin binding moiety specifically binds heparin with an affinity (KD) of about 100-1000 nM, 100-1000 nM, 100-800 nM, 100-600 nM, or 100-500 nM as determined by an SPR assay.

In some embodiments, the IL-12 Fc fusion proteins comprise a heparin binding moiety that specifically binds heparin and does not specifically bind to one or more non-heparin extracellular matrix (ECM) components including, but not limited to, collagen, fibronectin, vitronectin, tenascin C, osteopontin and fibrinogen. In some embodiments, the heparin binding moiety binds to heparin with a lower KD than to one or more non-heparin ECM components. In some embodiments, the KD of the heparin binding moiety for heparin is less than the KD of the heparin binding moiety for an extracellular matrix component selected from collagen, fibronectin, vitronectin, osteopontin, tenascin C, or fibrinogen. In some embodiments, the heparin binding moiety binds to heparin with about 10 percent, about 20 percent, about 30 percent, about 40 percent, about 50 percent, about 60 percent, about 70 percent, about 80 percent, about 90 percent, about 99 percent lower KD than to one or more non-heparin ECM components. In some embodiments, the heparin binding moeity binds to heparin with about 2-fold, about 3-fold, about 4-fold, about 5-fold, about 10-fold lower KD than to one or more non-heparin ECM components.

In some embodiments, the heparin binding moiety competes with a reference heparin binding moiety for binding to heparin. In some embodiments, the reference heparin binding moiety comprises a peptide as disclosed e.g. by Luria-Pérez et al., (2019) Cytokine, 120:220-226.

In its broadest form, the disclosure provides for an IL-12 Fc fusion protein, wherein the heparin binding moiety binds to heparin.

In a further preferred embodiment, the disclosure provides IL-12 Fc fusion proteins, wherein the heparin binding moiety binds to heparin and has the sequence VRIQRKKEKMKET (SEQ ID NO:50).

In another embodiment, the disclosure provides an IL-12 Fc fusion protein, wherein the heparin binding moiety has a length of 20 amino acids (aa), 19aa, 18aa, 17aa, 16aa, 15aa, 14aa, 13aa, 12aa, 11aa, 10a, or 9aa and comprises the sequence VRIQRKKEKMKET (SEQ ID NO:50).

| Identifier | Sequence | SEQ ID NO: |
|---|---|---|
| Alpha 1 chain | MFSFVDLRLLLLLAATALLTHGQEEGQVEGQDEDIPPITCVQNGL | 38 |
| precursor of type I | RYHDRDVWKPEPCRICVCDNGKVLCDDVICDETKNCPGAEVPEGE | |
| collagen (human) | CCPVCPDGSESPTDQETTGVEGPKGDTGPRGPRGPAGPPGRDGIP | |
| >sp\|P02452.6\| | GQPGLPGPPGPPGPPGPPGLGGNFAPQLSYGYDEKSTGGISVPGP | |
| CO1A1_HUMAN | MGPSGPRGLPGPPGAPGPQGFQGPPGEPGEPGASGPMGPRGPPGP | |
| | PGKNGDDGEAGKPGRPGERGPPGPQGARGLPGTAGLPGMKGHRGF | |
| | SGLDGAKGDAGPAGPKGEPGSPGENGAPGQMGPRGLPGERGRPGA | |
| | PGPAGARGNDGATGAAGPPGPTGPAGPPGFPGAVGAKGEAGPQGP | |
| | RGSEGPQGVRGEPGPPGPAGAAGPAGNPGADGQPGAKGANGAPGI | |
| | AGAPGFPGARGPSGPQGPGGPPGPKGNSGEPGAPGSKGDTGAKGE | |
| | PGPVGVQGPPGPAGEEGKRGARGEPGPTGLPGPPGERGGPGSRGF | |
| | PGADGVAGPKGPAGERGSPGPAGPKGSPGEAGRPGEAGLPGAKGL | |
| | TGSPGSPGPDGKTGPPGPAGQDGRPGPPGPPGARGQAGVMGFPGP | |
| | KGAAGEPGKAGERGVPGPPGAVGPAGKDGEAGAQGPPGPAGPAGE | |
| | RGEQGPAGSPGFQGLPGPAGPPGEAGKPGEQGVPGDLGAPGPSGA | |
| | RGERGFPGERGVQGPPGPAGPRGANGAPGNDGAKGDAGAPGAPGS | |
| | QGAPGLQGMPGERGAAGLPGPKGDRGDAGPKGADGSPGKDGVRGL | |
| | TGPIGPPGPAGAPGDKGESGPSGPAGPTGARGAPGDRGEPGPPGP | |
| | AGFAGPPGADGQPGAKGEPGDAGAKGDAGPPGPAGPAGPPGPIGN | |
| | VGAPGAKGARGSAGPPGATGFPGAAGRVGPPGPSGNAGPPGPPGP | |
| | AGKEGGKGPRGETGPAGRPGEVGPPGPPGPAGEKGSPGADGPAGA | |
| | PGTPGPQGIAGQRGVVGLPGQRGERGFPGLPGPSGEPGKQGPSGA | |
| | SGERGPPGPMGPPGLAGPPGESGREGAPGAEGSPGRDGSPGAKGD | |
| | RGETGPAGPPGAPGAPGAPGPVGPAGKSGDRGETGPAGPAGPVGP | |
| | VGARGPAGPQGPRGDKGETGEQGDRGIKGHRGFSGLQGPPGPPGS | |
| | PGEQGPSGASGPAGPRGPPGSAGAPGKDGLNGLPGPIGPPGPRGR | |
| | TGDAGPVGPPGPPGPPGPPGPPSAGFDFSFLPQPPQEKAHDGGRY | |
| | YRADDANVVRDRDLEVDTTLKSLSQQIENIRSPEGSRKNPARTCR | |
| | DLKMCHSDWKSGEYWIDPNQGCNLDAIKVFCNMETGETCVYPTQP | |
| | SVAQKNWYISKNPKDKRHVWFGESMTDGFQFEYGGQGSDPADVAI | |
| | QLTFLRLMSTEASQNITYHCKNSVAYMDQQTGNLKKALLLQGSNE | |
| | IEIRAEGNSRFTYSVTVDGCTSHTGAWGKTVIEYKTTKTSRLPII | |
| | DVAPLDVGAPDQEFGFDVGPVCFL | |
| Alpha2 chain | MLSFVDTRTLLLLAVTLCLATCQSLQEETVRKGPAGDRGPRGERG | 39 |
| precursor of type I | PPGPPGRDGEDGPTGPPGPPGPPGPPGLGGNFAAQYDGKGVGLGP | |
| collagen (human) | GPMGLMGPRGPPGAAGAPGPQGFQGPAGEPGEPGQTGPAGARGPA | |
| >sp\|P08123.7\| | GPPGKAGEDGHPGKPGRPGERGVVGPQGARGFPGTPGLPGFKGIR | |
| CO1A2_HUMAN | GHNGLDGLKGQPGAPGVKGEPGAPGENGTPGQTGARGLPGERGRV | |
| | GAPGPAGARGSDGSVGPVGPAGPIGSAGPPGFPGAPGPKGEIGAV | |
| | GNAGPAGPAGPRGEVGLPGLSGPVGPPGNPGANGLTGAKGAAGLP | |
| | GVAGAPGLPGPRGIPGPVGAAGATGARGLVGEPGPAGSKGESGNK | |
| | GEPGSAGPQGPPGPSGEEGKRGPNGEAGSAGPPGPPGLRGSPGSR | |
| | GLPGADGRAGVMGPPGSRGASGPAGVRGPNGDAGRPGEPGLMGPR | |
| | GLPGSPGNIGPAGKEGPVGLPGIDGRPGPIGPAGARGEPGNIGFP | |
| | GPKGPTGDPGKNGDKGHAGLAGARGAPGPDGNNGAQGPPGPQGVQ | |
| | GGKGEQGPPGPPGFQGLPGPSGPAGEVGKPGERGLHGEFGLPGPA | |
| | GPRGERGPPGESGAAGPTGPIGSRGPSGPPGPDGNKGEPGVVGAV | |
| | GTAGPSGPSGLPGERGAAGIPGGKGEKGEPGLRGEIGNPGRDGAR | |
| | GAPGAVGAPGPAGATGDRGEAGAAGPAGPAGPRGSPGERGEVGPA | |
| | GPNGFAGPAGAAGQPGAKGERGAKGPKGENGVVGPTGPVGAAGPA | |
| | GPNGPPGPAGSRGDGGPPGMTGFPGAAGRTGPPGPSGISGPPGPP | |
| | GPAGKEGLRGPRGDQGPVGRTGEVGAVGPPGFAGEKGPSGEAGTA | |
| | GPPGTPGPQGLLGAPGILGLPGSRGERGLPGVAGAVGEPGPLGIA | |
| | GPPGARGPPGAVGSPGVNGAPGEAGRDGNPGNDGPPGRDGQPGHK | |
| | GERGYPGNIGPVGAAGAPGPHGPVGPAGKHGNRGETGPSGPVGPA | |
| | GAVGPRGPSGPQGIRGDKGEPGEKGPRGLPGLKGHNGLQGLPGIA | |
| | GHHGDQGAPGSVGPAGPRGPAGPSGPAGKDGRTGHPGTVGPAGIR | |
| | GPQGHQGPAGPPGPPGPPGPPGVSGGGYDFGYDGDFYRADQPRSA | |
| | PSLRPKDYEVDATLKSLNNQIETLLTPEGSRKNPARTCRDLRLSH | |
| | PEWSSGYYWIDPNQGCTMDAIKVYCDFSTGETCIRAQPENIPAKN | |
| | WYRSSKDKKHVWLGETINAGSQFEYNVEGVTSKEMATQLAFMRLL | |
| | ANYASQNITYHCKNSIAYMDEETGNLKKAVILQGSNDVELVAEGN | |
| | SRFTYTVLVDGCSKKTNEWGKTIIEYKTNKPSRLPFLDIAPLDIG | |
| | GADQEFFVDIGPVCFK | |
| Collagen IV binding | KLWVLPK | 40 |
| moiety | | |
| Collagen I binding | LxxLxLxxN, | 41 |
| moiety | wherein L is Leucine and N is Asparagine and | |
| | x is any amino acid | |
| Collagen I binding | LSELRLHEN | 42 |
| moiety | | |
| Collagen I binding | LTELHLDNN | 43 |
| moiety | | |
| Collagen I binding | LSELRLHNN | 44 |
| moiety | | |
| Collagen I binding | LSELRLHAN | 45 |
| moiety | | |
| Collagen I binding | LRELHLNNN | 46 |
| moiety | | |
| Collagen I binding | LRELHLDNN | 47 |
| moiety | | |
| Fibronectin | MLRGPGPGLLLLAVQCLGTAVPSTGASKSKRQAQQMVQPQSPVAV | 48 |
| >sp\|P02751.5\| | SQSKPGCYDNGKHYQINQQWERTYLGNALVCTCYGGSRGENCESK | |
| FINC_HUMAN | PEAEETCFDKYTGNTYRVGDTYERPKDSMIWDCTCIGAGRGRISC | |
| | TIANRCHEGGQSYKIGDTWRRPHETGGYMLECVCLGNGKGEWTCK | |
| | PIAEKCFDHAAGTSYVVGETWEKPYQGWMMVDCTCLGEGSGRITC | |
| | TSRNRCNDQDTRTSYRIGDTWSKKDNRGNLLQCICTGNGRGEWKC | |
| | ERHTSVQTTSSGSGPFTDVRAAVYQPQPHPQPPPYGHCVTDSGVV | |
| | YSVGMQWLKTQGNKQMLCTCLGNGVSCQETAVTQTYGGNSNGEPC | |
| | VLPFTYNGRTFYSCTTEGRQDGHLWCSTTSNYEQDQKYSFCTDHT | |
| | VLVQTRGGNSNGALCHFPFLYNNHNYTDCTSEGRRDNMKWCGTTQ | |
| | NYDADQKFGFCPMAAHEEICTTNEGVMYRIGDQWDKQHDMGHMMR | |
| | CTCVGNGRGEWTCIAYSQLRDQCIVDDITYNVNDTFHKRHEEGHM | |
| | LNCTCFGQGRGRWKCDPVDQCQDSETGTFYQIGDSWEKYVHGVRY | |
| | QCYCYGRGIGEWHCQPLQTYPSSSGPVEVFITETPSQPNSHPIQW | |
| | NAPQPSHISKYILRWRPKNSVGRWKEATIPGHLNSYTIKGLKPGV | |
| | VYEGQLISIQQYGHQEVTRFDFTTTSTSTPVTSNTVTGETTPFSP | |
| | LVATSESVTEITASSFVVSWVSASDTVSGFRVEYELSEEGDEPQY | |
| | LDLPSTATSVNIPDLLPGRKYIVNVYQISEDGEQSLILSTSQTTA | |
| | PDAPPDTTVDQVDDTSIVVRWSRPQAPITGYRIVYSPSVEGSSTE | |
| | LNLPETANSVTLSDLQPGVQYNITIYAVEENQESTPVVIQQETTG | |
| | TPRSDTVPSPRDLQFVEVTDVKVTIMWTPPESAVTGYRVDVIPVN | |
| | LPGEHGQRLPISRNTFAEVTGLSPGVTYYFKVFAVSHGRESKPLT | |
| | AQQTTKLDAPTNLQFVNETDSTVLVRWTPPRAQITGYRLTVGLTR | |
| | RGQPRQYNVGPSVSKYPLRNLQPASEYTVSLVAIKGNQESPKATG | |
| | VFTTLQPGSSIPPYNTEVTETTIVITWTPAPRIGFKLGVRPSQGG | |
| | EAPREVTSDSGSIVVSGLTPGVEYVYTIQVLRDGQERDAPIVNKV | |
| | VTPLSPPTNLHLEANPDTGVLTVSWERSTTPDITGYRITTTPTNG | |
| | QQGNSLEEVVHADQSSCTFDNLSPGLEYNVSVYTVKDDKESVPIS | |
| | DTIIPEVPQLTDLSFVDITDSSIGLRWTPLNSSTIIGYRITVVAA | |
| | GEGIPIFEDFVDSSVGYYTVTGLEPGIDYDISVITLINGGESAPT | |
| | TLTQQTAVPPPTDLRFTNIGPDTMRVTWAPPPSIDLTNFLVRYSP | |
| | VKNEEDVAELSISPSDNAVVLTNLLPGTEYVVSVSSVYEQHESTP | |
| | LRGRQKTGLDSPTGIDESDITANSFTVHWIAPRATITGYRIRHHP | |
| | EHFSGRPREDRVPHSRNSITLTNLTPGTEYVVSIVALNGREESPL | |
| | LIGQQSTVSDVPRDLEVVAATPTSLLISWDAPAVTVRYYRITYGE | |
| | TGGNSPVQEFTVPGSKSTATISGLKPGVDYTITVYAVTGRGDSPA | |
| | SSKPISINYRTEIDKPSQMQVTDVQDNSISVKWLPSSSPVTGYRV | |
| | TTTPKNGPGPTKTKTAGPDQTEMTIEGLQPTVEYVVSVYAQNPSG | |
| | ESQPLVQTAVTNIDRPKGLAFTDVDVDSIKIAWESPQGQVSRYRV | |
| | TYSSPEDGIHELFPAPDGEEDTAELQGLRPGSEYTVSVVALHDDM | |
| | ESQPLIGTQSTAIPAPTDLKFTQVTPTSLSAQWTPPNVQLTGYRV | |
| | RVTPKEKTGPMKEINLAPDSSSVVVSGLMVATKYEVSVYALKDTL | |
| | TSRPAQGVVTTLENVSPPRRARVTDATETTITISWRTKTETITGF | |
| | QVDAVPANGQTPIQRTIKPDVRSYTITGLQPGTDYKIYLYTLNDN | |
| | ARSSPVVIDASTAIDAPSNLRFLATTPNSLLVSWQPPRARITGYI | |
| | IKYEKPGSPPREVVPRPRPGVTEATITGLEPGTEYTIYVIALKNN | |
| | QKSEPLIGRKKTDELPQLVTLPHPNLHGPEILDVPSTVQKTPFVT | |
| | HPGYDTGNGIQLPGTSGQQPSVGQQMIFEEHGFRRTTPPTTATPI | |
| | RHRPRPYPPNVGEEIQIGHIPREDVDYHLYPHGPGLNPNASTGQE | |
| | ALSQTTISWAPFQDTSEYIISCHPVGTDEEPLQFRVPGTSTSATL | |
| | TGLTRGATYNVIVEALKDQQRHKVREEVVTVGNSVNEGLNQPTDD | |
| | SCFDPYTVSHYAVGDEWERMSESGFKLLCQCLGFGSGHFRCDSSR | |
| | WCHDNGVNYKIGEKWDRQGENGQMMSCTCLGNGKGEFKCDPHEAT | |
| | CYDDGKTYHVGEQWQKEYLGAICSCTCFGGQRGWRCDNCRRPGGE | |
| | PSPEGTTGQSYNQYSQRYHQRTNTNVNCPIECEMPLDVQADREDS | |
| | RE | |
| Fibronectin binding | GGWSHW | 49 |
| moiety | | |
| Heparin binding | VRIQRKKEKMKET | 50 |
| moiety | | |

### IL-12 Fc Fusion Proteins

Described and disclosed herein are IL-12 Fc fusion proteins, as well as compositions and articles of manufacture comprising IL-12 Fc fusion proteins of the present invention. The present inventors conceived IL-12 Fc fusion proteins of the invention as shown below and a selection of those fusion proteins were prepared and are discussed in the accompanying examples. The cleavage product comprising the IL-12 cytokine and the binding moiety after proteolytic cleavage is underlined in Table 5.

TABLE 5

| | | |
|---|---|---|
| BI-050 Fc Knob | EPKSSDKTHTCPPCPAPEAAGGPSVFLFPPKPKDTLMISRTPEVT | 208 |
| (human) | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGLRELHLDNNGGSIWELKKDVYVV | |
| | ELDWYPDAPGEMVVLTCDTPEEDGITWTLDQSSEVLGSGKTLTIQ | |
| | VKEFGDAGQYTCHKGGEVLSHSLLLLHKKEDGIWSTDILKDQKEP | |
| | KNKTFLRCEAKNYSGRFTCWWLTTISTDLTFSVKSSRGSSDPQGV | |
| | TCGAATLSAERVRGDNKEYEYSVECQEDSACPAAEESLPIEVMVD | |
| | AVHKLKYENYTSSFFIRDIIKPDPPKNLQLKPLKNSRQVEVSWEY | |
| | PDTWSTPHSYFSLTFCVQVQGKSKREKKDRVFTDKTSATVICRKN | |
| | ASISVRAQDRYYSSSWSEWASVPCSGGGGSGGGGSGGGGSRNLPV | |
| | ATPDPGMFPCLHHSQNLLRAVSNMLQKARQTLEFYPCTSEEIDHE | |
| | DITKDKTSTVEACLPLELTKNESCLNSRETSFITNGSCLASRKTS | |
| | FMMALCLSSIYEDLKMYQVEFKTMNAKLLMDPKRQIFLDQNMLAV | |
| | IDELMQALNFNSETVPQKSSLEEPDFYKTKIKLCILLHAFRIRAV | |
| | TIDRVMSYLNAS | |
| BI-050 Fc Hole | EPKSSDKTHTCPPCPAPEAAGGPSVFLFPPKPKDTLMISRTPEVT | 209 |
| (human) | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NTDYTTYDYWGQGTQVTVSSA | |
| BI-051 Fc Knob | EPKSSDKTHTCPPCPAPEAAGGPSVFLFPPKPKDTLMISRTPEVT | 210 |
| (human) | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGGGSGGGGSGGGGSRNLPVATPDPGM | |
| | FPCLHHSQNLLRAVSNMLQKARQTLEFYPCTSEEIDHEDITKDKT | |
| | STVEACLPLELTKNESCLNSRETSFITNGSCLASRKTSFMMALCL | |
| | SSIYEDLKMYQVEFKTMNAKLLMDPKRQIFLDQNMLAVIDELMQA | |
| | LNFNSETVPQKSSLEEPDFYKTKIKLCILLHAFRIRAVTIDRVMS | |
| | YLNASGSGGLRELHLDNN | |
| BI-051 Fc Hole | EPKSSDKTHTCPPCPAPEAAGGPSVFLFPPKPKDTLMISRTPEVT | 21 |
| (human) | CVVVDVSHEDPEVKENWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NTDYTTYDYWGQGTQVTVSSA | |
| BI-052 Fc Knob | EPKSSDKTHTCPPCPAPEAAGGPSVFLFPPKPKDTLMISRTPEVT | 212 |
| (human) | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGGGSGGGGSGGGGSLRELHLDNNGGS | |
| | RNLPVATPDPGMFPCLHHSQNLLRAVSNMLQKARQTLEFYPCTSE | |
| | EIDHEDITKDKTSTVEACLPLELTKNESCLNSRETSFITNGSCLA | |
| | SRKTSFMMALCLSSIYEDLKMYQVEFKTMNAKLLMDPKRQIFLDQ | |
| | NMLAVIDELMQALNFNSETVPQKSSLEEPDFYKTKIKLCILLHAF | |
| | RIRAVTIDRVMSYLNAS | |
| BI-052 Fc Hole | EPKSSDKTHTCPPCPAPEAAGGPSVFLFPPKPKDTLMISRTPEVT | 213 |
| (human) | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NTDYTTYDYWGQGTQVTVSSA | |
| BI-053 Fc Knob | EPKSSDKTHTCPPCPAPEAAGGPSVFLFPPKPKDTLMISRTPEVT | 214 |
| (human) | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGGGSGGGGSGGGGSRNLPVATPDPGM | |
| | FPCLHHSQNLLRAVSNMLQKARQTLEFYPCTSEEIDHEDITKDKT | |
| | STVEACLPLELTKNESCLNSRETSFITNGSCLASRKTSFMMALCL | |
| | SSIYEDLKMYQVEFKTMNAKLLMDPKRQIFLDQNMLAVIDELMQA | |
| | LNFNSETVPQKSSLEEPDFYKTKIKLCILLHAFRIRAVTIDRVMS | |
| | YLNASGSGGGGWSHW | |
| BI-053 Fc Hole | EPKSSDKTHTCPPCPAPEAAGGPSVFLFPPKPKDTLMISRTPEVT | 215 |
| (human) | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NTDYTTYDYWGQGTQVTVSSA | |
| BI-054 Fc Knob | EPKSSDKTHTCPPCPAPEAAGGPSVFLFPPKPKDTLMISRTPEVT | 216 |
| (human) | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGGGSGGGGSGGGGSRNLPVATPDPGM | |
| | FPCLHHSQNLLRAVSNMLQKARQTLEFYPCTSEEIDHEDITKDKT | |
| | STVEACLPLELTKNESCLNSRETSFITNGSCLASRKTSFMMALCL | |
| | SSIYEDLKMYQVEFKTMNAKLLMDPKRQIFLDQNMLAVIDELMQA | |
| | LNFNSETVPQKSSLEEPDFYKTKIKLCILLHAFRIRAVTIDRVMS | |
| | YLNASGSGGLRELHLDNN | |
| BI-054 Fc Hole | EPKSSDKTHTCPPCPAPEAAGGPSVFLFPPKPKDTLMISRTPEVT | 217 |
| (human) | CVVVDVSHEDPEVKENWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNREK | |
| | NREYTTYDYWGQGTQVTVSSA | |
| BI-055 Fc Knob | EPKSSDKTHTCPPCPAPEAAGGPSVFLFPPKPKDTLMISRTPEVT | 218 |
| (human) | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPCREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGGGSGGGGSGGGGSRNLPVATPDPGM | |
| | FPCLHHSQNLLRAVSNMLQKARQTLEFYPCTSEEIDHEDITKDKT | |
| | STVEACLPLELTKNESCLNSRETSFITNGSCLASRKTSFMMALCL | |
| | SSIYEDLKMYQVEFKTMNAKLLMDPKRQIFLDQNMLAVIDELMQA | |
| | LNFNSETVPQKSSLEEPDFYKTKIKLCILLHAFRIRAVTIDRVMS | |
| | YLNASGSGGLRELHLDN | |
| BI-055 Fc Hole | EPKSSDKTHTCPPCPAPEAAGGPSVFLFPPKPKDTLMISRTPEVT | 219 |
| (human) | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVCT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NTDYTTYDYWGQGTQVTVSSA | |
| BI-056 Fc Knob | EPKSSDKTHTCPPCPAPEAAGGPSVFLFPPKPKDTLMISRTPEVT | 220 |
| (chimeric) | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGLRELHLDNNGGSIWELKKDVYVV | |
| | ELDWYPDAPGEMVVLTCDTPEEDGITWTLDQSSEVLGSGKTLTIQ | |
| | VKEFGDAGQYTCHKGGEVLSHSLLLLHKKEDGIWSTDILKDQKEP | |
| | KNKTFLRCEAKNYSGRFTCWWLTTISTDLTFSVKSSRGSSDPQGV | |
| | TCGAATLSAERVRGDNKEYEYSVECQEDSACPAAEESLPIEVMVD | |
| | AVHKLKYENYTSSFFIRDIIKPDPPKNLQLKPLKNSRQVEVSWEY | |
| | PDTWSTPHSYFSLTFCVQVQGKSKREKKDRVFTDKTSATVICRKN | |
| | ASISVRAQDRYYSSSWSEWASVPCSGGGGSGGGGSGGGGSRVIPV | |
| | SGPARCLSQSRNLLKTTDDMVKTAREKLKHYSCTAEDIDHEDITR | |
| | DQTSTLKTCLPLELHKNESCLATRETSSTTRGSCLPPQKTSLMMT | |
| | LCLGSIYEDLKMYQTEFQAINAALQNHNHQQIILDKGMLVAIDEL | |
| | MQSLNHNGETLRQKPPVGEADPYRVKMKLCILLHAFSTRVVTINR | |
| | VMGYLSSA | |
| BI-056 Fc Hole | EPKSSDKTHTCPPCPAPEAAGGPSVFLFPPKPKDTLMISRTPEVT | 221 |
| (chimeric) | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NTDYTTYDYWGQGTQVTVSSA | |
| BI-057 Fc Knob | EPKSSDKTHTCPPCPAPEAAGGPSVFLFPPKPKDTLMISRTPEVT | 222 |
| (chimeric) | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGGGSGGGGSGGGGSRVIPVSGPARCL | |
| | SQSRNLLKTTDDMVKTAREKLKHYSCTAEDIDHEDITRDQTSTLK | |
| | TCLPLELHKNESCLATRETSSTTRGSCLPPQKTSLMMTLCLGSIY | |
| | EDLKMYQTEFQAINAALQNHNHQQIILDKGMLVAIDELMQSLNHN | |
| | GETLRQKPPVGEADPYRVKMKLCILLHAFSTRVVTINRVMGYLSS | |
| | AGSGGLRELHLDNN | |
| BI-057 Fc Hole | EPKSSDKTHTCPPCPAPEAAGGPSVFLFPPKPKDTLMISRTPEVT | 223 |
| (chimeric) | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNREK | |
| | NTDYTTYDYWGQGTQVTVSSA | |
| BI-058 Fc Knob | EPKSSDKTHTCPPCPAPEAAGGPSVFLFPPKPKDTLMISRTPEVT | 224 |
| (chimeric) | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGGGSGGGGSGGGGSLRELHLDNNGGS | |
| | RVIPVSGPARCLSQSRNLLKTTDDMVKTAREKLKHYSCTAEDIDH | |
| | EDITRDQTSTLKTCLPLELHKNESCLATRETSSTTRGSCLPPQKT | |
| | SLMMTLCLGSIYEDLKMYQTEFQAINAALQNHNHQQIILDKGMLV | |
| | AIDELMQSLNHNGETLRQKPPVGEADPYRVKMKLCILLHAFSTRV | |
| | VTINRVMGYLSSA | |
| BI-058 Fc Hole | EPKSSDKTHTCPPCPAPEAAGGPSVFLFPPKPKDTLMISRTPEVT | 225 |
| (chimeric) | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NTDYTTYDYWGQGTQVTVSSA | |
| BI-059 Fc Knob | EPKSSDKTHTCPPCPAPEAAGGPSVFLFPPKPKDTLMISRTPEVT | 226 |
| (chimeric) | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGGGSGGGGSGGGGSRVIPVSGPARCL | |
| | SQSRNLLKTTDDMVKTAREKLKHYSCTAEDIDHEDITRDQTSTLK | |
| | TCLPLELHKNESCLATRETSSTTRGSCLPPQKTSLMMTLCLGSIY | |
| | EDLKMYQTEFQAINAALQNHNHQQIILDKGMLVAIDELMQSLNHN | |
| | GETLRQKPPVGEADPYRVKMKLCILLHAFSTRVVTINRVMGYLSS | |
| | AGSGGGGWSHW | |
| BI-059 Fc Hole | EPKSSDKTHTCPPCPAPEAAGGPSVFLFPPKPKDTLMISRTPEVT | 227 |
| (chimeric) | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NTDYTTYDYWGQGTQVTVSSA | |
| BI-060 Fc Knob | EPKSSDKTHTCPPCPAPEAAGGPSVFLFPPKPKDTLMISRTPEVT | 228 |
| (chimeric) | CVVVDVSHEDPEVKENWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGGGSGGGGSGGGGSRVIPVSGPARCL | |
| | SQSRNLLKTTDDMVKTAREKLKHYSCTAEDIDHEDITRDQTSTLK | |
| | TCLPLELHKNESCLATRETSSTTRGSCLPPQKTSLMMTLCLGSIY | |
| | EDLKMYQTEFQAINAALQNHNHQQIILDKGMLVAIDELMQSLNHN | |
| | GETLRQKPPVGEADPYRVKMKLCILLHAFSTRVVTINRVMGYLSS | |
| | AGSGGLRELHLDNN | |
| BI-060 Fc Hole | EPKSSDKTHTCPPCPAPEAAGGPSVFLFPPKPKDTLMISRTPEVT | 229 |
| (chimeric) | CVVVDVSHEDPEVKENWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NREYTTYDYWGQGTQVTVSSA | |
| BI-061 Fc Knob | EPKSSDKTHTCPPCPAPEAAGGPSVFLFPPKPKDTLMISRTPEVT | 230 |
| (chimeric) | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPCREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGGGSGGGGSGGGGSRVIPVSGPARCL | |
| | SQSRNLLKTTDDMVKTAREKLKHYSCTAEDIDHEDITRDQTSTLK | |
| | TCLPLELHKNESCLATRETSSTTRGSCLPPQKTSLMMTLCLGSIY | |
| | EDLKMYQTEFQAINAALQNHNHQQIILDKGMLVAIDELMQSLNHN | |
| | GETLRQKPPVGEADPYRVKMKLCILLHAFSTRVVTINRVMGYLSS | |
| | AGSGGLRELHLDNN | |
| BI-061 Fc Hole | EPKSSDKTHTCPPCPAPEAAGGPSVFLFPPKPKDTLMISRTPEVT | 231 |
| (chimeric) | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVCT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NTDYTTYDYWGQGTQVTVSSA | |

In the broadest sense the Interleukin-12 (IL-12) Fc fusion protein comprises a first polypeptide chain and a second polypeptide chain, wherein a) the first polypeptide chain comprises a first Fc domain and an IL-12p35 subunit and an IL-12p40 subunit of IL-12, and b) the second polypeptide chain comprises a second Fc domain and a masking moiety that binds to the IL-12p35 and/or IL-12p40 subunit in the first polypeptide chain; wherein the first and second polypeptide chain are linked via the first Fc domain and the second Fc domain, wherein the IL-12p35 subunit or the IL-12p40 subunit is linked to the C-terminus of the first Fc domain via a first peptide linker, which first peptide linker is protease-cleavable, wherein the masking moiety is linked to the C-terminus of the second Fc domain via a second linker, preferably a peptide linker and wherein the first or the second polypeptide chain further comprises a binding moiety selected from the group consisting of: a collagen binding moiety, a heparin binding moiety, and a fibronectin binding moiety.

In one embodiment the Interleukin-12 (IL-12) Fc fusion protein comprises a first polypeptide chain and a second polypeptide chain, wherein a) the first polypeptide chain comprises a first Fc domain and an IL-12p35 subunit and an IL-12p40 subunit of IL-12, and b) the second polypeptide chain comprises a second Fc domain and a masking moiety that binds to the IL-12p35 and/or IL-12p40 subunit in the first polypeptide chain; wherein the first and second polypeptide chain are linked via the first Fc domain and the second Fc domain, wherein the IL-12p35 subunit or the IL-12p40 subunit is linked to the C-terminus of the first Fc domain via a first peptide linker, which first peptide linker is protease-cleavable, wherein the masking moiety is linked to the C-terminus of the second Fc domain via a second linker, preferably a peptide linker and wherein the first or the second polypeptide chain further comprises a collagen binding moiety, preferably selected from the group consisting of SEQ ID NOs:41-47.

In one embodiment the Interleukin-12 (IL-12) Fc fusion protein comprises a first polypeptide chain and a second polypeptide chain, wherein a) the first polypeptide chain comprises a first Fc domain and an IL-12p35 subunit and an IL-12p40 subunit of IL-12, and b) the second polypeptide chain comprises a second Fc domain and a masking moiety that binds to the IL-12p35 and/or IL-12p40 subunit in the first polypeptide chain, wherein the masking moiety is selected from the group consisting of any one of SEQ ID NOs:61-109; wherein the first and second polypeptide chain are linked via the first Fc domain and the second Fc domain, wherein the IL-12p35 subunit or the IL-12p40 subunit is linked to the C-terminus of the first Fc domain via a first peptide linker, which first peptide linker is protease-cleavable, wherein the masking moiety is linked to the C-terminus of the second Fc domain via a second linker, preferably a peptide linker and wherein the first or the second polypeptide chain further comprises a binding moiety selected from the group consisting of: a collagen binding moiety, a heparin binding moiety, and a fibronectin binding moiety. The masking moiety may further comprise an additional alanine attached to its C-terminus.

In one embodiment, the Interleukin-12 (IL-12) Fc fusion protein comprises a first polypeptide chain and a second polypeptide chain, wherein a) the first polypeptide chain comprises a first Fc domain and an IL-12p35 subunit and an IL-12p40 subunit of IL-12, and b) the second polypeptide chain comprises a second Fc domain and a masking moiety that binds to the IL-12p35 and/or IL-12p40 subunit in the first polypeptide chain; wherein the first and second polypeptide chain are linked via the first Fc domain and the second Fc domain, wherein the IL-12p35 subunit or the IL-12p40 subunit is linked to the C-terminus of the first Fc domain via a first peptide linker, which first peptide linker is protease-cleavable, wherein the protease-cleavable linker is selected from the group consisting of any one of the amino acid sequences of SEQ ID NOs:232-241, wherein the masking moiety is linked to the C-terminus of the second Fc domain via a second linker, preferably a peptide linker and wherein the first or the second polypeptide chain further comprises a binding moiety selected from the group consisting of: a collagen binding moiety, a heparin binding moiety, and a fibronectin binding moiety.

In another embodiment the Interleukin-12 (IL-12) Fc fusion protein comprises a first polypeptide chain and a second polypeptide chain, wherein a) the first polypeptide chain comprises a first Fc domain and an IL-12p35 subunit and an IL-12p40 subunit of IL-12, wherein the first Fc domain comprises the amino acid sequence of SEQ ID NO:15, and b) the second polypeptide chain comprises a second Fc domain and a masking moiety that binds to the IL-12p35 and/or IL-12p40 subunit in the first polypeptide chain, wherein the second Fc domain comprises the amino acid sequence of SEQ ID NO:16; wherein the first and second polypeptide chain are linked via the first Fc domain and the second Fc domain, wherein the IL-12p35 subunit or the IL-12p40 subunit is linked to the C-terminus of the first Fc domain via a first peptide linker, which first peptide linker is protease-cleavable, wherein the masking moiety is linked to the C-terminus of the second Fc domain via a second linker, preferably a peptide linker and wherein the first or the second polypeptide chain further comprises a binding moiety selected from the group consisting of: a collagen binding moiety, a heparin binding moiety, and a fibronectin binding moiety.

In another embodiment the Interleukin-12 (IL-12) Fc fusion protein comprises a first polypeptide chain and a second polypeptide chain, wherein a) the first polypeptide chain comprises a first Fc domain and an IL-12p35 subunit and an IL-12p40 subunit of IL-12, wherein the first Fc domain comprises the amino acid sequence of SEQ ID NO:17 and b) the second polypeptide chain comprises a second Fc domain and a masking moiety that binds to the IL-12p35 and/or IL-12p40 subunit in the first polypeptide chain, wherein the second Fc domain comprises the amino acid sequence of SEQ ID NO:18; wherein the first and second polypeptide chain are linked via the first Fc domain and the second Fc domain, wherein the IL-12p35 subunit or the IL-12p40 subunit is linked to the C-terminus of the first Fc domain via a first peptide linker, which first peptide linker is protease-cleavable, wherein the masking moiety is linked to the C-terminus of the second Fc domain via a second linker, preferably a peptide linker and wherein the first or the second polypeptide chain further comprises a binding moiety selected from the group consisting of: a collagen binding moiety, a heparin binding moiety, and a fibronectin binding moiety.

In one embodiment the Interleukin-12 (IL-12) Fc fusion protein comprises a first polypeptide chain and a second polypeptide chain, wherein a) the first polypeptide chain comprises a first Fc domain and an IL-12p35 subunit and an IL-12p40 subunit of IL-12, and b) the second polypeptide chain comprises a second Fc domain and a masking moiety that binds to the IL-12p35 and/or IL-12p40 subunit in the first polypeptide chain, wherein the masking moiety is selected from the group consisting of any one of SEQ ID NOs:61-109; wherein the first and second polypeptide chain are linked via the first Fc domain and the second Fc domain, wherein the IL-12p35 subunit or the IL-12p40 subunit is linked to the C-terminus of the first Fc domain via a first peptide linker, which first peptide linker is protease-cleavable, wherein the masking moiety is linked to the C-terminus of the second Fc domain via a second linker, preferably a peptide linker and wherein the first or the second polypeptide chain further comprises a collagen binding moiety, preferably selected from the group consisting of SEQ ID NOs:41-47. The masking moiety may further comprise an additional alanine attached to its C-terminus.

In one embodiment the Interleukin-12 (IL-12) Fc fusion protein comprises a first polypeptide chain and a second polypeptide chain, wherein a) the first polypeptide chain comprises a first Fc domain and an IL-12p35 subunit and an IL-12p40 subunit of IL-12, and b) the second polypeptide chain comprises a second Fc domain and a masking moiety that binds to the IL-12p35 and/or IL-12p40 subunit in the first polypeptide chain, wherein the masking moiety is selected from the group consisting of any one of SEQ ID NOs:61-109; wherein the first and second polypeptide chain are linked via the first Fc domain and the second Fc domain, wherein the IL-12p35 subunit or the IL-12p40 subunit is linked to the C-terminus of the first Fc domain via a first peptide linker, which first peptide linker is protease-cleavable, wherein the protease-cleavable linker is selected from the group consisting of any one of the amino acid sequences of SEQ ID NOs:232-241, wherein the masking moiety is linked to the C-terminus of the second Fc domain via a second linker, preferably a peptide linker and wherein the first or the second polypeptide chain further comprises a collagen binding moiety, preferably selected from the group consisting of SEQ ID NOs:41-47. The masking moiety may further comprise an additional alanine attached to its C-terminus.

In one embodiment the Interleukin-12 (IL-12) Fc fusion protein comprises a first polypeptide chain and a second polypeptide chain, wherein a) the first polypeptide chain comprises a first Fc domain and an IL-12p35 subunit and an IL-12p40 subunit of IL-12, wherein the first Fc domain comprises the amino acid sequence of SEQ ID NO:15, and b) the second polypeptide chain comprises a second Fc domain and a masking moiety that binds to the IL-12p35 and/or IL-12p40 subunit in the first polypeptide chain, wherein the second Fc domain comprises the amino acid sequence of SEQ ID NO:16 and the masking moiety is selected from the group consisting of any one of SEQ ID NOs:61-109; wherein the first and second polypeptide chain are linked via the first Fc domain and the second Fc domain, wherein the IL-12p35 subunit or the IL-12p40 subunit is linked to the C-terminus of the first Fc domain via a first peptide linker, which first peptide linker is protease-cleavable, wherein the protease-cleavable linker is selected from the group consisting of any one of the amino acid sequences of SEQ ID NOs:232-241, wherein the masking moiety is linked to the C-terminus of the second Fc domain via a second linker, preferably a peptide linker and wherein the first or the second polypeptide chain further comprises a collagen binding moiety, preferably selected from the group consisting of SEQ ID NOs:41-47. The masking moiety may further comprise an additional alanine attached to its C-terminus.

In one embodiment the Interleukin-12 (IL-12) Fc fusion protein comprises a first polypeptide chain and a second polypeptide chain, wherein a) the first polypeptide chain comprises a first Fc domain and an IL-12p35 subunit and an IL-12p40 subunit of IL-12, wherein the first Fc domain comprises the amino acid sequence of SEQ ID NO:17, and b) the second polypeptide chain comprises a second Fc domain and a masking moiety that binds to the IL-12p35 and/or IL-12p40 subunit in the first polypeptide chain, wherein the second Fc domain comprises the amino acid sequence of SEQ ID NO:18 and the masking moiety is selected from the group consisting of any one of SEQ ID NOs:61-109; wherein the first and second polypeptide chain are linked via the first Fc domain and the second Fc domain, wherein the IL-12p35 subunit or the IL-12p40 subunit is linked to the C-terminus of the first Fc domain via a first peptide linker, which first peptide linker is protease-cleavable, wherein the protease-cleavable linker is selected from the group consisting of any one of the amino acid sequences of SEQ ID NOs:232-241, wherein the masking moiety is linked to the C-terminus of the second Fc domain via a second linker, preferably a peptide linker and wherein the first or the second polypeptide chain further comprises a collagen binding moiety, preferably selected from the group consisting of SEQ ID NOs:41-47. The masking moiety may further comprise an additional alanine attached to its C-terminus.

In one embodiment the Interleukin-12 (IL-12) Fc fusion protein comprises a first polypeptide chain and a second polypeptide chain, wherein a) the first polypeptide chain comprises a first Fc domain and an IL-12p35 subunit and an IL-12p40 subunit of IL-12, wherein the first Fc domain comprises the amino acid sequence of SEQ ID NO:17, and b) the second polypeptide chain comprises a second Fc domain and a masking moiety that binds to the IL-12p35 and/or IL-12p40 subunit in the first polypeptide chain, wherein the second Fc domain comprises the amino acid sequence of SEQ ID NO:18 and the masking moiety is selected from the group consisting of any one of SEQ ID NOs:61-109; wherein the first and second polypeptide chain are linked via the first Fc domain and the second Fc domain, wherein the IL-12p35 subunit or the IL-12p40 subunit is linked to the C-terminus of the first Fc domain via a first peptide linker, which first peptide linker is protease-cleavable, wherein the protease-cleavable linker is selected from the group consisting of any one of the amino acid sequences of SEQ ID NOs:232-241, wherein the masking moiety is linked to the C-terminus of the second Fc domain via a second linker, preferably a peptide linker and wherein the first or the second polypeptide chain further comprises a collagen binding moiety having the amino acid sequence of SEQ ID NO:41. The masking moiety may further comprise an additional alanine attached to its C-terminus.

In one embodiment the Interleukin-12 (IL-12) Fc fusion protein comprises a first polypeptide chain and a second polypeptide chain, wherein a) the first polypeptide chain comprises a first Fc domain and an IL-12p35 subunit and an IL-12p40 subunit of IL-12, wherein the first Fc domain comprises the amino acid sequence of SEQ ID NO:17, and b) the second polypeptide chain comprises a second Fc domain and a masking moiety that binds to the IL-12p35 and/or IL-12p40 subunit in the first polypeptide chain, wherein the second Fc domain comprises the amino acid sequence of SEQ ID NO:18 and the masking moiety is selected from the group consisting of any one of SEQ ID NOs:61-109; wherein the first and second polypeptide chain are linked via the first Fc domain and the second Fc domain, wherein the IL-12p35 subunit or the IL-12p40 subunit is linked to the C-terminus of the first Fc domain via a first peptide linker, which first peptide linker is protease-cleavable, wherein the protease-cleavable linker is selected from the group consisting of any one of the amino acid sequences of SEQ ID NOs:232-241, wherein the masking moiety is linked to the C-terminus of the second Fc domain via a second linker, preferably a peptide linker and wherein the first or the second polypeptide chain further comprises a collagen binding moiety having the amino acid sequence of SEQ ID NO:42. The masking moiety may further comprise an additional alanine attached to its C-terminus.

In one embodiment the Interleukin-12 (IL-12) Fc fusion protein comprises a first polypeptide chain and a second polypeptide chain, wherein a) the first polypeptide chain comprises a first Fc domain and an IL-12p35 subunit and an IL-12p40 subunit of IL-12, wherein the first Fc domain comprises the amino acid sequence of SEQ ID NO:17, and b) the second polypeptide chain comprises a second Fc domain and a masking moiety that binds to the IL-12p35 and/or IL-12p40 subunit in the first polypeptide chain, wherein the second Fc domain comprises the amino acid sequence of SEQ ID NO:18 and the masking moiety is selected from the group consisting of any one of SEQ ID NOs:61-109; wherein the first and second polypeptide chain are linked via the first Fc domain and the second Fc domain, wherein the IL-12p35 subunit or the IL-12p40 subunit is linked to the C-terminus of the first Fc domain via a first peptide linker, which first peptide linker is protease-cleavable, wherein the protease-cleavable linker is selected from the group consisting of any one of the amino acid sequences of SEQ ID NOs:232-241, wherein the masking moiety is linked to the C-terminus of the second Fc domain via a second linker, preferably a peptide linker and wherein the first or the second polypeptide chain further comprises a collagen binding moiety having the amino acid sequence of SEQ ID NO:43. The masking moiety may further comprise an additional alanine attached to its C-terminus.

In one embodiment the Interleukin-12 (IL-12) Fc fusion protein comprises a first polypeptide chain and a second polypeptide chain, wherein a) the first polypeptide chain comprises a first Fc domain and an IL-12p35 subunit and an IL-12p40 subunit of IL-12, wherein the first Fc domain comprises the amino acid sequence of SEQ ID NO:17, and b) the second polypeptide chain comprises a second Fc domain and a masking moiety that binds to the IL-12p35 and/or IL-12p40 subunit in the first polypeptide chain, wherein the second Fc domain comprises the amino acid sequence of SEQ ID NO:18 and the masking moiety is selected from the group consisting of any one of SEQ ID NOs:61-109; wherein the first and second polypeptide chain are linked via the first Fc domain and the second Fc domain, wherein the IL-12p35 subunit or the IL-12p40 subunit is linked to the C-terminus of the first Fc domain via a first peptide linker, which first peptide linker is protease-cleavable, wherein the protease-cleavable linker is selected from the group consisting of any one of the amino acid sequences of SEQ ID NOs:232-241, wherein the masking moiety is linked to the C-terminus of the second Fc domain via a second linker, preferably a peptide linker and wherein the first or the second polypeptide chain further comprises a collagen binding moiety having the amino acid sequence of SEQ ID NO:44. The masking moiety may further comprise an additional alanine attached to its C-terminus.

In one embodiment the Interleukin-12 (IL-12) Fc fusion protein comprises a first polypeptide chain and a second polypeptide chain, wherein a) the first polypeptide chain comprises a first Fc domain and an IL-12p35 subunit and an IL-12p40 subunit of IL-12, wherein the first Fc domain comprises the amino acid sequence of SEQ ID NO:17, and b) the second polypeptide chain comprises a second Fc domain and a masking moiety that binds to the IL-12p35 and/or IL-12p40 subunit in the first polypeptide chain, wherein the second Fc domain comprises the amino acid sequence of SEQ ID NO:18 and the masking moiety is selected from the group consisting of any one of SEQ ID NOs:61-109; wherein the first and second polypeptide chain are linked via the first Fc domain and the second Fc domain, wherein the IL-12p35 subunit or the IL-12p40 subunit is linked to the C-terminus of the first Fc domain via a first peptide linker, which first peptide linker is protease-cleavable, wherein the protease-cleavable linker is selected from the group consisting of any one of the amino acid sequences of SEQ ID NOs:232-241, wherein the masking moiety is linked to the C-terminus of the second Fc domain via a second linker, preferably a peptide linker and wherein the first or the second polypeptide chain further comprises a collagen binding moiety having the amino acid sequence of SEQ ID NO:45. The masking moiety may further comprise an additional alanine attached to its C-terminus.

In one embodiment the Interleukin-12 (IL-12) Fc fusion protein comprises a first polypeptide chain and a second polypeptide chain, wherein a) the first polypeptide chain comprises a first Fc domain and an IL-12p35 subunit and an IL-12p40 subunit of IL-12, wherein the first Fc domain comprises the amino acid sequence of SEQ ID NO:17, and b) the second polypeptide chain comprises a second Fc domain and a masking moiety that binds to the IL-12p35 and/or IL-12p40 subunit in the first polypeptide chain, wherein the second Fc domain comprises the amino acid sequence of SEQ ID NO:18 and the masking moiety is selected from the group consisting of any one of SEQ ID NOs:61-109; wherein the first and second polypeptide chain are linked via the first Fc domain and the second Fc domain, wherein the IL-12p35 subunit or the IL-12p40 subunit is linked to the C-terminus of the first Fc domain via a first peptide linker, which first peptide linker is protease-cleavable, wherein the protease-cleavable linker is selected from the group consisting of any one of the amino acid sequences of SEQ ID NOs:232-241, wherein the masking moiety is linked to the C-terminus of the second Fc domain via a second linker, preferably a peptide linker and wherein the first or the second polypeptide chain further comprises a collagen binding moiety having the amino acid sequence of SEQ ID NO:46. The masking moiety may further comprise an additional alanine attached to its C-terminus.

In one embodiment the Interleukin-12 (IL-12) Fc fusion protein comprises a first polypeptide chain and a second polypeptide chain, wherein a) the first polypeptide chain comprises a first Fc domain and an IL-12p35 subunit and an IL-12p40 subunit of IL-12, wherein the first Fc domain comprises the amino acid sequence of SEQ ID NO:17, and b) the second polypeptide chain comprises a second Fc domain and a masking moiety that binds to the IL-12p35 and/or IL-12p40 subunit in the first polypeptide chain, wherein the second Fc domain comprises the amino acid sequence of SEQ ID NO:18 and the masking moiety is selected from the group consisting of any one of SEQ ID NOs:61-109; wherein the first and second polypeptide chain are linked via the first Fc domain and the second Fc domain, wherein the IL-12p35 subunit or the IL-12p40 subunit is linked to the C-terminus of the first Fc domain via a first peptide linker, which first peptide linker is protease-cleavable, wherein the protease-cleavable linker is selected from the group consisting of any one of the amino acid sequences of SEQ ID NOs:232-241, wherein the masking moiety is linked to the C-terminus of the second Fc domain via a second linker, preferably a peptide linker and wherein the first or the second polypeptide chain further comprises a collagen binding moiety having the amino acid sequence of SEQ ID NO:47. The masking moiety may further comprise an additional alanine attached to its C-terminus.

In one embodiment the Interleukin-12 (IL-12) Fc fusion protein comprises a first polypeptide chain and a second polypeptide chain, wherein a) the first polypeptide chain comprises a first Fc domain and an IL-12p35 subunit and an IL-12p40 subunit of IL-12, and b) the second polypeptide chain comprises a second Fc domain and a masking moiety that binds to the IL-12p35 and/or IL-12p40 subunit in the first polypeptide chain, wherein the masking moiety is selected from the group consisting of any one of SEQ ID NOs:61-109; wherein the first and second polypeptide chain are linked via the first Fc domain and the second Fc domain, wherein the IL-12p35 subunit or the IL-12p40 subunit is linked to the C-terminus of the first Fc domain via a first peptide linker, which first peptide linker is protease-cleavable, wherein the protease-cleavable linker is selected from the group consisting of any one of the amino acid sequences of SEQ ID NOs:232-241, wherein the masking moiety is linked to the C-terminus of the second Fc domain via a second linker, preferably a peptide linker and wherein the first or the second polypeptide chain further comprises a collagen binding moiety having the amino acid sequence of SEQ ID NO:41. The masking moiety may further comprise an additional alanine attached to its C-terminus.

In one embodiment the Interleukin-12 (IL-12) Fc fusion protein comprises a first polypeptide chain and a second polypeptide chain, wherein a) the first polypeptide chain comprises a first Fc domain and an IL-12p35 subunit and an IL-12p40 subunit of IL-12, and b) the second polypeptide chain comprises a second Fc domain and a masking moiety that binds to the IL-12p35 and/or IL-12p40 subunit in the first polypeptide chain, wherein the masking moiety is selected from the group consisting of any one of SEQ ID NOs:61-109; wherein the first and second polypeptide chain are linked via the first Fc domain and the second Fc domain, wherein the IL-12p35 subunit or the IL-12p40 subunit is linked to the C-terminus of the first Fc domain via a first peptide linker, which first peptide linker is protease-cleavable, wherein the protease-cleavable linker is selected from the group consisting of any one of the amino acid sequences of SEQ ID NOs:232-241, wherein the masking moiety is linked to the C-terminus of the second Fc domain via a second linker, preferably a peptide linker and wherein the first or the second polypeptide chain further comprises a collagen binding moiety having the amino acid sequence of SEQ ID NO:42. The masking moiety may further comprise an additional alanine attached to its C-terminus.

In one embodiment the Interleukin-12 (IL-12) Fc fusion protein comprises a first polypeptide chain and a second polypeptide chain, wherein a) the first polypeptide chain comprises a first Fc domain and an IL-12p35 subunit and an IL-12p40 subunit of IL-12, and b) the second polypeptide chain comprises a second Fc domain and a masking moiety that binds to the IL-12p35 and/or IL-12p40 subunit in the first polypeptide chain, wherein the masking moiety is selected from the group consisting of any one of SEQ ID NOs:61-109; wherein the first and second polypeptide chain are linked via the first Fc domain and the second Fc domain, wherein the IL-12p35 subunit or the IL-12p40 subunit is linked to the C-terminus of the first Fc domain via a first peptide linker, which first peptide linker is protease-cleavable, wherein the protease-cleavable linker is selected from the group consisting of any one of the amino acid sequences of SEQ ID NOs:232-241, wherein the masking moiety is linked to the C-terminus of the second Fc domain via a second linker, preferably a peptide linker and wherein the first or the second polypeptide chain further comprises a collagen binding moiety having the amino acid sequence of SEQ ID NO:43. The masking moiety may further comprise an additional alanine attached to its C-terminus.

In one embodiment the Interleukin-12 (IL-12) Fc fusion protein comprises a first polypeptide chain and a second polypeptide chain, wherein a) the first polypeptide chain comprises a first Fc domain and an IL-12p35 subunit and an IL-12p40 subunit of IL-12, and b) the second polypeptide chain comprises a second Fc domain and a masking moiety that binds to the IL-12p35 and/or IL-12p40 subunit in the first polypeptide chain, wherein the masking moiety is selected from the group consisting of any one of SEQ ID NOs:61-109; wherein the first and second polypeptide chain are linked via the first Fc domain and the second Fc domain, wherein the IL-12p35 subunit or the IL-12p40 subunit is linked to the C-terminus of the first Fc domain via a first peptide linker, which first peptide linker is protease-cleavable, wherein the protease-cleavable linker is selected from the group consisting of any one of the amino acid sequences of SEQ ID NOs:232-241, wherein the masking moiety is linked to the C-terminus of the second Fc domain via a second linker, preferably a peptide linker and wherein the first or the second polypeptide chain further comprises a collagen binding moiety having the amino acid sequence of SEQ ID NO:44. The masking moiety may further comprise an additional alanine attached to its C-terminus.

In one embodiment the Interleukin-12 (IL-12) Fc fusion protein comprises a first polypeptide chain and a second polypeptide chain, wherein a) the first polypeptide chain comprises a first Fc domain and an IL-12p35 subunit and an IL-12p40 subunit of IL-12, and b) the second polypeptide chain comprises a second Fc domain and a masking moiety that binds to the IL-12p35 and/or IL-12p40 subunit in the first polypeptide chain, wherein the masking moiety is selected from the group consisting of any one of SEQ ID NOs:61-109; wherein the first and second polypeptide chain are linked via the first Fc domain and the second Fc domain, wherein the IL-12p35 subunit or the IL-12p40 subunit is linked to the C-terminus of the first Fc domain via a first peptide linker, which first peptide linker is protease-cleavable, wherein the protease-cleavable linker is selected from the group consisting of any one of the amino acid sequences of SEQ ID NOs:232-241, wherein the masking moiety is linked to the C-terminus of the second Fc domain via a second linker, preferably a peptide linker and wherein the first or the second polypeptide chain further comprises a collagen binding moiety having the amino acid sequence of SEQ ID NO:45. The masking moiety may further comprise an additional alanine attached to its C-terminus.

In one embodiment the Interleukin-12 (IL-12) Fc fusion protein comprises a first polypeptide chain and a second polypeptide chain, wherein a) the first polypeptide chain comprises a first Fc domain and an IL-12p35 subunit and an IL-12p40 subunit of IL-12, and b) the second polypeptide chain comprises a second Fc domain and a masking moiety that binds to the IL-12p35 and/or IL-12p40 subunit in the first polypeptide chain, wherein the masking moiety is selected from the group consisting of any one of SEQ ID NOs:61-109; wherein the first and second polypeptide chain are linked via the first Fc domain and the second Fc domain, wherein the IL-12p35 subunit or the IL-12p40 subunit is linked to the C-terminus of the first Fc domain via a first peptide linker, which first peptide linker is protease-cleavable, wherein the protease-cleavable linker is selected from the group consisting of any one of the amino acid sequences of SEQ ID NOs:232-241, wherein the masking moiety is linked to the C-terminus of the second Fc domain via a second linker, preferably a peptide linker and wherein the first or the second polypeptide chain further comprises a collagen binding moiety having the amino acid sequence of SEQ ID NO:46. The masking moiety may further comprise an additional alanine attached to its C-terminus.

In one embodiment the Interleukin-12 (IL-12) Fc fusion protein comprises a first polypeptide chain and a second polypeptide chain, wherein a) the first polypeptide chain comprises a first Fc domain and an IL-12p35 subunit and an IL-12p40 subunit of IL-12, and b) the second polypeptide chain comprises a second Fc domain and a masking moiety that binds to the IL-12p35 and/or IL-12p40 subunit in the first polypeptide chain, wherein the masking moiety is selected from the group consisting of any one of SEQ ID NOs:61-109; wherein the first and second polypeptide chain are linked via the first Fc domain and the second Fc domain, wherein the IL-12p35 subunit or the IL-12p40 subunit is linked to the C-terminus of the first Fc domain via a first peptide linker, which first peptide linker is protease-cleavable, wherein the protease-cleavable linker is selected from the group consisting of any one of the amino acid sequences of SEQ ID NOs:232-241, wherein the masking moiety is linked to the C-terminus of the second Fc domain via a second linker, preferably a peptide linker and wherein the first or the second polypeptide chain further comprises a collagen binding moiety having the amino acid sequence of SEQ ID NO:47. The masking moiety may further comprise an additional alanine attached to its C-terminus.

In one embodiment the Interleukin-12 (IL-12) Fc fusion protein comprises a first polypeptide chain and a second polypeptide chain, wherein a) the first polypeptide chain comprises a first Fc domain and an IL-12p35 subunit and an IL-12p40 subunit of IL-12, and b) the second polypeptide chain comprises a second Fc domain and a masking moiety that binds to the IL-12p35 and/or IL-12p40 subunit in the first polypeptide chain, wherein the masking moiety is selected from the group consisting of any one of SEQ ID NOs:61-109; wherein the first and second polypeptide chain are linked via the first Fc domain and the second Fc domain, wherein the IL-12p35 subunit or the IL-12p40 subunit is linked to the C-terminus of the first Fc domain via a first peptide linker, which first peptide linker is protease-cleavable, wherein the protease-cleavable linker comprises or consists of the amino acid sequence of SEQ ID NO:232, wherein the masking moiety is linked to the C-terminus of the second Fc domain via a second linker preferably a peptide linker, and wherein the first or the second polypeptide chain further comprises a collagen binding moiety having the amino acid sequence of SEQ ID NO:41. The masking moiety may further comprise an additional alanine attached to its C-terminus.

In one embodiment the Interleukin-12 (IL-12) Fc fusion protein comprises a first polypeptide chain and a second polypeptide chain, wherein a) the first polypeptide chain comprises a first Fc domain and an IL-12p35 subunit and an IL-12p40 subunit of IL-12, and b) the second polypeptide chain comprises a second Fc domain and a masking moiety that binds to the IL-12p35 and/or IL-12p40 subunit in the first polypeptide chain, wherein the masking moiety is selected from the group consisting of any one of SEQ ID NOs:61-109; wherein the first and second polypeptide chain are linked via the first Fc domain and the second Fc domain, wherein the IL-12p35 subunit or the IL-12p40 subunit is linked to the C-terminus of the first Fc domain via a first peptide linker, which first peptide linker is protease-cleavable, wherein the protease-cleavable linker comprises or consists of the amino acid sequence of SEQ ID NO:232, wherein the masking moiety is linked to the C-terminus of the second Fc domain via a second linker, preferably a peptide linker and wherein the first or the second polypeptide chain further comprises a collagen binding moiety having the amino acid sequence of SEQ ID NO:42. The masking moiety may further comprise an additional alanine attached to its C-terminus.

In one embodiment the Interleukin-12 (IL-12) Fc fusion protein comprises a first polypeptide chain and a second polypeptide chain, wherein a) the first polypeptide chain comprises a first Fc domain and an IL-12p35 subunit and an IL-12p40 subunit of IL-12, and b) the second polypeptide chain comprises a second Fc domain and a masking moiety that binds to the IL-12p35 and/or IL-12p40 subunit in the first polypeptide chain, wherein the masking moiety is selected from the group consisting of any one of SEQ ID NOs:61-109; wherein the first and second polypeptide chain are linked via the first Fc domain and the second Fc domain, wherein the IL-12p35 subunit or the IL-12p40 subunit is linked to the C-terminus of the first Fc domain via a first peptide linker, which first peptide linker is protease-cleavable, wherein the protease-cleavable linker comprises or consists of the amino acid sequence of SEQ ID NO:232, wherein the masking moiety is linked to the C-terminus of the second Fc domain via a second linker, preferably a peptide linker and wherein the first or the second polypeptide chain further comprises a collagen binding moiety having the amino acid sequence of SEQ ID NO:43. The masking moiety may further comprise an additional alanine attached to its C-terminus.

In one embodiment the Interleukin-12 (IL-12) Fc fusion protein comprises a first polypeptide chain and a second polypeptide chain, wherein a) the first polypeptide chain comprises a first Fc domain and an IL-12p35 subunit and an IL-12p40 subunit of IL-12, and b) the second polypeptide chain comprises a second Fc domain and a masking moiety that binds to the IL-12p35 and/or IL-12p40 subunit in the first polypeptide chain, wherein the masking moiety is selected from the group consisting of any one of SEQ ID NOs:61-109; wherein the first and second polypeptide chain are linked via the first Fc domain and the second Fc domain, wherein the IL-12p35 subunit or the IL-12p40 subunit is linked to the C-terminus of the first Fc domain via a first peptide linker, which first peptide linker is protease-cleavable, wherein the protease-cleavable linker comprises or consists of the amino acid sequence of SEQ ID NO:232, wherein the masking moiety is linked to the C-terminus of the second Fc domain via a second linker, preferably a peptide linker and wherein the first or the second polypeptide chain further comprises a collagen binding moiety having the amino acid sequence of SEQ ID NO:44. The masking moiety may further comprise an additional alanine attached to its C-terminus.

In one embodiment the Interleukin-12 (IL-12) Fc fusion protein comprises a first polypeptide chain and a second polypeptide chain, wherein a) the first polypeptide chain comprises a first Fc domain and an IL-12p35 subunit and an IL-12p40 subunit of IL-12, and b) the second polypeptide chain comprises a second Fc domain and a masking moiety that binds to the IL-12p35 and/or IL-12p40 subunit in the first polypeptide chain, wherein the masking moiety is selected from the group consisting of any one of SEQ ID NOs:61-109; wherein the first and second polypeptide chain are linked via the first Fc domain and the second Fc domain, wherein the IL-12p35 subunit or the IL-12p40 subunit is linked to the C-terminus of the first Fc domain via a first peptide linker, which first peptide linker is protease-cleavable, wherein the protease-cleavable linker comprises or consists of the amino acid sequence of SEQ ID NO:232, wherein the masking moiety is linked to the C-terminus of the second Fc domain via a second linker, preferably a peptide linker and wherein the first or the second polypeptide chain further comprises a collagen binding moiety having the amino acid sequence of SEQ ID NO:45. The masking moiety may further comprise an additional alanine attached to its C-terminus.

In one embodiment the Interleukin-12 (IL-12) Fc fusion protein comprises a first polypeptide chain and a second polypeptide chain, wherein a) the first polypeptide chain comprises a first Fc domain and an IL-12p35 subunit and an IL-12p40 subunit of IL-12, and b) the second polypeptide chain comprises a second Fc domain and a masking moiety that binds to the IL-12p35 and/or IL-12p40 subunit in the first polypeptide chain, wherein the masking moiety is selected from the group consisting of any one of SEQ ID NOs:61-109; wherein the first and second polypeptide chain are linked via the first Fc domain and the second Fc domain, wherein the IL-12p35 subunit or the IL-12p40 subunit is linked to the C-terminus of the first Fc domain via a first peptide linker, which first peptide linker is protease-cleavable, wherein the protease-cleavable linker comprises or consists of the amino acid sequence of SEQ ID NO:232, wherein the masking moiety is linked to the C-terminus of the second Fc domain via a second linker, preferably a peptide linker and wherein the first or the second polypeptide chain further comprises a collagen binding moiety having the amino acid sequence of SEQ ID NO:46. The masking moiety may further comprise an additional alanine attached to its C-terminus.

In one embodiment the Interleukin-12 (IL-12) Fc fusion protein comprises a first polypeptide chain and a second polypeptide chain, wherein a) the first polypeptide chain comprises a first Fc domain and an IL-12p35 subunit and an IL-12p40 subunit of IL-12, and b) the second polypeptide chain comprises a second Fc domain and a masking moiety that binds to the IL-12p35 and/or IL-12p40 subunit in the first polypeptide chain, wherein the masking moiety is selected from the group consisting of any one of SEQ ID NOs:61-109; wherein the first and second polypeptide chain are linked via the first Fc domain and the second Fc domain, wherein the IL-12p35 subunit or the IL-12p40 subunit is linked to the C-terminus of the first Fc domain via a first peptide linker, which first peptide linker is protease-cleavable, wherein the protease-cleavable linker comprises or consists of the amino acid sequence of SEQ ID NO:232, wherein the masking moiety is linked to the C-terminus of the second Fc domain via a second linker, preferably a peptide linker and wherein the first or the second polypeptide chain further comprises a collagen binding moiety having the amino acid sequence of SEQ ID NO:47. The masking moiety may further comprise an additional alanine attached to its C-terminus.

In one embodiment relating to any of the foregoing embodiments, the IL-12 activity of the uncleaved IL-12 Fc fusion protein is at least 50-fold, 75-fold, 100-fold, 125-fold, 150-fold, 175-fold, 200-fold, 225-fold, 250-fold, 275-fold, 300-fold, 325-fold, 350-fold, 375-fold, 400-fold, 425-fold, 450-fold, 475-fold, 500-fold, 525-fold, 550-fold, 575-fold, or 600-fold lower compared to the IL-12 activity of the IL-12 Fc fusion protein after cleavage of the cleavable linker. In other words, the delta EC₅₀ of the uncleaved IL-12 Fc fusion protein and the cleaved IL-12 Fc fusion protein as measured in an IL-12 bioassay (EC₅₀ uncleaved IL-12 Fc fusion protein: EC₅₀ cleaved IL-12 Fc fusion protein), e.g. as in the Promega IL-12 Bioassay described in the examples, is at least 50, 75, 100, 125, 150, 175, 200, 225, 250, 275, 300, 325, 350, 400, 425, 450, 475, 500, 525, 550, 575, or 600.

In the following table further IL-12 Fc fusion proteins or VHH masking moieties are disclosed which have been conceived or generated, e.g. as intermediate molecules during optimization or as further alternatives. It will be understood that each IL-12 Fc fusion protein is made up of two chains identified by the appropriate identifiers, e.g. BI-062 Knob is only paired with BI-062 Hole; or Knob Chain BI-067 is only paired with Hole Chain BI-067. Hence, in another embodiment the invention relates to an IL-12 Fc fusion protein comprising or consisting of two polypeptide chains identified by their appropriate identifier pairs as disclosed in TABLE 6, e.g SEQ ID NOs:242 and 243; SEQ ID NOs:245 and 246; SEQ ID NOs:247 and 248, and so forth.

TABLE 6

| Identifier | Sequence | SEQ ID NO: |
|---|---|---|
| BI-062 Fc Knob | EPKSSDKTHTCPPCPAPEAAGGPSVFLFPPKPKDTLMISRTPEVT | 242 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGGGSGGGGSGGGGSRNLPVATPDPGM | |
| | FPCLHHSQNLLRAVSNMLQKARQTLEFYPCTSEEIDHEDITKDKT | |
| | STVEACLPLELTKNESCLNSRETSFITNGSCLASRKTSFMMALCL | |
| | SSIYEDLKMYQVEFKTMNAKLLMDPKRQIFLDQNMLAVIDELMQA | |
| | LNFNSETVPQKSSLEEPDFYKTKIKLCILLHAFRIRAVTIDRVMS | |
| | YLNAS | |
| BI-062 Fc Hole | EPKSSDKTHTCPPCPAPEAAGGPSVFLFPPKPKDTLMISRTPEVT | 243 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSQVQLVESGGGLVQA | |
| | GGSLRLSCAASGRTFETIAMGWFRQVPGKEREFVAVIRGSGVATY | |
| | YPDSVKGRFTISKDSAKNTVYLQMNNLKPEDTAVYYCAATTNREK | |
| | NTDYTTYDYWGQGTQVTVSSSG | |
| BI-063 | EVQLVESGGGLVQPGGSLRLSCAASGRTFETIAMGWFRQAPGKER | 244 |
| VHH | EFVAVIRGSGVATYYPDSVKGRFTISKDSSKNTVYLQMNSLRAED | |
| | TAVYYCAATTNRFKNTDYTTYDYWGQGTQVTVSS | |
| Knob Chain BI-067 | EPKSSDKTHTCPPCPAPEAAGGPSVFLFPPKPKDTLMISRTPEVT | 245 |
| | CVVVDVSHEDPEVKENWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGSEGKSSGSGSESKSTGGSRNLPVAT | |
| | PDPGMFPCLHHSQNLLRAVSNMLQKARQTLEFYPCTSEEIDHEDI | |
| | TKDKTSTVEACLPLELTKNESCLNSRETSFITNGSCLASRKTSFM | |
| | MALCLSSIYEDLKMYQVEFKTMNAKLLMDPKRQIFLDQNMLAVID | |
| | ELMQALNFNSETVPQKSSLEEPDFYKTKIKLCILLHAFRIRAVTI | |
| | DRVMSYLNASGSGGLRELHLDNN | |
| Hole Chain BI-067 | EPKSSDKTHTCPPCPAPEAAGGPSVFLFPPKPKDTLMISRTPEVT | 246 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NTDYTTYDYWGQGTQVTVSSA | |
| Knob Chain BI-068 | EPKSSDKTHTCPPCPAPEAAGGPSVFLFPPKPKDTLMISRTPEVT | 247 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGSEGKSSGSGSESKSTGGSRNLPVAT | |
| | PDPGMFPCLHHSQNLLRAVSNMLQKARQTLEFYPCTSEEIDHEDI | |
| | TKDKTSTVEACLPLELTKNESCLNSRETSFITNGSCLASRKTSEM | |
| | MALCLSSIYEDLKMYQVEFKTMNAKLLMDPKRQIFLDQNMLAVID | |
| | ELMQALNENSETVPQKSSLEEPDFYKTKIKLCILLHAFRIRAVTI | |
| | DRVMSYLNASGSGGLRELHLDNN | |
| Hole Chain BI-068 | EPKSSDKTHTCPPCPAPEAAGGPSVFLFPPKPKDTLMISRTPEVT | 248 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NREYTTYDYWGQGTQVTVSSA | |
| Knob Chain BI-069 | EPKSSDKTHTCPPCPAPEAAGGPSVFLFPPKPKDTLMISRTPEVT | 249 |
| | CVVVDVSHEDPEVKENWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGGGSGGGGSGGGGSLRELHLDNNGGS | |
| | RNLPVATPDPGMFPCLHHSQNLLRAVSNMLQKARQTLEFYPCTSE | |
| | EIDHEDITKDKTSTVEACLPLELTKNESCLNSRETSFITNGSCLA | |
| | SRKTSFMMALCLSSIYEDLKMYQVEFKTMNAKLLMDPKRQIFLDQ | |
| | NMLAVIDELMQALNENSETVPQKSSLEEPDFYKTKIKLCILLHAF | |
| | RIRAVTIDRVMSYLNAS | |
| Hole Chain BI-069 | EPKSSDKTHTCPPCPAPEAAGGPSVFLFPPKPKDTLMISRTPEVT | 250 |
| | CVVVDVSHEDPEVKENWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NREYTTYDYWGQGTQVTVSSA | |
| Knob Chain BI-070 | EPKSSDKTHTCPPCPAPEAAGGPSVFLFPPKPKDTLMISRTPEVT | 251 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGSEGKSSGSGSESKSTGGSLRELHLD | |
| | NNGGSRNLPVATPDPGMFPCLHHSQNLLRAVSNMLQKARQTLEFY | |
| | PCTSEEIDHEDITKDKTSTVEACLPLELTKNESCLNSRETSFITN | |
| | GSCLASRKTSFMMALCLSSIYEDLKMYQVEFKTMNAKLLMDPKRQ | |
| | IFLDQNMLAVIDELMQALNENSETVPQKSSLEEPDFYKTKIKLCI | |
| | LLHAFRIRAVTIDRVMSYLNAS | |
| Hole Chain BI-070 | EPKSSDKTHTCPPCPAPEAAGGPSVFLFPPKPKDTLMISRTPEVT | 252 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NTDYTTYDYWGQGTQVTVSSA | |
| Knob Chain BI-071 | EPKSSDKTHTCPPCPAPEAAGGPSVFLFPPKPKDTLMISRTPEVT | 253 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGSEGKSSGSGSESKSTGGSLRELHLD | |
| | NNGGSRNLPVATPDPGMFPCLHHSQNLLRAVSNMLQKARQTLEFY | |
| | PCTSEEIDHEDITKDKTSTVEACLPLELTKNESCLNSRETSFITN | |
| | GSCLASRKTSFMMALCLSSIYEDLKMYQVEFKTMNAKLLMDPKRQ | |
| | IFLDQNMLAVIDELMQALNENSETVPQKSSLEEPDFYKTKIKLCI | |
| | LLHAFRIRAVTIDRVMSYLNAS | |
| Hole Chain BI-071 | EPKSSDKTHTCPPCPAPEAAGGPSVFLFPPKPKDTLMISRTPEVT | 254 |
| | CVVVDVSHEDPEVKENWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NREYTTYDYWGQGTQVTVSSA | |
| Knob Chain BI-072 | EPKSSDKTHTCPPCPAPEAAGGPSVFLFPPKPKDTLMISRTPEVT | 255 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGLRELHLDNNGGSIWELKKDVYVV | |
| | ELDWYPDAPGEMVVLTCDTPEEDGITWTLDQSSEVLGSGKTLTIQ | |
| | VKEFGDAGQYTCHKGGEVLSHSLLLLHKKEDGIWSTDILKDQKEP | |
| | KNKTFLRCEAKNYSGRFTCWWLTTISTDLTFSVKSSRGSSDPQGV | |
| | TCGAATLSAERVRGDNKEYEYSVECQEDSACPAAEESLPIEVMVD | |
| | AVHKLKYENYTSSFFIRDIIKPDPPKNLQLKPLKNSRQVEVSWEY | |
| | PDTWSTPHSYFSLTFCVQVQGKSKREKKDRVFTDKTSATVICRKN | |
| | ASISVRAQDRYYSSSWSEWASVPCSGGGGSGGGGSGGGGSRNLPV | |
| | ATPDPGMFPCLHHSQNLLRAVSNMLQKARQTLEFYPCTSEEIDHE | |
| | DITKDKTSTVEACLPLELTKNESCLNSRETSFITNGSCLASRKTS | |
| | FMMALCLSSIYEDLKMYQVEFKTMNAKLLMDPKRQIFLDQNMLAV | |
| | IDELMQALNFNSETVPQKSSLEEPDFYKTKIKLCILLHAFRIRAV | |
| | TIDRVMSYLNAS | |
| Hole Chain BI-072 | EPKSSDKTHTCPPCPAPEAAGGPSVFLFPPKPKDTLMISRTPEVT | 256 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NREYTTYDYWGQGTQVTVSSA | |
| Knob Chain BI-073 | EPKSSDKTHTCPPCPAPEAAGGPSVFLFPPKPKDTLMISRTPEVT | 257 |
| | CVVVDVSHEDPEVKENWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGLRELHLDNNGGSIWELKKDVYVV | |
| | ELDWYPDAPGEMVVLTCDTPEEDGITWTLDQSSEVLGSGKTLTIQ | |
| | VKEFGDAGQYTCHKGGEVLSHSLLLLHKKEDGIWSTDILKDQKEP | |
| | KNKTFLRCEAKNYSGRFTCWWLTTISTDLTFSVKSSRGSSDPQGV | |
| | TCGAATLSAERVRGDNKEYEYSVECQEDSACPAAEESLPIEVMVD | |
| | AVHKLKYENYTSSFFIRDIIKPDPPKNLQLKPLKNSRQVEVSWEY | |
| | PDTWSTPHSYFSLTFCVQVQGKSKREKKDRVFTDKTSATVICRKN | |
| | ASISVRAQDRYYSSSWSEWASVPCSGGSEGKSSGSGSESKSTGGS | |
| | RNLPVATPDPGMFPCLHHSQNLLRAVSNMLQKARQTLEFYPCTSE | |
| | EIDHEDITKDKTSTVEACLPLELTKNESCLNSRETSFITNGSCLA | |
| | SRKTSFMMALCLSSIYEDLKMYQVEFKTMNAKLLMDPKRQIFLDQ | |
| | NMLAVIDELMQALNENSETVPQKSSLEEPDFYKTKIKLCILLHAF | |
| | RIRAVTIDRVMSYLNAS | |
| Hole Chain BI-073 | EPKSSDKTHTCPPCPAPEAAGGPSVFLFPPKPKDTLMISRTPEVT | 258 |
| | CVVVDVSHEDPEVKENWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNREK | |
| | NTDYTTYDYWGQGTQVTVSSA | |
| Knob Chain BI-074 | EPKSSDKTHTCPPCPAPEAAGGPSVFLFPPKPKDTLMISRTPEVT | 259 |
| | CVVVDVSHEDPEVKENWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGLRELHLDNNGGSIWELKKDVYVV | |
| | ELDWYPDAPGEMVVLTCDTPEEDGITWTLDQSSEVLGSGKTLTIQ | |
| | VKEFGDAGQYTCHKGGEVLSHSLLLLHKKEDGIWSTDILKDQKEP | |
| | KNKTFLRCEAKNYSGRFTCWWLTTISTDLTFSVKSSRGSSDPQGV | |
| | TCGAATLSAERVRGDNKEYEYSVECQEDSACPAAEESLPIEVMVD | |
| | AVHKLKYENYTSSFFIRDIIKPDPPKNLQLKPLKNSRQVEVSWEY | |
| | PDTWSTPHSYFSLTFCVQVQGKSKREKKDRVFTDKTSATVICRKN | |
| | ASISVRAQDRYYSSSWSEWASVPCSGGSEGKSSGSGSESKSTGGS | |
| | RNLPVATPDPGMFPCLHHSQNLLRAVSNMLQKARQTLEFYPCTSE | |
| | EIDHEDITKDKTSTVEACLPLELTKNESCLNSRETSFITNGSCLA | |
| | SRKTSFMMALCLSSIYEDLKMYQVEFKTMNAKLLMDPKRQIFLDQ | |
| | NMLAVIDELMQALNFNSETVPQKSSLEEPDFYKTKIKLCILLHAF | |
| | RIRAVTIDRVMSYLNAS | |
| Hole Chain BI-074 | EPKSSDKTHTCPPCPAPEAAGGPSVFLFPPKPKDTLMISRTPEVT | 260 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNREK | |
| | NREYTTYDYWGQGTQVTVSSA | |
| Knob Chain BI-075 | EPKSSDKTHTCPPCPAPEAAGGPSVFLFPPKPKDTLMISRTPEVT | 261 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGGGSGGGGSGGGGSRNLPVATPDPGM | |
| | FPCLHHSQNLLRAVSNMLQKARQTLEFYPCTSEEIDHEDITKDKT | |
| | STVEACLPLELTKNESCLNSRETSFITNGSCLASRKTSEMMALCL | |
| | SSIYEDLKMYQVEFKTMNAKLLMDPKRQIFLDQNMLAVIDELMQA | |
| | LNFNSETVPQKSSLEEPDFYKTKIKLCILLHAFRIRAVTIDRVMS | |
| | YLNASGSGGGGWSHW | |
| Hole Chain BI-075 | EPKSSDKTHTCPPCPAPEAAGGPSVFLFPPKPKDTLMISRTPEVT | 262 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NREYTTYDYWGQGTQVTVSSA | |
| Knob Chain BI-076 | EPKSSDKTHTCPPCPAPEAAGGPSVFLFPPKPKDTLMISRTPEVT | 263 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGSEGKSSGSGSESKSTGGSRNLPVAT | |
| | PDPGMFPCLHHSQNLLRAVSNMLQKARQTLEFYPCTSEEIDHEDI | |
| | TKDKTSTVEACLPLELTKNESCLNSRETSFITNGSCLASRKTSFM | |
| | MALCLSSIYEDLKMYQVEFKTMNAKLLMDPKRQIFLDQNMLAVID | |
| | ELMQALNFNSETVPQKSSLEEPDFYKTKIKLCILLHAFRIRAVTI | |
| | DRVMSYLNASGSGGGGWSHW | |
| Hole Chain BI-076 | EPKSSDKTHTCPPCPAPEAAGGPSVFLFPPKPKDTLMISRTPEVT | 264 |
| | CVVVDVSHEDPEVKENWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NTDYTTYDYWGQGTQVTVSSA | |
| Knob Chain BI-077 | EPKSSDKTHTCPPCPAPEAAGGPSVFLFPPKPKDTLMISRTPEVT | 265 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGSEGKSSGSGSESKSTGGSRNLPVAT | |
| | PDPGMFPCLHHSQNLLRAVSNMLQKARQTLEFYPCTSEEIDHEDI | |
| | TKDKTSTVEACLPLELTKNESCLNSRETSFITNGSCLASRKTSFM | |
| | MALCLSSIYEDLKMYQVEFKTMNAKLLMDPKRQIFLDQNMLAVID | |
| | ELMQALNFNSETVPQKSSLEEPDFYKTKIKLCILLHAFRIRAVTI | |
| | DRVMSYLNASGSGGGGWSHW | |
| Hole Chain BI-077 | EPKSSDKTHTCPPCPAPEAAGGPSVFLFPPKPKDTLMISRTPEVT | 266 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NREYTTYDYWGQGTQVTVSSA | |
| Knob Chain BI-078 | EPKSSDKTHTCPPCPAPEAAGGPSVFLFPPKPKDTLMISRTPEVT | 267 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSSGGGGSGGGGSGGGGSGGWSHWGGSRN | |
| | LPVATPDPGMFPCLHHSQNLLRAVSNMLQKARQTLEFYPCTSEEI | |
| | DHEDITKDKTSTVEACLPLELTKNESCLNSRETSFITNGSCLASR | |
| | KTSFMMALCLSSIYEDLKMYQVEFKTMNAKLLMDPKRQIFLDQNM | |
| | LAVIDELMQALNFNSETVPQKSSLEEPDFYKTKIKLCILLHAFRI | |
| | RAVTIDRVMSYLNAS | |
| Hole Chain BI-078 | EPKSSDKTHTCPPCPAPEAAGGPSVFLFPPKPKDTLMISRTPEVT | 268 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NTDYTTYDYWGQGTQVTVSSA | |
| Knob Chain BI-079 | EPKSSDKTHTCPPCPAPEAAGGPSVFLFPPKPKDTLMISRTPEVT | 269 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSSGGGGSGGGGSGGGGSGGWSHWGGSRN | |
| | LPVATPDPGMFPCLHHSQNLLRAVSNMLQKARQTLEFYPCTSEEI | |
| | DHEDITKDKTSTVEACLPLELTKNESCLNSRETSFITNGSCLASR | |
| | KTSFMMALCLSSIYEDLKMYQVEFKTMNAKLLMDPKRQIFLDQNM | |
| | LAVIDELMQALNFNSETVPQKSSLEEPDFYKTKIKLCILLHAFRI | |
| | RAVTIDRVMSYLNAS | |
| Hole Chain BI-079 | EPKSSDKTHTCPPCPAPEAAGGPSVFLFPPKPKDTLMISRTPEVT | 270 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NREYTTYDYWGQGTQVTVSSA | |
| Knob Chain BI-080 | EPKSSDKTHTCPPCPAPEAAGGPSVFLFPPKPKDTLMISRTPEVT | 271 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGSEGKSSGSGSESKSTGGSGGWSHWG | |
| | GSRNLPVATPDPGMFPCLHHSQNLLRAVSNMLQKARQTLEFYPCT | |
| | SEEIDHEDITKDKTSTVEACLPLELTKNESCLNSRETSFITNGSC | |
| | LASRKTSFMMALCLSSIYEDLKMYQVEFKTMNAKLLMDPKRQIFL | |
| | DQNMLAVIDELMQALNFNSETVPQKSSLEEPDFYKTKIKLCILLH | |
| | AFRIRAVTIDRVMSYLNAS | |
| Hole Chain BI-080 | EPKSSDKTHTCPPCPAPEAAGGPSVFLFPPKPKDTLMISRTPEVT | 272 |
| | CVVVDVSHEDPEVKENWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NTDYTTYDYWGQGTQVTVSSA | |
| Knob Chain BI-081 | EPKSSDKTHTCPPCPAPEAAGGPSVFLFPPKPKDTLMISRTPEVT | 273 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGSEGKSSGSGSESKSTGGSGGWSHWG | |
| | GSRNLPVATPDPGMFPCLHHSQNLLRAVSNMLQKARQTLEFYPCT | |
| | SEEIDHEDITKDKTSTVEACLPLELTKNESCLNSRETSFITNGSC | |
| | LASRKTSFMMALCLSSIYEDLKMYQVEFKTMNAKLLMDPKRQIFL | |
| | DQNMLAVIDELMQALNFNSETVPQKSSLEEPDFYKTKIKLCILLH | |
| | AFRIRAVTIDRVMSYLNAS | |
| Hole Chain BI-081 | EPKSSDKTHTCPPCPAPEAAGGPSVFLFPPKPKDTLMISRTPEVT | 274 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NREYTTYDYWGQGTQVTVSSA | |
| Knob Chain BI-082 | EPKSSDKTHTCPPCPAPEAAGGPSVFLFPPKPKDTLMISRTPEVT | 275 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGSGGWSHWGGSIWELKKDVYVV | |
| | ELDWYPDAPGEMVVLTCDTPEEDGITWTLDQSSEVLGSGKTLTIQ | |
| | VKEFGDAGQYTCHKGGEVLSHSLLLLHKKEDGIWSTDILKDQKEP | |
| | KNKTFLRCEAKNYSGRFTCWWLTTISTDLTFSVKSSRGSSDPQGV | |
| | TCGAATLSAERVRGDNKEYEYSVECQEDSACPAAEESLPIEVMVD | |
| | AVHKLKYENYTSSFFIRDIIKPDPPKNLQLKPLKNSRQVEVSWEY | |
| | PDTWSTPHSYFSLTFCVQVQGKSKREKKDRVFTDKTSATVICRKN | |
| | ASISVRAQDRYYSSSWSEWASVPCSGGGGSGGGGSGGGGSRNLPV | |
| | ATPDPGMFPCLHHSQNLLRAVSNMLQKARQTLEFYPCTSEEIDHE | |
| | DITKDKTSTVEACLPLELTKNESCLNSRETSFITNGSCLASRKTS | |
| | FMMALCLSSIYEDLKMYQVEFKTMNAKLLMDPKRQIFLDQNMLAV | |
| | IDELMQALNENSETVPQKSSLEEPDFYKTKIKLCILLHAFRIRAV | |
| | TIDRVMSYLNAS | |
| Hole Chain BI-082 | EPKSSDKTHTCPPCPAPEAAGGPSVFLFPPKPKDTLMISRTPEVT | 276 |
| | CVVVDVSHEDPEVKENWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NTDYTTYDYWGQGTQVTVSSA | |
| Knob Chain BI-083 | EPKSSDKTHTCPPCPAPEAAGGPSVFLFPPKPKDTLMISRTPEVT | 277 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGSGGWSHWGGSIWELKKDVYVV | |
| | ELDWYPDAPGEMVVLTCDTPEEDGITWTLDQSSEVLGSGKTLTIQ | |
| | VKEFGDAGQYTCHKGGEVLSHSLLLLHKKEDGIWSTDILKDQKEP | |
| | KNKTFLRCEAKNYSGRFTCWWLTTISTDLTFSVKSSRGSSDPQGV | |
| | TCGAATLSAERVRGDNKEYEYSVECQEDSACPAAEESLPIEVMVD | |
| | AVHKLKYENYTSSFFIRDIIKPDPPKNLQLKPLKNSRQVEVSWEY | |
| | PDTWSTPHSYFSLTFCVQVQGKSKREKKDRVFTDKTSATVICRKN | |
| | ASISVRAQDRYYSSSWSEWASVPCSGGGGSGGGGSGGGGSRNLPV | |
| | ATPDPGMFPCLHHSQNLLRAVSNMLQKARQTLEFYPCTSEEIDHE | |
| | DITKDKTSTVEACLPLELTKNESCLNSRETSFITNGSCLASRKTS | |
| | FMMALCLSSIYEDLKMYQVEFKTMNAKLLMDPKRQIFLDQNMLAV | |
| | IDELMQALNFNSETVPQKSSLEEPDFYKTKIKLCILLHAFRIRAV | |
| | TIDRVMSYLNAS | |
| Hole Chain BI-083 | EPKSSDKTHTCPPCPAPEAAGGPSVFLFPPKPKDTLMISRTPEVT | 278 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NREYTTYDYWGQGTQVTVSSA | |
| Knob Chain BI-084 | EPKSSDKTHTCPPCPAPEAAGGPSVFLFPPKPKDTLMISRTPEVT | 279 |
| | CVVVDVSHEDPEVKENWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGSGGWSHWGGSIWELKKDVYVV | |
| | ELDWYPDAPGEMVVLTCDTPEEDGITWTLDQSSEVLGSGKTLTIQ | |
| | VKEFGDAGQYTCHKGGEVLSHSLLLLHKKEDGIWSTDILKDQKEP | |
| | KNKTFLRCEAKNYSGRFTCWWLTTISTDLTFSVKSSRGSSDPQGV | |
| | TCGAATLSAERVRGDNKEYEYSVECQEDSACPAAEESLPIEVMVD | |
| | AVHKLKYENYTSSFFIRDIIKPDPPKNLQLKPLKNSRQVEVSWEY | |
| | PDTWSTPHSYFSLTFCVQVQGKSKREKKDRVFTDKTSATVICRKN | |
| | ASISVRAQDRYYSSSWSEWASVPCSGGSEGKSSGSGSESKSTGGS | |
| | RNLPVATPDPGMFPCLHHSQNLLRAVSNMLQKARQTLEFYPCTSE | |
| | EIDHEDITKDKTSTVEACLPLELTKNESCLNSRETSFITNGSCLA | |
| | SRKTSFMMALCLSSIYEDLKMYQVEFKTMNAKLLMDPKRQIFLDQ | |
| | NMLAVIDELMQALNFNSETVPQKSSLEEPDFYKTKIKLCILLHAF | |
| | RIRAVTIDRVMSYLNAS | |
| Hole Chain BI-084 | EPKSSDKTHTCPPCPAPEAAGGPSVFLFPPKPKDTLMISRTPEVT | 280 |
| | CVVVDVSHEDPEVKENWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NTDYTTYDYWGQGTQVTVSSA | |
| Knob Chain BI-085 | EPKSSDKTHTCPPCPAPEAAGGPSVFLFPPKPKDTLMISRTPEVT | 28 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGSGGWSHWGGSIWELKKDVYVV | |
| | ELDWYPDAPGEMVVLTCDTPEEDGITWTLDQSSEVLGSGKTLTIQ | |
| | VKEFGDAGQYTCHKGGEVLSHSLLLLHKKEDGIWSTDILKDQKEP | |
| | KNKTFLRCEAKNYSGRFTCWWLTTISTDLTFSVKSSRGSSDPQGV | |
| | TCGAATLSAERVRGDNKEYEYSVECQEDSACPAAEESLPIEVMVD | |
| | AVHKLKYENYTSSFFIRDIIKPDPPKNLQLKPLKNSRQVEVSWEY | |
| | PDTWSTPHSYFSLTFCVQVQGKSKREKKDRVFTDKTSATVICRKN | |
| | ASISVRAQDRYYSSSWSEWASVPCSGGSEGKSSGSGSESKSTGGS | |
| | RNLPVATPDPGMFPCLHHSQNLLRAVSNMLQKARQTLEFYPCTSE | |
| | EIDHEDITKDKTSTVEACLPLELTKNESCLNSRETSFITNGSCLA | |
| | SRKTSFMMALCLSSIYEDLKMYQVEFKTMNAKLLMDPKRQIFLDQ | |
| | NMLAVIDELMQALNENSETVPQKSSLEEPDFYKTKIKLCILLHAF | |
| | RIRAVTIDRVMSYLNAS | |
| Hole Chain BI-085 | EPKSSDKTHTCPPCPAPEAAGGPSVFLFPPKPKDTLMISRTPEVT | 282 |
| | CVVVDVSHEDPEVKENWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NREYTTYDYWGQGTQVTVSSA | |
| Knob Chain BI-086 | EPKSSDKTHTCPPCPAPEAAGGPSVFLFPPKPKDTLMISRTPEVT | 283 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPCREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGGGSGGGGSGGGGSRNLPVATPDPGM | |
| | FPCLHHSQNLLRAVSNMLQKARQTLEFYPCTSEEIDHEDITKDKT | |
| | STVEACLPLELTKNESCLNSRETSFITNGSCLASRKTSFMMALCL | |
| | SSIYEDLKMYQVEFKTMNAKLLMDPKRQIFLDQNMLAVIDELMQA | |
| | LNFNSETVPQKSSLEEPDFYKTKIKLCILLHAFRIRAVTIDRVMS | |
| | YLNASGSGGLRELHLDNN | |
| Hole Chain BI-086 | EPKSSDKTHTCPPCPAPEAAGGPSVFLFPPKPKDTLMISRTPEVT | 284 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVCT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NREYTTYDYWGQGTQVTVSSA | |
| Knob Chain BI-087 | EPKSSDKTHTCPPCPAPEAAGGPSVFLFPPKPKDTLMISRTPEVT | 285 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPCREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGSEGKSSGSGSESKSTGGSRNLPVAT | |
| | PDPGMFPCLHHSQNLLRAVSNMLQKARQTLEFYPCTSEEIDHEDI | |
| | TKDKTSTVEACLPLELTKNESCLNSRETSFITNGSCLASRKTSEM | |
| | MALCLSSIYEDLKMYQVEFKTMNAKLLMDPKRQIFLDQNMLAVID | |
| | ELMQALNFNSETVPQKSSLEEPDFYKTKIKLCILLHAFRIRAVTI | |
| | DRVMSYLNASGSGGLRELHLDNN | |
| Hole Chain BI-087 | EPKSSDKTHTCPPCPAPEAAGGPSVFLFPPKPKDTLMISRTPEVT | 286 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVCT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NTDYTTYDYWGQGTQVTVSSA | |
| Knob Chain BI-088 | EPKSSDKTHTCPPCPAPEAAGGPSVFLFPPKPKDTLMISRTPEVT | 287 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPCREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGSEGKSSGSGSESKSTGGSRNLPVAT | |
| | PDPGMFPCLHHSQNLLRAVSNMLQKARQTLEFYPCTSEEIDHEDI | |
| | TKDKTSTVEACLPLELTKNESCLNSRETSFITNGSCLASRKTSFM | |
| | MALCLSSIYEDLKMYQVEFKTMNAKLLMDPKRQIFLDQNMLAVID | |
| | ELMQALNENSETVPQKSSLEEPDFYKTKIKLCILLHAFRIRAVTI | |
| | DRVMSYLNASGSGGLRELHLDNN | |
| Hole Chain BI-088 | EPKSSDKTHTCPPCPAPEAAGGPSVFLFPPKPKDTLMISRTPEVT | 288 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVCT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NREYTTYDYWGQGTQVTVSSA | |
| Knob Chain BI-089 | EPKSSDKTHTCPPCPAPEAAGGPSVFLFPPKPKDTLMISRTPEVT | 289 |
| | CVVVDVSHEDPEVKENWYVDGVEVHNAKTKPREEQYNSTYRVVSV | |
| | LTVLHQDWLNGKEYKCKVSNKALPAPIEKTISKAKGQPREPQVYT | |
| | LPPCREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGGGSGGGGSGGGGSLRELHLDNNGGS | |
| | RNLPVATPDPGMFPCLHHSQNLLRAVSNMLQKARQTLEFYPCTSE | |
| | EIDHEDITKDKTSTVEACLPLELTKNESCLNSRETSFITNGSCLA | |
| | SRKTSFMMALCLSSIYEDLKMYQVEFKTMNAKLLMDPKRQIFLDQ | |
| | NMLAVIDELMQALNFNSETVPQKSSLEEPDFYKTKIKLCILLHAF | |
| | RIRAVTIDRVMSYLNAS | |
| Hole Chain BI-089 | EPKSSDKTHTCPPCPAPEAAGGPSVFLFPPKPKDTLMISRTPEVT | 290 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVCT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NTDYTTYDYWGQGTQVTVSSA | |
| Knob Chain BI-090 | EPKSSDKTHTCPPCPAPEAAGGPSVFLFPPKPKDTLMISRTPEVT | 291 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPCREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGGGSGGGGSGGGGSLRELHLDNNGGS | |
| | RNLPVATPDPGMFPCLHHSQNLLRAVSNMLQKARQTLEFYPCTSE | |
| | EIDHEDITKDKTSTVEACLPLELTKNESCLNSRETSFITNGSCLA | |
| | SRKTSFMMALCLSSIYEDLKMYQVEFKTMNAKLLMDPKRQIFLDQ | |
| | NMLAVIDELMQALNENSETVPQKSSLEEPDFYKTKIKLCILLHAF | |
| | RIRAVTIDRVMSYLNAS | |
| Hole Chain BI-090 | EPKSSDKTHTCPPCPAPEAAGGPSVFLFPPKPKDTLMISRTPEVT | 292 |
| | CVVVDVSHEDPEVKENWYVDGVEVHNAKTKPREEQYNSTYRVVSV | |
| | LTVLHQDWLNGKEYKCKVSNKALPAPIEKTISKAKGQPREPQVCT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNREK | |
| | NREYTTYDYWGQGTQVTVSSA | |
| Knob Chain BI-091 | EPKSSDKTHTCPPCPAPEAAGGPSVFLFPPKPKDTLMISRTPEVT | 293 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPCREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGSEGKSSGSGSESKSTGGSLRELHLD | |
| | NNGGSRNLPVATPDPGMFPCLHHSQNLLRAVSNMLQKARQTLEFY | |
| | PCTSEEIDHEDITKDKTSTVEACLPLELTKNESCLNSRETSFITN | |
| | GSCLASRKTSFMMALCLSSIYEDLKMYQVEFKTMNAKLLMDPKRQ | |
| | IFLDQNMLAVIDELMQALNENSETVPQKSSLEEPDFYKTKIKLCI | |
| | LLHAFRIRAVTIDRVMSYLNAS | |
| Hole Chain BI-091 | EPKSSDKTHTCPPCPAPEAAGGPSVFLFPPKPKDTLMISRTPEVT | 294 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVCT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NTDYTTYDYWGQGTQVTVSSA | |
| Knob Chain BI-092 | EPKSSDKTHTCPPCPAPEAAGGPSVFLFPPKPKDTLMISRTPEVT | 295 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPCREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGSEGKSSGSGSESKSTGGSLRELHLD | |
| | NNGGSRNLPVATPDPGMFPCLHHSQNLLRAVSNMLQKARQTLEFY | |
| | PCTSEEIDHEDITKDKTSTVEACLPLELTKNESCLNSRETSFITN | |
| | GSCLASRKTSFMMALCLSSIYEDLKMYQVEFKTMNAKLLMDPKRQ | |
| | IFLDQNMLAVIDELMQALNENSETVPQKSSLEEPDFYKTKIKLCI | |
| | LLHAFRIRAVTIDRVMSYLNAS | |
| Hole Chain BI-092 | EPKSSDKTHTCPPCPAPEAAGGPSVFLFPPKPKDTLMISRTPEVT | 296 |
| | CVVVDVSHEDPEVKENWYVDGVEVHNAKTKPREEQYNSTYRVVSV | |
| | LTVLHQDWLNGKEYKCKVSNKALPAPIEKTISKAKGQPREPQVCT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NREYTTYDYWGQGTQVTVSSA | |
| Knob Chain BI-093 | EPKSSDKTHTCPPCPAPEAAGGPSVFLFPPKPKDTLMISRTPEVT | 297 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPCREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGLRELHLDNNGGSIWELKKDVYVV | |
| | ELDWYPDAPGEMVVLTCDTPEEDGITWTLDQSSEVLGSGKTLTIQ | |
| | VKEFGDAGQYTCHKGGEVLSHSLLLLHKKEDGIWSTDILKDQKEP | |
| | KNKTFLRCEAKNYSGRFTCWWLTTISTDLTFSVKSSRGSSDPQGV | |
| | TCGAATLSAERVRGDNKEYEYSVECQEDSACPAAEESLPIEVMVD | |
| | AVHKLKYENYTSSFFIRDIIKPDPPKNLQLKPLKNSRQVEVSWEY | |
| | PDTWSTPHSYFSLTFCVQVQGKSKREKKDRVFTDKTSATVICRKN | |
| | ASISVRAQDRYYSSSWSEWASVPCSGGGGSGGGGSGGGGSRNLPV | |
| | ATPDPGMFPCLHHSQNLLRAVSNMLQKARQTLEFYPCTSEEIDHE | |
| | DITKDKTSTVEACLPLELTKNESCLNSRETSFITNGSCLASRKTS | |
| | FMMALCLSSIYEDLKMYQVEFKTMNAKLLMDPKRQIFLDQNMLAV | |
| | IDELMQALNENSETVPQKSSLEEPDFYKTKIKLCILLHAFRIRAV | |
| | TIDRVMSYLNAS | |
| Hole Chain BI-093 | EPKSSDKTHTCPPCPAPEAAGGPSVFLFPPKPKDTLMISRTPEVT | 298 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVCT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NTDYTTYDYWGQGTQVTVSSA | |
| Knob Chain BI-094 | EPKSSDKTHTCPPCPAPEAAGGPSVFLFPPKPKDTLMISRTPEVT | 299 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPCREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGLRELHLDNNGGSIWELKKDVYVV | |
| | ELDWYPDAPGEMVVLTCDTPEEDGITWTLDQSSEVLGSGKTLTIQ | |
| | VKEFGDAGQYTCHKGGEVLSHSLLLLHKKEDGIWSTDILKDQKEP | |
| | KNKTFLRCEAKNYSGRFTCWWLTTISTDLTFSVKSSRGSSDPQGV | |
| | TCGAATLSAERVRGDNKEYEYSVECQEDSACPAAEESLPIEVMVD | |
| | AVHKLKYENYTSSFFIRDIIKPDPPKNLQLKPLKNSRQVEVSWEY | |
| | PDTWSTPHSYFSLTFCVQVQGKSKREKKDRVFTDKTSATVICRKN | |
| | ASISVRAQDRYYSSSWSEWASVPCSGGGGSGGGGSGGGGSRNLPV | |
| | ATPDPGMFPCLHHSQNLLRAVSNMLQKARQTLEFYPCTSEEIDHE | |
| | DITKDKTSTVEACLPLELTKNESCLNSRETSFITNGSCLASRKTS | |
| | FMMALCLSSIYEDLKMYQVEFKTMNAKLLMDPKRQIFLDQNMLAV | |
| | IDELMQALNENSETVPQKSSLEEPDFYKTKIKLCILLHAFRIRAV | |
| | TIDRVMSYLNAS | |
| Hole Chain BI-094 | EPKSSDKTHTCPPCPAPEAAGGPSVFLFPPKPKDTLMISRTPEVT | 300 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVCT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NREYTTYDYWGQGTQVTVSSA | |
| Knob Chain BI-095 | EPKSSDKTHTCPPCPAPEAAGGPSVFLFPPKPKDTLMISRTPEVT | 301 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPCREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGLRELHLDNNGGSIWELKKDVYVV | |
| | ELDWYPDAPGEMVVLTCDTPEEDGITWTLDQSSEVLGSGKTLTIQ | |
| | VKEFGDAGQYTCHKGGEVLSHSLLLLHKKEDGIWSTDILKDQKEP | |
| | KNKTFLRCEAKNYSGRFTCWWLTTISTDLTFSVKSSRGSSDPQGV | |
| | TCGAATLSAERVRGDNKEYEYSVECQEDSACPAAEESLPIEVMVD | |
| | AVHKLKYENYTSSFFIRDIIKPDPPKNLQLKPLKNSRQVEVSWEY | |
| | PDTWSTPHSYFSLTFCVQVQGKSKREKKDRVFTDKTSATVICRKN | |
| | ASISVRAQDRYYSSSWSEWASVPCSGGSEGKSSGSGSESKSTGGS | |
| | RNLPVATPDPGMFPCLHHSQNLLRAVSNMLQKARQTLEFYPCTSE | |
| | EIDHEDITKDKTSTVEACLPLELTKNESCLNSRETSFITNGSCLA | |
| | SRKTSFMMALCLSSIYEDLKMYQVEFKTMNAKLLMDPKRQIFLDQ | |
| | NMLAVIDELMQALNFNSETVPQKSSLEEPDFYKTKIKLCILLHAF | |
| | RIRAVTIDRVMSYLNAS | |
| Hole Chain BI-095 | EPKSSDKTHTCPPCPAPEAAGGPSVFLFPPKPKDTLMISRTPEVT | 302 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVCT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNREK | |
| | NTDYTTYDYWGQGTQVTVSSA | |
| Knob Chain BI-096 | EPKSSDKTHTCPPCPAPEAAGGPSVFLFPPKPKDTLMISRTPEVT | 303 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPCREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGLRELHLDNNGGSIWELKKDVYVV | |
| | ELDWYPDAPGEMVVLTCDTPEEDGITWTLDQSSEVLGSGKTLTIQ | |
| | VKEFGDAGQYTCHKGGEVLSHSLLLLHKKEDGIWSTDILKDQKEP | |
| | KNKTFLRCEAKNYSGRFTCWWLTTISTDLTFSVKSSRGSSDPQGV | |
| | TCGAATLSAERVRGDNKEYEYSVECQEDSACPAAEESLPIEVMVD | |
| | AVHKLKYENYTSSFFIRDIIKPDPPKNLQLKPLKNSRQVEVSWEY | |
| | PDTWSTPHSYFSLTFCVQVQGKSKREKKDRVFTDKTSATVICRKN | |
| | ASISVRAQDRYYSSSWSEWASVPCSGGSEGKSSGSGSESKSTGGS | |
| | RNLPVATPDPGMFPCLHHSQNLLRAVSNMLQKARQTLEFYPCTSE | |
| | EIDHEDITKDKTSTVEACLPLELTKNESCLNSRETSFITNGSCLA | |
| | SRKTSFMMALCLSSIYEDLKMYQVEFKTMNAKLLMDPKRQIFLDQ | |
| | NMLAVIDELMQALNFNSETVPQKSSLEEPDFYKTKIKLCILLHAF | |
| | RIRAVTIDRVMSYLNAS | |
| Hole Chain BI-096 | EPKSSDKTHTCPPCPAPEAAGGPSVFLFPPKPKDTLMISRTPEVT | 304 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVCT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NREYTTYDYWGQGTQVTVSSA | |
| Knob Chain BI-097 | EPKSSDKTHTCPPCPAPEAAGGPSVFLFPPKPKDTLMISRTPEVT | 305 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPCREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGGGSGGGGSGGGGSRNLPVATPDPGM | |
| | FPCLHHSQNLLRAVSNMLQKARQTLEFYPCTSEEIDHEDITKDKT | |
| | STVEACLPLELTKNESCLNSRETSFITNGSCLASRKTSFMMALCL | |
| | SSIYEDLKMYQVEFKTMNAKLLMDPKRQIFLDQNMLAVIDELMQA | |
| | LNFNSETVPQKSSLEEPDFYKTKIKLCILLHAFRIRAVTIDRVMS | |
| | YLNASGSGGGGWSHW | |
| Hole Chain BI-097 | EPKSSDKTHTCPPCPAPEAAGGPSVFLFPPKPKDTLMISRTPEVT | 306 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVCT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NTDYTTYDYWGQGTQVTVSSA | |
| Knob Chain BI-098 | EPKSSDKTHTCPPCPAPEAAGGPSVFLFPPKPKDTLMISRTPEVT | 307 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPCREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGGGSGGGGSGGGGSRNLPVATPDPGM | |
| | FPCLHHSQNLLRAVSNMLQKARQTLEFYPCTSEEIDHEDITKDKT | |
| | STVEACLPLELTKNESCLNSRETSFITNGSCLASRKTSFMMALCL | |
| | SSIYEDLKMYQVEFKTMNAKLLMDPKRQIFLDQNMLAVIDELMQA | |
| | LNFNSETVPQKSSLEEPDFYKTKIKLCILLHAFRIRAVTIDRVMS | |
| | YLNASGSGGGGWSHW | |
| Hole Chain BI-098 | EPKSSDKTHTCPPCPAPEAAGGPSVFLFPPKPKDTLMISRTPEVT | 308 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVCT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NREYTTYDYWGQGTQVTVSSA | |
| Knob Chain BI-099 | EPKSSDKTHTCPPCPAPEAAGGPSVFLFPPKPKDTLMISRTPEVT | 309 |
| | CVVVDVSHEDPEVKENWYVDGVEVHNAKTKPREEQYNSTYRVVSV | |
| | LTVLHQDWLNGKEYKCKVSNKALPAPIEKTISKAKGQPREPQVYT | |
| | LPPCREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGSEGKSSGSGSESKSTGGSRNLPVAT | |
| | PDPGMFPCLHHSQNLLRAVSNMLQKARQTLEFYPCTSEEIDHEDI | |
| | TKDKTSTVEACLPLELTKNESCLNSRETSFITNGSCLASRKTSFM | |
| | MALCLSSIYEDLKMYQVEFKTMNAKLLMDPKRQIFLDQNMLAVID | |
| | ELMQALNENSETVPQKSSLEEPDFYKTKIKLCILLHAFRIRAVTI | |
| | DRVMSYLNASGSGGGGWSHW | |
| Hole Chain BI-099 | EPKSSDKTHTCPPCPAPEAAGGPSVFLFPPKPKDTLMISRTPEVT | 310 |
| | CVVVDVSHEDPEVKENWYVDGVEVHNAKTKPREEQYNSTYRVVSV | |
| | LTVLHQDWLNGKEYKCKVSNKALPAPIEKTISKAKGQPREPQVCT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NTDYTTYDYWGQGTQVTVSSA | |
| Knob Chain BI-100 | EPKSSDKTHTCPPCPAPEAAGGPSVFLFPPKPKDTLMISRTPEVT | 311 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPCREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGSEGKSSGSGSESKSTGGSRNLPVAT | |
| | PDPGMFPCLHHSQNLLRAVSNMLQKARQTLEFYPCTSEEIDHEDI | |
| | TKDKTSTVEACLPLELTKNESCLNSRETSFITNGSCLASRKTSFM | |
| | MALCLSSIYEDLKMYQVEFKTMNAKLLMDPKRQIFLDQNMLAVID | |
| | ELMQALNENSETVPQKSSLEEPDFYKTKIKLCILLHAFRIRAVTI | |
| | DRVMSYLNASGSGGGGWSHW | |
| Hole Chain BI-100 | EPKSSDKTHTCPPCPAPEAAGGPSVFLFPPKPKDTLMISRTPEVT | 312 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVCT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NREYTTYDYWGQGTQVTVSSA | |
| Knob Chain BI-101 | EPKSSDKTHTCPPCPAPEAAGGPSVFLFPPKPKDTLMISRTPEVT | 313 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPCREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSSGGGGSGGGGSGGGGSGGWSHWGGSRN | |
| | LPVATPDPGMFPCLHHSQNLLRAVSNMLQKARQTLEFYPCTSEEI | |
| | DHEDITKDKTSTVEACLPLELTKNESCLNSRETSFITNGSCLASR | |
| | KTSFMMALCLSSIYEDLKMYQVEFKTMNAKLLMDPKRQIFLDQNM | |
| | LAVIDELMQALNFNSETVPQKSSLEEPDFYKTKIKLCILLHAFRI | |
| | RAVTIDRVMSYLNAS | |
| Hole Chain BI-101 | EPKSSDKTHTCPPCPAPEAAGGPSVFLFPPKPKDTLMISRTPEVT | 314 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVCT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NTDYTTYDYWGQGTQVTVSSA | |
| Knob Chain BI-102 | EPKSSDKTHTCPPCPAPEAAGGPSVFLFPPKPKDTLMISRTPEVT | 315 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPCREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSSGGGGSGGGGSGGGGSGGWSHWGGSRN | |
| | LPVATPDPGMFPCLHHSQNLLRAVSNMLQKARQTLEFYPCTSEEI | |
| | DHEDITKDKTSTVEACLPLELTKNESCLNSRETSFITNGSCLASR | |
| | KTSFMMALCLSSIYEDLKMYQVEFKTMNAKLLMDPKRQIFLDQNM | |
| | LAVIDELMQALNENSETVPQKSSLEEPDFYKTKIKLCILLHAFRI | |
| | RAVTIDRVMSYLNAS | |
| Hole Chain BI-102 | EPKSSDKTHTCPPCPAPEAAGGPSVFLFPPKPKDTLMISRTPEVT | 316 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVCT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NREYTTYDYWGQGTQVTVSSA | |
| Knob Chain BI-103 | EPKSSDKTHTCPPCPAPEAAGGPSVFLFPPKPKDTLMISRTPEVT | 317 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPCREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGSEGKSSGSGSESKSTGGSGGWSHWG | |
| | GSRNLPVATPDPGMFPCLHHSQNLLRAVSNMLQKARQTLEFYPCT | |
| | SEEIDHEDITKDKTSTVEACLPLELTKNESCLNSRETSFITNGSC | |
| | LASRKTSFMMALCLSSIYEDLKMYQVEFKTMNAKLLMDPKRQIFL | |
| | DQNMLAVIDELMQALNFNSETVPQKSSLEEPDFYKTKIKLCILLH | |
| | AFRIRAVTIDRVMSYLNAS | |
| Hole Chain BI-103 | EPKSSDKTHTCPPCPAPEAAGGPSVFLFPPKPKDTLMISRTPEVT | 318 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVCT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NTDYTTYDYWGQGTQVTVSSA | |
| Knob Chain BI-104 | EPKSSDKTHTCPPCPAPEAAGGPSVFLFPPKPKDTLMISRTPEVT | 319 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPCREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGSEGKSSGSGSESKSTGGSGGWSHWG | |
| | GSRNLPVATPDPGMFPCLHHSQNLLRAVSNMLQKARQTLEFYPCT | |
| | SEEIDHEDITKDKTSTVEACLPLELTKNESCLNSRETSFITNGSC | |
| | LASRKTSFMMALCLSSIYEDLKMYQVEFKTMNAKLLMDPKRQIFL | |
| | DQNMLAVIDELMQALNFNSETVPQKSSLEEPDFYKTKIKLCILLH | |
| | AFRIRAVTIDRVMSYLNAS | |
| Hole Chain BI-104 | EPKSSDKTHTCPPCPAPEAAGGPSVFLFPPKPKDTLMISRTPEVT | 320 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVCT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NREYTTYDYWGQGTQVTVSSA | |
| Knob Chain BI-105 | EPKSSDKTHTCPPCPAPEAAGGPSVFLFPPKPKDTLMISRTPEVT | 321 |
| | CVVVDVSHEDPEVKENWYVDGVEVHNAKTKPREEQYNSTYRVVSV | |
| | LTVLHQDWLNGKEYKCKVSNKALPAPIEKTISKAKGQPREPQVYT | |
| | LPPCREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGSGGWSHWGGSIWELKKDVYVV | |
| | ELDWYPDAPGEMVVLTCDTPEEDGITWTLDQSSEVLGSGKTLTIQ | |
| | VKEFGDAGQYTCHKGGEVLSHSLLLLHKKEDGIWSTDILKDQKEP | |
| | KNKTFLRCEAKNYSGRFTCWWLTTISTDLTFSVKSSRGSSDPQGV | |
| | TCGAATLSAERVRGDNKEYEYSVECQEDSACPAAEESLPIEVMVD | |
| | AVHKLKYENYTSSFFIRDIIKPDPPKNLQLKPLKNSRQVEVSWEY | |
| | PDTWSTPHSYFSLTFCVQVQGKSKREKKDRVFTDKTSATVICRKN | |
| | ASISVRAQDRYYSSSWSEWASVPCSGGGGSGGGGSGGGGSRNLPV | |
| | ATPDPGMFPCLHHSQNLLRAVSNMLQKARQTLEFYPCTSEEIDHE | |
| | DITKDKTSTVEACLPLELTKNESCLNSRETSFITNGSCLASRKTS | |
| | FMMALCLSSIYEDLKMYQVEFKTMNAKLLMDPKRQIFLDQNMLAV | |
| | IDELMQALNFNSETVPQKSSLEEPDFYKTKIKLCILLHAFRIRAV | |
| | TIDRVMSYLNAS | |
| Hole Chain BI-105 | EPKSSDKTHTCPPCPAPEAAGGPSVFLFPPKPKDTLMISRTPEVT | 322 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVCT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NTDYTTYDYWGQGTQVTVSSA | |
| Knob Chain BI-106 | EPKSSDKTHTCPPCPAPEAAGGPSVFLFPPKPKDTLMISRTPEVT | 323 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPCREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGSGGWSHWGGSIWELKKDVYVV | |
| | ELDWYPDAPGEMVVLTCDTPEEDGITWTLDQSSEVLGSGKTLTIQ | |
| | VKEFGDAGQYTCHKGGEVLSHSLLLLHKKEDGIWSTDILKDQKEP | |
| | KNKTFLRCEAKNYSGRFTCWWLTTISTDLTFSVKSSRGSSDPQGV | |
| | TCGAATLSAERVRGDNKEYEYSVECQEDSACPAAEESLPIEVMVD | |
| | AVHKLKYENYTSSFFIRDIIKPDPPKNLQLKPLKNSRQVEVSWEY | |
| | PDTWSTPHSYFSLTFCVQVQGKSKREKKDRVFTDKTSATVICRKN | |
| | ASISVRAQDRYYSSSWSEWASVPCSGGGGSGGGGSGGGGSRNLPV | |
| | ATPDPGMFPCLHHSQNLLRAVSNMLQKARQTLEFYPCTSEEIDHE | |
| | DITKDKTSTVEACLPLELTKNESCLNSRETSFITNGSCLASRKTS | |
| | FMMALCLSSIYEDLKMYQVEFKTMNAKLLMDPKRQIFLDQNMLAV | |
| | IDELMQALNFNSETVPQKSSLEEPDFYKTKIKLCILLHAFRIRAV | |
| | TIDRVMSYLNAS | |
| Hole Chain BI-106 | EPKSSDKTHTCPPCPAPEAAGGPSVFLFPPKPKDTLMISRTPEVT | 324 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVCT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NREYTTYDYWGQGTQVTVSSA | |
| Knob Chain BI-107 | EPKSSDKTHTCPPCPAPEAAGGPSVFLFPPKPKDTLMISRTPEVT | 325 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPCREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGSGGWSHWGGSIWELKKDVYVV | |
| | ELDWYPDAPGEMVVLTCDTPEEDGITWTLDQSSEVLGSGKTLTIQ | |
| | VKEFGDAGQYTCHKGGEVLSHSLLLLHKKEDGIWSTDILKDQKEP | |
| | KNKTFLRCEAKNYSGRFTCWWLTTISTDLTFSVKSSRGSSDPQGV | |
| | TCGAATLSAERVRGDNKEYEYSVECQEDSACPAAEESLPIEVMVD | |
| | AVHKLKYENYTSSFFIRDIIKPDPPKNLQLKPLKNSRQVEVSWEY | |
| | PDTWSTPHSYFSLTFCVQVQGKSKREKKDRVFTDKTSATVICRKN | |
| | ASISVRAQDRYYSSSWSEWASVPCSGGSEGKSSGSGSESKSTGGS | |
| | RNLPVATPDPGMFPCLHHSQNLLRAVSNMLQKARQTLEFYPCTSE | |
| | EIDHEDITKDKTSTVEACLPLELTKNESCLNSRETSFITNGSCLA | |
| | SRKTSFMMALCLSSIYEDLKMYQVEFKTMNAKLLMDPKRQIFLDQ | |
| | NMLAVIDELMQALNFNSETVPQKSSLEEPDFYKTKIKLCILLHAF | |
| | RIRAVTIDRVMSYLNAS | |
| Hole Chain BI-107 | EPKSSDKTHTCPPCPAPEAAGGPSVFLFPPKPKDTLMISRTPEVT | 326 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVCT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NTDYTTYDYWGQGTQVTVSSA | |
| Knob Chain BI-108 | EPKSSDKTHTCPPCPAPEAAGGPSVFLFPPKPKDTLMISRTPEVT | 327 |
| | CVVVDVSHEDPEVKENWYVDGVEVHNAKTKPREEQYNSTYRVVSV | |
| | LTVLHQDWLNGKEYKCKVSNKALPAPIEKTISKAKGQPREPQVYT | |
| | LPPCREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGSGGWSHWGGSIWELKKDVYVV | |
| | ELDWYPDAPGEMVVLTCDTPEEDGITWTLDQSSEVLGSGKTLTIQ | |
| | VKEFGDAGQYTCHKGGEVLSHSLLLLHKKEDGIWSTDILKDQKEP | |
| | KNKTFLRCEAKNYSGRFTCWWLTTISTDLTFSVKSSRGSSDPQGV | |
| | TCGAATLSAERVRGDNKEYEYSVECQEDSACPAAEESLPIEVMVD | |
| | AVHKLKYENYTSSFFIRDIIKPDPPKNLQLKPLKNSRQVEVSWEY | |
| | PDTWSTPHSYFSLTFCVQVQGKSKREKKDRVFTDKTSATVICRKN | |
| | ASISVRAQDRYYSSSWSEWASVPCSGGSEGKSSGSGSESKSTGGS | |
| | RNLPVATPDPGMFPCLHHSQNLLRAVSNMLQKARQTLEFYPCTSE | |
| | EIDHEDITKDKTSTVEACLPLELTKNESCLNSRETSFITNGSCLA | |
| | SRKTSFMMALCLSSIYEDLKMYQVEFKTMNAKLLMDPKRQIFLDQ | |
| | NMLAVIDELMQALNFNSETVPQKSSLEEPDFYKTKIKLCILLHAF | |
| | RIRAVTIDRVMSYLNAS | |
| Hole Chain BI-108 | EPKSSDKTHTCPPCPAPEAAGGPSVFLFPPKPKDTLMISRTPEVT | 328 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVCT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NREYTTYDYWGQGTQVTVSSA | |
| BI-064 Knob | EPKSSDKTHTCPPCPAPEAAGGPSVFLFPPKPKDTLMISRTPEVT | 329 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGGGSGGGGSGGGGSRNLPVATPDPGM | |
| | FPCLHHSQNLLRAVSNMLQKARQTLEFYPCTSEEIDHEDITKDKT | |
| | STVEACLPLELTKNESCLNSRETSFITNGSCLASRKTSFMMALCL | |
| | SSIYEDLKMYQVEFKTMNAKLLMDPKRQIFLDQNMLAVIDELMQA | |
| | LNFNSETVPQKSSLEEPDFYKTKIKLCILLHAFRIRAVTIDRVMS | |
| | YLNAS | |
| BI-064 Hole | EPKSSDKTHTCPPCPAPEAAGGPSVFLFPPKPKDTLMISRTPEVT | 330 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSQVQLVESGGGLVQA | |
| | GGSLRLSCAASGRTFETIAMGWFRQVPGKEREFVAVIRGSGVATY | |
| | YPDSVKGRFTISKDSAKNTVYLQMNNLKPEDTAVYYCAATTNRFK | |
| | NTDYTTYDYWGQGTQVTVSSA | |
| BI-065 Knob | EPKSSDKTHTCPPCPAPEAAGGPSVFLFPPKPKDTLMISRTPEVT | 331 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLWCLVKGFYPSDIAVEWESNGQPENNYKTTP | |
| | PVLDSDGSFFLYSKLTVDKSRWQQGNVFSCSVMHEALHNHYTQKS | |
| | LSLSPGGGGGSGGGGGPLGVRGGGGGSIWELKKDVYVVELDWYPD | |
| | APGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATL | |
| | SAERVRGDNKEYEYSVECQEDSACPAAEESLPIEVMVDAVHKLKY | |
| | ENYTSSFFIRDIIKPDPPKNLQLKPLKNSRQVEVSWEYPDTWSTP | |
| | HSYFSLTFCVQVQGKSKREKKDRVFTDKTSATVICRKNASISVRA | |
| | QDRYYSSSWSEWASVPCSGGGGSGGGGSGGGGSRVIPVSGPARCL | |
| | SQSRNLLKTTDDMVKTAREKLKHYSCTAEDIDHEDITRDQTSTLK | |
| | TCLPLELHKNESCLATRETSSTTRGSCLPPQKTSLMMTLCLGSIY | |
| | EDLKMYQTEFQAINAALQNHNHQQIILDKGMLVAIDELMQSLNHN | |
| | GETLRQKPPVGEADPYRVKMKLCILLHAFSTRVVTINRVMGYLSS | |
| | A | |
| BI-065 Hole | EPKSSDKTHTCPPCPAPEAAGGPSVFLFPPKPKDTLMISRTPEVT | 332 |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLSCAVKGFYPSDIAVEWESNGQPENNYKTTP | |
| | PVLDSDGSFFLVSKLTVDKSRWQQGNVFSCSVMHEALHNRFTQKS | |
| | LSLSPGGGGGSGGGGSGGGGSGGGGSGGGGSEVQLVESGGGLVQP | |
| | GGSLRLSCAASGRTFETIAMGWFRQAPGKGLEFVAVIRGSGVATY | |
| | YAESVKGRFTISKDSSKNTVYLQMNSLRAEDTAVYYCAATTNRFK | |
| | NTDYTTYDYWGQGTQVTVSSA | |

### Method of Treatment

In its broadest form the invention provides an IL-12 Fc fusion protein for use in medicine.

The IL-12 Fc fusion protein of the invention is useful in cancer immunotherapy and beneficial in controlling tumor growth by activating anti-tumor cytotoxic immune responses. Accordingly, the IL-12 Fc fusion protein of the invention are useful for the treatment and/or prevention of cancer.

In a further aspect, the IL-12 Fc fusion protein of the invention can be used in a method for treating and/or preventing cancer and/or reducing the incidence of cancer, comprising administering a therapeutically effective amount of an IL-12 Fc fusion protein to an individual suffering from cancer, thereby ameliorating one or more symptoms of cancer.

In yet a further aspect the invention further provides for the use of an IL-12 Fc fusion protein according to the invention for the manufacture of a medicament for treatment and/or prevention of cancer.

In yet a further aspect, the IL-12 Fc fusion protein of the invention can be used in a method for treating and/or preventing and/or reducing the incidence of melanoma, non-small cell lung cancer (NSCLC), cutaneous squamous cell carcinoma (cSCC), or bladder cancer, comprising administering a therapeutically effective amount of an IL-12 Fc fusion protein to an individual suffering from melanoma, non-small cell lung cancer (NSCLC), cutaneous squamous cell carcinoma (cSCC), or bladder cancer thereby ameliorating one or more symptoms of melanoma, non-small cell lung cancer (NSCLC), cutaneous squamous cell carcinoma (cSCC), or bladder cancer.

For the prevention or treatment of a disease, the appropriate dosage of the IL-12 Fc fusion protein will depend on a variety of factors such as the type of disease to be treated, as defined above, the severity and course of the disease, whether the IL-12 Fc fusion protein is administered for preventive or therapeutic purposes, previous therapy, the patient's clinical history and response to the IL-12 Fc fusion protein, and the discretion of the attending physician. The IL-12 Fc fusion protein is suitably administered to the patient at one time or over a series of treatments.

In one aspect, the cancer is a solid tumor. In another aspect, the cancer is a lymphoma. In another aspect the cancer is a relapsed or refractory advanced or metastatic solid tumor or lymphoma. In one aspect, the lymphoma is Non-Hodgkin lymphoma or Hodgkin lymphoma. In another aspect, the lymphoma is cutaneous T-cell lymphoma (CTCL) or Sezáry syndrome/disease,

In one aspect, the cancer is a skin cancer, lung cancer or head & neck cancer, brain cancer, gastrointestinal cancer, endometrial cancer, vaginal cancer, HPV positive tumor, HPV-positive cervical cancer, HPV-positive oropharyngeal cancer, HPV-positive anal cancer, HPV-positive penile cancer, HPV-positive vaginal cancer, HPV-positive vulvar cancer, anal cancer, colorectal cancer, oropharyngeal squamous cell carcinoma, squamous cell carcinoma, gastric cancer, gastroesophageal junction adenocarcinoma, esophageal carcinoma, cutaneous t-cell lymphoma, hepatocellular carcinoma, pancreatic adenocarcinoma, pancreatic carcinoma, cholangiocarcinoma, bladder urothelial carcinoma, urothelial carcinoma, renal cancer, metastatic melanoma, prostate carcinoma, breast carcinoma, ovarian cancer, a head and neck squamous-cell carcinoma (HNSCC), glioblastoma, non-small cell lung cancer, brain tumor or small cell lung cancer. Preferred is the treatment of melanoma, non-small cell lung cancer (NSCLC), cutaneous squamous cell carcinoma (cSCC), urothelial carcinoma or bladder cancer.

In another aspect the IL-12 Fc fusion protein is useful to treat patients having failed or not adequately responding to previous PD-1 or PD-L1 inhibitor treatment (e.g. immunotherapy resistant advanced or metastatic solid tumors or lymphoma).

In another aspect the IL-12 Fc fusion protein is useful for the treatment of patients which have completed checkpoint inhibitor therapy with either a PD-1 or PD-L1 inhibitor, e.g. antagonistic antibodies to PD-1 or PD-L1.

The IL-12 Fc fusion protein is administered by any suitable means, including oral, parenteral, subcutaneous, intratumoral, intravenous, intradermal, intraperitoneal, intrapulmonary, and intranasal. Parenteral infusions include intramuscular, intravenous, intraarterial, intraperitoneal, or subcutaneous administration. In addition, the IL-12 Fc fusion protein is suitably administered by pulse infusion. In one aspect, the dosing is given by injections, most preferably intravenous or subcutaneous injections, depending in part on whether the administration is brief or chronic.

Depending on the specific IL-12 Fc fusion protein of the invention and its specific pharmacokinetic and other properties, it may be administered daily, every second, third, fourth, fifth or sixth day, weekly, monthly, and the like. An administration regimen could include long-term, weekly treatment. By “long-term” is meant at least two weeks and preferably months, or years of duration.

The treatment schedule may include various regimens and in typical will require multiple doses administered to the patient over a period of one, two, three or four weeks optionally followed by one or more further rounds of treatment.

The term “suppression” is used herein in the same context as “amelioration” and “alleviation” to mean a lessening or diminishing of one or more characteristics of the disease. The IL-12 Fc fusion protein or pharmaceutical composition of the invention will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the IL-12 Fc fusion protein to be administered will be governed by such considerations, and is the minimum amount necessary to prevent, ameliorate, or treat clinical symptoms of cancer, in particular the minimum amount which is effective to these disorders.

In another aspect the IL-12 Fc fusion protein of the invention can be administered multiple times and in several doses.

In another aspect, the IL-12 Fc fusion protein is administered intravenously. In another aspect, the IL-12 Fc fusion protein is administered subcutaneously.

As stated above, the IL-12 Fc fusion protein of the invention have much utility for stimulating an immune response against cancer cells. The strong immune activating potential was observed to be restricted to the tumor microenvironment. Thus, in a preferred aspect, the IL-12 Fc fusion protein of the invention may be administered systemically to a patient. Systemic applicability is a crucial attribute, as many cancers are highly metastasized and it will permit the treatment of difficult to access as well as non-accessible tumor lesions. Due to this unique immune stimulating properties the IL-12 Fc fusion proteins according to the invention are especially useful for treatment of metastasizing tumors.

Some patients develop resistance to checkpoint inhibitor therapy and it was observed that such patients seem to accumulate mutations in the IFN pathway. Therefore in one aspect, the IL-12 Fc fusion protein of the invention is useful for the treatment of patients who developed a resistance to checkpoint inhibitor therapy. Due to the unique immune promoting properties of the IL-12 Fc fusion such treated patients may become eligible for continuation of checkpoint inhibitor therapy.

In a preferred embodiment, the IL-12 Fc fusion protein of the invention and is useful for the treatment of patients with non-small cell lung cancer which have completed checkpoint inhibitor therapy with either a PD-1 or PD-L1 inhibitor, e.g. antagonistic antibodies to PD-1 or PD-L1.

It is understood that any of the above pharmaceutical formulations or therapeutic methods may be carried out using any one of the inventive IL-12 Fc fusion proteins or pharmaceutical compositions.

### Combinations

The present invention also provides combination treatments/methods providing certain advantages compared to treatments/methods currently used and/or known in the prior art. These advantages may include in vivo efficacy (e.g. improved clinical response, extend of the response, increase of the rate of response, duration of response, disease stabilization rate, duration of stabilization, time to disease progression, progression free survival (PFS) and/or overall survival (OS), later occurrence of resistance and the like), safe and well tolerated administration and reduced frequency and severity of adverse events.

The IL-12 Fc fusion proteins of the invention may be used in combination with other pharmacologically active ingredients, such as state-of-the-art or standard-of-care compounds, such as e.g. cytostatic or cytotoxic substances, cell proliferation inhibitors, anti-angiogenic substances, steroids, immune modulators/checkpoint inhibitors, and the like. The IL-12 Fc fusion proteins of the invention may also be used in combination with radiotherapy.

Cytostatic and/or cytotoxic active substances which may be administered in combination with the IL-12 Fc fusion proteins of the invention include, without being restricted thereto, hormones, hormone analogues and antihormones, aromatase inhibitors, LHRH agonists and antagonists, inhibitors of growth factors (growth factors such as for example platelet derived growth factor (PDGF), fibroblast growth factor (FGF), vascular endothelial growth factor (VEGF), epidermal growth factor (EGF), insulin-like growth factors (IGF), human epidermal growth factor (HER, e.g. HER2, HER3, HER4) and hepatocyte growth factor (HGF)), inhibitors are for example (anti-)growth factor antibodies, (anti-)growth factor receptor antibodies and tyrosine kinase inhibitors, such as for example cetuximab, gefitinib, afatinib, nintedanib, imatinib, lapatinib, bosutinib and trastuzumab; antimetabolites (e.g. antifolates such as methotrexate, raltitrexed, pyrimidine analogues such as 5-fluorouracil (5-FU), gemcitabine, irinotecan, doxorubicin, TAS-102, capecitabine and gemcitabine, purine and adenosine analogues such as mercaptopurine, thioguanine, cladribine and pentostatin, cytarabine (ara C), fludarabine); antitumor antibiotics (e.g. anthracyclins); platinum derivatives (e.g. cisplatin, oxaliplatin, carboplatin); alkylation agents (e.g. estramustin, meclorethamine, melphalan, chlorambucil, busulphan, dacarbazin, cyclophosphamide, ifosfamide, temozolomide, nitrosoureas such as for example carmustin and lomustin, thiotepa); antimitotic agents (e.g. Vinca alkaloids such as for example vinblastine, vindesin, vinorelbin and vincristine; and taxanes such as paclitaxel, docetaxel); angiogenesis inhibitors, including bevacizumab, ramucirumab and aflibercept, tubuline inhibitors; DNA synthesis inhibitors, PARP inhibitors, topoisomerase inhibitors (e.g. epipodophyllotoxins such as for example etoposide and etopophos, teniposide, amsacrin, topotecan, irinotecan, mitoxantrone), serine/threonine kinase inhibitors (e.g. PDK1 inhibitors, Raf inhibitors, A-Raf inhibitors, B-Raf inhibitors, C-Raf inhibitors, mTOR inhibitors, mTORC1/2 inhibitors, PI3K inhibitors, PI3Kα inhibitors, dual mTOR/PI3K inhibitors, STK33 inhibitors, AKT inhibitors, PLK1 inhibitors (such as volasertib), inhibitors of CDKs, including CDK9 inhibitors, Aurora kinase inhibitors), tyrosine kinase inhibitors (e.g. PTK2/FAK inhibitors), protein protein interaction inhibitors, MEK inhibitors, ERK inhibitors, FLT3 inhibitors, BRD4 inhibitors, IGF-1R inhibitors, Bcl-XL inhibitors, Bcl-2 inhibitors, Bcl-2/Bcl-xL inhibitors, ErbB receptor inhibitors, BCR ABL inhibitors, ABL inhibitors, Src inhibitors, rapamycin analogs (e.g. everolimus, temsirolimus, ridaforolimus, sirolimus), androgen synthesis inhibitors, androgen receptor inhibitors, DNMT inhibitors, HDAC inhibitors, ANG1/2 inhibitors, CYP17 inhibitors, radiopharmaceuticals, immunotherapeutic agents such as immune checkpoint inhibitors (e.g. CTLA4, PD1, PD-L1, LAG3, and TIM3 binding molecules/immunoglobulins, such as ipilimumab, nivolumab, pembrolizumab) and various chemotherapeutic agents such as amifostin, anagrelid, clodronat, filgrastin, interferon, interferon alpha, leucovorin, rituximab, procarbazine, levamisole, mesna, mitotane, pamidronate and porfimer; proteasome inhibitors (such as Bortezomib); Smac and BH3 mimetics; agents restoring p53 functionality including mdm2-p53 antagonist; inhibitors of the Wnt/beta-catenin signaling pathway; Flt3L as well as Flt3-stimulating antibodies or ligand mimetics; SIRPalpha & CD47 blocking therapeutics; and/or cyclin-dependent kinase 9 inhibitors.

Furthermore, the potential conversion of immunological “cold” into “hot” tumors, myeloid/dendritic cell activation in conjunction with T-cell activation further favourably interacts with therapeutic modalities, such as T-cell engagers. Thus, in one embodiment the IL-12 Fc fusion proteins of the invention can be used in combination treatment with one or more T-cell engagers.

The IL-12 Fc fusion proteins of the invention can be used in combination treatment with cancer vaccines or oncolytic viruses. Such a combined treatment may be given as a non-fixed (e.g. free) combination of the substances or in the form of a fixed combination, including kit-of-parts. In one embodiment, the oncolytic virus is a vesicular stomatitis virus. In a preferred embodiment, the vesicular stomatitis virus is a vesicular stomatitis virus with the glycoprotein GP of the lymphocytic choriomeningitis virus (LCMV), preferably with the strain WE-HPI. Such VSV is for example described in WO2010/040526 and named VSV-GP.

In yet another embodiment, any of the disclosed IL-12 Fc fusion proteins can be encoded in an appropriate viral vector, e.g. such as in an oncolytic viral vector and preferably in a vesicular stomatitis virus or more preferably a vesicular stomatitis virus with the glycoprotein GP of the lymphocytic choriomeningitis virus (LCMV), preferably with the strain WE-HPI. Such VSV is for example described in WO2010/040526 and named VSV-GP. Such viral vectors could then be used to deliver the IL-12 Fc fusion protein (encoded in the genome of the viral vector). The IL-12 Fc fusion protein would then be transcribed/translated in the patient and the polypeptide chains would assemble in the human body to form the complete prodrug.

The IL-12 Fc fusion proteins of the invention can be used in combination treatment with a PD-1 pathway inhibitor. Such a combined treatment may be given as a non-fixed (e.g. free) combination of the substances or in the form of a fixed combination, including kit-of-parts.

In this context, “combination” or “combined” within the meaning of this invention includes, without being limited, a product that results from the mixing or combining of more than one active agent and includes both fixed and non-fixed (e.g. free) combinations (including kits) and uses, such as e.g. the simultaneous, concurrent, sequential, successive, alternate or separate use of the components or agents. The term “fixed combination” means that the active agents are both administered to a patient simultaneously in the form of a single entity or dosage. The term “non-fixed combination” means that the active agents are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active agents.

The invention provides for an IL-12 Fc fusion protein in combination with a PD-1 pathway inhibitor for use in the treatment of cancers as described herein, preferably for the treatment of solid cancers.

The invention also provides for the use of an IL-12 Fc fusion protein in combination with a PD-1 pathway inhibitor for the manufacture of a medicament for treatment and/or prevention of cancers as described herein, preferably for the treatment of solid cancers.

The invention further provides for a method for treating and/or preventing cancer, comprising administering a therapeutically effective amount of an IL-12 Fc fusion protein of the invention, and a PD-1 pathway inhibitor to an individual suffering from cancer, thereby ameliorating one or more symptoms of cancer. The IL-12 Fc fusion protein of the invention and the PD-1 pathway inhibitor may be administered concomitantly, sequentially or alternately.

The IL-12 Fc fusion protein of the invention and the PD-1 pathway inhibitor may be administered by the same administration routes or via different administration routes. Preferably, the PD-1 pathway inhibitor is administered intravenously and the IL-12 Fc fusion proteins of the invention is administered intravenously or subcutaneously.

Particularly preferred are treatments with the IL-12 Fc fusion proteins of the invention in combination with (immunotherapeutic agents, including anti-PD-1 and anti-PD-L1 agents and anti LAG3 agents, such as pembrolizumab and nivolumab and antibodies as disclosed in WO2017/198741.

A combination as herein provided comprises (i) an IL-12 Fc fusion protein of the invention and (ii) a PD-1 pathway inhibitor, preferably an antagonistic antibody which is directed against PD-1 or PD-L1. Further provided is the use of such a combination comprising (i) and (ii) for the treatment of cancers as described herein.

In another aspect a combination treatment is provided comprising the use of (i) an IL-12 Fc fusion proteins of the invention and (ii) a PD-1 pathway inhibitor. In such combination treatment the IL-12 Fc fusion proteins of the invention may be administered concomitantly, sequentially or alternately with the PD-1 pathway inhibitor.

For example, “concomitant” administration includes administering the active agents within the same general time period, for example on the same day(s) but not necessarily at the same time. Alternate administration includes administration of one agent during a time period, for example over the course of a few days or a week, followed by administration of the other agent during a subsequent period of time, for example over the course of a few days or a week, and then repeating the pattern for one or more cycles. Sequential or successive administration includes administration of one agent during a first time period (for example over the course of a few days or a week) using one or more doses, followed by administration of the other agent during a second time period (for example over the course of a few days or a week) using one or more doses. An overlapping schedule may also be employed, which includes administration of the active agents on different days over the treatment period, not necessarily according to a regular sequence. Variations on these general guidelines may also be employed, e.g. according to the agents used and the condition of the subject.

Sequential treatment schedules include administration of the IL-12 Fc fusion protein of the invention followed by administration of the PD-1 pathway inhibitor. Sequential treatment schedules also include administration of the PD-1 pathway inhibitor followed by administration of the IL-12 Fc fusion protein of the invention. Sequential treatment schedules may include administrations 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or 31 days after each other.

A PD-1 pathway inhibitor within the meaning of this invention and all of its embodiments is a compound that inhibits the interaction of PD-1 with its receptor(s). A PD-1 pathway inhibitor is capable to impair the PD-1 pathway signaling, preferably mediated by the PD-1 receptor. The PD-1 inhibitor may be any inhibitor directed against any member of the PD-1 pathway capable of antagonizing PD-1 pathway signaling. The inhibitor may be an antagonistic antibody targeting any member of the PD-1 pathway, preferably directed against PD-1 receptor, PD-L1 or PD-L2. Also, the PD-1 pathway inhibitor may be a fragment of the PD-1 receptor or the PD-1 receptor blocking the activity of PD1 ligands.

PD-1 antagonists are well-known in the art, e.g. reviewed by Li et al., Int. J. Mol. Sci. 2016, 17, 1151 (incorporated herein by reference). Any PD-1 antagonist, especially antibodies, such as those disclosed by Li et al. as well as the further antibodies disclosed herein below, can be used according to the invention. Preferably, the PD-1 antagonist of this invention and all its embodiments is selected from the group consisting of the following antibodies:
ezabenlimab (BI754091);
pembrolizumab (anti-PD-1 antibody);
nivolumab (anti-PD-1 antibody);
pidilizumab (anti-PD-1 antibody);
PDR-001 (anti-PD-1 antibody);
PD1-1, PD1-2, PD1-3, PD1-4, and PD1-5 as disclosed herein below (anti-PD-1 antibodies)
atezolizumab (anti-PD-L1 antibody);
avelumab (anti-PD-L1 antibody);
durvalumab (anti-PD-L1 antibody).

Pembrolizumab (formerly also known as lambrolizumab; trade name Keytruda; also known as MK-3475) disclosed e.g. in Hamid, O. et al. (2013) New England Journal of Medicine 369(2):134-44, is a humanized IgG4 monoclonal antibody that binds to PD-1; it contains a mutation at C228P designed to prevent Fc-mediated cytotoxicity. Pembrolizumab is e.g. disclosed in U.S. Pat. No. 8,354,509 and WO2009/114335. It is approved by the FDA for the treatment of patients suffering from unresectable or metastatic melanoma and patients with metastatic NSCLC.

Nivolumab (CAS Registry Number: 946414-94-4; BMS-936558 or MDX1106b) is a fully human IgG4 monoclonal antibody which specifically blocks PD-1, lacking detectable antibody-dependent cellular toxicity (ADCC). Nivolumab is e.g. disclosed in U.S. Pat. No. 8,008,449 and WO2006/121168. It has been approved by the FDA for the treatment of patients suffering from unresectable or metastatic melanoma, metastatic NSCLC and advanced renal cell carcinoma.

Pidilizumab (CT-011; Cure Tech) is a humanized IgG1k monoclonal antibody that binds to PD-1. Pidilizumab is e.g. disclosed in WO2009/101611.

PDR-001 or PDR001 is a high-affinity, ligand-blocking, humanized anti-PD-1 IgG4 antibody that blocks the binding of PD-L1 and PD-L2 to PD-1. PDR-001 is disclosed in WO2015/112900 and WO2017/019896.

Antibodies PD1-1 to PD1-5 are antibody molecules defined by the sequences as shown in Table 7, wherein HC denotes the (full length) heavy chain and LC denotes the (full length) light chain:

TABLE 7

| SEQ | | |
|---|---|---|
| ID | Sequence | |
| NO: | name | Amino acid sequence |
| 51 | HC of | EVMLVESGGGLVQPGGSLRLSCTASGFTFSASAMSWVRQAPGKGLEWVAYISGGGGDTYYSSS |
| | PD1-1 | VKGRFTISRDNAKNSLYLQMNSLRAEDTAVYYCARHSNVNYYAMDYWGQGTLVTVSSASTKGP |
| | | SVFPLAPCSRSTSESTAALGCLVKDYFPEPVTVSWNSGALTSGVHTFPAVLQSSGLYSLSSVV |
| | | TVPSSSLGTKTYTCNVDHKPSNTKVDKRVESKYGPPCPPCPAPEFLGGPSVFLFPPKPKDTLM |
| | | ISRTPEVTCVVVDVSQEDPEVQFNWYVDGVEVHNAKTKPREEQFNSTYRVVSVLTVLHQDWLN |
| | | GKEYKCKVSNKGLPSSIEKTISKAKGQPREPQVYTLPPSQEEMTKNQVSLTCLVKGFYPSDIA |
| | | VEWESNGQPENNYKTTPPVLDSDGSFFLYSRLTVDKSRWQEGNVFSCSVMHEALHNHYTQKSL |
| | | SLSLG |
| 52 | LC of | EIVLTQSPATLSLSPGERATMSCRASENIDTSGISFMNWYQQKPGQAPKLLIYVASNQGSGIP |
| | PD1-1 | ARFSGSGSGTDFTLTISRLEPEDFAVYYCQQSKEVPWTFGQGTKLEIKRTVAAPSVFIFPPSD |
| | | EQLKSGTASVVCLLNNFYPREAKVQWKVDNALQSGNSQESVTEQDSKDSTYSLSSTLTLSKAD |
| | | YEKHKVYACEVTHQGLSSPVTKSENRGEC |
| 53 | HC of | EVMLVESGGGLVQPGGSLRLSCTASGFTFSASAMSWVRQAPGKGLEWVAYISGGGGDTYYSSS |
| | PD1-2 | VKGRFTISRDNAKNSLYLQMNSLRAEDTAVYYCARHSNPNYYAMDYWGQGTLVTVSSASTKGP |
| | | SVFPLAPCSRSTSESTAALGCLVKDYFPEPVTVSWNSGALTSGVHTFPAVLQSSGLYSLSSVV |
| | | TVPSSSLGTKTYTCNVDHKPSNTKVDKRVESKYGPPCPPCPAPEFLGGPSVFLFPPKPKDTLM |
| | | ISRTPEVTCVVVDVSQEDPEVQFNWYVDGVEVHNAKTKPREEQFNSTYRVVSVLTVLHQDWLN |
| | | GKEYKCKVSNKGLPSSIEKTISKAKGQPREPQVYTLPPSQEEMTKNQVSLTCLVKGFYPSDIA |
| | | VEWESNGQPENNYKTTPPVLDSDGSFFLYSRLTVDKSRWQEGNVFSCSVMHEALHNHYTQKSL |
| | | SLSLG |
| 54 | LC of | EIVLTQSPATLSLSPGERATMSCRASENIDTSGISFMNWYQQKPGQAPKLLIYVASNQGSGIP |
| | PD1-2 | ARFSGSGSGTDFTLTISRLEPEDFAVYYCQQSKEVPWTFGQGTKLEIKRTVAAPSVFIFPPSD |
| | | EQLKSGTASVVCLLNNFYPREAKVQWKVDNALQSGNSQESVTEQDSKDSTYSLSSTLTLSKAD |
| | | YEKHKVYACEVTHQGLSSPVTKSENRGEC |
| 55 | HC of | EVMLVESGGGLVQPGGSLRLSCTASGFTFSKSAMSWVRQAPGKGLEWVAYISGGGGDTYYSSS |
| | PD1-3 | VKGRFTISRDNAKNSLYLQMNSLRAEDTAVYYCARHSNVNYYAMDYWGQGTLVTVSSASTKGP |
| | | SVFPLAPCSRSTSESTAALGCLVKDYFPEPVTVSWNSGALTSGVHTFPAVLQSSGLYSLSSVV |
| | | TVPSSSLGTKTYTCNVDHKPSNTKVDKRVESKYGPPCPPCPAPEFLGGPSVFLFPPKPKDTLM |
| | | ISRTPEVTCVVVDVSQEDPEVQFNWYVDGVEVHNAKTKPREEQFNSTYRVVSVLTVLHQDWLN |
| | | GKEYKCKVSNKGLPSSIEKTISKAKGQPREPQVYTLPPSQEEMTKNQVSLTCLVKGFYPSDIA |
| | | VEWESNGQPENNYKTTPPVLDSDGSFFLYSRLTVDKSRWQEGNVFSCSVMHEALHNHYTQKSL |
| | | SLSLG |
| 56 | LC of | EIVLTQSPATLSLSPGERATMSCRASENIDVSGISFMNWYQQKPGQAPKLLIYVASNQGSGIP |
| | PD1-3 | ARFSGSGSGTDFTLTISRLEPEDFAVYYCQQSKEVPWTFGQGTKLEIKRTVAAPSVFIFPPSD |
| | | EQLKSGTASVVCLLNNFYPREAKVQWKVDNALQSGNSQESVTEQDSKDSTYSLSSTLTLSKAD |
| | | YEKHKVYACEVTHQGLSSPVTKSENRGEC |
| 57 | HC of | EVMLVESGGGLVQPGGSLRLSCTASGFTFSKSAMSWVRQAPGKGLEWVAYISGGGGDTYYSSS |
| | PD1-4 | VKGRFTISRDNAKNSLYLQMNSLRAEDTAVYYCARHSNVNYYAMDYWGQGTLVTVSSASTKGP |
| | | SVFPLAPCSRSTSESTAALGCLVKDYFPEPVTVSWNSGALTSGVHTFPAVLQSSGLYSLSSVV |
| | | TVPSSSLGTKTYTCNVDHKPSNTKVDKRVESKYGPPCPPCPAPEFLGGPSVFLFPPKPKDTLM |
| | | ISRTPEVTCVVVDVSQEDPEVQFNWYVDGVEVHNAKTKPREEQFNSTYRVVSVLTVLHQDWLN |
| | | GKEYKCKVSNKGLPSSIEKTISKAKGQPREPQVYTLPPSQEEMTKNQVSLTCLVKGFYPSDIA |
| | | VEWESNGQPENNYKTTPPVLDSDGSFFLYSRLTVDKSRWQEGNVFSCSVMHEALHNHYTQKSL |
| | | SLSLG |
| 58 | LC of | EIVLTQSPATLSLSPGERATMSCRASENIDVSGISFMNWYQQKPGQAPKLLIYVASNQGSGIP |
| | PD1-4 | ARFSGSGSGTDFTLTISRLEPEDFAVYYCQQSKEVPWTFGQGTKLEIKRTVAAPSVFIFPPSD |
| | | EQLKSGTASVVCLLNNFYPREAKVQWKVDNALQSGNSQESVTEQDSKDSTYSLSSTLTLSKAD |
| | | YEKHKVYACEVTHQGLSSPVTKSENRGEC |
| 59 | HC of | EVMLVESGGGLVQPGGSLRLSCTASGFTFSKSAMSWVRQAPGKGLEWVAYISGGGGDTYYSSS |
| | PD1-5 | VKGRFTISRDNAKNSLYLQMNSLRAEDTAVYYCARHSNVNYYAMDYWGQGTLVTVSSASTKGP |
| | | SVFPLAPCSRSTSESTAALGCLVKDYFPEPVTVSWNSGALTSGVHTFPAVLQSSGLYSLSSVV |
| | | TVPSSSLGTKTYTCNVDHKPSNTKVDKRVESKYGPPCPPCPAPEFLGGPSVFLFPPKPKDTLM |
| | | ISRTPEVTCVVVDVSQEDPEVQFNWYVDGVEVHNAKTKPREEQFNSTYRVVSVLTVLHQDWLN |
| | | GKEYKCKVSNKGLPSSIEKTISKAKGQPREPQVYTLPPSQEEMTKNQVSLTCLVKGFYPSDIA |
| | | VEWESNGQPENNYKTTPPVLDSDGSFFLYSRLTVDKSRWQEGNVFSCSVMHEALHNHYTQKSL |
| | | SLSLG |
| 60 | LC of | EIVLTQSPATLSLSPGERATMSCRASENIDVSGISFMNWYQQKPGQAPKLLIYVASNQGSGIP |
| | PD1-5 | ARFSGSGSGTDFTLTISRLEPEDFAVYYCQQSKEVPWTFGQGTKLEIKRTVAAPSVFIFPPSD |
| | | EQLKSGTASVVCLLNNFYPREAKVQWKVDNALQSGNSQESVTEQDSKDSTYSLSSTLTLSKAD |
| | | YEKHKVYACEVTHQGLSSPVTKSENRGEC |

Specifically, the anti-PD-1 antibody molecule described herein above has:
(PD1-1:) a heavy chain comprising the amino acid sequence of SEQ ID NO:51 and a light chain comprising the amino acid sequence of SEQ ID NO:52; or
(PD1-2:) a heavy chain comprising the amino acid sequence of SEQ ID NO:53 and a light chain comprising the amino acid sequence of SEQ ID NO:54; or
(PD1-3:) a heavy chain comprising the amino acid sequence of SEQ ID NO:55 and a light chain comprising the amino acid sequence of SEQ ID NO:56; or
(PD1-4:) a heavy chain comprising the amino acid sequence of SEQ ID NO:57 and a light chain comprising the amino acid sequence of SEQ ID NO:58; or
(PD1-5:) a heavy chain comprising the amino acid sequence of SEQ ID NO:59 and a light chain comprising the amino acid sequence of SEQ ID NO:60.

Atezolizumab (Tecentriq, also known as MPDL3280A) is a phage-derived human IgG1k monoclonal antibody targeting PD-L1 and is described e.g. in Deng et al. mAbs 2016; 8:593-603. It has been approved by the FDA for the treatment of patients suffering from urothelial carcinoma.

Avelumab is a fully human anti-PD-L1 IgG1 monoclonal antibody and described in e.g. Boyerinas et al. Cancer Immunol. Res. 2015; 3:1148-1157.

Durvalumab (MEDI4736) is a human IgG1k monoclonal antibody with high specificity to PD-L1 and described in e.g. Stewart et al. Cancer Immunol. Res. 2015; 3:1052-1062 or in Ibrahim et al. Semin. Oncol. 2015; 42:474-483.

Further PD-1 antagonists disclosed by Li et al. (supra), or known to be in clinical trials, such as AMP-224, MEDI0680 (AMP-514), REGN2810, BMS-936559, JS001-PD-1, SHR-1210, BMS-936559, TSR-042, JNJ-63723283, MEDI4736, MPDL3280A, and MSB0010718C, may be used as alternative or in addition to the above mentioned antagonists.

The INNs as used herein are meant to also encompass all biosimilar antibodies having the same, or substantially the same, amino acid sequences as the originator antibody, including but not limited to those biosimilar antibodies authorized under 42 USC § 262 subsection (k) in the US and equivalent regulations in other jurisdictions.

PD-1 antagonists listed above are known in the art with their respective manufacture, therapeutic use and properties.

In one embodiment the PD-1 antagonist is ezabenlimab.

In one embodiment the PD-1 antagonist is pembrolizumab.

In another embodiment the PD-1 antagonist is nivolumab.

In another embodiment the PD-1 antagonist is pidilizumab.

In another embodiment the PD-1 antagonist is atezolizumab.

In another embodiment the PD-1 antagonist is avelumab.

In another embodiment the PD-1 antagonist is durvalumab.

In another embodiment the PD-1 antagonist is PDR-001.

In another embodiment the PD-1 antagonist is PD1-1.

In another embodiment the PD-1 antagonist is PD1-2.

In another embodiment the PD-1 antagonist is PD1-3.

In another embodiment the PD-1 antagonist is PD1-4.

In another embodiment the PD-1 antagonist is PD1-5.

### Pharmaceutical Composition & Formulation

The invention further relates to pharmaceutical compositions for the treatment of a disease (as specified in more detail below), wherein such compositions comprise at least one IL-12 Fc fusion protein of the invention. The invention further encompasses methods of treating a disease (as specified in more detail below) using at least one IL-12 Fc fusion protein of the invention or pharmaceutical composition as set out below, and further encompasses the preparation of a medicament for the treatment of such disease by using such IL-12 Fc fusion protein of the invention or pharmaceutical composition.

The IL-12 Fc fusion protein of the invention (e.g., any as shown in the disclosed sequences) and/or the compositions comprising the same can be administered to a patient in need thereof in any suitable manner, depending on the specific pharmaceutical formulation or composition to be used. Thus, the IL-12 Fc fusion proteins of the invention and/or the compositions comprising the same can for example be administered intravenously (i.v.), subcutaneously (s.c.), intramuscularly (i.m.), intraperitoneally (i.p.), transdermally, orally, sublingually (e.g. in the form of a sublingual tablet, spray or drop placed under the tongue and adsorbed through the mucus membranes into the capillary network under the tongue), (intra-)nasally (e.g. in the form of a nasal spray and/or as an aerosol), topically, by means of a suppository, by inhalation, or any other suitable manner in an effective amount or dose. The IL-12 Fc fusion protein can be administered by infusion, bolus or injection. In preferred embodiments, the administration is by intravenous infusion or subcutaneous injection.

The IL-12 Fc fusion protein of the invention and/or the compositions comprising the same are administered according to a regimen of treatment that is suitable for treating and/or alleviating the disease, disorder or condition to be treated or alleviated. The clinician will generally be able to determine a suitable treatment regimen, depending on factors such as the disease, disorder or condition to be treated or alleviated, the severity of the disease, the severity of the symptoms thereof, the specific binding protein of the invention to be used, the specific route of administration and pharmaceutical formulation or composition to be used, the age, gender, weight, diet, general condition of the patient, and similar factors well known to the clinician. Generally, the treatment regimen will comprise the administration of the IL-12 Fc fusion protein of the invention, or of one or more compositions comprising the same, in therapeutically effective amounts or doses.

Generally, for the treatment and/or alleviation of the diseases, disorders and conditions mentioned herein and depending on the specific disease, disorder or condition to be treated, the potency of the specific IL-12 Fc fusion protein of the invention to be used, the specific route of administration and the specific pharmaceutical formulation or composition used, the IL-12 Fc fusion protein of the invention will generally be administered in an amount between 0.005 and 20.0 mg per kilogram of body weight and dose, preferably between 0.05 and 10.0 mg/kg/dose, either continuously (e.g. by infusion) or more preferably as single doses (such as e.g. twice a week, weekly, or monthly doses; cf. below), but can significantly vary, especially, depending on the before-mentioned parameters. Thus, in some cases it may be sufficient to use less than the minimum dose given above, whereas in other cases the upper limit may have to be exceeded. When administering large amounts it may be advisable to divide them up into a number of smaller doses spread over the day.

Depending on the specific IL-12 Fc fusion protein of the invention and its specific pharmacokinetic and other properties, it may be administered daily, every second, third, fourth, fifth or sixth day, weekly, monthly, and the like. An administration regimen could include long-term, weekly treatment. By “long-term” is meant at least two weeks and preferably months, or years of duration.

The efficacy of the IL-12 Fc fusion protein of the invention, and of compositions comprising the same, can be tested using any suitable in vitro assay, cell-based assay, in vivo assay and/or animal model known per se, or any combination thereof, depending on the specific disease involved. Suitable assays and animal models will be clear to the skilled person, and for example include the assays and animal models used in the Examples below.

For pharmaceutical use, the IL-12 Fc fusion protein of the invention may be formulated as a pharmaceutical preparation comprising (i) at least one IL-12 Fc fusion protein of the invention (e.g., any one as shown in the disclosed sequences) and (ii) at least one pharmaceutically acceptable carrier, diluent, excipient, adjuvant, and/or stabilizer, and (iii) optionally one or more further pharmacologically active polypeptides and/or compounds. By “pharmaceutically acceptable” is meant that the respective material does not show any biological or otherwise undesirable effects when administered to an individual and does not interact in a deleterious manner with any of the other components of the pharmaceutical composition (such as e.g. the pharmaceutically active ingredient) in which it is contained. Specific examples can be found in standard handbooks, such as e.g. Remington's Pharmaceutical Sciences, 18th Ed., Mack Publishing Company, USA (1990). For example, the IL-12 Fc fusion protein of the invention may be formulated and administered in any manner known per se for conventional antibodies and antibody fragments and other pharmaceutically active proteins and fusion proteins. Thus, according to a further embodiment, the invention relates to a pharmaceutical composition or preparation that contains at least one IL-12 Fc fusion protein of the invention and at least one pharmaceutically acceptable carrier, diluent, excipient, adjuvant and/or stabilizer, and optionally one or more further pharmacologically active substances, in the form of lyophilized or otherwise dried formulations or aqueous or non-aqueous solutions or suspensions.

Pharmaceutical preparations for parenteral administration, such as intravenous, intramuscular, subcutaneous injection or intravenous infusion may for example be sterile solutions, suspensions, dispersions, emulsions, or powders which comprise the active ingredient and which are suitable, optionally after a further dissolution or dilution step, for infusion or injection. Suitable carriers or diluents for such preparations for example include, without limitation, sterile water and pharmaceutically acceptable aqueous buffers and solutions such as physiological phosphate-buffered saline, Ringer's solutions, dextrose solution, and Hank's solution; water oils; glycerol; ethanol; glycols such as propylene glycol, as well as mineral oils, animal oils and vegetable oils, for example peanut oil, soybean oil, as well as suitable mixtures thereof.

Solutions of the IL-12 Fc fusion protein of the invention may also contain a preservative to prevent the growth of microorganisms, such as antibacterial and antifungal agents, for example, p-hydroxybenzoates, parabens, chlorobutanol, phenol, sorbic acid, thiomersal, (alkali metal salts of) ethylenediamine tetraacetic acid, and the like. In many cases, it will be preferable to include isotonic agents, for example, sugars, buffers or sodium chloride. Optionally, emulsifiers and/or dispersants may be used. The proper fluidity can be maintained, for example, by the formation of liposomes, by the maintenance of the required particle size in the case of dispersions or by the use of surfactants. Other agents delaying absorption, for example, aluminum monostearate and gelatin, may also be added. The solutions may be filled into injection vials, ampoules, infusion bottles, and the like.

In all cases, the ultimate dosage form must be sterile, fluid and stable under the conditions of manufacture and storage. Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Usually, aqueous solutions or suspensions will be preferred. Generally, suitable formulations for therapeutic proteins such as the IL-12 Fc fusion proteins of the invention are buffered protein solutions, such as solutions including the protein in a suitable concentration (such as from 0.001 to 400 mg/ml, preferably from 0.005 to 200 mg/ml, more preferably 0.01 to 200 mg/ml, more preferably 1.0-100 mg/ml, such as 1.0 mg/ml (i.v. administration) or 100 mg/ml (s.c. administration) and an aqueous buffer such as:
phosphate buffered saline, pH 7.4,
other phosphate buffers, pH 6.2 to 8.2,
acetate buffers, pH 3.2 to 7.5, preferably pH 4.8 to 5.5
histidine buffers, pH 5.5 to 7.0,
succinate buffers, pH 3.2 to 6.6, and
citrate buffers, pH 2.1 to 6.2,
and, optionally, salts (e.g. NaCl) and/or sugars (such as e.g. sucrose and trehalose) and/or other polyalcohols (such as e.g. mannitol and glycerol) for providing isotonicity of the solution.

In addition, other agents such as a detergent, e.g. 0.02% Tween-20 or Tween-80, may be included in such solutions. Formulations for subcutaneous application may include significantly higher concentrations of the IL-12 Fc fusion proteins of the invention, such as up to 100 mg/ml or even above 100 mg/ml. However, it will be clear to the person skilled in the art that the ingredients and the amounts thereof as given above do only represent one, preferred option. Alternatives and variations thereof will be immediately apparent to the skilled person, or can easily be conceived starting from the above disclosure. The above described formulations can optionally be provided as lyophilized formulation that is to be reconstituted in a solution, e.g. in water for injection (WFI).

According to a further aspect of the invention, an IL-12 Fc fusion protein of the invention may be used in combination with a device useful for the administration of protein, such as a syringe, injector pen, micropump, or other device.

### Kits

The invention also encompasses kits comprising at least an IL-12 Fc fusion protein of the invention (e.g., any as shown in the disclosed sequences) and optionally one or more other components selected from the group consisting of other drugs used for the treatment of the diseases and disorders as described above.

In one embodiment, the kit includes a composition containing an effective amount of an IL-12 Fc fusion protein of the invention in unit dosage form.

The invention also encompasses kits comprising at least an IL-12 Fc fusion protein of the invention, and one or more other components selected from the group consisting of other drugs used for the treatment of the diseases and disorders as described above.

In one embodiment, the kit includes a composition containing an effective amount of an IL-12 Fc fusion protein of the invention in unit dosage form. In a further embodiment the kit includes both a composition containing an effective amount of an IL-12 Fc fusion protein of the invention in unit dosage form and a composition containing an effective amount of a PD-1 antagonist in unit dosage form, such as an anti PD-1 antibody, most preferably PD1-1, PD1-2, PD1-3, PD1-4, and PD1-5 as described herein (e.g. Table 7) and in WO2017/198741.

In some embodiments, the kit comprises a sterile container which contains such a composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments. Further, the kit may comprise the pharmaceutical composition in a first container with the IL-12 Fc fusion protein of the invention in lyophilized form and a second container with a pharmaceutically acceptable diluent (e.g., sterile water) for injection. The pharmaceutically acceptable diluent can be used for reconstitution or dilution of the IL-12 Fc fusion protein.

If desired, an IL-12 Fc fusion protein of the invention, is provided together with instructions for administering the IL-12 Fc fusion protein to a subject having cancer. The instructions will generally include information about the use of the composition for the treatment or prevention of a cancer. In other embodiments, the instructions include at least one of the following: description of the therapeutic agent; dosage schedule and administration for treatment or prevention of cancer or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### Method of Production & Purification

A further aspect of the invention provides a method of production of the IL-12 Fc fusion protein as described herein, comprising:
(a) cultivating the host cell of the invention under conditions allowing expression of the molecule; and,
(b) recovering the molecule; and optionally

An embodiment of this aspect of the invention is wherein the method of production further comprises step (c) further purifying and/or modifying and/or formulating the Fc fusion protein.

For producing the IL-12 Fc fusion protein, in practice, the DNA molecule encoding the first polypeptide chain is inserted into an expression vector such that the sequences are operatively linked to transcriptional and translational control sequences. The DNA molecule encoding the second polypeptide chain may be inserted either within the same expression vector or in a different expression vector. The vectors comprise the customary elements needed for expression of the polypeptide in cells, such as promoters, regulatory elements, or elements for selection. After introducing the vector(s) into appropriate host cells both chains will be individually expressed from the vector(s) and secreted by the cells. Both the first and the second polypeptide chain will then associate via their respective Fc domains to form the complete IL-12 Fc fusion protein.

For manufacturing the IL-12 Fc fusion protein, the skilled artisan may choose from a great variety of expression systems well known in the art, e.g. those reviewed by Kipriyanov and Le Gall, Curr Opin Drug Discov Devel. 2004 March; 7(2):233-42.

Expression vectors include plasmids, retroviruses, cosmids, EBV-derived episomes, and the like. The expression vector and expression control sequences are selected to be compatible with the host cell. As outlined, the first and the second polypeptide chain sequences can be inserted into separate vectors. In certain embodiments, both DNA sequences, first and second polypeptide chain sequences, are inserted into the same expression vector. Convenient vectors are those that encode a functionally complete human first and second polypeptide sequence, with appropriate restriction sites engineered so that any first or second polypeptide sequence can be easily inserted and expressed, as described above.

The recombinant expression vector may also encode a signal peptide that facilitates secretion of the polypeptide chains from a host cell. The DNA encoding the polypeptide chains may be cloned into the vector such that the signal peptide is linked in-frame to the amino terminus of the mature first and/or second polypeptide chain DNA. The signal peptide may be an immunoglobulin signal peptide or a heterologous peptide from a non-immunoglobulin protein. Alternatively, the DNA sequence encoding the first or second polypeptide chain may already contain a signal peptide sequence.

In addition to the DNA sequences encoding the IL-12 Fc fusion protein chains, the recombinant expression vectors carry regulatory sequences including promoters, enhancers, termination and polyadenylation signals and other expression control elements that control the expression of the IL-12 Fc fusion protein chains in a host cell. Examples for promoter sequences (exemplified for expression in mammalian cells) are promoters and/or enhancers derived from (CMV) (such as the CMV Simian Virus 40 (SV40) (such as the SV40 promoter/enhancer), adenovirus, (e. g., the adenovirus major late promoter (AdMLP)), polyoma and strong mammalian promoters such as native immunoglobulin and actin promoters. Examples for polyadenylation signals are BGH polyA, SV40 late or early polyA; alternatively, 3′UTRs of immunoglobulin genes etc. can be used.

The recombinant expression vectors may also carry sequences that regulate replication of the vector in host cells (e. g. origins of replication) and selectable marker genes. Nucleic acid molecules encoding the IL-12 Fc fusion protein chains described herein, and vectors comprising these DNA molecules can be introduced into host cells, e.g. bacterial cells or higher eukaryotic cells, e.g. mammalian cells, according to transfection methods well known in the art, including liposome-mediated transfection, polycation-mediated transfection, protoplast fusion, microinjections, calcium phosphate precipitation, electroporation or transfer by viral vectors.

Preferably, the nucleic acid molecules encoding the IL-12 Fc fusion protein chains described herein are both inserted on one vector which is transfected into the host cell, preferably a mammalian cell.

Hence a further aspect provides a host cell comprising an expression vector comprising a nucleic acid molecule encoding the IL-12 Fc fusion protein chains as described herein.

Mammalian cell lines available as hosts for expression are well known in the art and include, inter alia, Chinese hamster ovary (CHO, CHO-DG44) cells, NSO, SP2/0 cells, Hela cells, baby hamster kidney (BHK) cells, monkey kidney cells (COS), human carcinoma cells (e.g., Hep G2), A549 cells, 3T3 cells, HEK, HEK293 or the derivatives/progenies of any such cell line. Other mammalian cells, including but not limited to human, mice, rat, monkey and rodent cells lines, or other eukaryotic cells, including but not limited to yeast, insect and plant cells, or prokaryotic cells such as bacteria may be used. The IL-12 Fc fusion protein of the invention are produced by culturing the host cells for a period of time sufficient to allow for expression of the IL-12 Fc fusion protein in the host cells.

IL-12 Fc fusion proteins as described herein are preferably recovered from the culture medium as a secreted polypeptide or it can be recovered from host cell lysates if for example expressed without a secretory signal. It is necessary to purify the IL-12 Fc fusion protein described herein using standard protein purification methods used for recombinant proteins and host cell proteins in a way that substantially homogenous preparations of the IL-12 Fc fusion protein as described herein are obtained. By way of example, state-of-the art purification methods useful for obtaining the IL-12 Fc fusion protein of the invention include, as a first step, removal of cells and/or particulate cell debris from the culture medium or lysate. The IL-12 Fc fusion protein is then purified from contaminant soluble proteins, polypeptides and nucleic acids, for example, by fractionation on immunoaffinity or ion-exchange columns, ethanol precipitation, reverse phase HPLC, Sephadex chromatography, chromatography on silica or on a cation exchange resin. As a final step in the process for obtaining an IL-12 Fc fusion protein as described herein, the purified IL-12 Fc fusion protein may be dried, e.g. lyophilized, as described below for therapeutic applications.

It will be understood that the immunoglobulin single variable domain molecules, and preferably the VHH's as described herein can be produced and purified mutatis mutandis with the described methods.

### Nucleic Acids

A further aspect of the invention provides isolated nucleic acid molecules that encode the IL-12 Fc fusion protein chains of the invention and/or the immunoglobulin single variable domain molecules of the invention, or an expression vector comprising such a nucleic acid molecule(s).

As can be appreciated by the skilled person, nucleic acid molecules can be readily prepared which encode the first and/or the second polypeptide chains of the IL-12 Fc fusion protein, or the immunoglobulin single variable domain sequence.

Nucleic acid molecules coding for the first and/or the second polypeptide chains of the IL-12 Fc fusion protein, or the immunoglobulin single variable domain sequence, may be synthesized chemically and enzymatically by Polymerase Chain Reaction (PCR) using standard methods. First, suitable oligonucleotides can be synthesized with methods known in the art (e.g. Gait, 1984), which can be used to produce a synthetic gene. Methods to generate synthetic genes from oligonucleotides are known in the art (e.g. Stemmer et al., 1995; Ye et al., 1992; Hayden et Mandecki, 1988; Frank et al., 1987).

The nucleic acid molecules of the invention include, but are not limited to, the DNA molecules encoding the polypeptide sequences shown in the sequence listing. Also, the present invention also relates to nucleic acid molecules that hybridize to the DNA molecules encoding the polypeptide sequences shown in the sequence listing under high stringency binding and washing conditions, as defined in WO 2007/042309. Preferred molecules (from an mRNA perspective) are those that have at least 75% or 80% (preferably at least 85%, more preferably at least 90% and most preferably at least 95%) homology or sequence identity with one of the DNA molecules described herein. By way of example, in view of expressing the IL-12 Fc fusion proteins in eukaryotic cells, the DNA sequences shown in the sequence listing have been designed to match codon usage in eukaryotic cells. If it is desired to express the IL-12 Fc fusion proteins in E. coli, these sequences can be changed to match E. coli codon usage. Variants of DNA molecules of the invention can be constructed in several different ways, as described e.g. in WO 2007/042309.

In another embodiment, any of the disclosed IL-12 Fc fusion proteins can be encoded in an appropriate mRNA sequence. The mRNA sequence could encode both polypeptide chains of the IL-12 Fc fusion protein or two separate mRNA molecules each encoding for one of the two polypeptide chains could be constructed, optionally including at least one secretion signal linked to either or both chains. Such mRNA's could be used to treat patients for any of the treatments as described herein. After delivery of the mRNA(s) to the patient the mRNA would be translated and the polypeptide chains would assemble in the human body to form the complete prodrug.

### EXAMPLES

### Materials & Methods

In the following the materials and methods which have been used in the Examples are described.

### MMP Cleavage Assay:

Recombinant MMP9 (R&D Systems) is activated with p-aminophenylmercuric acetate. Activated MMP9 is incubated with IL-12 Fc fusion protein (prepared in TCNB buffer: 50 mM Tris, 10 mM CaCl₂), 150 mM NaCl, 0.05% Brij-35 (w/v), pH 7.5) for 24 h at 37° C. Digested protein is aliquoted and stored at −80° C. prior to testing in SDS-PAGE, Western blot and IL-12 functional assay.

### MMP Cleavage Assay (Peptide Only):

Recombinant MMPs are activated as per manufacturer's recommendations (R&D). Activated MMP (2.5 nM final conc.) is then added to Dabcyl/Edans Peptides (2.5 μM final conc.) in TCNB buffer. Plates are read at excitation 340 nm/emission 490 nm at 37 ºC for 2 hours with 5 min intervals with the BioTek Synergy H1 Hybrid Multi-Mode Reader (BioTek Instruments). Gain 80 is used. Specific activity is then calculated based on parameters derived from kinetic cleavage curves.

### IL-12 Functional Assays

### NK-92 IL-12 Activity Assay:

NK-92 cells (ATCC CRL-2407) are seeded at a density of 200,000 cells per well in a 96-well plate and 100 μL of medium is added containing varying concentrations of MMP9-cleaved or uncleaved IL-12 Fc fusion protein. The total volume per well is 200 μL which corresponds to a cell density of 100,000 cells/mL. After 24 h of incubation at 37° C., 5% CO₂, the concentration of IFN-γ in the cell culture supernatant is determined by ELISA (Invitrogen). An IFN-γ standard is included in the ELISA and linear curve fitting using the GraphPad Prism 7.0 a software is used to derive the IFN-γ concentration in the samples from the absorbance at 450 nm (A450). The data is fitted using the [Agonist] vs. response (three parameters) fit of the GraphPad Prism 7.0 a software to estimate the EC₅₀.

### Promega IL-12 Bioassay:

The IL-12 Bioassay (Cat. #JA2601, JA2605) is a bioluminescent cell-based assay designed to measure IL-12 stimulation or inhibition. The assay is performed according to manufacturer's protocol. Briefly, cells are thawed, resuspended and 50 μl of solution is pipetted into 96-well plates. 25 μl of serially diluted cleaved and uncleaved IL-12 Fc fusion protein is added to the cells followed by 6 h incubation at 37° C. After incubation, plates are removed from the incubator and after reaching ambient temperature (10-15 min) 75 μl of Bio-Glo™ reagent is added to each well for 5-10 min at RT followed by luminescence measurement. Fold induction is calculated using following formula: Fold induction=RLU (sample-background)/RLU (no drug control-background). Data are plotted using GraphPad Prism software and EC₅₀ is determined.

### TME Linker Binding

### ELISA:

IL-12 Fc fusion protein (in amounts indicated in figure legends) is diluted in PBS and added to collagen I-coated 96-well plates (Corning) for 10-30 min. The plates are blocked with 2% BSA before addition of proteins to minimize unspecific binding. Next, plates are washed 3× followed by incubation with biotinylated anti-human Fc antibody (Invitrogen). After additional washing step, Streptavidin-HRP and substrate are added and plates are read in a Tecan reader.

### Scar in a Jar Assay:

Primary human lung fibroblasts are plated in a poly-D-lysine coated 384 CellCarrier microtiter plate from PerkinElmer in FBM with FGM-2TM Single Quots (Lonza, Basel, Switzerland) at a density of 1000 cells per well. After 24 hours, the medium is replaced by the same medium containing no serum (starvation medium). Forty-eight hours after cell seeding, the starvation medium is replaced with starvation medium containing a mixture of Ficoll 70 and 400 (37.5 mg/mL and 25 mg/mL, respectively), 200 UM of vitamin C, and IPF-RC (1:1000 dilution). After 72 h, the cell culture medium is removed and cells are fixed with 100% of ice-cold methanol for 30 minutes. Next, cells are washed with PBS and the plate is decellularized, IL-12 Fc fusion protein (5 μg and 50 μg) is added to the wells for 2 h incubation. Next, plates are washed and blocked for 30 minutes with 3% of BSA in PBS. After an additional wash step, collagen I is stained using a monoclonal antibody (SAB4200678, Sigma-Aldrich). For primary antibody detection, cells are washed and incubated for 30 minutes at 37° C. with Goat anti mouse IgG1 Alexa Fluor 568 secondary antibody. Following a final wash step, images are acquired in an Opera Phenix (Perkin Elmer), and images are transferred to the Columbus Image Storage and Analysis system (Perkin Elmer).

### In Vivo Models:

C57BL/6 mice are injected in the left flank with MC38 or B16.F10 cells. Treatment starts when tumor reach ca. 70-100 mm³. Mice are treated 2× per week with up to 6 injections. Doses of IL-12 Fc fusion protein and specific proteins used are provided in the Figures. Tumor volume and body weight is monitored 2-3× per week. Statistical analysis is performed using GraphPad Prism software. The differences between groups are analyzed using t-test with or without Welch's correction, depending on the data distribution. Analysis of grouped data is performed using two-way ANOVA or Kruskal-Wallis Test.

### Generation of Stable Pools

Final molecules are cloned into a mammalian expression system, encoding the knob and hole chain on one plasmid, driven by separate CMV promoters and a metabolic selection marker. CHO-K1 GS−/− host cells are transfected with respective expression plasmids and stable pools are generated and banked for cell culture processes.

### Production and Purification

Final molecules are produced from stable transfected and characterized CHO cell pools in bioreactors. Cell culture process is performed under controlled conditions. Cell culture harvest is processed in an automated downstream process including Protein A capture, acid treatment, cation exchange and anionic mixed mode chromatography polishing and different filtration steps. Product quality of each construct is then determined.

### Production and Purification

Expression is performed in 3 L bioreactors with stable transfected CHO cell pools. Cell culture is harvested after 14 days in culture. Titer is determined with Protein A HPLC. Purification is performed with an automated representative multi-step process train. Protein A capture with subsequent virus inactivation at low pH is performed followed by cation exchange and mixed-mode chromatography. Constructs are finally concentrated using ultrafiltration/diafiltration.

### Product Quality Assessment

Purity of all constructs is assessed using state-of-the-art size exclusion chromatography and non-reducing capillary gel electrophoresis. Product quality is determined by hydrophilic liquid interaction chromatography on-line coupled to mass spectrometry (HILIC-MS).

### Overall Design of an IL-12 Fusion Protein

In the following examples the design path for the IL-12 Fc fusion proteins is laid out. In the context of the examples said molecules are also referred to sometimes as prodrug(s) or protease-activatable prodrug(s).

### Example 1

### Molecular Components

Various arrangements of protease-activatable prodrugs were evaluated that were desired to include the following components:

1) The cytokine itself: IL-12 is a heterodimer, consisting of the p40 and p35 subunits, however, these two domains may be linked together via a connecting peptide linker to form a functional single-chain cytokine.

2) A masking moiety that can block the function of the cytokine/payload while part of the prodrug, but would also release the cytokine/payload upon enzymatic activation of the prodrug.

3) An enzymatically cleavable linker, whose sequence composition is recognized by enzymes that are upregulated within the tumor microenvironment.

4) Optionally, the prodrug may contain additional components, such as the constant region (Fc) for an antibody, to extend half-life of the prodrug. The Fc allows for creating heterodimeric Fc's through technologies such as Knob-in-Hole. Here, the Fc can act as a heterodimerization domain that allows the cytokine to be produced on one arm and the mask to be produced on the other arm, “in parallel.” Conversely, wild-type Fc could also be used, where the cytokine is directly linked to the masking domain, followed by the Fc, or some combination thereof, to form a symmetric prodrug “in series.”

5) Optionally, the prodrug may contain a tumor targeting domain, which may be a domain that targets the prodrug specifically to e.g a tumor antigen or as a further alternative preferentially to tumor related structures in the vicinity of the tumor. The effects of such a tumor targeting domain could be three-fold: A) to anchor the prodrug within the tumor, allowing for increased exposure of the prodrug to upregulated enzyme activity, enhancing conversion of the prodrug to active drug, or B) to anchor the active cytokine within the tumor, enhancing its residency time and/or half-life within the tumor microenvironment and C) to anchor the active cytokine within the tumor to decrease potential toxicity associated with systemic exposure.

### Format Scouting of Prodrug Molecules

### FIGS. 1A-1H

In order to explore functional inhibition of IL-12 in the prodrug form, the above considerations were put into practice by producing molecules having different orientations and components and measuring the effect of the prodrug format on its ability to create a delta EC₅₀ (ΔEC₅₀) of IL-12 activity in functional assays, comparing the prodrug form to the active drug after cleaving the prodrugs enzymatically cleavable linker, e.g. with MMP9.

Permutations of the following parameters were altered to generate eight (8) initial molecules:
a. Fusion of the cytokine and/or masking domain to the N-terminus of the Fc, or optionally, to the C-terminus of the Fc.
b. Utilization of an Fc that naturally homodimerizes (wild type interface), where the cytokine and masking domain are “in series”, or utilization of an Fc that was engineered to heterodimerize, such as using Knob In Hole technology, and the cytokine residued on an Fc chain opposite the masking domain, “in parallel.”
c. Utilization of either single-chain IL-12 or a version where the p35 domain was linked directly to the Fc and the p40 domain was co-transfected simultaneously, and IL12 forms intracellularly during protein production, and the two domains were covalently linked through a naturally-occurring disulfide bond.

Functional readouts of the format scouting using a NK-92 cell based assay are shown in below table. The masking domain was in each case an scFv tool molecule against IL-12 with an affinity towards IL-12 with around ˜250 pM.

TABLE 8

| | Functional Readouts of Format Scouting using NK-92 Cell Based Assay | | | | | | | |
|---|---|---|---|---|---|---|---|---|
| | | | | | | EC50 | EC50 | |
| Construct | Figure | Fusion | Cleavabe | | | (pM), | (pM), | |
| Name | Reference | Location | linker | IgG | IL12 | uncleaved | cleaved | ΔEC₅₀ |
| BI-109 | 1A | N- | Between | Knob-in- | Single-chain | 292.7 | 20.4 | 14.4 |
| | | terminus | IL12 and | Hole | via Gly/Ser | | | |
| | | of Fc | Fc | | linker | | | |
| BI-110 | 1B | C- | Between | Knob-in- | Single-chain | 1489.0 | 36.8 | 40.5 |
| | | terminus | IL12 and | Hole | via Gly/Ser | | | |
| | | of Fc | Fc | | linker | | | |
| BI-111 | 1E | N- | Between | Wild | Single-chain | 743.9 | 143.6 | 5.2 |
| | | terminus | IL12 and | Type | via Gly/Ser | | | |
| | | of Fc | Fc | | linker | | | |
| BI-112 | 1F | C- | Between | Wild | Single-chain | 766.9 | 48.8 | 15.7 |
| | | terminus | IL12 and | Type | via Gly/Ser | | | |
| | | of Fc | Fc | | linker | | | |
| BI-113 | 1C | N- | Between | Knob-in- | Heterodimeric, | 197.0 | 15.3 | 12.9 |
| | | terminus | IL12 and | Hole | with p35 | | | |
| | | of Fc | masking | | fused to Fc | | | |
| | | | domain | | and p40 as an | | | |
| | | | | | independently | | | |
| | | | | | expressed | | | |
| | | | | | chain | | | |
| BI-114 | 1D | C- | Between | Knob-in- | Heterodimeric, | 447.2 | 5.8 | 77.0 |
| | | terminus | IL12 and | Hole | with p35 | | | |
| | | of Fc | masking | | fused to Fc | | | |
| | | | domain | | and p40 as an | | | |
| | | | | | independently | | | |
| | | | | | expressed | | | |
| | | | | | chain | | | |
| BI-115 | 1G | N- | Between | Wild | Heterodimeric, | 798.9 | 32.2 | 24.8 |
| | | terminus | IL12 and | Type | with p35 | | | |
| | | of Fc | masking | | fused to Fc | | | |
| | | | domain | | and p40 as an | | | |
| | | | | | independently | | | |
| | | | | | expressed | | | |
| | | | | | chain | | | |
| BI-116 | 1H | C- | Between | Wild | Heterodimeric, | Unstable | 447.1 | Not |
| | | terminus | IL12 and | Type | with p35 | | | Calculated |
| | | of Fc | masking | | fused to Fc | | | |
| | | | domain | | and p40 as an | | | |
| | | | | | independently | | | |
| | | | | | expressed | | | |

### Example 2

### Masking Moiety (Generation of VHH)

### FIG. 2

The masking moiety could be selected from a wide range of inhibitory molecules to IL-12: receptor fragments, antibodies or antibody fragments (Fab, scFab, scFv, VHH, as examples), or peptides. The masking moiety could be a direct inhibitor of IL-12 activity, in which the mask may block IL-12 signaling through its receptor in a functional assay. Masking moieties may also be derived from IL-12 binding, or IL-12 fusions, that do not directly inhibit IL-12, but instead indirectly inhibit IL-12 through steric interactions in the context of the prodrug assembly.

Ensuring that the cytokine does not signal systemically, creating an effective ΔEC₅₀ between the prodrug in systemic circulation, and active cytokine in the tumor microenvironment was considered a critical feature of designing such molecules.

Antibody selections to attempt to identify a masking domain were undertaken. Both in vitro and in vivo techniques were used to generate antibodies against IL-12: phage panning of a synthetic or immune-derived VHH-based antibody libraries were performed. From the immunized Llamas the plasma cells were collected and converted into phage display libraries, and were further panned to directly isolate binding fragments. The selection of an appropriate antibody fragment for a masking domain was based on functional inhibition of IL-12, affinity of binding to IL-12, and the creation of a large therapeutic window between the prodrug and activated IL-12, measured in the Promega IL-12 Bioassay.

Phage panning of the synthetic VHH-based antibody library was unsuccessful to identify any functional blockers of IL-12. Although over 50 VHH-based masking domains from the synthetic VHH-based antibody library were tested none of them did show any functional activity in the Promega IL-12 Bioassay (data not shown) suggesting an additional hurdle to select an optimal masking molecule.

From the immune-derived VHH-based antibody library 47 initial binders were identified and further characterized.

TABLE 9

| | |
|---|---|
| BI-001 | QVQLVESGGGLVQPGGSLRLSCAASGREFTSVSMAWFRQR |
| | PGKEREFVAFSARISETTEYADFVKGRFTIWRDNANAKVTV |
| | YLQMNNLKPEDTGVYYCAASEGPATIRQNTYPDWGQGTQV |
| | TVSS |
| BI-002 | EVQLVESGGGLVQPGGSLRLSCAASGFTFSSYYMYWIRQAP |
| | GKGLEWVSTIKPNGSGIYGNSVAGRFTISRDNAKNMLYLQM |
| | NMLRPEDTALYYCARDVRGTVRGQGTQVTVSS |
| BI-003 | QVQLVESGGGLVQPGGSLRLSCAASGNQLSLYNMGWYRQ |
| | APGKQRELVASISRAGRSSYGDSVKGRFTISRDNAKNTVYL |
| | QMSSLKPEDTAVYYCKASFLDDYWGQGTQVTVSS |
| BI-004 | EVQLVESGGGLVQPGGSLRLSCAASGFSFSSSWMFWVRQPP |
| | GKGLEWVSTISPSGDYSRYADSVQGRFTISRDNTKNTLSLQ |
| | MNSLKPEDTALYYCARDLRGTMRGQGTQVTVSS |
| BI-005 | QVQLVESGGGLVQPGGSLRLSCAASGFTFSNFVMKWYHQA |
| | PGKERDLVASIDTTHFTNYADSVKGRFTISRDNSKNTVYLQ |
| | MNSLKSEDTAVYYCKVRRRDYEDYWGQGTQVTVSS |
| BI-006 | EVQLVESGGGLVQAGGSLRLSCAASGRTFSRYNMGWFRQA |
| | PGKEREFVAAIIWSGGVINYADSVKGRFTISRDNAKNTVYL |
| | QMNSLKPEDTARYYCAADDKYYDRIVRGTADYWGQGTQV |
| | TVSS |
| BI-007 | EVQLVESGGGLVQPGGSLRLSCVASGSIGSVVSWGWYRQA |
| | PGLERELVASDASGGRPNYQDSVKGRFTISRDSAKNTVYLQ |
| | MNSLKPEDTAVYYCNLRGLQLDMGLYDSWGQGTQVTVSS |
| BI-008 | EVQLVESGGGLVQAGDSLKLSCATSGRPSRDYAMGWFRQA |
| | PGKKRDFVAGISSGGGFTNYADSVKARFTISKDNAKNTVYL |
| | QMNSLKPEDTAVYYCAAQSGTNYISRTSPPYWGQGTQVTV |
| | SS |
| BI-009 | EVQLVESGGGLVHPGGSLRLSCVASGFRFTPYTMGWYRQA |
| | PGKQRELVASISSVYSTNYADSVKGRFTVSRDNVKGTVSLQ |
| | MNSLKPEDTAVYYCNAPGLLHEEGTDYWGQGTQVTVSS |
| BI-010 | QVQLVESGGGSVQVGGSLRLSCVGSGRTLNMYNMGWFRQ |
| | APGKEREFVAAISGKGLISDYRDSVKGRFTISRDNARNTMYL |
| | QMNSLKPEDTAVYHCAAGQWSAGPFTRERSYEYWGQGTQ |
| | VTVSS |
| BI-011 | EVQLVESGGGLVQAGGSLRLSCAASGRTFSRWTMAWFRQA |
| | PGKERDFVAAVGWWNDSTYYADSVKGRFTISRDNNENTLY |
| | LQMNSLKPEDTAVYICASTEKYGLGQPNPRRYDYWGQGTQ |
| | VTVSS |
| BI-012 | QVQLVESGGGLVQPGGSLRLSCAASGRTLSSYTMAWFRQA |
| | PGKEREFVATISPVGFIMDYADSVKGRFTISRDNTKNTVYLQ |
| | MNSLKHEDTALYYCATDLGRKLGTQSREYGYWGQGTQVT |
| | VSS |
| BI-013 | EVQLVESGGGLVQAGGSLRLSCAASGRTFSTYAVGWFRQA |
| | PGKEREFVAAISWGGGTVRYADSVKGRSTISRDDAKNTVYL |
| | QMNSLKPEDTAIYYCAARVLHIATKAVDFGSWGQGTQVTV |
| | SS |
| BI-014 | EVQLVESGGGSVQVGGSLRLSCVASGRTFNMYVMGWFRQ |
| | APGKEREFVAAISGEGLISDYRDSVKGRFTISRDNAKNTMYL |
| | QMNSLKPEDTAVYHCAAGQWTNGPFTRERTYEYWGQGTQ |
| | VTVSS |
| BI-015 | EVQLVESGGGLVQPGGSLRLSCAASGRDFDRSTMAWYRQA |
| | PGKQRELVASIPSDIGTKYADSVKGRFFISRVYAKNTNTVYL |
| | QMNSLKPEDTAVYYCYAHIDSDYWGQGTQVTVSS |
| BI-016 | EVQLVESGGGLVQPGGSLRLSCAASGRTFSSRAMGWFRQA |
| | PGKEREFVAAISFGGGTIRYADSVKGRFTISRDDAKNTVYLQ |
| | MNSLKPEDTAVYYCAARRLHIATLAADFDSWGQGTQVTVS |
| | S |
| BI-017 | EVQLVESGGGLVQAGGSLRLSCSASGRSLNDYIVGWFRQAP |
| | GKERELVAAISSGGYIQHYIDSVKGRFTISRDNAKNTVYLQ |
| | MNGLKPEDTAVYYCAANQLNGVARKKIESDDYDYWGQGT |
| | QVTVSS |
| BI-018 | EVQLVESGGGLVEAGGSLRVSCAASGGTFSEYAMGWIRQA |
| | PGKEREFVAGISRGAGRTVYADSVKGRFTISRDNHKNTVYL |
| | QMNSLKPEDTAVYYCGADDVSYNRVTTAPGEYDYWGQGI |
| | QVTVSS |
| BI-019 | EVQLVESGGGAVQAGGALKLSCAYSGRAFSRSLMGWFRQA |
| | PGKEREFVAAISWVSVTPDYGDSVKGRFTISRDNAKSTVTL |
| | QMNSLKPEDTAVYYCAASERYGTPRRRPNDYDYWGQGTQ |
| | VTVSS |
| BI-020 | EVQLVESGGGLVQPGGSLRLSCVASGSIGSITSMAWYRQGT |
| | GKQRELVASISSGGRPSYQDSVKGRFTISRDNAENTVYLQM |
| | NSLKPEDTAVYHCNVRGLHLDTGLYESWGQGTQVTVSS |
| BI-021 | EVQLVESGGGLVQPGGSLRLSCASPGSISTLYVMGWYRQAP |
| | GKQRDLVARITRGGSTSYANSVKGRFTISRDNVNNTINLQM |
| | NSLKPEDTAVYYCYAQTAVGPDYWGQGTQVTVSS |
| BI-022 | QVQLVESGGGLVQAGGSLRLSCAASGSIFSTLNAIGWYRQA |
| | PGKQAELVARITHDGRIVYGDSVKGRFTISRDNAKNTAYLQ |
| | MNSLNPEDTAVYFCVAPGMVRGQGTQVTVSS |
| BI-023 | QVQLVESGGGLVQAGDSLRLSCTASGRTLTLSMVTVGWFR |
| | QGSGKEREFVAAISWRGGRSYVADDVKGRFTISRDNARNT |
| | VYLQMNSVKPEDTAVYYCAAREAIQDLAWTANDFTYWGQ |
| | GTQVTVSS |
| BI-024 | QVQLVESGGGSVQAGGSLRLSCAASGRTFNTKAIGWFRQAP |
| | GKEREFVAAISWGGGTIRYADSVKGRVTISRDDAKNTVYLQ |
| | MNSLKPEDTAVYYFATRQLHIATLAADFDSRRQGTQVTVSS |
| BI-025 | EVQLVESGGGLVQPGGSLRLSCAASGRTLSSYTMAWFRQA |
| | PGKEREFVATISPVGFIMDYADSVKGRFTISRDNTKNTVYLQ |
| | MNSLKHEDTALYYCATDLGRKLGTQSCEYGYWGQGTQVT |
| | VSS |
| BI-026 | EVQLVESGGGLVQPGGSLRLSCVASGSTGSITSMAWYRQAP |
| | GKQRELVASINSGGRPNYQESVKGRFTISRDNAENTLYLQM |
| | NSLSPEDTAVYLCNLRGLRLDTGLYESWGQGTQVTVSS |
| BI-027 | QVQLVESGGGSVQAGGSLRLSCAASGRTFSSRAMGWFRQA |
| | PGKEREFVAAISFGGGTIRYADSVKGRFTISRDDAKNTVYLQ |
| | MNSLKPEDTAVYYCAARRLHIATLAADFDSWGQGTQVTVS |
| | S |
| BI-028 | EVQLVESGGGLVQTGGSLRLSCAASGLTNGYVMAWFRQAP |
| | GKEREFVSGIGWGSSRTYYADSVKGRFTISRDNAINTVALQ |
| | MNSLKPEDTAVYYCAAQGRISPIYTRANEYPYWGQGTQVT |
| | VSS |
| BI-029 | QVQLVESGGGLVQPGGSLRLSCAASGFTFSSYGMSWYRQA |
| | PGKEHELVAGISAGSTKYADSVKGRFTISRDNAKKTVYLQM |
| | NSLKPEDTAVYYCSRWPRLFEDWGQGTQVTVSS |
| BI-030 | EVQLVESGGGLVQPGGSLRLSCVASGDIGSMTSTGWYRQAP |
| | GKQRDLVASINSGGRPNYQDSVKGRFTISRDSAKNTVYLQM |
| | NSLKPEDTAVYYCNLRGLVLSTGLYESWGQGTQVTVSS |
| BI-031 | QVQLVESGGGLVQPGGSLRLSCAAPGSIATLYVMGWYRQA |
| | PGKQRELVARITRGGSTSYANAVKGRFTISRDNAKNTVNLQ |
| | MNSLKPEDTAIYYCNAQTAVGPDYWGQGTQVTVSS |
| BI-032 | EVQLVESGGGLVQAGGSLRPSCAASGRTFSNYNMGWFRQA |
| | PGKERESVATISRSGVITDYADSVKGRFTISRDNAKNTVYLQ |
| | MDSLKPEDTAVYYCAAARSPVWGRGPDEYDTWGQGTQVT |
| | VSS |
| BI-033 | EVQLVESGGGLVQAGGSLRLSCAASGRTFSAYVMGWFRQT |
| | PGKGREFVAAISTGGQISDYANSVKGRFTISRDNAKNTAYL |
| | QMNNLKPEDTAVYYCAANRENFLNRGAGDYEYWGQGTQ |
| | VTVSS |
| BI-034 | QVQLVESGGGLVQPGGSLRLSCVASGSTGSITSMAWYRQAP |
| | GKQRELVASINSGGRPNYQESVKGRFTISRDNAENTLYLQM |
| | NSLSPEDTAVYLCNLRGLRLDTGLYESWGQGTQVTVSS |
| BI-035 | QVQLVESGGGLVQPGGSLRLSCAASGSIAEIYVMGWYRQAP |
| | GKQREIVATTPSSGRTNIADSVKGRFIISRDFVKNTVALQMN |
| | SLKPEDTAVYYCYARLTPTSVASWGPGTQVTVSS |
| BI-036 | EVQLVESGGGSVQAGGSLRLSCATSGRGFSTYAMGWFRQA |
| | PGKEREFVAAISFGGGTVRYVDSVKGRFTISRDDAKGTVYL |
| | QMNSLKPEDTAVYYCAARRLHIATLAADFGSWGQGTQVTV |
| | SS |
| BI-037 | EVQLVESGGGLVQAGDSLRLSCAASGRTFSTYVMGWFRQA |
| | PGKEREFVAYISTGGLISDHADSVKGRFTISRDNAKNTVYLQ |
| | MNSLRPEDTAVYYCAAASRTRRPIATIKDEYDYWGQGTQV |
| | TVSS |
| BI-038 | EVQLVESGGGLVQAGDSLRLSCTASGRTLTLSMVTVGWFR |
| | QGSGKEREFVAAISWRGGRSSVADDVKGRFTISRDNARNTV |
| | YLQMNSVKPEDTAVYYCAARTAIQDLAWTANDFTYWGQG |
| | TQVTVSS |
| BI-039 | QVQLVESGGGLVQAGGSLRLSCAASGRTFETIAMGWFRQV |
| | PGKEREFVAVIRGSGVATYYPDSVKGRFTISKDSAKNTVYL |
| | QMNNLKPEDTAVYYCAATTNRFKNTDYTTYDYWGQGTQV |
| | TVSS |
| BI-040 | EVQLVESGGGSVQVGGFLRLSCVGSGRTLNMYNMGWFRQ |
| | APGKEREFVAAISGKGLISDYRDSVKGRFTISRDNARNTMYL |
| | QMNSLKPEDTAVYHCAAGQWSAGPFTRERSYEYWGQGTQ |
| | VTVSS |
| BI-041 | EVQLVESGGGLVQAGGSLRLSCASSGRTFSNYNMGWFRQA |
| | PGKERESVATISRSGVITDYADYVKGRFTISRDNAKNTVYLQ |
| | MDSLKPEDTAVYYCAAARSPVWGRGPDEYDTWGQGTQVT |
| | VSS |
| BI-042 | EVQLVESGGGSVQAGGSLRLSCAASGRTFNTKAIGWFRQAP |
| | GKEREFVAAISWGGGTIRYADSVKGRVTISRDDAKNTVYLQ |
| | MNSLKPEDTAVYYCAARQLHIATLAADFDSWGQGTQVTVS |
| | S |
| BI-043 | EVQLVESGGGLVQAGGSLRLSCAASGRTYSTVAMGWFRQA |
| | PGKEREFVGAITWSVGNTAVADSVKGRFAISRDSAKNTVYL |
| | QMNSLKVEDTAVYYCASRTNIGAFNLFRENHYNYWGQGT |
| | QVTVSS |
| BI-044 | EVQLVESGGGLVQPGGSLRLSCKASGSIGSVTSMGWYRQAP |
| | GKQRDLVASADSNGRTTFQDFVQGRFTISRDSAKNTWYLQ |
| | MNSLKPEDTAVYYCHLRGLQLTMGLYESWGQGTQVTVSS |
| BI-045 | EVQLVESGGGAVQAGGALNVSCAASGRAFSRTLMGWFRQ |
| | APGKEREFVAGISWVSVTPDYGNSVKGRFTISRDNAKSTVY |
| | LQMNSLKPEDTAVYYCAASQRYGTPRRRPNDYEYWGQGIQ |
| | VTVSS |
| BI-046 | EVQLVESGGGSVQTGGSLRLSCKVSEGSFMRYNMGWFRQA |
| | PGKERDFVAAMSGALALIRYADSVKGRFTISRDNSKNTVYL |
| | DMNSLKPEDTAVYYCAADLEPQYWTKAGQRDTYDVWGQ |
| | GTQVTVSS |
| BI-047 | EVQLVESGGGLVQAGGSLRLSCAASGSISSNIMGWFRQAPG |
| | KEREFVAVISRRGLILDYGDSVKGRFTMSRDNAKKAVYLQ |
| | MNSLKPEDTAVYYCAVGKTTDRFSEIPSDYDYWGQGTQVT |
| | VSS |

The majority of the VHH did not show functional activity in the Promega IL-12 Bioassay. Only one functional VHH binder (p40 binder) was identified and further pursued for optimization and humanization (BI-039). Although the other identified VHH binders did not show activity in the functional assay they still find utility in other applications requiring binding to IL-12.

The masking domain selected (BI-048) was shown to compete with ustekinumab, a known inhibitor of IL-12 & IL-23, by blocking domain 1 of the p40 domain. Selecting a masking domain that was specific for the p40 domain was an important consideration in the design of this molecule, as human IL-12 does not signal through mouse IL-12 receptor; however, a chimeric IL-12 that utilizes human IL-12 p40 and mouse IL-12 p35 can signal. Therefore, a masking domain that is specific for the p40 domain of IL-12 allowed for the creation of surrogate prodrugs without the need for species cross-reactivity of the masking domain itself. This masking domain went through three rounds of humanization and was able to maintain its potency and efficacy against IL-12. The selected VHH and its humanized variant both had affinities of 3.5 nM and an IC₅₀ of approximately 500 pM (TABLE 10).

TABLE 10
Variations of BI-039:

| Name | Identifier | K_{D} (nM) | IC50 [nM] |
|---|---|---|---|
| 61-Gft-106_D62E | BI-117 | 3.0 | 0.36 |
| 61-Gft-106-DE_Mix13 | BI-048 | 3.7 | 0.44 |
| BI-039 | BI-039 | 3.5 | 0.61 |
| 61-Gft-106 | BI-063 | 3.4 | 0.89 |
| 61-Gft-106-DE_Mix17 | BI-118 | 6.3 | 1.5 |
| 61-Gft-106-DE_T106R | BI-119 | 8.5 | 2.4 |
| 61-Gft-106-DE_D107E | BI-120 | 4.1 | 2.5 |
| 61-Gft-106-DE_Mix15 | BI-049 | 12 | 5.7 |
| 61-Gft-106-DE_Mix16 | BI-121 | 9.2 | 6.5 |
| 61-Gft-106-DE_Mix7 | BI-122 | 9.5 | 7.9 |
| 61-Gft-106-DE_Y59S | BI-123 | 30 | 10 |
| 61-Gft-106-DE_Mix12 | BI-124 | 12 | 10 |
| 61-Gft-106-DE_Mix26 | BI-125 | 36 | 16 |
| 61-Gft-106-DE_Lia1 | BI-126 | 13 | 18 |
| 61-Gft-106-DE_Mix22 | BI-127 | 29 | 20 |
| 61-Gft-106-DE_Mix21 | BI-128 | 51 | 56 |

### Example 3

### MMP Expression and Cleavable Linker

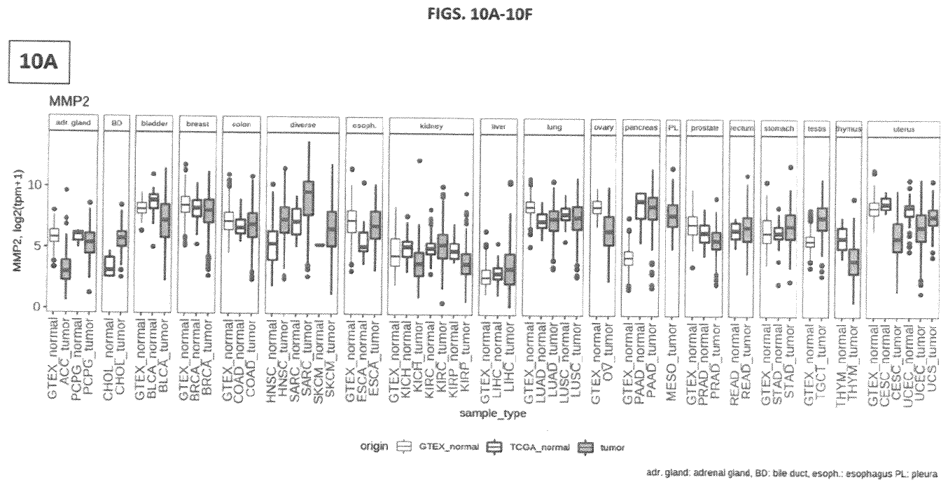

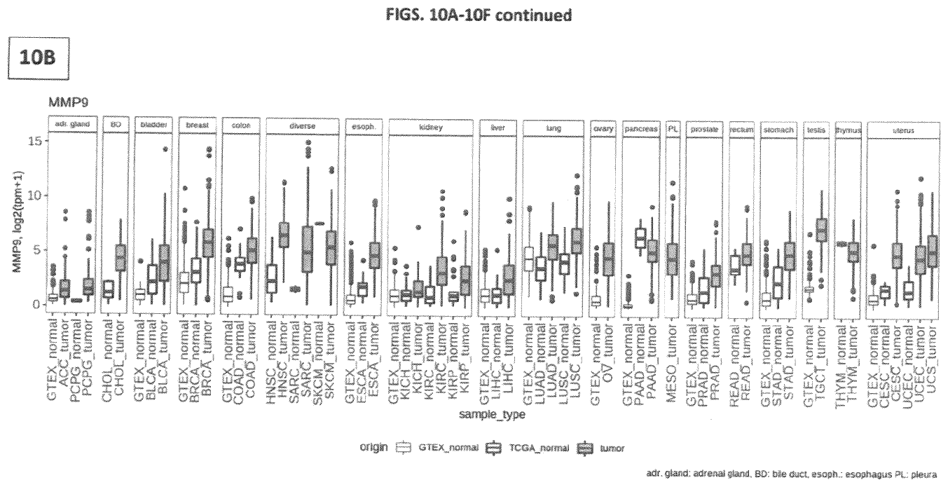

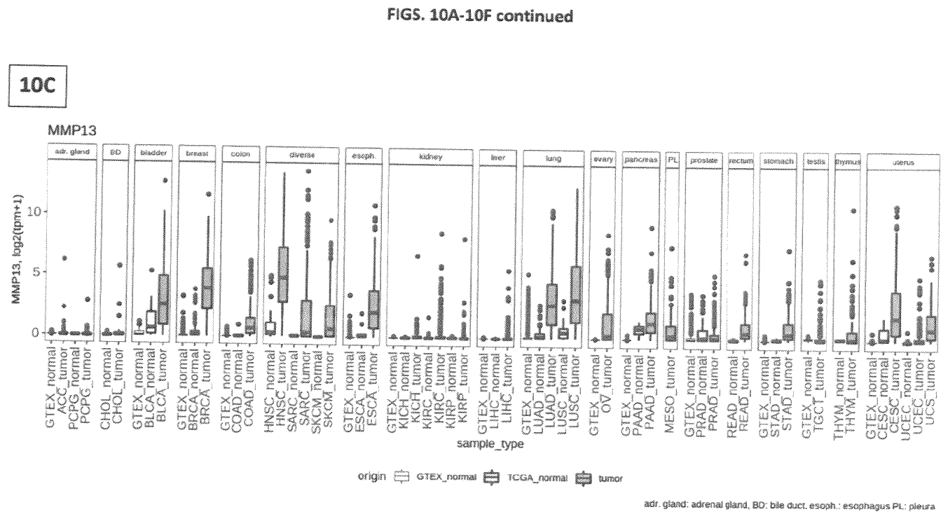

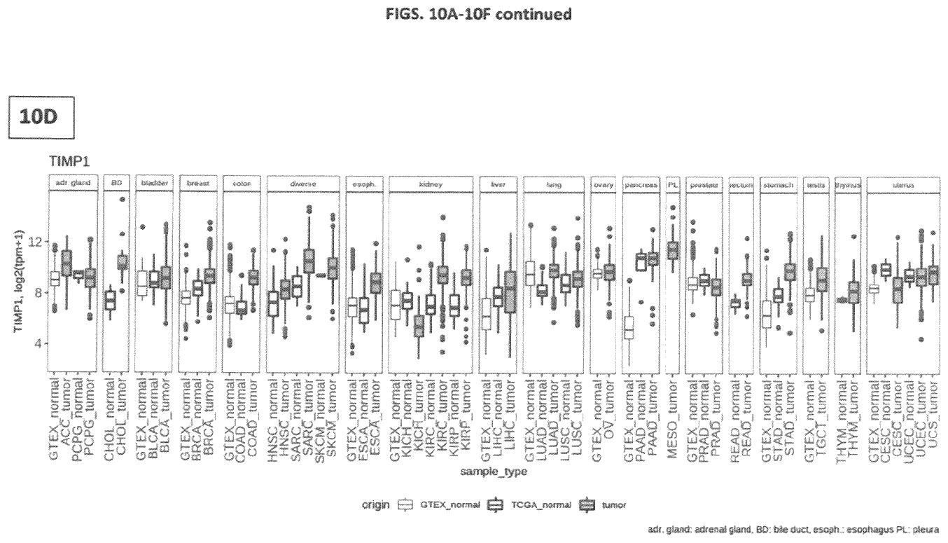

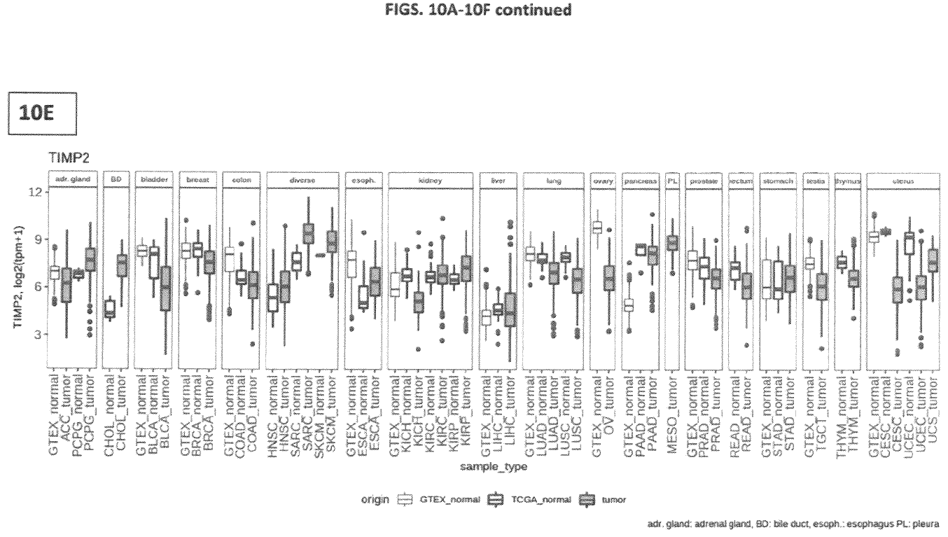

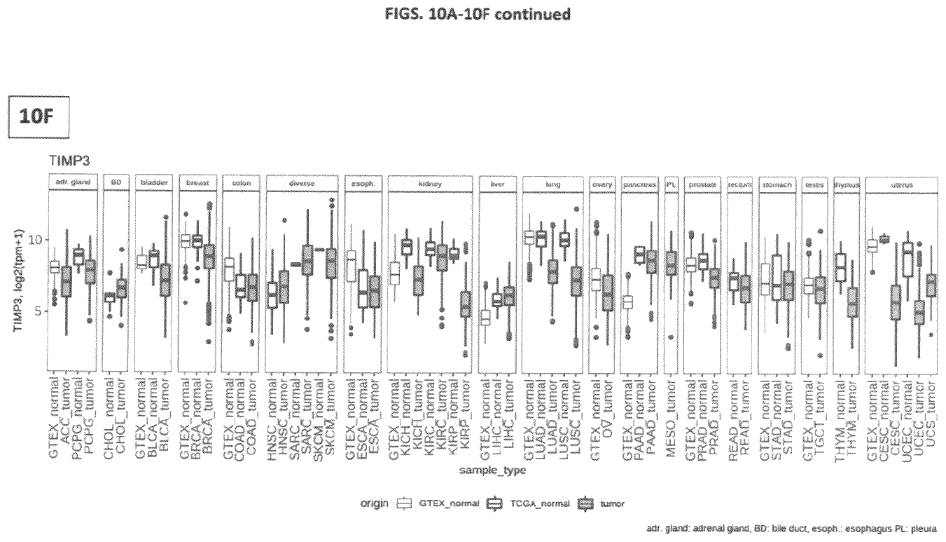

### FIGS. 10A-10F

MMPs are often tightly regulated systemically by their natural inhibitors, Tissue Inhibitor of Metalloproteinases, otherwise known as TIMPS. However, within the tumor microenvironment, not only are MMP transcripts upregulated, their activity is often aided by a reduction in TIMP activity, therefore, allowing for higher MMP within the tumor itself. Expression level of various MMPs and TIMPs in a variety of human tumors was evaluated and are presented in FIGS. 10A-10F. Expression of MMPs, in particular MMP2, MMP9 and MMP13 was clearly upregulated in tumor tissues compared to the adjacent normal or healthy organs. In addition, in many tissues TIMPs (especially TIMP3) were strongly upregulated in healthy tissues compared to tumor tissues suggesting higher MMP activity within tumors compared to the normal tissue.

In addition to expression level, activity of MMPs needed to be evaluated to confirm the functionality of those enzymes in the biological samples. To this end, tumor samples (and adjacent normal) were collected from patients undergoing surgical removal of tumor. The samples were evaluated with regard to expression level of various MMPs as well as in functional assay to confirm proteolytic activity of enzymes. As presented in Table 11 MMP2 and MMP9 were abundantly expressed in tumor tissue. In addition, MMP2 and MMP9 were upregulated in some adjacent normal tissue; however, this level of expression did not correlate with the enzymatic activity possibly due to activity of inhibitory proteins (TIMPs). Contrary to MMP2 and MMP9, MMP12 and MMP13 were almost exclusively expressed in tumor tissue.

TABLE 11
Patient data are shown comparing the MMP activity of tumor samples vs. adjacent normal tissue
from patients that underwent surgical removal of tumor. The individual patient data are presented
in pairs, i.e. the tumor sample and the normal sample from the same patient are presented
directly below each other. The MMP expression level is shown in pg per mg lysate and the
specific activity is expressed in pmol (of peptide cleaved)/min/μg of lysate.

| | | | | | | | | | | Tissue |
|---|---|---|---|---|---|---|---|---|---|---|
| T/N | MMP1 | MMP2 | MMP7 | MMP9 | MMP10 | MMP3 | MMP12 | MMP13 | Activity | Type |
| Tumor 1 | 1514 | 3376 | 4804 | 1728 | <27 | <206 | 776 | 3878 | 0.21 | Lung |
| Normal 1 | 69 | 9188 | 183 | >10000 | <27 | <206 | <137.2 | <82.3 | 0.09 | Lung |
| Tumor 2 | 338 | 12309 | 9391 | 8145 | <27 | <206 | <137.2 | 174 | 0.18 | Lung |
| Normal 2 | 247 | 14400 | 556 | 6832 | <27 | <206 | <137.2 | <82.3 | 0.08 | Lung |
| Tumor 3 | 6297 | 6724 | 16734 | 1290 | <27 | <206 | 406 | 115 | 0.21 | Lung |
| Normal 3 | <27 | 6364 | 434 | 4047 | <27 | <206 | <137.2 | <82.3 | 0.09 | Lung |
| Tumor 4 | 1823 | 11745 | 11810 | 6386 | <27 | <206 | 37480 | 150 | 0.20 | Lung |
| Normal 4 | 54 | 7809 | 524 | >10000 | <27 | <206 | <137.2 | <82.3 | 0.08 | Lung |
| Tumor 5 | 15202 | 26265 | 51866 | 2630 | 176 | 1769 | 1822 | 35599 | 0.24 | Lung |
| Normal 5 | 188 | 12821 | 1252 | 6790 | <27 | <206 | <137.2 | <82.3 | 0.09 | Lung |
| Tumor 6 | 564 | 3562 | 78 | 2641 | 76 | 213 | 411 | <82.3 | 0.13 | Colon |
| Normal 6 | 232 | 3043 | <548 | 3350 | 33 | <206 | <137.2 | <82.3 | 0.09 | Colon |
| Tumor 7 | 5950 | 10372 | 11441 | 2054 | 43 | 913 | 1093 | 203 | 0.20 | Colon |
| Normal 7 | 167 | 3650 | <548 | 4348 | <27 | 235 | <137.2 | <82.3 | 0.04 | Colon |
| Tumor 8 | 1538 | 2327 | 402 | 7948 | 41 | 653 | 619 | 161 | 0.09 | Colon |
| Normal 8 | 446 | 3144 | <548 | 9037 | <27 | 448 | <137.2 | 150 | 0.10 | Colon |
| Tumor 9 | 9371 | 3536 | 2331 | >10000 | 393 | 4062 | 1752 | <82.3 | 0.31 | Colon |
| Normal 9 | 133 | 2110 | <548 | >10000 | <27 | <206 | <137.2 | <82.3 | 0.09 | Colon |
| Tumor 10 | 876 | 8449 | 9959 | 3408 | 35 | 784 | 553 | 381 | 0.07 | Colon |
| Normal 10 | 412 | 2785 | 1215 | 2148 | <27 | 293 | 294 | 141 | 0.10 | Colon |

As shown in Table 11, there was an intra-patient variability with regard to various MMP expression. Having a cleavable linker that is reactive against several MMPs, such as MMPs −2, −9, and −13, can help mitigate relative differences in upregulation on a patient-to-patient level, and therefore a broadly cleavable peptide was favored.

As presented in Table 12, linkers that can be included in the fusion proteins have broad specificity which shall mitigate patient-to-patient variability in MMP expression. To this end, several short peptides were tested for their MMP-mediated cleavage specificity. Due to its broad specificity peptide 5 was used in the Fc fusion construct(s).

TABLE 12

| | | Specific Activity (pmol/min/μg) | | | |
|---|---|---|---|---|---|
| | | Peptide 5 | | | |
| Species | MMP | (GPLGVRG) | Peptide 8 | Peptide 10 | Control |
| Human | rhMMP1 | 10 | 10.8 | 10 | 10 |
| | rhMMP2 | 260.5 | 259.7 | 162.7 | 10 |
| | rhMMP3 | 14.2 | 51.2 | 18.3 | 10 |
| | rhMMP7 | 10 | 70.9 | 98.4 | 10 |
| | rhMMP9 | 199.7 | 123.1 | 63.6 | 10 |
| | rhMMP13 | 361.6 | 344.3 | 156.1 | 10 |
| Mouse | rmMMP2 | 165.3 | 233 | 98.9 | 10 |
| | rmMMP9 | 292.1 | 157.9 | 111 | 10 |

### Example 4

### Generation of Masked IL-12 Fc Fusion Proteins

Based on the initial format scouting, the screening and optimization of a masking moiety as well as appropriate cleavable linker selection several optimized, masked IL-12 Fc fusion proteins were generated and further tested.

TABLE 13

| | |
|---|---|
| BI-050 Fc Knob | EPKSSDKTHTCPPCPAPEAAGGPSVFLFPPKPKDTLMISRTPEVTCVVVDVSHEDPEV |
| (human) | KFNWYVDGVEVHNAKTKPREEQYNSTYRVVSVLTVLHQDWLNGKEYKCKVSNKA |
| SEQ ID NO: 208 | LPAPIEKTISKAKGQPREPQVYTLPPSREEMTKNQVSLWCLVKGFYPSDIAVEWESN |
| | GQPENNYKTTPPVLDSDGSFFLYSKLTVDKSRWQQGNVFSCSVMHEALHNHYTQKS |
| | LSLSPGGGGGSGGGGGPLGVRGLRELHLDNNGGSIWELKKDVYVVELDWYPDAPG |
| | EMVVLTCDTPEEDGITWTLDQSSEVLGSGKTLTIQVKEFGDAGQYTCHKGGEVLSHS |
| | LLLLHKKEDGIWSTDILKDQKEPKNKTFLRCEAKNYSGRFTCWWLTTISTDLTFSVK |
| | SSRGSSDPQGVTCGAATLSAERVRGDNKEYEYSVECQEDSACPAAEESLPIEVMVDA |
| | VHKLKYENYTSSFFIRDIIKPDPPKNLQLKPLKNSRQVEVSWEYPDTWSTPHSYFSLT |
| | FCVQVQGKSKREKKDRVFTDKTSATVICRKNASISVRAQDRYYSSSWSEWASVPCS |
| | GGGGSGGGGSGGGGSRNLPVATPDPGMFPCLHHSQNLLRAVSNMLQKARQTLEFY |
| | PCTSEEIDHEDITKDKTSTVEACLPLELTKNESCLNSRETSFITNGSCLASRKTSFMMA |
| | LCLSSIYEDLKMYQVEFKTMNAKLLMDPKRQIFLDQNMLAVIDELMQALNFNSETV |
| | PQKSSLEEPDFYKTKIKLCILLHAFRIRAVTIDRVMSYLNAS |
| BI-050 Fc Hole | EPKSSDKTHTCPPCPAPEAAGGPSVFLFPPKPKDTLMISRTPEVTCVVVDVSHEDPEV |
| (human) | KFNWYVDGVEVHNAKTKPREEQYNSTYRVVSVLTVLHQDWLNGKEYKCKVSNKA |
| SEQ ID NO: 209 | LPAPIEKTISKAKGQPREPQVYTLPPSREEMTKNQVSLSCAVKGFYPSDIAVEWESNG |
| | QPENNYKTTPPVLDSDGSFFLVSKLTVDKSRWQQGNVFSCSVMHEALHNRFTQKSL |
| | SLSPGGGGGSGGGGSGGGGSGGGGSGGGGSEVQLVESGGGLVQPGGSLRLSCAASG |
| | RTFETIAMGWFRQAPGKGLEFVAVIRGSGVATYYAESVKGRFTISKDSSKNTVYLQ |
| | MNSLRAEDTAVYYCAATTNRFK |
| | NTDYTTYDYWGQGTQVTVSSA |
| BI-051 Fc Knob | EPKSSDKTHTCPPCPAPEAAGGPSVFLFPPKPKDTLMISRTPEVTCVVVDVSHEDPEV |
| (human) | KFNWYVDGVEVHNAKTKPREEQYNSTYRVVSVLTVLHQDWLNGKEYKCKVSNKA |
| SEQ ID NO: 210 | LPAPIEKTISKAKGQPREPQVYTLPPSREEMTKNQVSLWCLVKGFYPSDIAVEWESN |
| | GQPENNYKTTPPVLDSDGSFFLYSKLTVDKSRWQQGNVFSCSVMHEALHNHYTQKS |
| | LSLSPGGGGGSGGGGGPLGVRGGGGGSIWELKKDVYVVELDWYPDAPGEMVVLTC |
| | DTPEEDGITWTLDQSSEVLGSGKTLTIQVKEFGDAGQYTCHKGGEVLSHSLLLLHKK |
| | EDGIWSTDILKDQKEPKNKTFLRCEAKNYSGRFTCWWLTTISTDLTFSVKSSRGSSDP |
| | QGVTCGAATLSAERVRGDNKEYEYSVECQEDSACPAAEESLPIEVMVDAVHKLKYE |
| | NYTSSFFIRDIIKPDPPKNLQLKPLKNSRQVEVSWEYPDTWSTPHSYFSLTFCVQVQG |
| | KSKREKKDRVFTDKTSATVICRKNASISVRAQDRYYSSSWSEWASVPCSGGGGSGG |
| | GGSGGGGSRNLPVATPDPGMFPCLHHSQNLLRAVSNMLQKARQTLEFYPCTSEEIDH |
| | EDITKDKTSTVEACLPLELTKNESCLNSRETSFITNGSCLASRKTSFMMALCLSSIYED |
| | LKMYQVEFKTMNAKLLMDPKRQIFLDQNMLAVIDELMQALNFNSETVPQKSSLEEP |
| | DFYKTKIKLCILLHAFRIRAVTIDRVMSYLNASGSGGLRELHLDNN |
| BI-051 Fc Hole | EPKSSDKTHTCPPCPAPEAAGGPSVFLFPPKPKDTLMISRTPEVTCVVVDVSHEDPEV |
| (human) | KFNWYVDGVEVHNAKTKPREEQYNSTYRVVSVLTVLHQDWLNGKEYKCKVSNKA |
| SEQ ID NO: 211 | LPAPIEKTISKAKGQPREPQVYTLPPSREEMTKNQVSLSCAVKGFYPSDIAVEWESNG |
| | QPENNYKTTPPVLDSDGSFFLVSKLTVDKSRWQQGNVFSCSVMHEALHNRFTQKSL |
| | SLSPGGGGGSGGGGSGGGGSGGGGSGGGGSEVQLVESGGGLVQPGGSLRLSCAASG |
| | RTFETIAMGWFRQAPGKGLEFVAVIRGSGVATYYAESVKGRFTISKDSSKNTVYLQ |
| | MNSLRAEDTAVYYCAATTNRFKNTDYTTYDYWGQGTQVTVSSA |
| BI-052 Fc Knob | EPKSSDKTHTCPPCPAPEAAGGPSVFLFPPKPKDTLMISRTPEVTCVVVDVSHEDPEV |
| (human) | KFNWYVDGVEVHNAKTKPREEQYNSTYRVVSVLTVLHQDWLNGKEYKCKVSNKA |
| SEQ ID NO: 212 | LPAPIEKTISKAKGQPREPQVYTLPPSREEMTKNQVSLWCLVKGFYPSDIAVEWESN |
| | GQPENNYKTTPPVLDSDGSFFLYSKLTVDKSRWQQGNVFSCSVMHEALHNHYTQKS |
| | LSLSPGGGGGSGGGGGPLGVRGGGGGSIWELKKDVYVVELDWYPDAPGEMVVLTC |
| | DTPEEDGITWTLDQSSEVLGSGKTLTIQVKEFGDAGQYTCHKGGEVLSHSLLLLHKK |
| | EDGIWSTDILKDQKEPKNKTFLRCEAKNYSGRFTCWWLTTISTDLTFSVKSSRGSSDP |
| | QGVTCGAATLSAERVRGDNKEYEYSVECQEDSACPAAEESLPIEVMVDAVHKLKYE |
| | NYTSSFFIRDIIKPDPPKNLQLKPLKNSRQVEVSWEYPDTWSTPHSYFSLTFCVQVQG |
| | KSKREKKDRVFTDKTSATVICRKNASISVRAQDRYYSSSWSEWASVPCSGGGGSGG |
| | GGSGGGGSLRELHLDNNGGSRNLPVATPDPGMFPCLHHSQNLLRAVSNMLQKARQ |
| | TLEFYPCTSEEIDHEDITKDKTSTVEACLPLELTKNESCLNSRETSFITNGSCLASRKTS |
| | FMMALCLSSIYEDLKMYQVEFKTMNAKLLMDPKRQIFLDQNMLAVIDELMQALNF |
| | NSETVPQKSSLEEPDFYKTKIKLCILLHAFRIRAVTIDRVMSYLNAS |
| BI-052 Fc Hole | EPKSSDKTHTCPPCPAPEAAGGPSVFLFPPKPKDTLMISRTPEVTCVVVDVSHEDPEV |
| (human) | KFNWYVDGVEVHNAKTKPREEQYNSTYRVVSVLTVLHQDWLNGKEYKCKVSNKA |
| SEQ ID NO: 213 | LPAPIEKTISKAKGQPREPQVYTLPPSREEMTKNQVSLSCAVKGFYPSDIAVEWESNG |
| | QPENNYKTTPPVLDSDGSFFLVSKLTVDKSRWQQGNVFSCSVMHEALHNRFTQKSL |
| | SLSPGGGGGSGGGGSGGGGSGGGGSGGGGSEVQLVESGGGLVQPGGSLRLSCAASG |
| | RTFETIAMGWFRQAPGKGLEFVAVIRGSGVATYYAESVKGRFTISKDSSKNTVYLQ |
| | MNSLRAEDTAVYYCAATTNRFKNTDYTTYDYWGQGTQVTVSSA |
| BI-053 Fc Knob | EPKSSDKTHTCPPCPAPEAAGGPSVFLFPPKPKDTLMISRTPEVTCVVVDVSHEDPEV |
| (human) | KFNWYVDGVEVHNAKTKPREEQYNSTYRVVSVLTVLHQDWLNGKEYKCKVSNKA |
| SEQ ID NO: 214 | LPAPIEKTISKAKGQPREPQVYTLPPSREEMTKNQVSLWCLVKGFYPSDIAVEWESN |
| | GQPENNYKTTPPVLDSDGSFFLYSKLTVDKSRWQQGNVFSCSVMHEALHNHYTQKS |
| | LSLSPGGGGGSGGGGGPLGVRGGGGGSIWELKKDVYVVELDWYPDAPGEMVVLTC |
| | DTPEEDGITWTLDQSSEVLGSGKTLTIQVKEFGDAGQYTCHKGGEVLSHSLLLLHKK |
| | EDGIWSTDILKDQKEPKNKTFLRCEAKNYSGRFTCWWLTTISTDLTFSVKSSRGSSDP |
| | QGVTCGAATLSAERVRGDNKEYEYSVECQEDSACPAAEESLPIEVMVDAVHKLKYE |
| | NYTSSFFIRDIIKPDPPKNLQLKPLKNSRQVEVSWEYPDTWSTPHSYFSLTFCVQVQG |
| | KSKREKKDRVFTDKTSATVICRKNASISVRAQDRYYSSSWSEWASVPCSGGGGSGG |
| | GGSGGGGSRNLPVATPDPGMFPCLHHSQNLLRAVSNMLQKARQTLEFYPCTSEEIDH |
| | EDITKDKTSTVEACLPLELTKNESCLNSRETSFITNGSCLASRKTSFMMALCLSSIYED |
| | LKMYQVEFKTMNAKLLMDPKRQIFLDQNMLAVIDELMQALNFNSETVPQKSSLEEP |
| | DFYKTKIKLCILLHAFRIRAVTIDRVMSYLNASGSGGGGWSHW |
| BI-053 Fc Hole | EPKSSDKTHTCPPCPAPEAAGGPSVFLFPPKPKDTLMISRTPEVTCVVVDVSHEDPEV |
| (human) | KFNWYVDGVEVHNAKTKPREEQYNSTYRVVSVLTVLHQDWLNGKEYKCKVSNKA |
| SEQ ID NO: 215 | LPAPIEKTISKAKGQPREPQVYTLPPSREEMTKNQVSLSCAVKGFYPSDIAVEWESNG |
| | QPENNYKTTPPVLDSDGSFFLVSKLTVDKSRWQQGNVFSCSVMHEALHNRFTQKSL |
| | SLSPGGGGGSGGGGSGGGGSGGGGSGGGGSEVQLVESGGGLVQPGGSLRLSCAASG |
| | RTFETIAMGWFRQAPGKGLEFVAVIRGSGVATYYAESVKGRFTISKDSSKNTVYLQ |
| | MNSLRAEDTAVYYCAATTNRFKNTDYTTYDYWGQGTQVTVSSA |
| BI-054 Fc Knob | EPKSSDKTHTCPPCPAPEAAGGPSVFLFPPKPKDTLMISRTPEVTCVVVDVSHEDPEV |
| (human) | KFNWYVDGVEVHNAKTKPREEQYNSTYRVVSVLTVLHQDWLNGKEYKCKVSNKA |
| SEQ ID NO: 216 | LPAPIEKTISKAKGQPREPQVYTLPPSREEMTKNQVSLWCLVKGFYPSDIAVEWESN |
| | GQPENNYKTTPPVLDSDGSFFLYSKLTVDKSRWQQGNVFSCSVMHEALHNHYTQKS |
| | LSLSPGGGGGSGGGGGPLGVRGGGGGSIWELKKDVYVVELDWYPDAPGEMVVLTC |
| | DTPEEDGITWTLDQSSEVLGSGKTLTIQVKEFGDAGQYTCHKGGEVLSHSLLLLHKK |
| | EDGIWSTDILKDQKEPKNKTFLRCEAKNYSGRFTCWWLTTISTDLTFSVKSSRGSSDP |
| | QGVTCGAATLSAERVRGDNKEYEYSVECQEDSACPAAEESLPIEVMVDAVHKLKYE |
| | NYTSSFFIRDIIKPDPPKNLQLKPLKNSRQVEVSWEYPDTWSTPHSYFSLTFCVQVQG |
| | KSKREKKDRVFTDKTSATVICRKNASISVRAQDRYYSSSWSEWASVPCSGGGGSGG |
| | GGSGGGGSRNLPVATPDPGMFPCLHHSQNLLRAVSNMLQKARQTLEFYPCTSEEIDH |
| | EDITKDKTSTVEACLPLELTKNESCLNSRETSFITNGSCLASRKTSFMMALCLSSIYED |
| | LKMYQVEFKTMNAKLLMDPKRQIFLDQNMLAVIDELMQALNFNSETVPQKSSLEEP |
| | DFYKTKIKLCILLHAFRIRAVTIDRVMSYLNASGSGGLRELHLDNN |
| BI-054 Fc Hole | EPKSSDKTHTCPPCPAPEAAGGPSVFLFPPKPKDTLMISRTPEVTCVVVDVSHEDPEV |
| (human) | KFNWYVDGVEVHNAKTKPREEQYNSTYRVVSVLTVLHQDWLNGKEYKCKVSNKA |
| SEQ ID NO: 217 | LPAPIEKTISKAKGQPREPQVYTLPPSREEMTKNQVSLSCAVKGFYPSDIAVEWESNG |
| | QPENNYKTTPPVLDSDGSFFLVSKLTVDKSRWQQGNVFSCSVMHEALHNRFTQKSL |
| | SLSPGGGGGSGGGGSGGGGSGGGGSGGGGSEVQLVESGGGLVQPGGSLRLSCAASG |
| | RTFETIAMGWFRQAPGKGLEFVAVIRGSGVATYYAESVKGRFTISKDSSKNTVYLQ |
| | MNSLRAEDTAVYYCAATTNRFKNREYTTYDYWGQGTQVTVSSA |
| BI-055 Fc Knob | EPKSSDKTHTCPPCPAPEAAGGPSVFLFPPKPKDTLMISRTPEVTCVVVDVSHEDPEV |
| (human) | KFNWYVDGVEVHNAKTKPREEQYNSTYRVVSVLTVLHQDWLNGKEYKCKVSNKA |
| SEQ ID NO: 218 | LPAPIEKTISKAKGQPREPQVYTLPPCREEMTKNQVSLWCLVKGFYPSDIAVEWESN |
| | GQPENNYKTTPPVLDSDGSFFLYSKLTVDKSRWQQGNVFSCSVMHEALHNHYTQKS |
| | LSLSPGGGGGSGGGGGPLGVRGGGGGSIWELKKDVYVVELDWYPDAPGEMVVLTC |
| | DTPEEDGITWTLDQSSEVLGSGKTLTIQVKEFGDAGQYTCHKGGEVLSHSLLLLHKK |
| | EDGIWSTDILKDQKEPKNKTFLRCEAKNYSGRFTCWWLTTISTDLTFSVKSSRGSSDP |
| | QGVTCGAATLSAERVRGDNKEYEYSVECQEDSACPAAEESLPIEVMVDAVHKLKYE |
| | NYTSSFFIRDIIKPDPPKNLQLKPLKNSRQVEVSWEYPDTWSTPHSYFSLTFCVQVQG |
| | KSKREKKDRVFTDKTSATVICRKNASISVRAQDRYYSSSWSEWASVPCSGGGGSGG |
| | GGSGGGGSRNLPVATPDPGMFPCLHHSQNLLRAVSNMLQKARQTLEFYPCTSEEIDH |
| | EDITKDKTSTVEACLPLELTKNESCLNSRETSFITNGSCLASRKTSFMMALCLSSIYED |
| | LKMYQVEFKTMNAKLLMDPKRQIFLDQNMLAVIDELMQALNFNSETVPQKSSLEEP |
| | DFYKTKIKLCILLHAFRIRAVTIDRVMSYLNASGSGGLRELHLDN |
| BI-055 Fc Hole | EPKSSDKTHTCPPCPAPEAAGGPSVFLFPPKPKDTLMISRTPEVTCVVVDVSHEDPEV |
| (human) | KFNWYVDGVEVHNAKTKPREEQYNSTYRVVSVLTVLHQDWLNGKEYKCKVSNKA |
| SEQ ID NO: 219 | LPAPIEKTISKAKGQPREPQVCTLPPSREEMTKNQVSLSCAVKGFYPSDIAVEWESNG |
| | QPENNYKTTPPVLDSDGSFFLVSKLTVDKSRWQQGNVFSCSVMHEALHNRFTQKSL |
| | SLSPGGGGGSGGGGSGGGGSGGGGSGGGGSEVQLVESGGGLVQPGGSLRLSCAASG |
| | RTFETIAMGWFRQAPGKGLEFVAVIRGSGVATYYAESVKGRFTISKDSSKNTVYLQ |
| | MNSLRAEDTAVYYCAATTNRFKNTDYTTYDYWGQGTQVTVSSA |
| BI-056 Fc Knob | EPKSSDKTHTCPPCPAPEAAGGPSVFLFPPKPKDTLMISRTPEVTCVVVDVSHEDPEV |
| (chimeric) | KFNWYVDGVEVHNAKTKPREEQYNSTYRVVSVLTVLHQDWLNGKEYKCKVSNKA |
| SEQ ID NO: 220 | LPAPIEKTISKAKGQPREPQVYTLPPSREEMTKNQVSLWCLVKGFYPSDIAVEWESN |
| | GQPENNYKTTPPVLDSDGSFFLYSKLTVDKSRWQQGNVFSCSVMHEALHNHYTQKS |
| | LSLSPGGGGGSGGGGGPLGVRGLRELHLDNNGGSIWELKKDVYVVELDWYPDAPG |
| | EMVVLTCDTPEEDGITWTLDQSSEVLGSGKTLTIQVKEFGDAGQYTCHKGGEVLSHS |
| | LLLLHKKEDGIWSTDILKDQKEPKNKTFLRCEAKNYSGRFTCWWLTTISTDLTFSVK |
| | SSRGSSDPQGVTCGAATLSAERVRGDNKEYEYSVECQEDSACPAAEESLPIEVMVDA |
| | VHKLKYENYTSSFFIRDIIKPDPPKNLQLKPLKNSRQVEVSWEYPDTWSTPHSYFSLT |
| | FCVQVQGKSKREKKDRVFTDKTSATVICRKNASISVRAQDRYYSSSWSEWASVPCS |
| | GGGGSGGGGSGGGGSRVIPVSGPARCLSQSRNLLKTTDDMVKTAREKLKHYSCTAE |
| | DIDHEDITRDQTSTLKTCLPLELHKNESCLATRETSSTTRGSCLPPQKTSLMMTLCLG |
| | SIYEDLKMYQTEFQAINAALQNHNHQQIILDKGMLVAIDELMQSLNHNGETLRQKPP |
| | VGEADPYRVKMKLCILLHAFSTRVVTINRVMGYLSSA |
| BI-056 Fc Hole | EPKSSDKTHTCPPCPAPEAAGGPSVFLFPPKPKDTLMISRTPEVTCVVVDVSHEDPEV |
| (chimeric) | KFNWYVDGVEVHNAKTKPREEQYNSTYRVVSVLTVLHQDWLNGKEYKCKVSNKA |
| SEQ ID NO: 221 | LPAPIEKTISKAKGQPREPQVYTLPPSREEMTKNQVSLSCAVKGFYPSDIAVEWESNG |
| | QPENNYKTTPPVLDSDGSFFLVSKLTVDKSRWQQGNVFSCSVMHEALHNRFTQKSL |
| | SLSPGGGGGSGGGGSGGGGSGGGGSGGGGSEVQLVESGGGLVQPGGSLRLSCAASG |
| | RTFETIAMGWFRQAPGKGLEFVAVIRGSGVATYYAESVKGRFTISKDSSKNTVYLQ |
| | MNSLRAEDTAVYYCAATTNRFKNTDYTTYDYWGQGTQVTVSSA |
| BI-057 Fc Knob | EPKSSDKTHTCPPCPAPEAAGGPSVFLFPPKPKDTLMISRTPEVTCVVVDVSHEDPEV |
| (chimeric) | KFNWYVDGVEVHNAKTKPREEQYNSTYRVVSVLTVLHQDWLNGKEYKCKVSNKA |
| SEQ ID NO: 222 | LPAPIEKTISKAKGQPREPQVYTLPPSREEMTKNQVSLWCLVKGFYPSDIAVEWESN |
| | GQPENNYKTTPPVLDSDGSFFLYSKLTVDKSRWQQGNVFSCSVMHEALHNHYTQKS |
| | LSLSPGGGGGSGGGGGPLGVRGGGGGSIWELKKDVYVVELDWYPDAPGEMVVLTC |
| | DTPEEDGITWTLDQSSEVLGSGKTLTIQVKEFGDAGQYTCHKGGEVLSHSLLLLHKK |
| | EDGIWSTDILKDQKEPKNKTFLRCEAKNYSGRFTCWWLTTISTDLTFSVKSSRGSSDP |
| | QGVTCGAATLSAERVRGDNKEYEYSVECQEDSACPAAEESLPIEVMVDAVHKLKYE |
| | NYTSSFFIRDIIKPDPPKNLQLKPLKNSRQVEVSWEYPDTWSTPHSYFSLTFCVQVQG |
| | KSKREKKDRVFTDKTSATVICRKNASISVRAQDRYYSSSWSEWASVPCSGGGGSGG |
| | GGSGGGGSRVIPVSGPARCLSQSRNLLKTTDDMVKTAREKLKHYSCTAEDIDHEDIT |
| | RDQTSTLKTCLPLELHKNESCLATRETSSTTRGSCLPPQKTSLMMTLCLGSIYEDLKM |
| | YQTEFQAINAALQNHNHQQIILDKGMLVAIDELMQSLNHNGETLRQKPPVGEADPY |
| | RVKMKLCILLHAFSTRVVTINRVMGYLSSAGSGGLRELHLDNN |
| BI-057 Fc Hole | EPKSSDKTHTCPPCPAPEAAGGPSVFLFPPKPKDTLMISRTPEVTCVVVDVSHEDPEV |
| (chimeric) | KFNWYVDGVEVHNAKTKPREEQYNSTYRVVSVLTVLHQDWLNGKEYKCKVSNKA |
| SEQ ID NO: 223 | LPAPIEKTISKAKGQPREPQVYTLPPSREEMTKNQVSLSCAVKGFYPSDIAVEWESNG |
| | QPENNYKTTPPVLDSDGSFFLVSKLTVDKSRWQQGNVFSCSVMHEALHNRFTQKSL |
| | SLSPGGGGGSGGGGSGGGGSGGGGSGGGGSEVQLVESGGGLVQPGGSLRLSCAASG |
| | RTFETIAMGWFRQAPGKGLEFVAVIRGSGVATYYAESVKGRFTISKDSSKNTVYLQ |
| | MNSLRAEDTAVYYCAATTNRFKNTDYTTYDYWGQGTQVTVSSA |
| BI-058 Fc Knob | EPKSSDKTHTCPPCPAPEAAGGPSVFLFPPKPKDTLMISRTPEVTCVVVDVSHEDPEV |
| (chimeric) | KFNWYVDGVEVHNAKTKPREEQYNSTYRVVSVLTVLHQDWLNGKEYKCKVSNKA |
| SEQ ID NO: 224 | LPAPIEKTISKAKGQPREPQVYTLPPSREEMTKNQVSLWCLVKGFYPSDIAVEWESN |
| | GQPENNYKTTPPVLDSDGSFFLYSKLTVDKSRWQQGNVFSCSVMHEALHNHYTQKS |
| | LSLSPGGGGGSGGGGGPLGVRGGGGGSIWELKKDVYVVELDWYPDAPGEMVVLTC |
| | DTPEEDGITWTLDQSSEVLGSGKTLTIQVKEFGDAGQYTCHKGGEVLSHSLLLLHKK |
| | EDGIWSTDILKDQKEPKNKTFLRCEAKNYSGRFTCWWLTTISTDLTFSVKSSRGSSDP |
| | QGVTCGAATLSAERVRGDNKEYEYSVECQEDSACPAAEESLPIEVMVDAVHKLKYE |
| | NYTSSFFIRDIIKPDPPKNLQLKPLKNSRQVEVSWEYPDTWSTPHSYFSLTFCVQVQG |
| | KSKREKKDRVFTDKTSATVICRKNASISVRAQDRYYSSSWSEWASVPCSGGGGSGG |
| | GGSGGGGSLRELHLDNNGGSRVIPVSGPARCLSQSRNLLKTTDDMVKTAREKLKHY |
| | SCTAEDIDHEDITRDQTSTLKTCLPLELHKNESCLATRETSSTTRGSCLPPQKTSLMM |
| | TLCLGSIYEDLKMYQTEFQAINAALQNHNHQQIILDKGMLVAIDELMQSLNHNGETL |
| | RQKPPVGEADPYRVKMKLCILLHAFSTRVVTINRVMGYLSSA |
| BI-058 Fc Hole | EPKSSDKTHTCPPCPAPEAAGGPSVFLFPPKPKDTLMISRTPEVTCVVVDVSHEDPEV |
| (chimeric) | KFNWYVDGVEVHNAKTKPREEQYNSTYRVVSVLTVLHQDWLNGKEYKCKVSNKA |
| SEQ ID NO: 225 | LPAPIEKTISKAKGQPREPQVYTLPPSREEMTKNQVSLSCAVKGFYPSDIAVEWESNG |
| | QPENNYKTTPPVLDSDGSFFLVSKLTVDKSRWQQGNVFSCSVMHEALHNRFTQKSL |
| | SLSPGGGGGSGGGGSGGGGSGGGGSGGGGSEVQLVESGGGLVQPGGSLRLSCAASG |
| | RTFETIAMGWFRQAPGKGLEFVAVIRGSGVATYYAESVKGRFTISKDSSKNTVYLQ |
| | MNSLRAEDTAVYYCAATTNRFKNTDYTTYDYWGQGTQVTVSSA |
| BI-059 Fc Knob | EPKSSDKTHTCPPCPAPEAAGGPSVFLFPPKPKDTLMISRTPEVTCVVVDVSHEDPEV |
| (chimeric) | KFNWYVDGVEVHNAKTKPREEQYNSTYRVVSVLTVLHQDWLNGKEYKCKVSNKA |
| SEQ ID NO: 226 | LPAPIEKTISKAKGQPREPQVYTLPPSREEMTKNQVSLWCLVKGFYPSDIAVEWESN |
| | GQPENNYKTTPPVLDSDGSFFLYSKLTVDKSRWQQGNVFSCSVMHEALHNHYTQKS |
| | LSLSPGGGGGSGGGGGPLGVRGGGGGSIWELKKDVYVVELDWYPDAPGEMVVLTC |
| | DTPEEDGITWTLDQSSEVLGSGKTLTIQVKEFGDAGQYTCHKGGEVLSHSLLLLHKK |
| | EDGIWSTDILKDQKEPKNKTFLRCEAKNYSGRFTCWWLTTISTDLTFSVKSSRGSSDP |
| | QGVTCGAATLSAERVRGDNKEYEYSVECQEDSACPAAEESLPIEVMVDAVHKLKYE |
| | NYTSSFFIRDIIKPDPPKNLQLKPLKNSRQVEVSWEYPDTWSTPHSYFSLTFCVQVQG |
| | KSKREKKDRVFTDKTSATVICRKNASISVRAQDRYYSSSWSEWASVPCSGGGGSGG |
| | GGSGGGGSRVIPVSGPARCLSQSRNLLKTTDDMVKTAREKLKHYSCTAEDIDHEDIT |
| | RDQTSTLKTCLPLELHKNESCLATRETSSTTRGSCLPPQKTSLMMTLCLGSIYEDLKM |
| | YQTEFQAINAALQNHNHQQIILDKGMLVAIDELMQSLNHNGETLRQKPPVGEADPY |
| | RVKMKLCILLHAFSTRVVTINRVMGYLSSAGSGGGGWSHW |
| BI-059 Fc Hole | EPKSSDKTHTCPPCPAPEAAGGPSVFLFPPKPKDTLMISRTPEVTCVVVDVSHEDPEV |
| (chimeric) | KFNWYVDGVEVHNAKTKPREEQYNSTYRVVSVLTVLHQDWLNGKEYKCKVSNKA |
| SEQ ID NO: 227 | LPAPIEKTISKAKGQPREPQVYTLPPSREEMTKNQVSLSCAVKGFYPSDIAVEWESNG |
| | QPENNYKTTPPVLDSDGSFFLVSKLTVDKSRWQQGNVFSCSVMHEALHNRFTQKSL |
| | SLSPGGGGGSGGGGSGGGGSGGGGSGGGGSEVQLVESGGGLVQPGGSLRLSCAASG |
| | RTFETIAMGWFRQAPGKGLEFVAVIRGSGVATYYAESVKGRFTISKDSSKNTVYLQ |
| | MNSLRAEDTAVYYCAATTNRFKNTDYTTYDYWGQGTQVTVSSA |
| BI-060 Fc Knob | EPKSSDKTHTCPPCPAPEAAGGPSVFLFPPKPKDTLMISRTPEVTCVVVDVSHEDPEV |
| (chimeric) | KFNWYVDGVEVHNAKTKPREEQYNSTYRVVSVLTVLHQDWLNGKEYKCKVSNKA |
| SEQ ID NO: 228 | LPAPIEKTISKAKGQPREPQVYTLPPSREEMTKNQVSLWCLVKGFYPSDIAVEWESN |
| | GQPENNYKTTPPVLDSDGSFFLYSKLTVDKSRWQQGNVFSCSVMHEALHNHYTQKS |
| | LSLSPGGGGGSGGGGGPLGVRGGGGGSIWELKKDVYVVELDWYPDAPGEMVVLTC |
| | DTPEEDGITWTLDQSSEVLGSGKTLTIQVKEFGDAGQYTCHKGGEVLSHSLLLLHKK |
| | EDGIWSTDILKDQKEPKNKTFLRCEAKNYSGRFTCWWLTTISTDLTFSVKSSRGSSDP |
| | QGVTCGAATLSAERVRGDNKEYEYSVECQEDSACPAAEESLPIEVMVDAVHKLKYE |
| | NYTSSFFIRDIIKPDPPKNLQLKPLKNSRQVEVSWEYPDTWSTPHSYFSLTFCVQVQG |
| | KSKREKKDRVFTDKTSATVICRKNASISVRAQDRYYSSSWSEWASVPCSGGGGSGG |
| | GGSGGGGSRVIPVSGPARCLSQSRNLLKTTDDMVKTAREKLKHYSCTAEDIDHEDIT |
| | RDQTSTLKTCLPLELHKNESCLATRETSSTTRGSCLPPQKTSLMMTLCLGSIYEDLKM |
| | YQTEFQAINAALQNHNHQQIILDKGMLVAIDELMQSLNHNGETLRQKPPVGEADPY |
| | RVKMKLCILLHAFSTRVVTINRVMGYLSSAGSGGLRELHLDNN |
| BI-060 Fc Hole | EPKSSDKTHTCPPCPAPEAAGGPSVFLFPPKPKDTLMISRTPEVTCVVVDVSHEDPEV |
| (chimeric) | KFNWYVDGVEVHNAKTKPREEQYNSTYRVVSVLTVLHQDWLNGKEYKCKVSNKA |
| SEQ ID NO: 229 | LPAPIEKTISKAKGQPREPQVYTLPPSREEMTKNQVSLSCAVKGFYPSDIAVEWESNG |
| | QPENNYKTTPPVLDSDGSFFLVSKLTVDKSRWQQGNVFSCSVMHEALHNRFTQKSL |
| | SLSPGGGGGSGGGGSGGGGSGGGGSGGGGSEVQLVESGGGLVQPGGSLRLSCAASG |
| | RTFETIAMGWFRQAPGKGLEFVAVIRGSGVATYYAESVKGRFTISKDSSKNTVYLQ |
| | MNSLRAEDTAVYYCAATTNRFKNREYTTYDYWGQGTQVTVSSA |
| BI-061 Fc Knob | EPKSSDKTHTCPPCPAPEAAGGPSVFLFPPKPKDTLMISRTPEVTCVVVDVSHEDPEV |
| (chimeric) | KFNWYVDGVEVHNAKTKPREEQYNSTYRVVSVLTVLHQDWLNGKEYKCKVSNKA |
| SEQ ID NO: 230 | LPAPIEKTISKAKGQPREPQVYTLPPCREEMTKNQVSLWCLVKGFYPSDIAVEWESN |
| | GQPENNYKTTPPVLDSDGSFFLYSKLTVDKSRWQQGNVFSCSVMHEALHNHYTQKS |
| | LSLSPGGGGGSGGGGGPLGVRGGGGGSIWELKKDVYVVELDWYPDAPGEMVVLTC |
| | DTPEEDGITWTLDQSSEVLGSGKTLTIQVKEFGDAGQYTCHKGGEVLSHSLLLLHKK |
| | EDGIWSTDILKDQKEPKNKTFLRCEAKNYSGRFTCWWLTTISTDLTFSVKSSRGSSDP |
| | QGVTCGAATLSAERVRGDNKEYEYSVECQEDSACPAAEESLPIEVMVDAVHKLKYE |
| | NYTSSFFIRDIIKPDPPKNLQLKPLKNSRQVEVSWEYPDTWSTPHSYFSLTFCVQVQG |
| | KSKREKKDRVFTDKTSATVICRKNASISVRAQDRYYSSSWSEWASVPCSGGGGSGG |
| | GGSGGGGSRVIPVSGPARCLSQSRNLLKTTDDMVKTAREKLKHYSCTAEDIDHEDIT |
| | RDQTSTLKTCLPLELHKNESCLATRETSSTTRGSCLPPQKTSLMMTLCLGSIYEDLKM |
| | YQTEFQAINAALQNHNHQQIILDKGMLVAIDELMQSLNHNGETLRQKPPVGEADPY |
| | RVKMKLCILLHAFSTRVVTINRVMGYLSSAGSGGLRELHLDNN |
| BI-061 Fc Hole | EPKSSDKTHTCPPCPAPEAAGGPSVFLFPPKPKDTLMISRTPEVTCVVVDVSHEDPEV |
| (chimeric) | KFNWYVDGVEVHNAKTKPREEQYNSTYRVVSVLTVLHQDWLNGKEYKCKVSNKA |
| SEQ ID NO: 231 | LPAPIEKTISKAKGQPREPQVCTLPPSREEMTKNQVSLSCAVKGFYPSDIA VEWESNG |
| | QPENNYKTTPPVLDSDGSFFLVSKLTVDKSRWQQGNVFSCSVMHEALHNRFTQKSL |
| | SLSPGGGGGSGGGGSGGGGSGGGGSGGGGSEVQLVESGGGLVQPGGSLRLSCAASG |
| | RTFETIAMGWFRQAPGKGLEFVAVIRGSGVATYYAESVKGRFTISKDSSKNTVYLQ |
| | MNSLRAEDTAVYYCAATTNRFKNTDYTTYDYWGQGTQVTVSSA |
| BI-062 Fc Knob | EPKSSDKTHTCPPCPAPEAAGGPSVFLFPPKPKDTLMISRTPEVTCVVVDVSHEDPEV |
| SEQ ID NO: 242 | KFNWYVDGVEVHNAKTKPREEQYNSTYRVVSVLTVLHQDWLNGKEYKCKVSNKA |
| | LPAPIEKTISKAKGQPREPQVYTLPPSREEMTKNQVSLWCLVKGFYPSDIAVEWESN |
| | GQPENNYKTTPPVLDSDGSFFLYSKLTVDKSRWQQGNVFSCSVMHEALHNHYTQKS |
| | LSLSPGGGGGSGGGGGPLGVRGGGGGSIWELKKDVYVVELDWYPDAPGEMVVLTC |
| | DTPEEDGITWTLDQSSEVLGSGKTLTIQVKEFGDAGQYTCHKGGEVLSHSLLLLHKK |
| | EDGIWSTDILKDQKEPKNKTFLRCEAKNYSGRFTCWWLTTISTDLTFSVKSSRGSSDP |
| | QGVTCGAATLSAERVRGDNKEYEYSVECQEDSACPAAEESLPIEVMVDAVHKLKYE |
| | NYTSSFFIRDIIKPDPPKNLQLKPLKNSRQVEVSWEYPDTWSTPHSYFSLTFCVQVQG |
| | KSKREKKDRVFTDKTSATVICRKNASISVRAQDRYYSSSWSEWASVPCSGGGGSGG |
| | GGSGGGGSRNLPVATPDPGMFPCLHHSQNLLRAVSNMLQKARQTLEFYPCTSEEIDH |
| | EDITKDKTSTVEACLPLELTKNESCLNSRETSFITNGSCLASRKTSFMMALCLSSIYED |
| | LKMYQVEFKTMNAKLLMDPKRQIFLDQNMLAVIDELMQALNFNSETVPQKSSLEEP |
| | DFYKTKIKLCILLHAFRIRAVTIDRVMSYLNAS |
| BI-062 Fc Hole | EPKSSDKTHTCPPCPAPEAAGGPSVFLFPPKPKDTLMISRTPEVTCVVVDVSHEDPEV |
| SEQ ID NO: 243 | KFNWYVDGVEVHNAKTKPREEQYNSTYRVVSVLTVLHQDWLNGKEYKCKVSNKA |
| | LPAPIEKTISKAKGQPREPQVYTLPPSREEMTKNQVSLSCAVKGFYPSDIAVEWESNG |
| | QPENNYKTTPPVLDSDGSFFLVSKLTVDKSRWQQGNVFSCSVMHEALHNRFTQKSL |
| | SLSPGGGGGSGGGGSGGGGSGGGGSGGGGSQVQLVESGGGLVQAGGSLRLSCAAS |
| | GRTFETIAMGWFRQVPGKEREFVAVIRGSGVATYYPDSVKGRFTISKDSAKNTVYLQ |
| | MNNLKPEDTAVYYCAATTNRFKNTDYTTYDYWGQGTQVTVSSSG |
| BI-064 Knob | EPKSSDKTHTCPPCPAPEAAGGPSVFLFPPKPKDTLMISRTPEVTCVVVDVSHEDPEVK |
| SEQ ID NO:329 | FNWYVDGVEVHNAKTKPREEQYNSTYRVVSVLTVLHQDWLNGKEYKCKVSNKALP |
| | APIEKTISKAKGQPREPQVYTLPPSREEMTKNQVSLWCLVKGFYPSDIAVEWESNGQP |
| | ENNYKTTPPVLDSDGSFFLYSKLTVDKSRWQQGNVFSCSVMHEALHNHYTQKSLSLS |
| | PGGGGGSGGGGGPLGVRGGGGGSIWELKKDVYVVELDWYPDAPGEMVVLTCDTPE |
| | EDGITWTLDQSSEVLGSGKTLTIQVKEFGDAGQYTCHKGGEVLSHSLLLLHKKEDGI |
| | WSTDILKDQKEPKNKTFLRCEAKNYSGRFTCWWLTTISTDLTFSVKSSRGSSDPQGVT |
| | CGAATLSAERVRGDNKEYEYSVECQEDSACPAAEESLPIEVMVDAVHKLKYENYTSS |
| | FFIRDIIKPDPPKNLQLKPLKNSRQVEVSWEYPDTWSTPHSYFSLTFCVQVQGKSKREK |
| | KDRVFTDKTSATVICRKNASISVRAQDRYYSSSWSEWASVPCSGGGGSGGGGSGGGG |
| | SRNLPVATPDPGMFPCLHHSQNLLRAVSNMLQKARQTLEFYPCTSEEIDHEDITKDKT |
| | STVEACLPLELTKNESCLNSRETSFITNGSCLASRKTSFMMALCLSSIYEDLKMYQVEF |
| | KTMNAKLLMDPKRQIFLDQNMLAVIDELMQALNFNSETVPQKSSLEEPDFYKTKIKL |
| | CILLHAFRIRAVTIDRVMSYLNAS |
| BI-064 Hole | EPKSSDKTHTCPPCPAPEAAGGPSVFLFPPKPKDTLMISRTPEVTCVVVDVSHEDPEVK |
| SEQ ID NO:330 | FNWYVDGVEVHNAKTKPREEQYNSTYRVVSVLTVLHQDWLNGKEYKCKVSNKALP |
| | APIEKTISKAKGQPREPQVYTLPPSREEMTKNQVSLSCAVKGFYPSDIAVEWESNGQPE |
| | NNYKTTPPVLDSDGSFFLVSKLTVDKSRWQQGNVFSCSVMHEALHNRFTQKSLSLSP |
| | GGGGGSGGGGSGGGGSGGGGSGGGGSQVQLVESGGGLVQAGGSLRLSCAASGRTFE |
| | TIAMGWFRQVPGKEREFVAVIRGSGVATYYPDSVKGRFTISKDSAKNTVYLQMNNLK |
| | PEDTAVYYCAATTNRFKNTDYTTYDYWGQGTQVTVSSA |
| BI-065 Knob | EPKSSDKTHTCPPCPAPEAAGGPSVFLFPPKPKDTLMISRTPEVTCVVVDVSHEDPEVK |
| SEQ ID NO:331 | FNWYVDGVEVHNAKTKPREEQYNSTYRVVSVLTVLHQDWLNGKEYKCKVSNKALP |
| | APIEKTISKAKGQPREPQVYTLPPSREEMTKNQVSLWCLVKGFYPSDIAVEWESNGQP |
| | ENNYKTTPPVLDSDGSFFLYSKLTVDKSRWQQGNVFSCSVMHEALHNHYTQKSLSLS |
| | PGGGGGSGGGGGPLGVRGGGGGSIWELKKDVYVVELDWYPDAPGEMVVLTCDTPE |
| | EDGITWTLDQSSEVLGSGKTLTIQVKEFGDAGQYTCHKGGEVLSHSLLLLHKKEDGI |
| | WSTDILKDQKEPKNKTFLRCEAKNYSGRFTCWWLTTISTDLTFSVKSSRGSSDPQGVT |
| | CGAATLSAERVRGDNKEYEYSVECQEDSACPAAEESLPIEVMVDAVHKLKYENYTSS |
| | FFIRDIIKPDPPKNLQLKPLKNSRQVEVSWEYPDTWSTPHSYFSLTFCVQVQGKSKREK |
| | KDRVFTDKTSATVICRKNASISVRAQDRYYSSSWSEWASVPCSGGGGSGGGGSGGGG |
| | SRVIPVSGPARCLSQSRNLLKTTDDMVKTAREKLKHYSCTAEDIDHEDITRDQTSTLK |
| | TCLPLELHKNESCLATRETSSTTRGSCLPPQKTSLMMTLCLGSIYEDLKMYQTEFQAIN |
| | AALQNHNHQQIILDKGMLVAIDELMQSLNHNGETLRQKPPVGEADPYRVKMKLCILL |
| | HAFSTRVVTINRVMGYLSSA |
| BI-065 Hole | EPKSSDKTHTCPPCPAPEAAGGPSVFLFPPKPKDTLMISRTPEVTCVVVDVSHEDPEVK |
| SEQ ID NO:332 | FNWYVDGVEVHNAKTKPREEQYNSTYRVVSVLTVLHQDWLNGKEYKCKVSNKALP |
| | APIEKTISKAKGQPREPQVYTLPPSREEMTKNQVSLSCAVKGFYPSDIAVEWESNGQPE |
| | NNYKTTPPVLDSDGSFFLVSKLTVDKSRWQQGNVFSCSVMHEALHNRFTQKSLSLSP |
| | GGGGGSGGGGSGGGGSGGGGSGGGGSEVQLVESGGGLVQPGGSLRLSCAASGRTFE |
| | TIAMGWFRQAPGKGLEFVAVIRGSGVATYYAESVKGRFTISKDSSKNTVYLQMNSLR |
| | AEDTAVYYCAATTNRFKNTDYTTYDYWGQGTQVTVSSA |

### Example 5

### Masking Domain—Functional Activity in the Context of Molecule—in Vitro

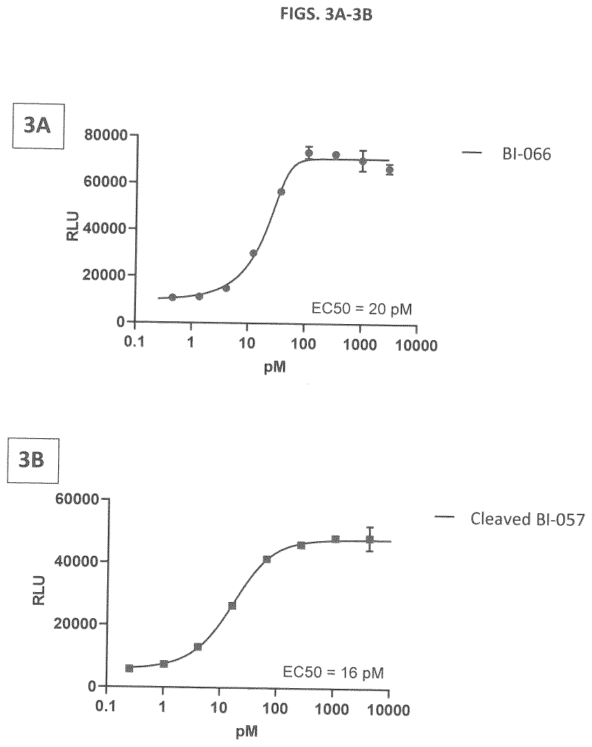

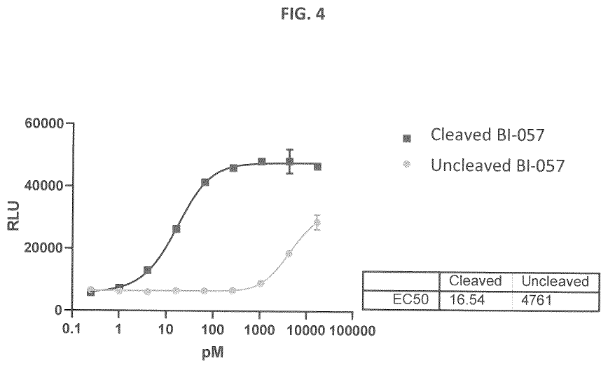

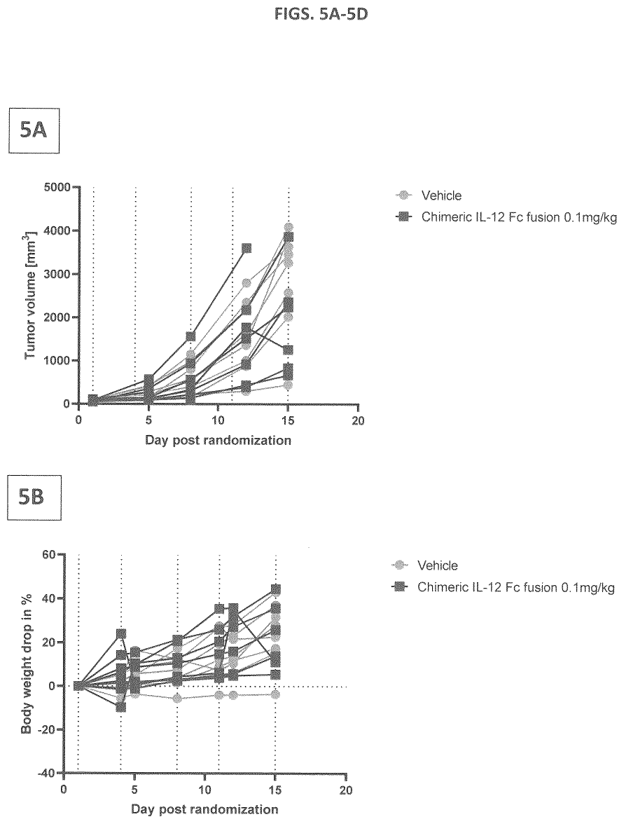

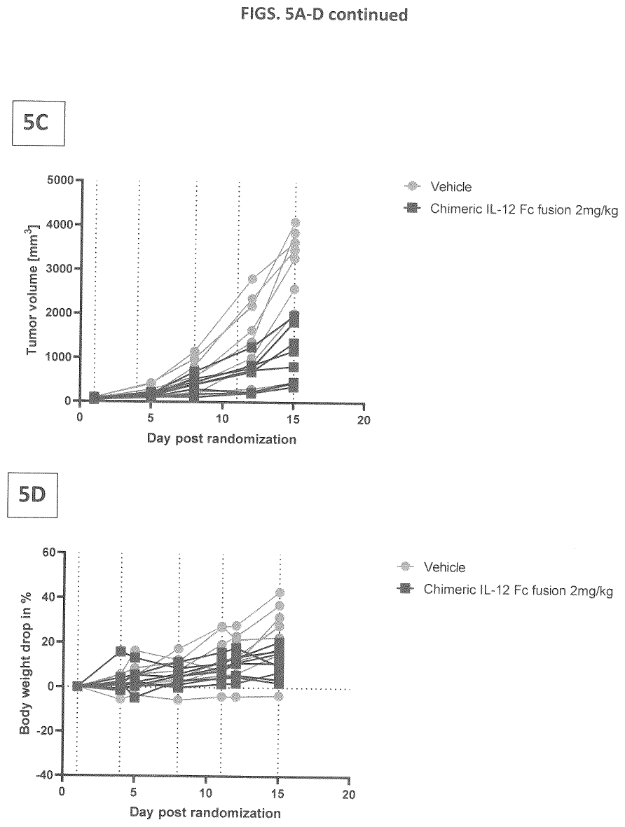

### FIGS. 3A-3B & 4

The prodrug efficiently inhibited IL-12 signaling while in the prodrug context, but effectively signaled upon MMP9 cleavage, both in in vitro assays, as well as in in vivo experiments (see Example 6 for in vivo).

IL-12 routinely showed an EC₅₀ of ˜15 pM in the Promega IL-12 Bioassay and this value was irrespective of whether the IL-12 was provided as a purified recombinant IL-12 or if the IL-12 was released from the prodrug in the presence of MMP9. FIG. 3A shows a chimeric single-chain IL-12 (human p40-GS linker-murine p35) (BI-066) in the Promega IL-12 Bioassay.

The prodrug BI-057 was also tested in this assay. The cleaved prodrug BI-057 after MMP9 digestion showed an EC₅₀ of ˜16 pM in the Promega IL-12 Bioassay (FIG. 3B).

In the absence of MMP9 digestion, the EC₅₀ of the molecule was in the ˜ 5 nM range indicating a functional shift of over 280-fold compared to recombinant IL-12 (FIG. 4). In summary, data presented suggests efficient masking of IL-12 in a prodrug form and full functionality of the cytokine while cleaved from the original molecule.

### Example 6

### Masking Domain—Functional Activity in the Context of Molecule: —In Vivo

### FIGS. 5A-5D, 6A-6B, 7A-7B, 8A-8B & 9A-9C

In an in vivo setting, a prodrug from the initial format scouting experiments having the configuration as shown in FIG. 1B was tested and showed improved safety profiles compared to unmasked IL-12. Said prodrug from the initial format scouting was based on a heterodimeric Fc (Knob-in-hole) with a single chain chimeric IL-12 (human p40-GS linker-murine p35) attached to the C-terminus of one Fc chain and a masking domain (scFv) against the human p40 domain attached to the other Fc chain. The cleavable linker was positioned between the Fc and the p40 domain.

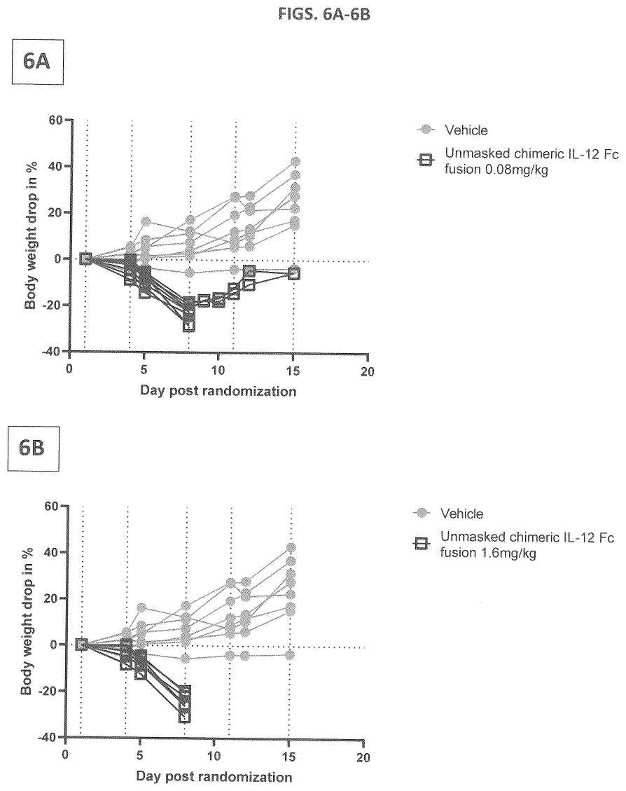

As shown in FIGS. 5A and 5C, the chimeric IL-12 Fc fusion protein inhibited tumor growth in a non-immunogenic and aggressive B16.F10 melanoma model in a dose dependent manner. In addition, B16.F10 model is characterized by relatively low MMPs activity thus providing additional hurdle for the tested molecule. The efficient blocking of IL-12 functionality in a prodrug format was confirmed by the lack of body weight loss even at a dose of 2 mg/kg which is 2000× higher than a murine toxic dose (in molar equivalent) (FIG. 5D). On the contrary, unmasked chimeric IL-12 Fc fusion protein (fusion protein which had all components but lacked the blocking moiety) caused severe toxicity in the majority of animals already at a concentration of 0.08 mg/kg (FIG. 6A). Higher concentrations (1.6 mg/kg which is a molar equivalent of 2 mg/kg dose of masked chimeric IL-12 Fc fusion protein) led to severe body weight loss in treated animals which resulted in death of all treated animals by day 8 (FIG. 6B). This data further confirmed efficient masking of IL-12 while in the prodrug form in the periphery. It also suggested that the molecule remained in a prodrug form while in the bloodstream increasing the therapeutic index of the molecule.

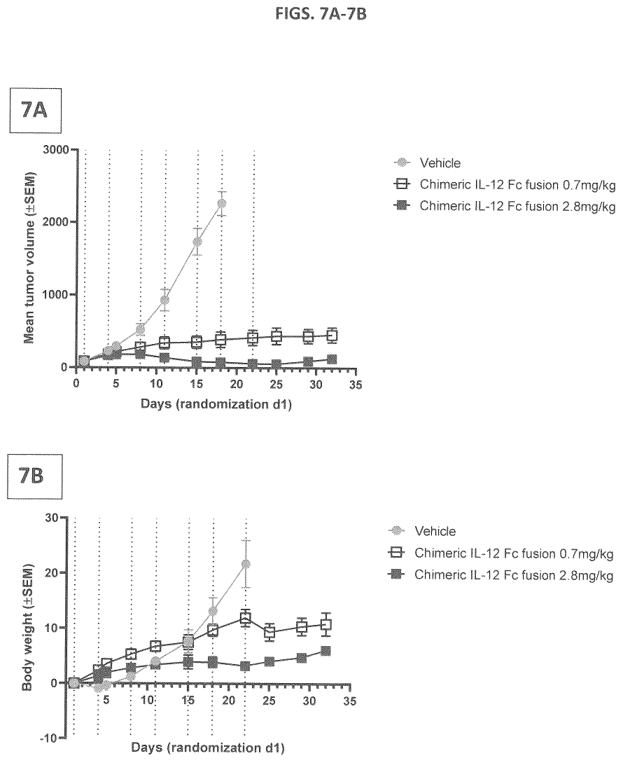

Safety and efficacy of chimeric IL-12 Fc fusion protein was further evaluated in the MC38 model, which confers high MMP activity. FIG. 7A shows dose-dependent tumor growth inhibition without body weight loss which is a surrogate of toxicity (FIG. 7B).

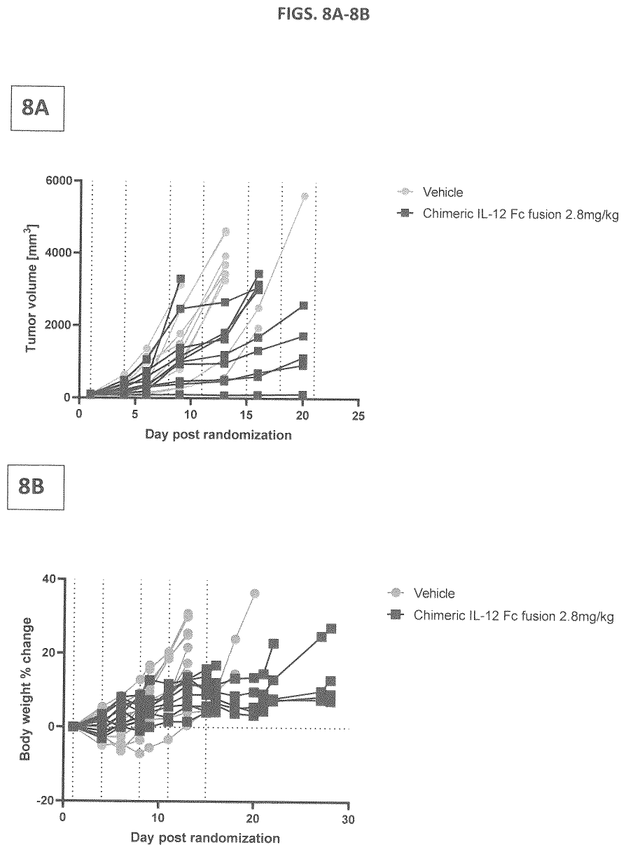

Similar trends were observed in the B16.F10 model (FIGS. 8A-B), which contained far lower MMP activity within the tumor, although, tumor growth inhibition was indeed lower which correlated with the lower MMP profile of this tumor. Importantly, no signs of toxicity were observed at the concentration used (2.8 mg/kg) further confirming efficient masking properties and lack of systemic IL-12-mediated inflammation.

To further confirm this, the level of proinflammatory cytokines in the sera of MC38-bearing mice treated with chimeric IL-12 Fc fusion protein was evaluated one day post second treatment. No proinflammatory cytokines were detectable in the sera of treated mice suggesting lack of systemic toxicity and confirming efficient masking (Table 14).

TABLE 14

| MC38 serum | Animal | | | | | | |
|---|---|---|---|---|---|---|---|
| pg/mL | ID | IL-4 | IL-5 | IL-6 | IFNg | IL-12p70 | TNFa |
| PBS-vehicle | 655-S | <1.36 | <2.18 | <5.51 | <2.18 | <2.34 | <3.66 |
| | 660-S | <1.36 | 2.42 | <5.51 | <2.18 | 2.92 | <3.66 |
| | 665-S | <1.36 | <2.18 | <5.51 | <2.18 | <2.34 | <3.66 |
| | 661-S | <1.36 | <2.18 | <5.51 | <2.18 | <2.34 | <3.66 |
| | 662-S | <1.36 | 19.00 | <5.51 | <2.18 | <2.34 | <3.66 |
| | 670-S | <1.36 | 5.30 | <5.51 | <2.18 | <2.34 | <3.66 |
| chimeric IL-12 | 651-S | <1.36 | <2.18 | <5.51 | <2.18 | <2.34 | <3.66 |
| Fc fusion | 668-S | <1.36 | <2.18 | <5.51 | <2.18 | <2.34 | <3.66 |
| protein | 673-S | <1.36 | <2.18 | <5.51 | <2.18 | <2.34 | <3.66 |
| (2.8 mg/kg) | 656-S | <1.36 | 2.82 | <5.51 | <2.18 | <2.34 | <3.66 |
| | 672-S | <1.36 | <2.18 | <5.51 | <2.18 | <2.34 | <3.66 |
| | 676-S | <1.36 | <2.18 | <5.51 | <2.18 | <2.34 | <3.66 |

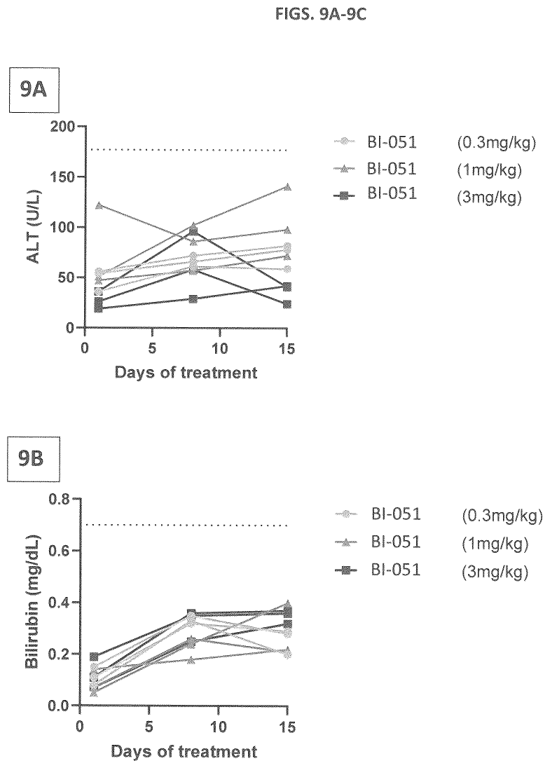

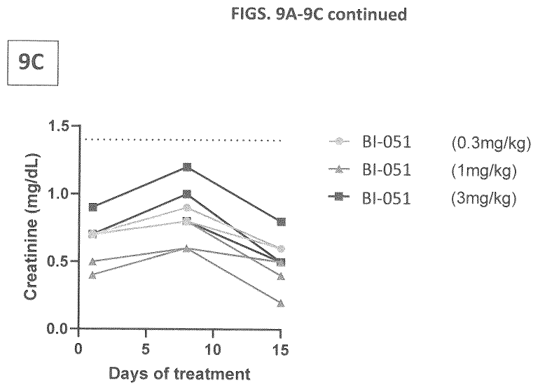

To further confirm IL-12 inhibiting properties of the masking molecule non-human primates (NHP) were injected with human IL-12 Fc fusion protein (BI-051) at doses up to 3 mg/kg. The NHP remained fit with no signs of clinical symptoms of toxicity. No increase in ALT, bilirubin or creatinine was detected suggesting efficient masking of IL-12 in the periphery when in context of prodrug molecule (FIGS. 9A-9C).

### Example 7

### Cleavage Site was Cleaved by MMPs—Functional Activity in the Context of Molecule—In Vitro:

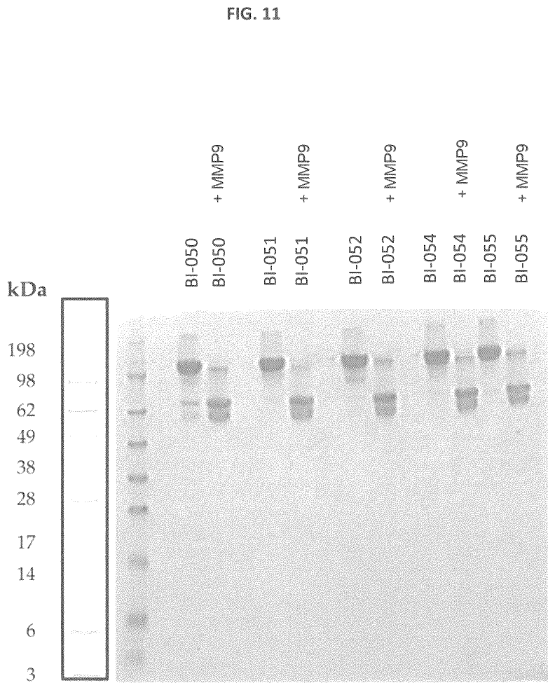

### FIGS. 11, 12, 13A-13E, 14A-14B

The MMP cleavable linker incorporated in the prodrug is susceptible to cleavage by proteases that are typically upregulated within the tumor microenvironment. For the purposes of the functional characterization of the prodrug, recombinant MMP9 was used to cleave and activate the IL-12 from the prodrug.

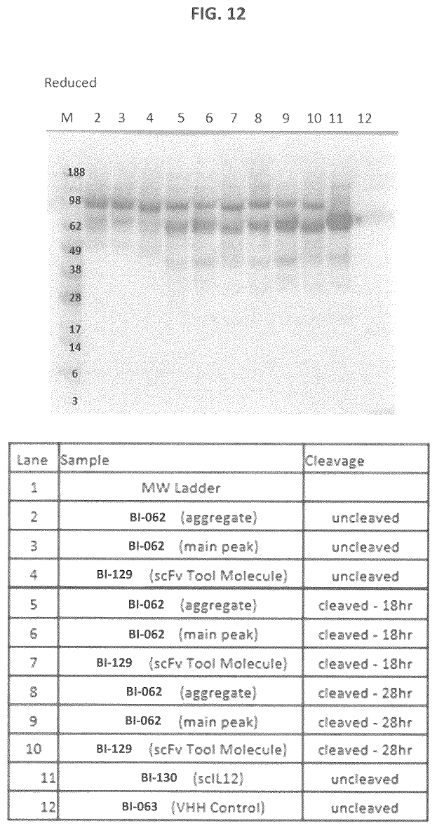

As presented in FIG. 11, activated MMP9 efficiently cleaved the prodrugs (BI-050, BI-051, BI-052, BI-054, BI-055) as shown by SDS-PAGE. Release of IL-12 was further confirmed by Western blot (FIG. 12) and MS analysis (not shown) for another prodrug molecule (BI-062).

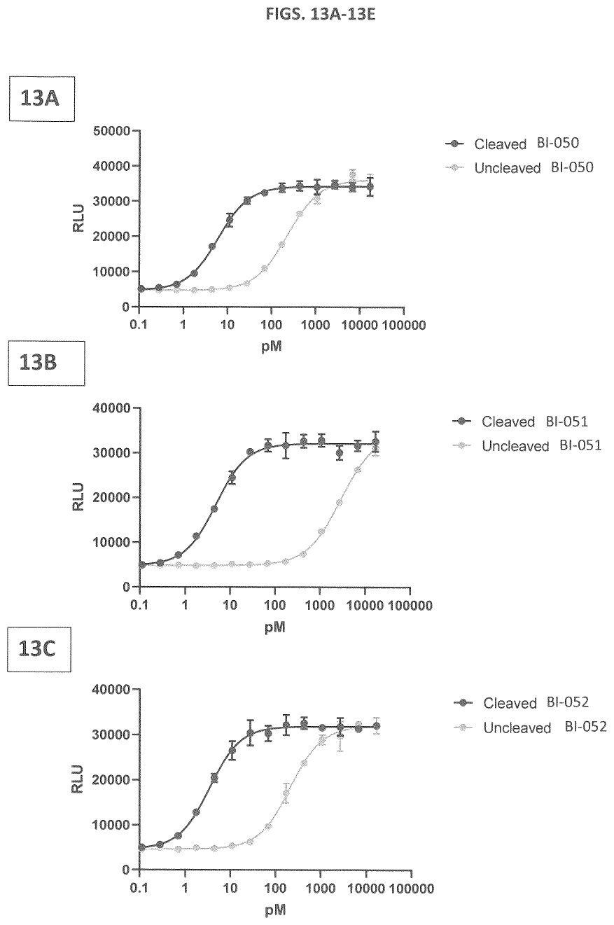

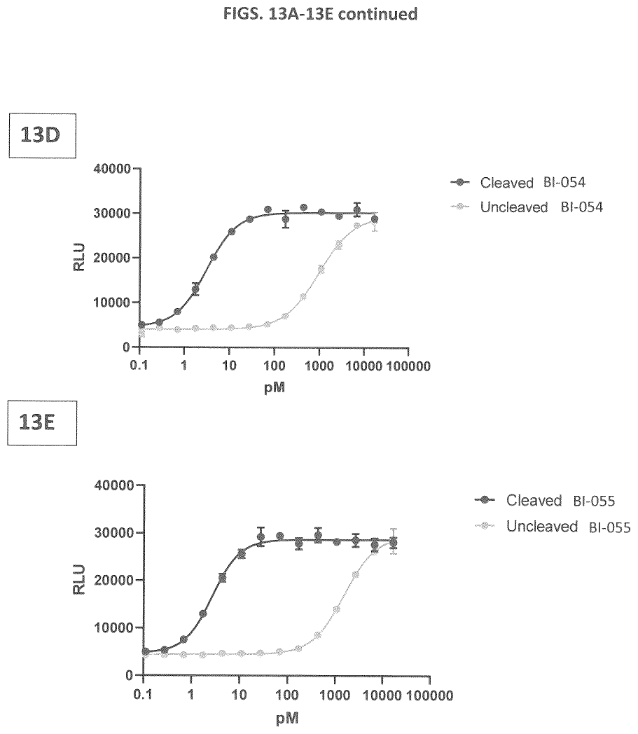

To further investigate functionality of prodrug-released IL-12, the cell-based Promega IL-12 Bioassay was employed. Proteolytically cleaved and sham-treated prodrug molecule were serially diluted and added to reporter cells to measure IL-12 activity. As presented in FIGS. 13A-13E all molecules tested (BI-050, BI-051, BI-052, BI-054, BI-055) had a lower EC₅₀ value upon cleavage compared to the intact molecules suggesting efficient release of biologically active IL-12. In addition, all molecules reached comparable EC₅₀ values upon cleavage with significantly different EC₅₀ values of sham-treated molecules which may suggested different masking properties of those otherwise very similar constructs (Table 15).

The ΔEC₅₀ was used to evaluate variants. The constructs tested were closely related to each other, differing in placement of the collagen I tumor retention peptide, at either the N-terminus of IL-12 (BI-050), the C-terminus of IL-12 (BI-051), or in the intra-IL-12 linker that connected the p40 and p35 domains (BI-052). Similarly, a lower affinity masking domain, differing from BI-051 by only two amino acids in the HCDR3 of the mask, with all other components the same showed decreased masking ability. Finally, construct BI-055 was also based off of BI-051, but contained additionally a S354C/Y349C CH3 stabilizing disulfide that was often used to drive heterodimerization of Knob-in-Hole formation, which showed similar masking ability.

The data clearly showed that there were direct impacts on the ΔEC₅₀ of the variants with respect to these various parameters and therefore, had implications of the safety profile.

TABLE 15

| | EC₅₀ of cleaved | EC₅₀ of uncleaved | |
|---|---|---|---|
| Construct | molecule (pM) | molecule (pM) | ΔEC₅₀ |
| BI-050 | 5.999 | 229 | 38 |
| BI-051 | 4.743 | 2869 | 605 |
| BI-052 | 3.446 | 211 | 61 |
| BI-054 | 3.076 | 952 | 313 |
| BI-055 | 2.718 | 1531 | 563 |

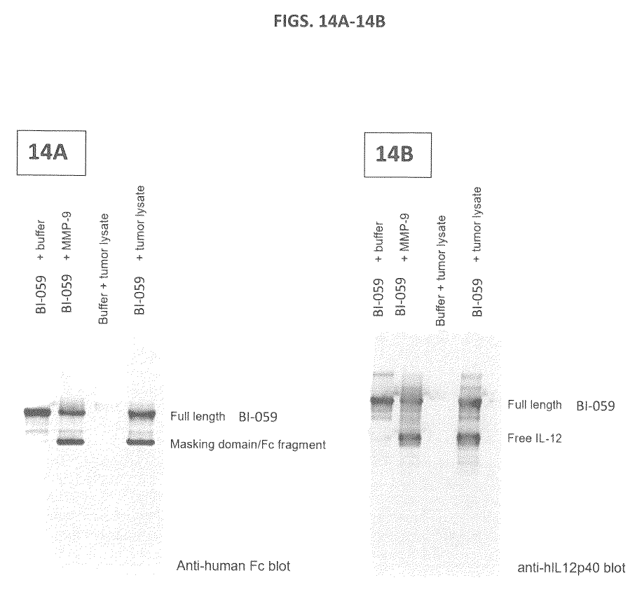

To further evaluate the feasibility of IL-12 release from the prodrug in more clinically relevant settings, lysates from human tumor tissues were prepared and used as a source of MMP. After 2 h incubation of chimeric molecule BI-059 with the lysates, the proteins were evaluated in Western blot. As presented in FIGS. 14A-14B, BI-059 was efficiently cleaved with the human tumor lysates to the level comparable to activated MMP9. This data showed the feasibility of MMP-mediated cleavage and cytokine release in the tumor microenvironment.

### Example 8

### Cleavage Site was Cleaved by MMPs—Functional Activity in the Context of Molecule—In Vivo:

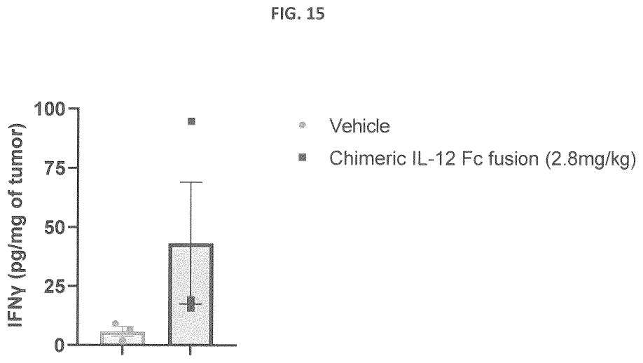

### FIGS. 15 & 16A-16F

IL-12 cytokine stimulates T-cells and NK cells, and those cells produce IFNγ in response to this stimulation. As shown in Table 14, there was no detectable levels of IFNγ in the periphery upon treatment with chimeric IL-12 Fc fusion protein suggesting efficient masking properties of the molecule. In further experiments using the same IL-12 Fc fusion protein as tested in Example 6, it was investigated whether this treatment would result in detectable levels of IFNγ within the tumors. To this end, MC38-bearing animals were treated with IL-12 Fc fusion protein (2.8 mg/kg) twice and tumors were collected 1 day after the second treatment. As shown in FIG. 15, there was a significant increase in intratumoral IFNγ in treated animals compared to vehicle-injected mice, suggesting protease mediated release of functional IL-12.

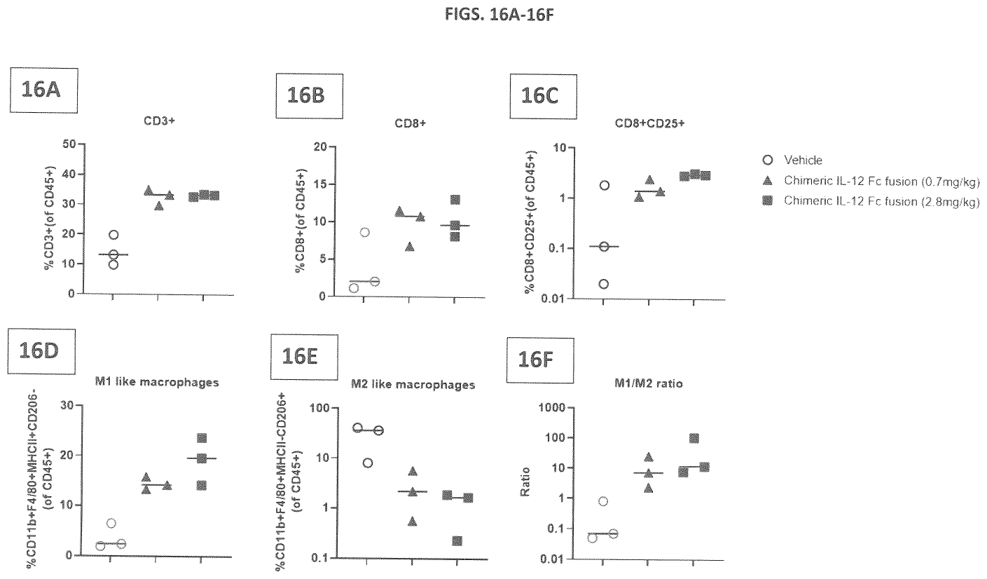

To further elucidate effects of active IL-12 released at the tumor site, changes within TME were investigated upon treatment with chimeric Fc fusion protein as described above. Significant changes were observed in the lymphocytic and myeloid cell infiltration upon treatment. As shown in FIGS. 16A-16F, treatment with the chimeric prodrug molecule resulted in a significant influx of CD3 T-cells (FIG. 16A), particularly CD8 T cells (FIG. 16B). Those T cells exhibited activated phenotype as indicated by CD25 expression (FIG. 16C). In addition, significant increase in M1-like macrophages was observed in the treated animals (FIG. 16D) which was accompanied by decrease in the M2-like macrophages (FIG. 16E). As a result, an increased M1/M2 ratio was observed (FIG. 16F) which is a sign of beneficial changes within tumor microenvironment. Note that FIGS. 160, 16E, and 16F use a logarithmic scale in order to more clearly show the results.

### Example 9

### TME Retention Linker—Functional Activity in the Context of Molecule In Vitro:

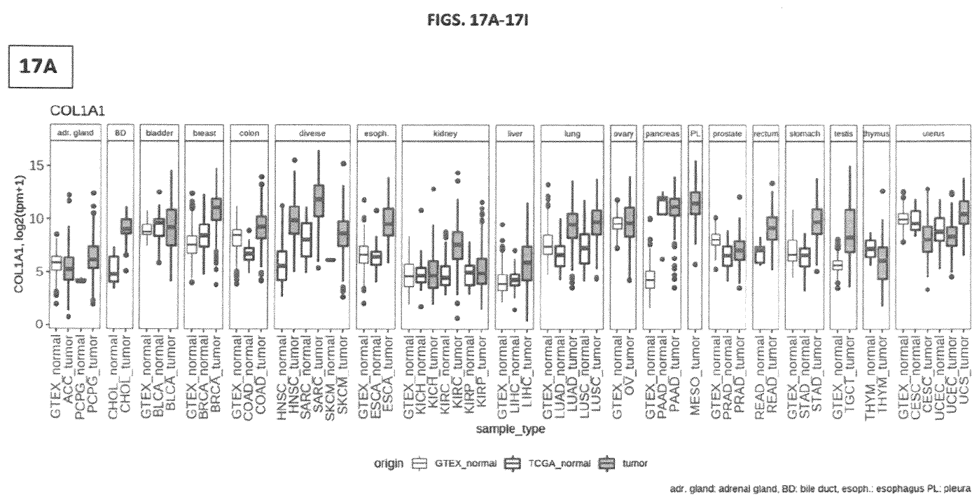

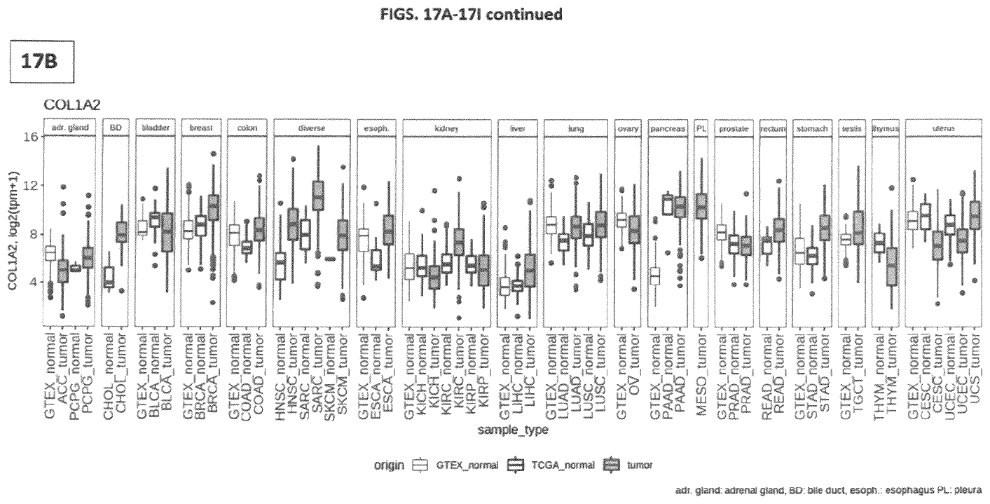

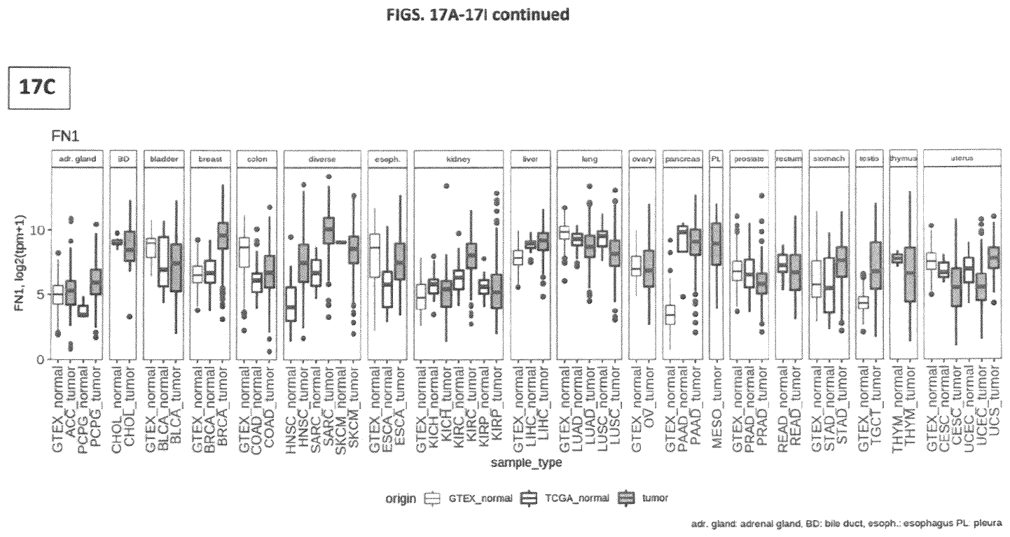

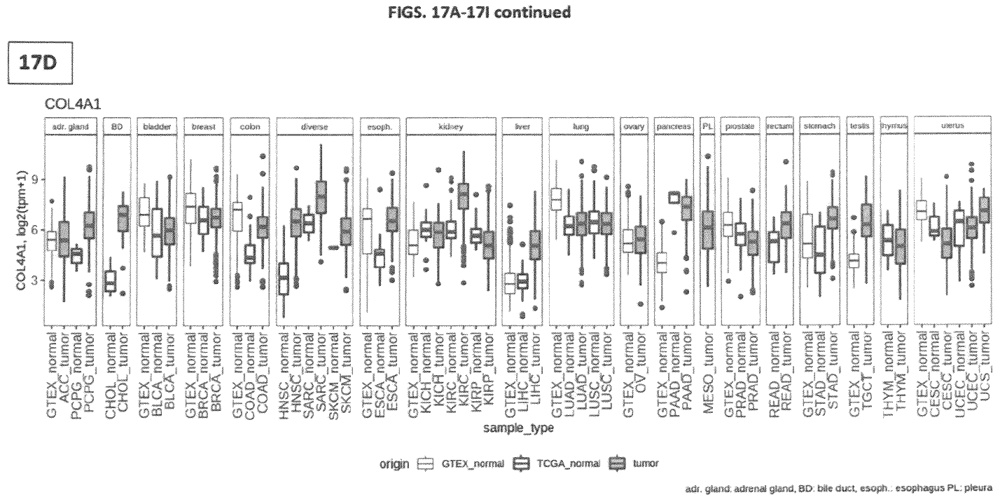

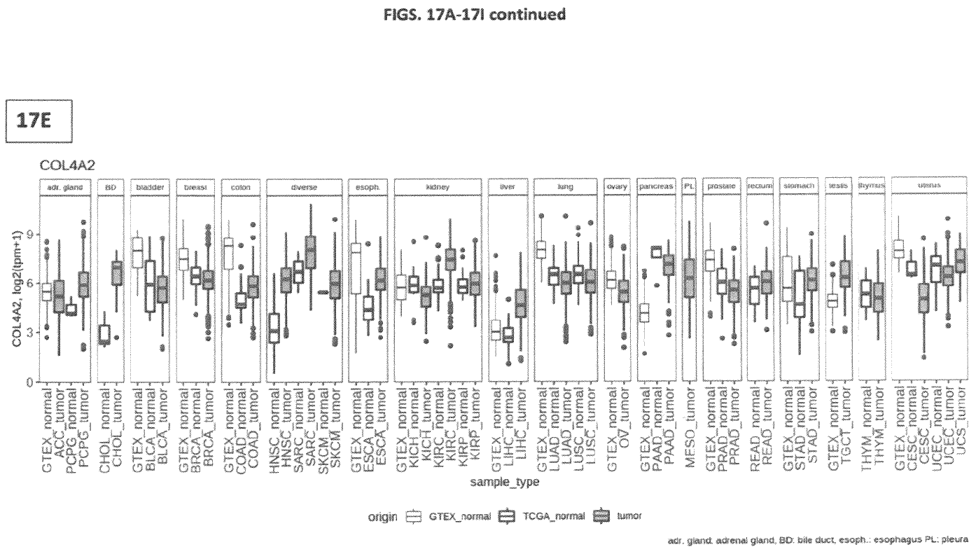

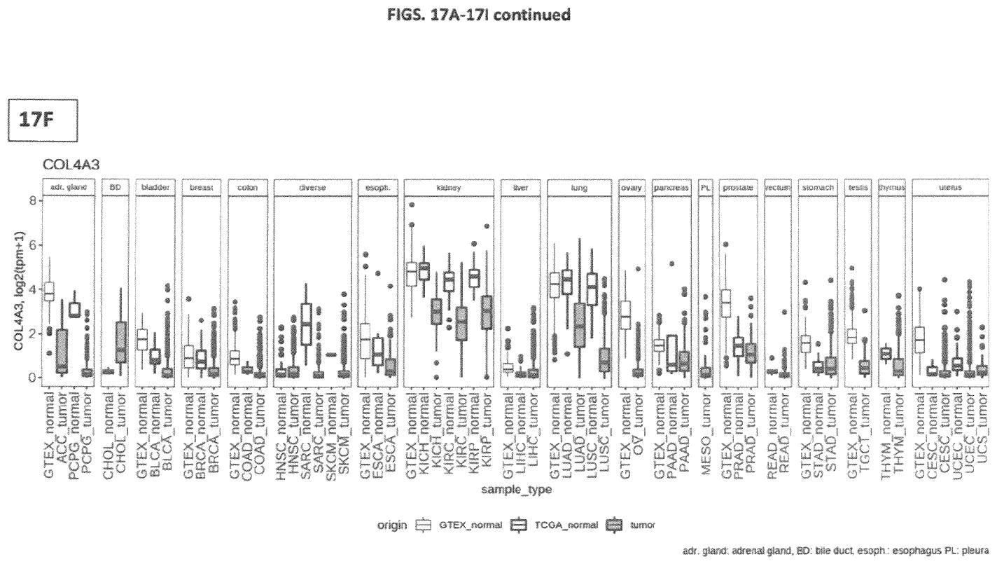

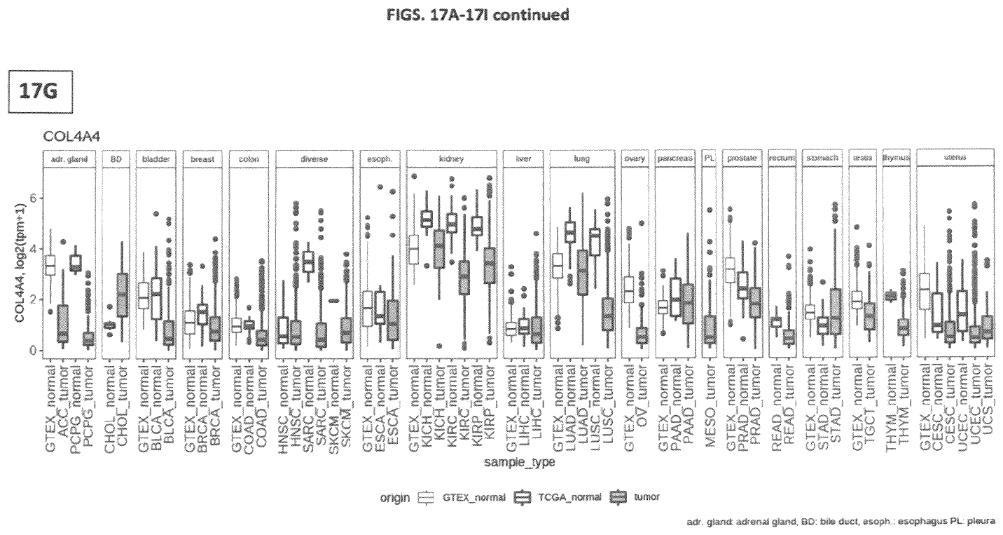

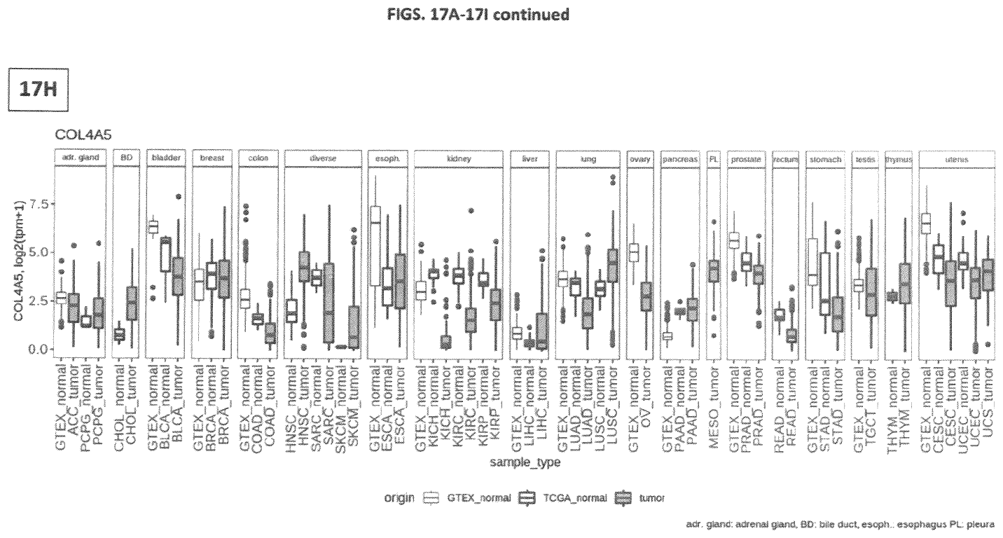

### FIGS. 17A-17I, 18A-18B, 19 & 20A-20D

A major driver for the prodrug approach was to limit the systemic toxicity of IL-12 and enabling greater safety profiles, while still harnessing the strong anti-tumor properties of the cytokine. This was initially performed through the creation of the prodrug with tumor activatable IL-12 release. However, IL-12 itself has a relatively short half life (<9 hours), and additional concerns may arise about the cytokine escaping from the tumor microenvironment. Due to such low levels of IL-12 required to trigger a toxicity response, the addition of peptide sequences to further capture the cytokine within the tumor microenvironment were explored.

The TME is rich in ECM proteins such as e.g. collagen I, collagen IV or fibronectin which can potentially serve as anchors for other fusion protein. ECM proteins were found to be upregulated in many tumor types (FIGS. 17A-17I). Therefore, it was hypothesized that the incorporation of tumor retention peptide onto IL-12 could allow for higher retention of IL-12, leading to longer exposure of immune cells to the cytokine providing further potency. Additionally, a linker which binds ECM proteins may also provide benefit for the prodrug. Circulating prodrug, with the TME linker fused to the IL-12, could potentially increase the residency time of the prodrug within the TME. The immediate impact of this would be longer exposure of the prodrug to enzymatic cleavage, enhancing the likelihood the prodrug is converted to active drug.

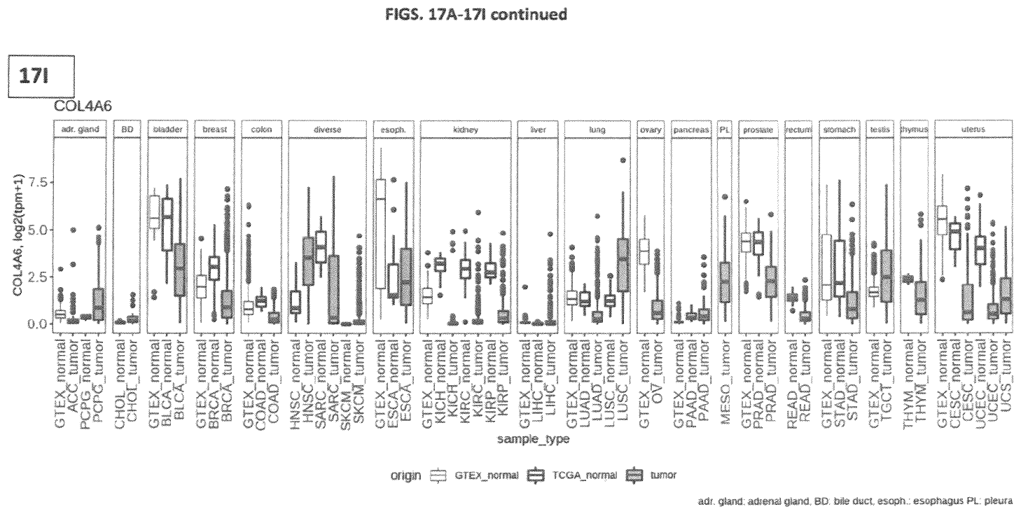

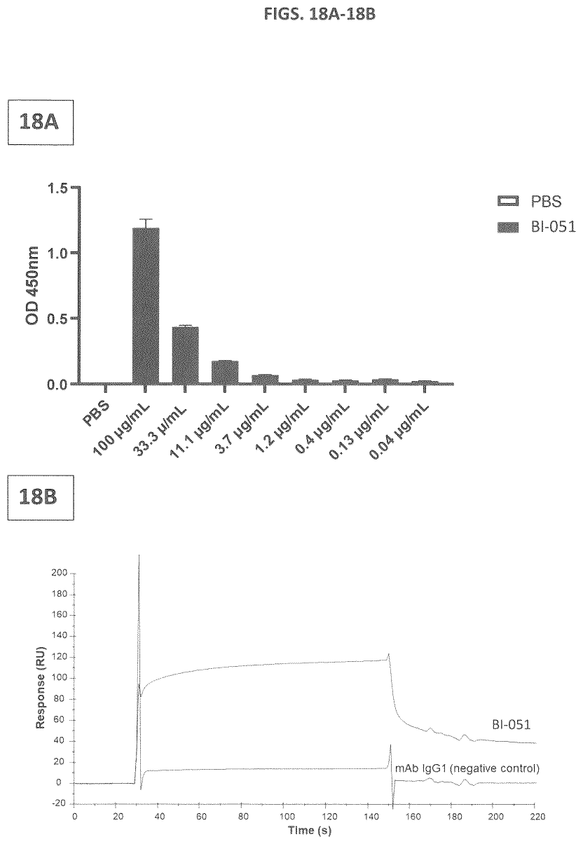

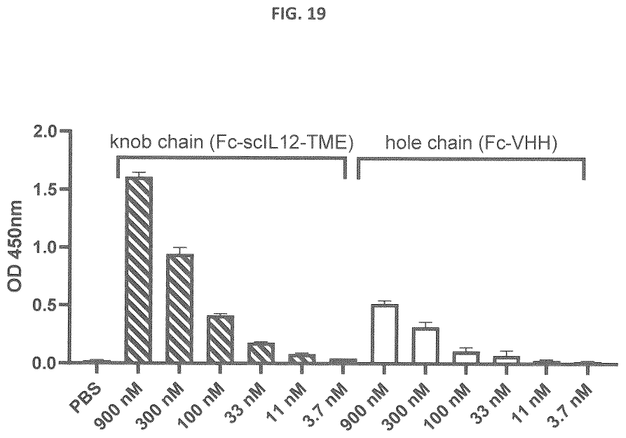

To test the ability of the peptide to bind to ECM protein while in the context of Fc fusion protein, the binding of BI-051 to collagen I was evaluated. As presented in FIG. 18A, dose-dependent binding of BI-051 to collagen I-coated plates were observed. This was further confirmed in an SPR experiment, as shown in FIG. 18B. To achieve the cytokine retention at the tumor site, tumor retention linker was attached to the cytokine (part of the knob chain). To further confirm that TME-binding linker conveyed the binding properties, the knob and hole chain of the molecule were tested in a separate ELISA-based assay on collagen-I-coated plates. As presented in FIG. 19, knob chain containing TME linker peptide bound the collagen-I-coated plates in a dose-dependent fashion and to a significantly higher degree than the hole chain which is void of the TME linker peptide.

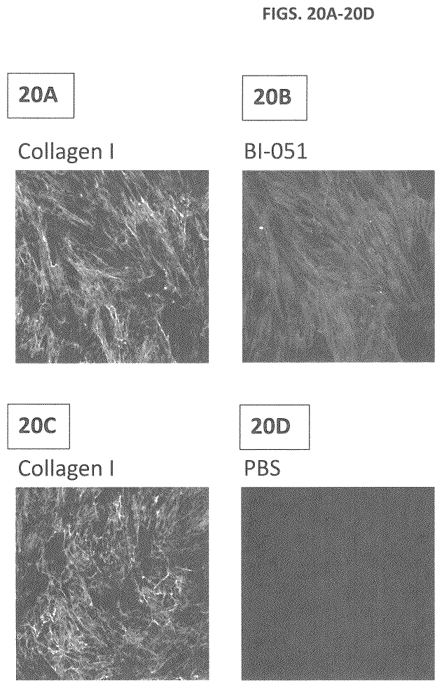

To further elucidate binding of BI-051 to collagen I in a more physiological conditions, a scar in a jar assay was employed. In this assay, fibroblasts were stimulated to produce different matrix proteins (in the present assay predominantly collagen I) in a form that closely resembled the natural extra cellular matrix. To such prepared plates, fluorescently-labeled IL-12 Fc fusion protein was added and its binding to collagen I was evaluated. As presented in FIG. 20B, BI-051 was clearly detectable along the collagen-I fibres suggesting binding of the molecule to this ECM protein.

### Example 10

### Molecule In Vivo Data

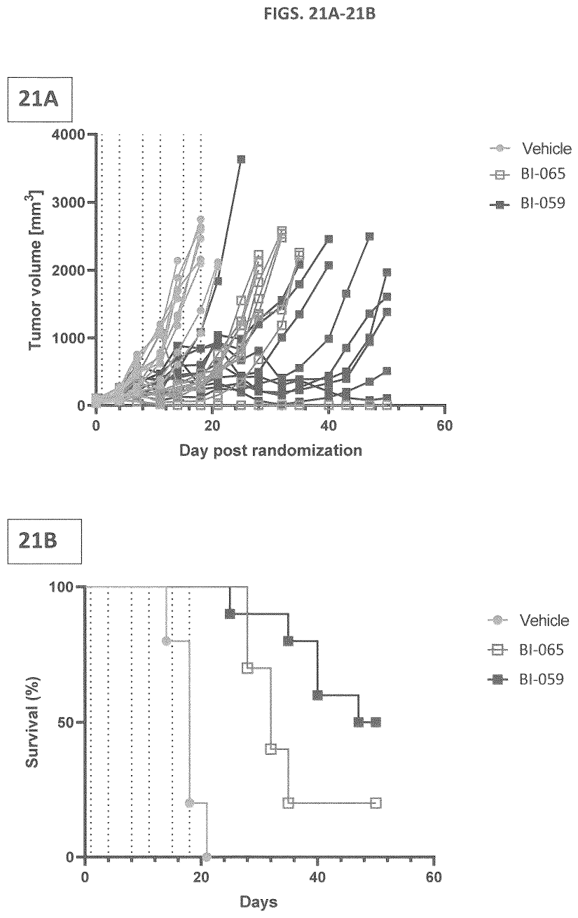

### FIGS. 21A-21B, 22, 23A-23B & 24A-24B

The chimeric molecules were further tested in in vivo settings. Due to a very low content of collagen I in syngeneic models (data not shown) which is in contrast to human tumors, testing of collagen-binding motifs in those models remains elusive. On the contrary, fibronectin expression in syngeneic models enabled testing of the TME linker concept in tumor-bearing mice. To this end, chimeric IL-12 Fc fusion protein which contained fibronectin-binding motif (BI-059) was tested in MC38 model. As shown in FIG. 21A, BI-059 significantly inhibited tumor growth compared to both vehicle as well as IL-12 Fc fusion without TME linker (BI-065). Inhibition of tumor growth led to a significant extension of survival within BI-059 treated animals with 50% of animals still alive at the end of the experiment (FIG. 21B).

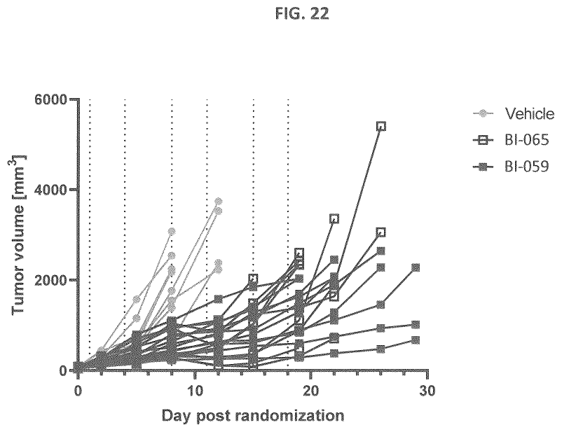

To further elucidate the ability of TME binding linker to improve anti-tumor efficacy and/or safety of the IL-12 Fc fusion, BI-059 was tested in a hard-to-treat B16.F10 melanoma model. This model is characterized by a low MMP activity and significantly lower level of fibronectin (8× according to expression level, data not shown). As shown in FIG. 22 despite the low MMP activity and low fibronectin expression, BI-059 animals significantly better controlled tumor growth in this aggressive model compared to the animals treated with IL-12 Fc fusion protein without the TME linker.

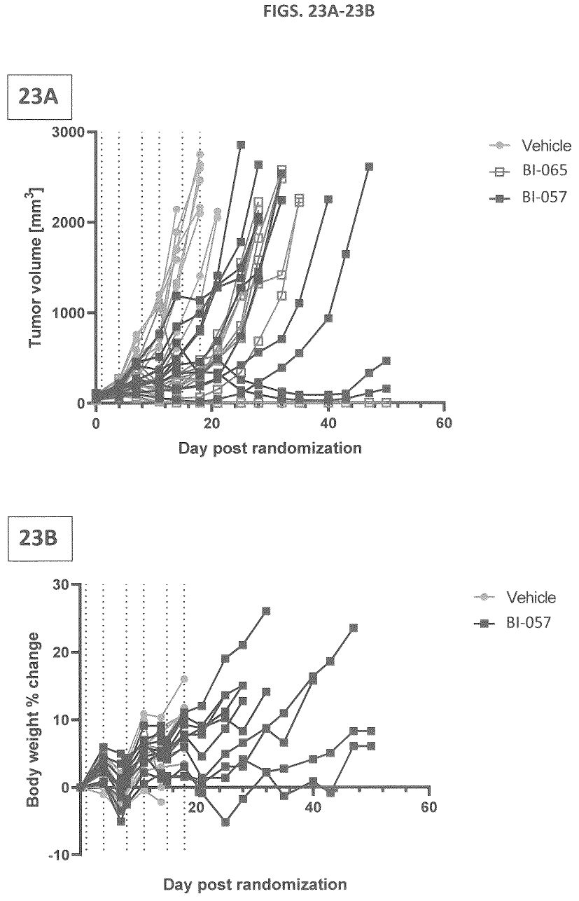

As mentioned above, syngeneic models were characterized by a poor stromal compartment and collagen I expression was usually limited to the outer membrane of those tumors. Despite that, significant delay of tumor formation was observed in BI-057 treated animals bearing MC38 tumors (FIG. 23A). 4/10 of animals in this group controlled tumor for a longer time compared to the 2/10 animals in the group which received IL-12 Fc fusion protein void of TME linker. Importantly, in accordance to in vitro data showing significant ΔEC₅₀ between cleaved and uncleaved molecule, there was no sign of toxicity observed in those animals (FIG. 23B).

BI-057 was further tested in B16.F10 model to further evaluate its efficacy (due to extremely low level of collagen I expression this model was not suitable to test the benefit of TME linker). As shown in FIG. 24A, BI-057 treatment resulted in significant delay in tumor growth compared to the vehicle-treated animals. In agreement with previously presented data, there was no signs of toxicity in the treated animals as shown by lack of body weight loss (FIG. 24B).

Those data showed that the molecules containing TME retention linker were efficacious and safe in these models. In an environment where ECM proteins are broadly present, which is typical for human tumors, it is expected that TME linker would even more significantly contribute to both efficacy and safety of the presented molecules.

### Example 11

In a further approach a genetically modified model characterized by increased levels of ECM proteins within the tumors is used to further analyze the effect of the IL-12 Fc fusion protein and in particularly the TME linker on retention, efficacy, mode of action and safety profile of the IL-12 Fc fusion proteins. To this end such a genetically modified model is treated with an IL-12 Fc fusion protein and TME linker retention, efficacy, mode of action and safety profile is assessed.

In a yet another approach different tumor models are chosen having different expression levels of ECM proteins in general, such as collagen (low, medium or high expression). The models are treated with the IL-12 Fc fusion protein and efficacy, potency, toxicity and/or retention is assessed.

### Example 12

### Product Quality

Different molecules (Table 16) were generated differing in the type and position of the tumor retention linker and the used VHH masking domain. Molecules were manufactured under controlled and representative conditions in stable cell pools in small scale. Results are summarized in Table 17. Expression in small-scale bioreactor was successful, harvest was performed on day 14 for all constructs. BI-051, BI-053, BI-054 and BI-055 had a similar upstream process performance. Productivity of BI-052 expressing cells and overall process performance was low and additional species were observed in size exclusion chromatography of harvested samples. Similarly, BI-050 showed a heterogenous size exclusion indicating instability of the construct.

Expressed constructs were then purified downstream as described and product quality was assessed (Table 18).

BI-050 and BI-052 had elevated low molecular weight species that could partially not be identified indicating instability of the constructs during manufacturing. BI-054 showed instabilities on process intermediates. BI-051 and BI-055 showed overall good product quality and reasonable manufacturability. However, during product quality analysis (HILIC-MS) it was observed that BI-055 fragmented due to molecular instability as a result of the introduction of an additional stabilizing disulfide bridge making the molecule more rigid and prone to fragmentation.

TABLE 16
Overview of variant molecules. Molecules differ in their TME linker choice and
position in the molecule relative to IL-12 and the used VHH masking domain.

| | Molecule code | | | | | |
|---|---|---|---|---|---|---|
| | BI-050 | BI-051 | BI-052 | BI-053 | BI-054 | BI-055 |
| Fc part | Knob into hole | | | | | |
| TME | Collagen I | Collagen I | Collagen I | Fibronectin | Collagen I | Collagen I |
| linker | | | | | | |
| TME | N-term | C-term | intra | C-term | C-term | C-term |
| position | IL-12 | IL-12 | IL-12 | IL-12 | IL-12 | IL-12 |
| VHH | Mix 13 | Mix 13 | Mix 13 | Mix 13 | Mix 15 | Mix 13 |

TABLE 17
Process performance of molecules in upstream processing.

| | BI-050 | BI-051 | BI-052 | BI-053 | BI-054 | BI-055 |
|---|---|---|---|---|---|---|
| Max. viable | 18.3 × 10⁶ | 17.0 × 10⁶ | 24.5 × 10⁶ | 14.1 × 10⁶ | 18.0 × 10⁶ | 19.5 × 10⁶ |
| cell density | cells/mL | cells/mL | cells/mL | cells/mL | cells/mL | cells/mL |
| Titer | 5.8 g/L | 5.2 g/L | 4.2 g/L | 5.2 g/L | 6.5 g/L | 6.6 g/L |
| Summary | Heterogenous | High | Low process | High | High | High |
| | size exclusion | process | performance. | process | process | process |
| | chromatography | performance | High cell | performance | performance | performance |
| | chromatogram | and | growth, but | and | and | and |
| | of capture pool | productivity. | low | productivity. | productivity. | productivity. |
| | with additional | | productivity. | | | |
| | species. | | Additional | | | |
| | | | species in size | | | |
| | | | exclusion | | | |
| | | | chromatography | | | |
| | | | observed. | | | |

TABLE 18
Process performance in downstream processing
and product quality of final molecules.

| | BI-050 | BI-051 | BI-052 | BI-053 | BI-054 | BI-055 |
|---|---|---|---|---|---|---|
| Total | 16.2% | 12.5% | 15.9% | n.a. | 15.9% | 22.9% |
| downstream | | | | | | |
| process yield | | | | | | |
| Monomer | 86.6% | 94.6% | 94.3% | n.a. | 90.4% | 94.7% |
| content by size | | | | | | |
| exclusion | | | | | | |
| chromatography | | | | | | |
| Low molecular | 5.3% | 2.3% | 2.9% | n.a. | 2.3% | 2.5% |
| weight species | | | | | | |
| by size | | | | | | |
| exclusion | | | | | | |
| chromatography | | | | | | |
| High molecular | 8.1% | 3.1% | 2.8% | n.a. | 7.3% | 2.8% |
| weight species | | | | | | |
| by size | | | | | | |
| exclusion | | | | | | |
| chromatography | | | | | | |
| Main peak by | 92.6% | 98.8% | 96.3% | n.a. | 98.5% | 99.3% |
| capillary gel | | | | | | |
| electrophoresis | | | | | | |
| Low molecular | 7.4% | 1.2% | 3.7% | n.a. | 1.5% | 0.7% |
| weight species | | | | | | |
| by capillary gel | | | | | | |
| electrophoresis | | | | | | |
| Product quality | Molecular | Molecular | Molecular | n.a. | Molecular | Molecular |
| by mass | mass | mass | mass | | mass | mass |
| spectrometry | confirmed; | confirmed. | confirmed; | | confirmed. | confirmed. |
| | additional | | additional | | | Clipping |
| | low | | unknown | | | variant |
| | molecular | | variant | | | without |
| | variant | | detected. | | | IL-12 |
| | detected. | | | | | detected. |

### Example 13

In Vitro Comparison of Molecules Containing Collagen I TME Linker and these Devoid of it.

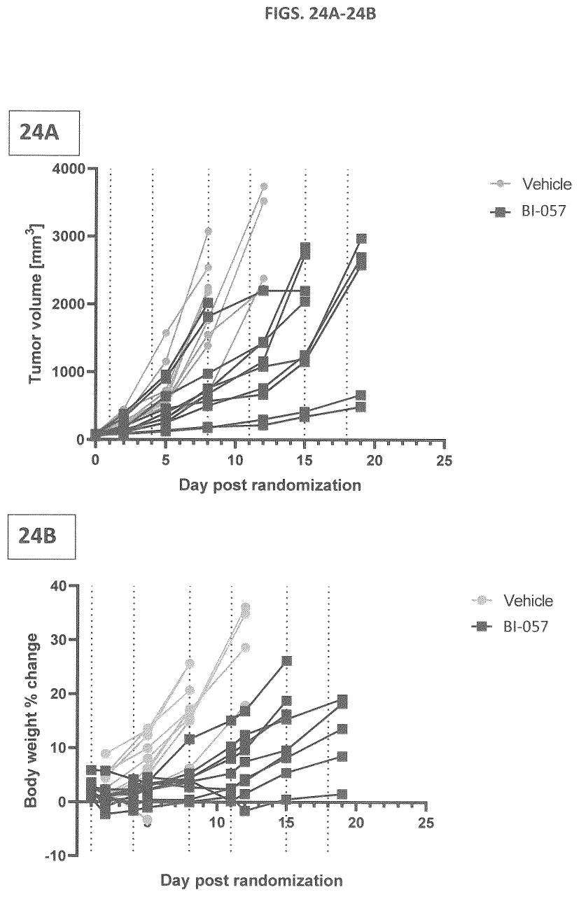

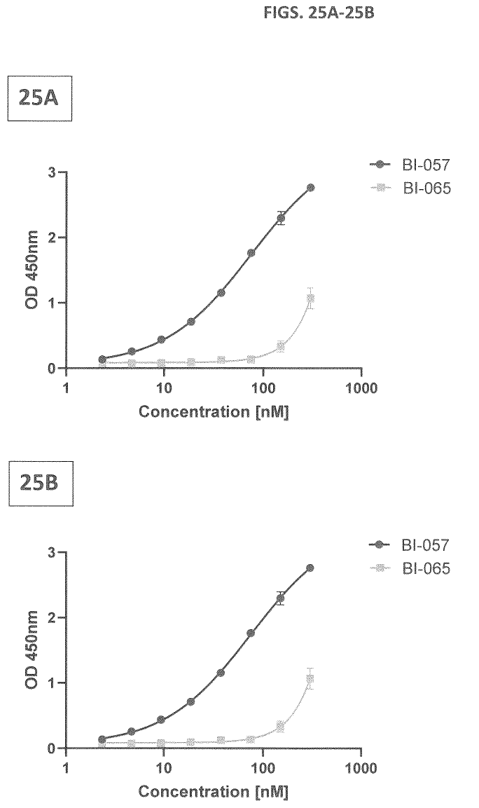

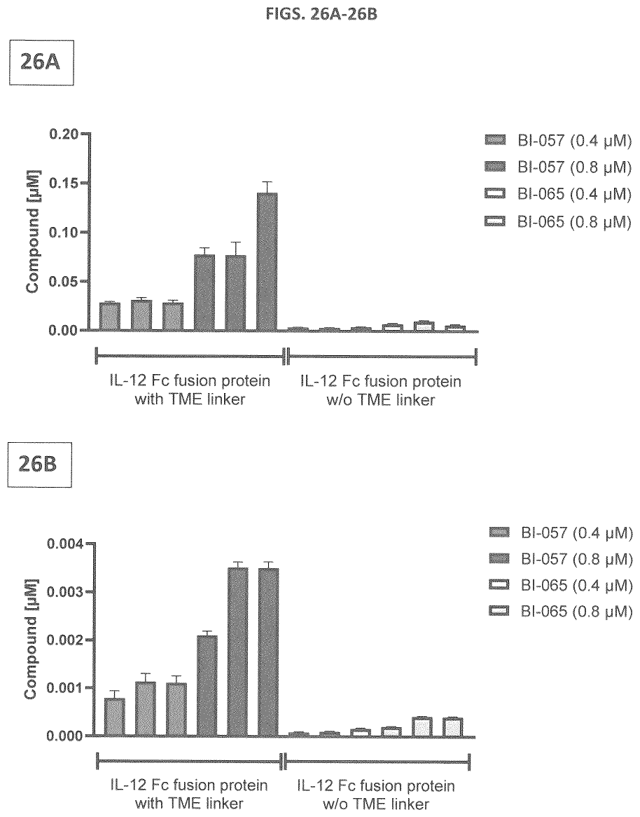

### FIGS. 25A-25B-26A-26B

Collagen binding properties were further evaluated in ELISA assay. Chimeric IL-12 Fc fusion protein BI-065 or chimeric IL-12 Fc fusion protein containing collagen I TME linker BI-057 were added to collagen I-coated plates. To this end, plates were coated either with rat collagen I (FIG. 25A) or with human skin collagen I (FIG. 25B).

Briefly, high binding microplates (greiner) are coated over night at 4° C. with rat collagen (Corning) or human collagen (Millipore/R&D) diluted in coating buffer (Invitrogen) with a concentration of 0.005 mg/mL. IL-12 Fc fusion proteins are diluted in PBS+1% BSA and added to collagen precoated plates for 2 hours. The plates are blocked with 1% BSA before addition of proteins to minimize unspecific binding. Next, plates are washed 3× followed by incubation with biotinylated anti-human Fc antibody (Invitrogen). After additional washing step, Streptavidin-HRP are added to the plates. Substrate are added to plates after a washing step and plates are read in ELISA reader (Tecan).

We observed dose-dependent binding of BI-057 to collagen-coated plates reaching EC50 at 26 nM or 75 nM for rat and human collagen, respectively. BI-065 did not bind to the collagen-coated plates and an increased binding was observed only at the highest concentration.

To further evaluate collagen-binding properties of IL-12 Fc fusion protein, precision cut liver slices of rat fibrotic livers were employed. Syngeneic tumor models consist of only very limited amounts of collagen, contrary to the human tumors, therefore fibrotic tissues constitute more relevant collagen architecture.

Briefly, tissue cores are cut from the fibrotic rat liver using a 5 mm cylindrical motorized tissue coring press (Alabama R&D, MD5000), and 300 μm thick tissue slices are cut using a tissue slicer (Alabama R&D, MD6000). All tissue slices are cultured with a floating culture system (60 rpm) in 12 well plates (Nunc) in a humidified incubator, 95% O₂/5% CO₂, 37° C. in 1.3 mL supplemented William's Medium E (Life Technologies). IL-12 Fc fusion proteins are added to the slices and incubated for 2 hours or 24 hours. After 2 hours of incubation the slices are transferred into fresh William's Medium E and incubated for further 22 hours as washing step before they are harvested, too. After the incubation the slices are washed 3 times with William's Medium E to remove unbound IL-12 Fc fusion protein. Single slices are collected for harvesting in Lysis Matrix tubes (snap frozen). The harvested slices are lysed with MSD Tris Lysis Buffer (MSD) and homogenized with Precellys Evolution Homogenizer (Bertin Technologies) at 6800 rpm for 2×30 s. Afterwards a centrifugation step is performed to get rid of cell debris. The amount of IL-12 Fc fusion protein of the supernatant of the homogenates is determined with the MSD U-PLEX Biomarker Assay and IL-12 Fc fusion protein diluted in MSD Lysis buffer (serial dilution 1:2) as standard. For the IL-12 Fc fusion protein detection the MSD Gold Small Spot Streptavidin plate is coated with the biotinylated anti-IL12 mouse capture antibody and the anti-IL12 human antibody with SulfoTag is used as detection antibody. MSD Read buffer is added before the plates are read with an MSD microplate reader.

Chimeric IL-12 Fc fusion protein BI-065 or chimeric IL-12 Fc fusion protein containing collagen I TME linker BI-057 were added to precision cut liver slices for 2 or 24 hours. After 2 hours, the slices were thoroughly washed and cultured in medium for additional 22 h. As shown in FIG. 26A, there was dose-dependent recovery of IL-12 Fc fusion protein containing collagen I TME linker. The amount of IL-12 Fc fusion protein BI-065 recovered from the slices remained at the background level and was more than 10× lower than the amounts of BI-057 suggesting binding of the later molecule to the collagen present in fibrotic livers. In addition, similar results were observed when slices were cultured in the presence of BI-057 or BI-065 for 2 h then thoroughly washed and remained in the culture for further 22 h. As shown in FIG. 26B, there was dose-dependent recovery of chimeric IL-12 Fc fusion protein containing collagen I TME linker BI-057 which was 10-fold higher than recovery of chimeric IL-12 Fc fusion protein BI-065 suggesting increased retention of BI-057 within the slices in comparison to BI-065.

### Example 14

In Vivo Comparison of Molecules Containing Collagen I TME Linker and these Devoid of it.

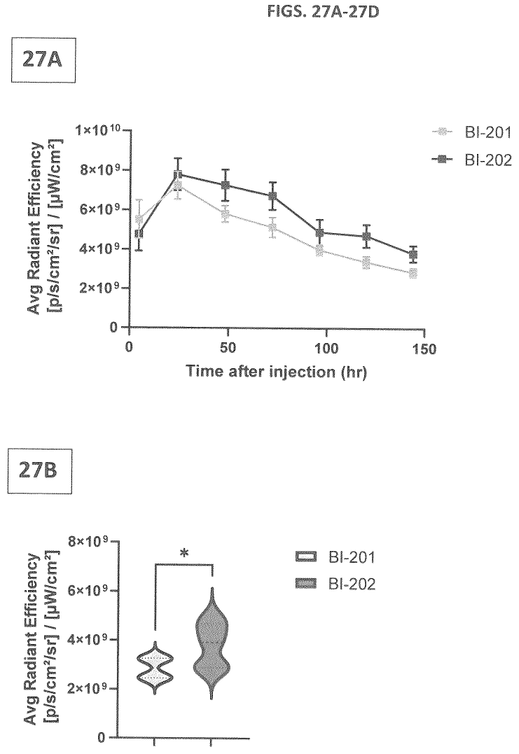

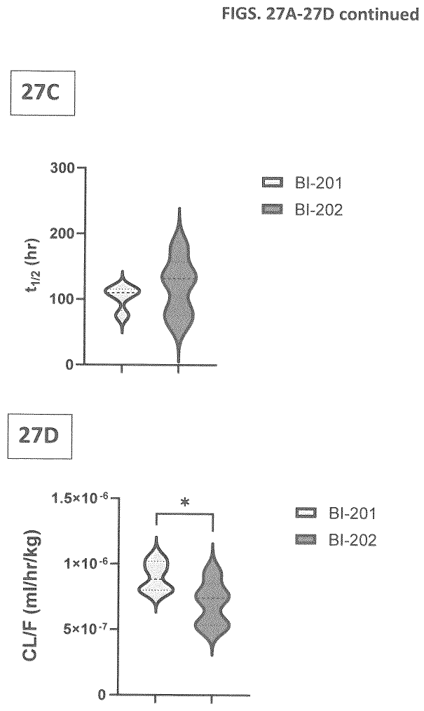

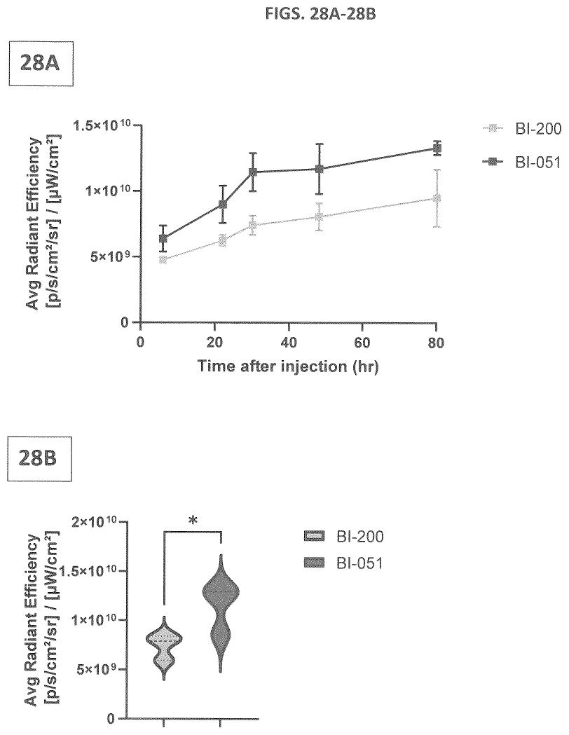

### FIGS. 27A-27D-28A-28B

Retention of molecules containing collagen I TME linker was further evaluated in vivo in syngeneic models. It is important to highlight, that syngeneic models contain a very low level of collagen contrary to human tumors. As a result, it remains elusive to observe a full potential of collagen I TME linkers in those models. To evaluate tumor retention in vivo, fluorescence measurement of Dylight650-labelled chimeric IL-12 Fc fusion protein BI-201 or chimeric IL-12 Fc fusion protein containing collagen I TME linker BI-202 was employed.

Briefly, fusion proteins are labeled with DyLight650 (Thermo Fisher Scientific). Excess dye is removed using a spin column with Purification Resin (Thermo Fisher Scientific), and degree of labeling for each protein is calculated. Proteins compared in pharmacokinetic and retention studies contain equimolar dye. To assess protein retention, mice are imaged with IVIS under auto-exposure epi-illumination fluorescence settings. During this time, mice are maintained on a chlorophyll low diet (Altromin) to minimize gastrointestinal background fluorescence. Image analysis to determine total radiant efficiency is performed using Living Image (Perkin Elmer).

Mice bearing KPCY tumors were injected intravenously with 30 μg of proteins and fluorescence was measured at different time points. As presented in FIG. 27A, up to 24 h the amount of proteins, as measured by fluorescence, in animals of both groups was similar. From 48 h onwards there was increased level of fluorescence in animals injected with chimeric IL-12 Fc fusion protein containing collagen I TME linker BI-202. The fluorescence level at the end of the experiment (at 144 h) in group injected with BI-202 was significantly higher than in group injected with BI-201 (FIG. 27B). In addition, pharmacokinetic measurement (based on fluorescence data) suggested trend towards increased half life and showed significantly reduced clearance of BI-202 in comparison to BI-201 (FIGS. 27C and D).

Retention of human molecules was also evaluated in EMT6-bearing animals. Mice were injected intravenously with 50 μg of Dylight650-labelled human IL-12 Fc fusion protein BI-200 or human IL-12 Fc fusion protein containing collagen I TME linker BI-051. Fluorescence was measured at different time points. As presented in FIG. 28A, already at 24 h after injections there was increased fluorescence in mice injected with BI-051, with the peak at 30 h. At this time point, there were significant differences in the level of fluorescence between mice injected with BI-051 and BI-200 suggesting increased retention of the former (FIG. 28B).

### Example 15

### In Vivo Effect of Collagen I TME Linker on Molecule Functionality/Safety.

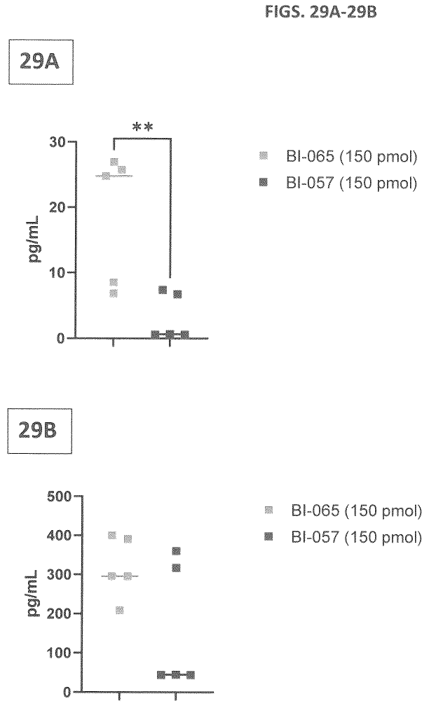

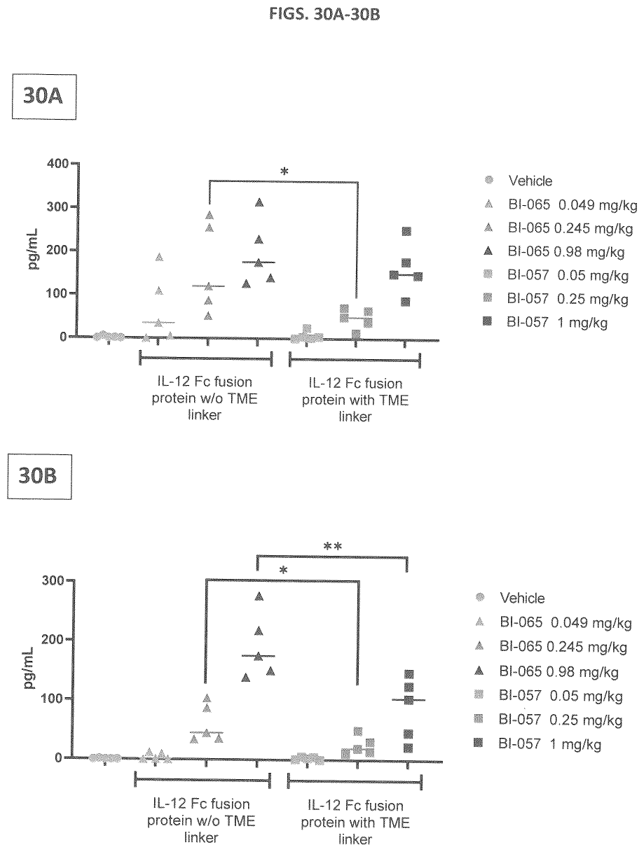

### FIGS. 29A-29B-30A-30B

To evaluate the effect of collagen I TME linker on function/safety of the molecule, mice bearing PDA30364 pancreatic tumors were injected intratumorally with 150 pmol of either chimeric IL-12 Fc fusion protein (BI-065) or chimeric IL-12 Fc fusion protein containing collagen I TME linker (BI-057). To determine safety profile of injected molecules, 48 h after injections blood was collected and IL-12-induced cytokines/chemokines were evaluated.

Briefly, cytokines in sera are measured using LegendPlex Mouse Cytokine Release Syndrome Panel (13-plex) (BioLegend), a bead based immunoassay. Each bead in a multiplex can be differentiated by size and internal fluorescence intensities. Beads are coated with specific antibodies on its surface and serve as the capture bead for that particular analyte. Premixed beads are incubated with the sample or serially diluted premixed standard for 2 h. To determine the concentration of a particular analyte after a washing step, a biotinylated detection antibody cocktail is added. The detection antibody binds to its specific analyte bound on the capture beads thus forming capture bead-analyte-detection antibody sandwiches. Streptavidin-phycoerythrin (SA-PE) is subsequently added which binds to the biotinylated detection antibodies. The fluorescent signal intensities are in proportion to the amount of bound analyte. The LegendPlex is measured on a BD LSR Fortesssa Cell Analyzer. The concentration of a particular analyte is determined using a standard curve. Analysis is done using FlowJo (LLC) and GraphPad Prism (GraphPad Software Inc.).

The cytokine responsible for IL-12-related adverse effects is IFNγ. As shown in FIG. 29A, injection of BI-065 resulted in 20-fold increased level of IFNγ in the periphery compared to animals treated with BI-057. In addition, CXCL10 which is IFNγ-induced chemokine was ca. 10-fold increased in BI-065-treated animals in comparison to mice injected with BI-057.

In further steps, the influence of chimeric molecules on induction of IFNγ was evaluated in animals bearing orthotopic mammary EMT6 tumors. To this end, animals were injected intravenously with different doses of either chimeric IL-12 Fc fusion protein (BI-065) or chimeric IL-12 Fc fusion protein containing collagen I TME linker (BI-057). Blood was collected at 24 h (FIG. 30A) and 72 h (FIG. 30B) after injections and levels of IFNγ was determined. As shown in FIG. 30A, already 24 h after injections there is a clear trend of increased levels of IFNγ in mice treated with BI-065 which reached statistical significance at the medium dose tested. Better safety profile of BI-065, as defined by lower levels of IFNγ, was also observed at the later time point (FIG. 30B).

TABLE 19
(additional molecues used in Examples 13-15)

| | |
|---|---|
| BI-200 Fc Knob | EPKSSDKTHTCPPCPAPEAAGGPSVFLFPPKPKDTLMISRTPEVTCVVVDVSHEDPE |
| SEQ ID NO:480 | VKFNWYVDGVEVHNAKTKPREEQYNSTYRVVSVLTVLHQDWLNGKEYKCKVSN |
| | KALPAPIEKTISKAKGQPREPQVYTLPPSREEMTKNQVSLWCLVKGFYPSDIAVEW |
| | ESNGQPENNYKTTPPVLDSDGSFFLYSKLTVDKSRWQQGNVFSCSVMHEALHNHY |
| | TQKSLSLSPGGGGGSGGGGGPLGVRGGGGGSIWELKKDVYVVELDWYPDAPGEM |
| | VVLTCDTPEEDGITWTLDQSSEVLGSGKTLTIQVKEFGDAGQYTCHKGGEVLSHSL |
| | LLLHKKEDGIWSTDILKDQKEPKNKTFLRCEAKNYSGRFTCWWLTTISTDLTFSVK |
| | SSRGSSDPQGVTCGAATLSAERVRGDNKEYEYSVECQEDSACPAAEESLPIEVMVD |
| | AVHKLKYENYTSSFFIRDIIKPDPPKNLQLKPLKNSRQVEVSWEYPDTWSTPHSYFS |
| | LTFCVQVQGKSKREKKDRVFTDKTSATVICRKNASISVRAQDRYYSSSWSEWASV |
| | PCSGGGGSGGGGSGGGGSRNLPVATPDPGMFPCLHHSQNLLRAVSNMLQKARQTL |
| | EFYPCTSEEIDHEDITKDKTSTVEACLPLELTKNESCLNSRETSFITNGSCLASRKTSF |
| | MMALCLSSIYEDLKMYQVEFKTMNAKLLMDPKRQIFLDQNMLAVIDELMQALNF |
| | NSETVPQKSSLEEPDFYKTKIKLCILLHAFRIRAVTIDRVMSYLNAS |
| BI-200 Fc Hole | EPKSSDKTHTCPPCPAPEAAGGPSVFLFPPKPKDTLMISRTPEVTCVVVDVSHEDPE |
| SEQ ID NO:481 | VKFNWYVDGVEVHNAKTKPREEQYNSTYRVVSVLTVLHQDWLNGKEYKCKVSN |
| | KALPAPIEKTISKAKGQPREPQVYTLPPSREEMTKNQVSLSCAVKGFYPSDIAVEWE |
| | SNGQPENNYKTTPPVLDSDGSFFLVSKLTVDKSRWQQGNVFSCSVMHEALHNRFT |
| | QKSLSLSPGGGGGSGGGGSGGGGSGGGGSGGGGSQVQLVESGGGLVQAGGSLRLS |
| | CAASGRTFETIAMGWFRQVPGKEREFVAVIRGSGVATYYPDSVKGRFTISKDSAKN |
| | TVYLQMNNLKPEDTAVYYCAATTNRFKNTDYTTYDYWGQGTQVTVSSSG |
| BI-201 Fc Knob | MGWSCIILFLVATATGVHSEPKSSDKTHTCPPCPAPEAAGGPSVFLFPPKPKDTLMIS |
| SEQ ID NO:482 | RTPEVTCVVVDVSHEDPEVKFNWYVDGVEVHNAKTKPREEQYNSTYRVVSVLTV |
| | LHQDWLNGKEYKCKVSNKALPAPIEKTISKAKGQPREPQVYTLPPSREEMTKNQVS |
| | LWCLVKGFYPSDIAVEWESNGQPENNYKTTPPVLDSDGSFFLYSKLTVDKSRWQQ |
| | GNVFSCSVMHEALHNRFTQKSLSLSPGGGGGSGGGGGVRLGPGGGGGSIWELKKD |
| | VYVVELDWYPDAPGEMVVLTCDTPEEDGITWTLDQSSEVLGSGKTLTIQVKEFGD |
| | AGQYTCHKGGEVLSHSLLLLHKKEDGIWSTDILKDQKEPKNKTFLRCEAKNYSGRF |
| | TCWWLTTISTDLTFSVKSSRGSSDPQGVTCGAATLSAERVRGDNKEYEYSVECQED |
| | SACPAAEESLPIEVMVDAVHKLKYENYTSSFFIRDIIKPDPPKNLQLKPLKNSRQVE |
| | VSWEYPDTWSTPHSYFSLTFCVQVQGKSKREKKDRVFTDKTSATVICRKNASISVR |
| | AQDRYYSSSWSEWASVPCSGGGGSGGGGSGGGGSRVIPVSGPARCLSQSRNLLKT |
| | TDDMVKTAREKLKHYSCTAEDIDHEDITRDQTSTLKTCLPLELHKNESCLATRETSS |
| | TTRGSCLPPQKTSLMMTLCLGSIYEDLKMYQTEFQAINAALQNHNHQQIILDKGML |
| | VAIDELMQSLNHNGETLRQKPPVGEADPYRVKMKLCILLHAFSTRVVTINRVMGY |
| | LSSA |
| BI-201 Fc Hole | MGWSCIILFLVATATGVHSEPKSSDKTHTCPPCPAPEAAGGPSVFLFPPKPKDTLMIS |
| SEQ ID NO:483 | RTPEVTCVVVDVSHEDPEVKFNWYVDGVEVHNAKTKPREEQYNSTYRVVSVLTV |
| | LHQDWLNGKEYKCKVSNKALPAPIEKTISKAKGQPREPQVYTLPPSREEMTKNQVS |
| | LSCAVKGFYPSDIAVEWESNGQPENNYKTTPPVLDSDGSFFLVSKLTVDKSRWQQ |
| | GNVFSCSVMHEALHNHYTQKSLSLSPGGGGGSGGGGSGGGGSGGGGSGGGGSEV |
| | QLVESGGGLVQPGGSLRLSCAASGRTFETIAMGWFRQAPGKGLEFVAVIRGSGVAT |
| | YYAESVKGRFTISKDSSKNTVYLQMNSLRAEDTAVYYCAATTNRFKNTDYTTYDY |
| | WGQGTQVTVSSAA |
| BI-202 Fc Knob | MGWSCIILFLVATATGVHSEPKSSDKTHTCPPCPAPEAAGGPSVFLFPPKPKDTLMIS |
| SEQ ID NO:484 | RTPEVTCVVVDVSHEDPEVKFNWYVDGVEVHNAKTKPREEQYNSTYRVVSVLTV |
| | LHQDWLNGKEYKCKVSNKALPAPIEKTISKAKGQPREPQVYTLPPSREEMTKNQVS |
| | LWCLVKGFYPSDIAVEWESNGQPENNYKTTPPVLDSDGSFFLYSKLTVDKSRWQQ |
| | GNVFSCSVMHEALHNRFTQKSLSLSPGGGGGSGGGGGVRLGPGGGGGSIWELKKD |
| | VYVVELDWYPDAPGEMVVLTCDTPEEDGITWTLDQSSEVLGSGKTLTIQVKEFGD |
| | AGQYTCHKGGEVLSHSLLLLHKKEDGIWSTDILKDQKEPKNKTFLRCEAKNYSGRF |
| | TCWWLTTISTDLTFSVKSSRGSSDPQGVTCGAATLSAERVRGDNKEYEYSVECQED |
| | SACPAAEESLPIEVMVDAVHKLKYENYTSSFFIRDIIKPDPPKNLQLKPLKNSRQVE |
| | VSWEYPDTWSTPHSYFSLTFCVQVQGKSKREKKDRVFTDKTSATVICRKNASISVR |
| | AQDRYYSSSWSEWASVPCSGGGGSGGGGSGGGGSRVIPVSGPARCLSQSRNLLKT |
| | TDDMVKTAREKLKHYSCTAEDIDHEDITRDQTSTLKTCLPLELHKNESCLATRETSS |
| | TTRGSCLPPQKTSLMMTLCLGSIYEDLKMYQTEFQAINAALQNHNHQQIILDKGML |
| | VAIDELMQSLNHNGETLRQKPPVGEADPYRVKMKLCILLHAFSTRVVTINRVMGY |
| | LSSAGSGGLRELHLDNN |
| BI-202 Fc Hole | MGWSCIILFLVATATGVHSEPKSSDKTHTCPPCPAPEAAGGPSVFLFPPKPKDTLMIS |
| SEQ ID NO:485 | RTPEVTCVVVDVSHEDPEVKFNWYVDGVEVHNAKTKPREEQYNSTYRVVSVLTV |
| | LHQDWLNGKEYKCKVSNKALPAPIEKTISKAKGQPREPQVYTLPPSREEMTKNQVS |
| | LSCAVKGFYPSDIAVEWESNGQPENNYKTTPPVLDSDGSFFLVSKLTVDKSRWQQ |
| | GNVFSCSVMHEALHNHYTQKSLSLSPGGGGGSGGGGSGGGGSGGGGSGGGGSEV |
| | QLVESGGGLVQPGGSLRLSCAASGRTFETIAMGWFRQAPGKGLEFVAVIRGSGVAT |
| | YYAESVKGRFTISKDSSKNTVYLQMNSLRAEDTAVYYCAATTNRFKNTDYTTYDY |
| | WGQGTQVTVSSAA |

1. An Interleukin-12 (IL-12) Fc fusion protein comprising a first polypeptide chain and a second polypeptide chain, wherein
a) the first polypeptide chain comprises a first Fc domain and an IL-12p35 subunit and an IL-12p40 subunit of IL-12, and
b) the second polypeptide chain comprises a second Fc domain and a masking moiety that binds to the IL-12p35 and/or IL-12p40 subunit in the first polypeptide chain;
wherein the first and second polypeptide chain are linked via the first Fc domain and the second Fc domain,
wherein the IL-12p35 subunit or the IL-12p40 subunit is linked to the C-terminus of the first Fc domain via a first peptide linker, which first peptide linker is protease-cleavable,
wherein the masking moiety is linked to the C-terminus of the second Fc domain via a second linker, preferably a peptide linker, and
wherein the first or the second polypeptide chain further comprises a binding moiety selected from the group consisting of: a collagen binding moiety, a heparin binding moiety, and a fibronectin binding moiety.

2. The IL-12 Fc fusion protein according to claim 1, wherein the binding moiety is linked to the C-terminus of the IL-12p35 subunit or to the C-terminus of the IL-12p40 subunit, or the binding moiety is linked to the C-terminus of the masking moiety, and in each case optionally via a third polypeptide linker.

3. The IL-12 Fc fusion protein according to claim 1, wherein the binding moiety is located between the IL-12p35 subunit and the IL-12p40 subunit, or the binding moiety is located between the C-terminus of the first Fc domain and the N-terminus of the IL-12p35 subunit or the N-terminus of the IL-12p40 subunit, and in either case the binding moiety may be optionally flanked on one or both sides by a linker or linkers, preferably a peptide linker.

4. The IL-12 Fc fusion protein according to claim 1, wherein the binding moiety is a collagen binding moiety.

5. The IL-12 Fc fusion protein according to claim 4, wherein the collagen binding moiety binds to collagen I.

6. The IL-12 Fc fusion protein according to claim 5, wherein the collagen binding moiety binds to collagen I and has the sequence LxxLxLxxN (SEQ ID NO:41), wherein L is Leucine and N is Asparagine and x is any amino acid.

7. The IL-12 Fc fusion protein according to claim 6, wherein the collagen binding moiety has a length of 20 amino acids (aa), 19aa, 18aa, 17aa, 16aa, 15aa, 14aa, 13aa, 12aa, 11aa, 10a, or 9aa.

8. The IL-12 Fc fusion protein according to claim 1, wherein the collagen binding moiety comprises or consists of any one of the amino acid sequences of SEQ ID NOs:40-47.

9. The IL-12 Fc fusion protein according to claim 1, wherein the binding moiety is a heparin binding moiety.

10. The IL-12 Fc fusion protein according to claim 9, wherein the heparin binding moiety has the sequence VRIQRKKEKMKET (SEQ ID NO:50).

11. The IL-12 Fc fusion protein according to claim 4, wherein the collagen binding moiety binds to collagen IV.

12. The IL-12 Fc fusion protein according to claim 11, wherein the collagen binding moiety has the sequence KLWVLPK (SEQ ID NO:40).

13. The IL-12 Fc fusion protein according to claim 1, wherein the binding moiety is a fibronectin binding moiety.

14. The IL-12 Fc fusion protein according to claim 13, wherein the fibronectin binding moiety has the sequence GGWSHW (SEQ ID NO:49).

15. The IL-12 Fc fusion protein according to claim 1, wherein the IL-12p35 subunit and the IL-12p40 subunit are human.

16. The IL-12 Fc fusion protein according to claim 1, wherein the IL-12p35 subunit comprises a polypeptide having at least 95% identity to SEQ ID NO:1 and the IL-12p40 subunit comprises a polypeptide having at least 95% identity to SEQ ID NO:2, preferably the IL-12p35 subunit comprises the polypeptide of SEQ ID NO:1 and the IL-12p40 subunit comprises the polypeptide of SEQ ID NO:2.

17. The IL-12 Fc fusion protein according to claim 1, wherein the IL-12p40 subunit and the IL-12p35 subunit are linked in a single-chain having the configuration IL-12p40-IL-12p35 or IL-12p35-IL-12p40.

18. The IL-12 Fc fusion protein according to claim 17, wherein the single-chain IL-12p40-IL-12p35 is linked via its IL-12p40 subunit to the C-terminus of the first Fc domain, or the single-chain IL-12p35-IL-12p40 is linked via its IL-12p35 subunit to the first Fc domain, and in both cases via the first peptide linker, which first peptide linker is protease-cleavable.

19. The IL-12 Fc fusion protein according to claim 17, wherein the IL-12p40 subunit and the IL-12p35 subunit are linked to each other via a linker that is rich in amino acid residues glycine and serine, preferably having a length of 5 to 20 amino acids and only including the amino acids glycine and serine, more preferably a glycine and serine linker having the amino acid sequence of SEQ ID NO:22.

20. The IL-12 Fc fusion protein according to claim 17, wherein the single-chain IL-12p40-IL-12p35 comprises a polypeptide having at least 95% identity to SEQ ID NO:8, or the single-chain IL-12p35-IL-12p40 comprises a polypeptide having at least 95% identity to SEQ ID NO:9.

21. The IL-12 Fc fusion protein according to claim 1, wherein the second peptide linker is not protease-cleavable.

22. The IL-12 Fc fusion protein according to claim 1, wherein the masking moiety binds to the IL-12p40 subunit and is selected from the group consisting of: an IL-12 receptor or an IL-12p40 binding fragment thereof, an scFv, or an immunoglobulin single variable domain, preferably a VHH.

23. The IL-12 Fc fusion protein according to claim 1, wherein the first and the second Fc domain each comprise one or more mutations that promote heterodimerization of the Fc domains.

24. The IL-12 Fc fusion protein according to claim 23, wherein (a) the first Fc domain is a human IgG₁ Fc domain comprising the mutation T366W and the second Fc domain is a human IgG₁ Fc domain comprising the mutations T366S, L368A and Y407V, or (b) the first Fc domain is a human IgG₁ Fc domain comprising the mutations T366S, L368A and Y407V and the second Fc domain is a human IgG₁ Fc domain comprising the mutation T366W.

25. The IL-12 Fc fusion protein according to claim 1, wherein the first and the second Fc domain are human IgG₁ Fc domains and one of the first or the second Fc domain comprises the mutations H435R and Y436F.

26. The IL-12 Fc fusion protein according to claim 1, wherein the first and the second Fc domain are human IgG₁ Fc domains and either the first Fc domain, or the second Fc domain, or both Fc domains comprise the mutations L234A and L235A.

27. The IL-12 Fc fusion protein according to claim 1, wherein the first Fc domain comprises the amino acid sequence of SEQ ID NO:15 and the second Fc domain comprises the amino acid sequence of SEQ ID NO:16, OR the first Fc domain comprises the amino acid sequence of SEQ ID NO:17 and the second Fc domain comprises the amino acid sequence of SEQ ID NO:18, OR the first Fc domain comprises the amino acid sequence of SEQ ID NO:16 and the second Fc domain comprises the amino acid sequence of SEQ ID NO:15, OR the first Fc domain comprises the amino acid sequence of SEQ ID NO:18 and the second Fc domain comprises the amino acid sequence of SEQ ID NO:17.

28. The IL-12 Fc fusion protein according to claim 1, wherein the protease-cleavable linker is cleavable by a matrix metalloproteinase (MMP), preferably an MMP-2, MMP-9, or MMP-13.

29. The IL-12 Fc fusion protein according to claim 28, wherein the protease-cleavable linker comprises or consists of any one of the amino acid sequences of SEQ ID NOs:232-241.

30. An IL-12 Fc fusion protein comprising a first polypeptide chain and a second polypeptide chain, wherein
a) the first polypeptide chain comprises or consists of the amino acid sequence of SEQ ID NO:208 and the second polypeptide chain comprises or consists of the amino acid sequence of SEQ ID NO:209,
b) the first polypeptide chain comprises or consists of the amino acid sequence of SEQ ID NO:210 and the second polypeptide chain comprises or consists of the amino acid sequence of SEQ ID NO:211,
c) the first polypeptide chain comprises or consists of the amino acid sequence of SEQ ID NO:212 and the second polypeptide chain comprises or consists of the amino acid sequence of SEQ ID NO:213,
d) the first polypeptide chain comprises or consists of the amino acid sequence of SEQ ID NO:214 and the second polypeptide chain comprises or consists of the amino acid sequence of SEQ ID NO:215,
e) the first polypeptide chain comprises or consists of the amino acid sequence of SEQ ID NO:216 and the second polypeptide chain comprises or consists of the amino acid sequence of SEQ ID NO:217,
f) the first polypeptide chain comprises or consists of the amino acid sequence of SEQ ID NO:218 and the second polypeptide chain comprises or consists of the amino acid sequence of SEQ ID NO:219,
g) the first polypeptide chain comprises or consists of the amino acid sequence of SEQ ID NO:220 and the second polypeptide chain comprises or consists of the amino acid sequence of SEQ ID NO:221,
h) the first polypeptide chain comprises or consists of the amino acid sequence of SEQ ID NO:222 and the second polypeptide chain comprises or consists of the amino acid sequence of SEQ ID NO:223,
i) the first polypeptide chain comprises or consists of the amino acid sequence of SEQ ID NO:224 and the second polypeptide chain comprises or consists of the amino acid sequence of SEQ ID NO:225,
j) the first polypeptide chain comprises or consists of the amino acid sequence of SEQ ID NO:226 and the second polypeptide chain comprises or consists of the amino acid sequence of SEQ ID NO:227,
k) the first polypeptide chain comprises or consists of the amino acid sequence of SEQ ID NO:228 and the second polypeptide chain comprises or consists of the amino acid sequence of SEQ ID NO:229,
l) the first polypeptide chain comprises or consists of the amino acid sequence of SEQ ID NO:230 and the second polypeptide chain comprises or consists of the amino acid sequence of SEQ ID NO:231, OR
m) the first polypeptide chain comprises or consists of the amino acid sequence of SEQ ID NO:242 and the second polypeptide chain comprises or consists of the amino acid sequence of SEQ ID NO:243.

31. The IL-12 Fc fusion protein according to claim 1, wherein the masking moiety comprises an IL-12 binding immunoglobulin single variable domain comprising the three CDRs contained within any one of the sequences of SEQ ID NOs:61-109.

32. The IL-12 Fc fusion protein according to claim 31, wherein said immunoglobulin single variable domain comprises any one of the amino acid sequences of SEQ ID NOs:61-109.

33. A cleavage product capable of binding to a human IL-12 receptor comprising the IL-12 cytokine after proteolytic cleavage of the cleavable linker as defined in the IL-12 Fc fusion protein of claim 1.

34. The cleavage product according to claim 33 comprising the IL-12 cytokine and the binding moiety.

35. The cleavage product according to claim 34 comprising or consisting of the amino acid sequence of any one of SEQ ID NOs:208, 210, 212, 214, 216, 218, 220, 222, 224, 226, 228, 230 or 242 after proteolytic cleavage of the cleavable linker.

36. An IL-12 binding immunoglobulin single variable domain comprising the three CDRs contained within any one of the sequences of SEQ ID NOs:61-109.

37. The IL-12 binding immunoglobulin single variable domain of claim 36, wherein said immunoglobulin single variable domain is a VHH.

38. The IL-12 binding immunoglobulin single variable domain of claim 36, wherein said immunoglobulin single variable domain comprises the amino acid sequence of any one of SEQ ID NOs:61-109.

39. A nucleic acid encoding at least one polypeptide of the IL-12 Fc fusion protein of claim 1, or a nucleic acid encoding one of the polypeptide chains of an IL-12 Fc fusion protein of claim 1.

40. A vector comprising the nucleic acid of claim 39, optionally wherein the vector comprises nucleic acids encoding both chains of the IL-12 Fc fusion protein.

41. A host cell comprising the nucleic acid of claim 39, optionally wherein the cell comprises one or more nucleic acids encoding both chains of the IL-12 Fc fusion protein.

42. A method of producing an IL-12 Fc fusion protein comprising culturing the host cell of claim 41 under a condition that produces the fusion protein and optionally purifying said IL-12 Fc fusion protein.

43. A composition comprising the IL-12 Fc fusion protein of claim 1.

44. A pharmaceutical composition comprising the IL-12 Fc fusion protein of claim 1 and a pharmaceutically acceptable carrier.

45. A kit comprising the IL-12 Fc fusion protein of claim 1.

46. (canceled)

47. A therapeutic method comprising administering an effective amount of the A cleavage product as defined in claim 33 to a patient in need thereof.

48. A method of treating or reducing the incidence of cancer in a subject, the method comprising administering to the subject an effective amount of an IL-12 Fc fusion protein according to claim 1.

49. (canceled)

50. (canceled)

51. (canceled)

52. A nucleic acid encoding a polypeptide comprising an IL-12 binding immunoglobulin single variable domain of claim 36.

53. A vector comprising the nucleic acid of claim 36.

54. A host cell comprising the nucleic acid of claim 36.

55. A method of producing a polypeptide comprising the IL-12 binding immunoglobulin single variable domain, the polypeptide comprising culturing the host cell of claim 54 under a condition that produces said polypeptide, and optionally purifying said polypeptide.

56. A composition comprising a polypeptide comprising the IL-12 binding immunoglobulin single variable domain of claim 36.

57. A method of treating or reducing the incidence of cancer in a subject, the method comprising administering to the subject an effective amount of a polypeptide comprising the IL-12 binding immunoglobulin single variable domain according to claim 36.


SEQUENCE LISTING (485 sequences)

SEQ ID NO: 1 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 2 - protein, 306 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 3 - protein, 503 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 4 - protein, 503 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 5 - protein, 193 aa, Mus musculus
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 6 - protein, 499 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 7 - protein, 499 aa, synthetic construct
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 8 - protein, 518 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 9 - protein, 518 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 10 - protein, 514 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 11 - protein, 514 aa, synthetic construct
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 12 - protein, 662 aa, Homo sapiens
MEPLVTWVVPLLFLFLLSRQGAACRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 13 - protein, 862 aa, Homo sapiens
MAHTFRGCSLAFMFIITWLLIKAKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGMLDVWYMKRHIDYSRQQISLFWKNLSVSEARGKILHYQVTLQELTGGKAMTQNITGHTSWTTVIPRTGNWAVAVSAANSKGSSLPTRINIMNLCEAGLLAPRQVSANSEGMDNILVTWQPPRKDPSAVQEYVVEWRELHPGGDTQVPLNWLRSRPYNVSALISENIKSYICYEIRVYALSGDQGGCSSILGNSKHKAPLSGPHINAITEEKGSILISWNSIPVQEQMGCLLHYRIYWKERDSNSQPQLCEIPYRVSQNSHPINSLQPRVTYVLWMTALTAAGESSHGNEREFCLQGKANWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 14 - protein, 231 aa, Homo sapiens
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 231 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 231 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 17 - protein, 231 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 231 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 19 - protein, 4 aa, synthetic construct
GGGS

SEQ ID NO: 20 - protein, 6 aa, synthetic construct
GGSGGS

SEQ ID NO: 21 - protein, 10 aa, synthetic construct
GGGGSGGGGS

SEQ ID NO: 22 - protein, 15 aa, synthetic construct
GGGGSGGGGSGGGGS

SEQ ID NO: 23 - protein, 20 aa, synthetic construct
GGGGSGGGGSGGGGSGGGGS

SEQ ID NO: 24 - protein, 9 aa, synthetic construct
GGGGSGGGG

SEQ ID NO: 25 - protein, 4 aa, synthetic construct
GSGG

SEQ ID NO: 26 - protein, 25 aa, synthetic construct
GGGGSGGGGSGGGGSGGGGSGGGGS

SEQ ID NO: 27 - protein, 5 aa, synthetic construct
GGGGS

SEQ ID NO: 28 - protein, 7 aa, synthetic construct
SGGSGGS

SEQ ID NO: 29 - protein, 8 aa, synthetic construct
GGGSGGGS

SEQ ID NO: 30 - protein, 9 aa, synthetic construct
GGGGSGGGS

SEQ ID NO: 31 - protein, 10 aa, synthetic construct
GGGGSGGGGS

SEQ ID NO: 32 - protein, 15 aa, synthetic construct
GGGGSGGGGSGGGGS

SEQ ID NO: 33 - protein, 18 aa, synthetic construct
GGGGSGGGGSGGGGGGGS

SEQ ID NO: 34 - protein, 20 aa, synthetic construct
GGGGSGGGGSGGGGSGGGGS

SEQ ID NO: 35 - protein, 25 aa, synthetic construct
GGGGSGGGGSGGGGSGGGGSGGGGS

SEQ ID NO: 36 - protein, 30 aa, synthetic construct
GGGGSGGGGSGGGGSGGGGSGGGGSGGGGS

SEQ ID NO: 37 - protein, 35 aa, synthetic construct
GGGGSGGGGSGGGGSGGGGSGGGGSGGGGSGGGGS

SEQ ID NO: 38 - protein, 1464 aa, Homo sapiens
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 39 - protein, 1366 aa, Homo sapiens
MLSFVDTRTLLLLAVTLCLATCQSLQEETVRKGPAGDRGPRGERGPPGPPGRDGEDGPTGPPGPPGPPGPPGLGGNFAAQYDGKGVGLGPGPMGLMGPRGPPGAAGAPGPQGFQGPAGEPGEPGQTGPAGARGPAGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPIGSAGPPGFPGAPGPKGEIGAVGNAGPAGPAGPRGEVGLPGLSGPVGPPGNPGANGLTGAKGAAGLPGVAGAPGLPGPRGIPGPVGAAGATGARGLVGEPGPAGSKGESGNKGEPGSAGPQGPPGPSGEEGKRGPNGEAGSAGPPGPPGLRGSPGSRGLPGADGRAGVMGPPGSRGASGPAGVRGPNGDAGRPGEPGLMGPRGLPGSPGNIGPAGKEGPVGLPGIDGRPGPIGPAGARGEPGNIGFPGPKGPTGDPGKNGDKGHAGLAGARGAPGPDGNNGAQGPPGPQGVQGGKGEQGPPGPPGFQGLPGPSGPAGEVGKPGERGLHGEFGLPGPAGPRGERGPPGESGAAGPTGPIGSRGPSGPPGPDGNKGEPGVVGAVGTAGPSGPSGLPGERGAAGIPGGKGEKGEPGLRGEIGNPGRDGARGAPGAVGAPGPAGATGDRGEAGAAGPAGPAGPRGSPGERGEVGPAGPNGFAGPAGAAGQPGAKGERGAKGPKGENGVVGPTGPVGAAGPAGPNGPPGPAGSRGDGGPPGMTGFPGAAGRTGPPGPSGISGPPGPPGPAGKEGLRGPRGDQGPVGRTGEVGAVGPPGFAGEKGPSGEAGTAGPPGTPGPQGLLGAPGILGLPGSRGERGLPGVAGAVGEPGPLGIAGPPGARGPPGAVGSPGVNGAPGEAGRDGNPGNDGPPGRDGQPGHKGERGYPGNIGPVGAAGAPGPHGPVGPAGKHGNRGETGPSGPVGPAGAVGPRGPSGPQGIRGDKGEPGEKGPRGLPGLKGHNGLQGLPGIAGHHGDQGAPGSVGPAGPRGPAGPSGPAGKDGRTGHPGTVGPAGIRGPQGHQGPAGPPGPPGPPGPPGVSGGGYDFGYDGDFYRADQPRSAPSLRPKDYEVDATLKSLNNQIETLLTPEGSRKNPARTCRDLRLSHPEWSSGYYWIDPNQGCTMDAIKVYCDFSTGETCIRAQPENIPAKNWYRSSKDKKHVWLGETINAGSQFEYNVEGVTSKEMATQLAFMRLLANYASQNITYHCKNSIAYMDEETGNLKKAVILQGSNDVELVAEGNSRFTYTVLVDGCSKKTNEWGKTIIEYKTNKPSRLPFLDIAPLDIGGADQEFFVDIGPVCFK

SEQ ID NO: 40 - protein, 7 aa, synthetic construct
KLWVLPK

SEQ ID NO: 41 - protein, 9 aa, synthetic construct
LXXLXLXXN

SEQ ID NO: 42 - protein, 9 aa, synthetic construct
LSELRLHEN

SEQ ID NO: 43 - protein, 9 aa, synthetic construct
LTELHLDNN

SEQ ID NO: 44 - protein, 9 aa, synthetic construct
LSELRLHNN

SEQ ID NO: 45 - protein, 9 aa, synthetic construct
LSELRLHAN

SEQ ID NO: 46 - protein, 9 aa, synthetic construct
LRELHLNNN

SEQ ID NO: 47 - protein, 9 aa, synthetic construct
LRELHLDNN

SEQ ID NO: 48 - protein, 2477 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPEVPQLTDLSFVDITDSSIGLRWTPLNSSTIIGYRITVVAAGEGIPIFEDFVDSSVGYYTVTGLEPGIDYDISVITLINGGESAPTTLTQQTAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 49 - protein, 6 aa, synthetic construct
GGWSHW

SEQ ID NO: 50 - protein, 13 aa, synthetic construct
VRIQRKKEKMKET

SEQ ID NO: 51 - protein, 446 aa, synthetic construct
EVMLVESGGGLVQPGGSLRLSCTASGFTFSASAMSWVRQAPGKGLEWVAYISGGGGDTYYSSSVKGRFTISRDNAKNSLYLQMNSLRAEDTAVYYCARHSNVNYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 52 - protein, 218 aa, synthetic construct
EIVLTQSPATLSLSPGERATMSCRASENIDTSGISFMNWYQQKPGQAPKLLIYVASNQGSGIPARFSGSGSGTDFTLTISRLEPEDFAVYYCQQSKEVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 446 aa, synthetic construct
EVMLVESGGGLVQPGGSLRLSCTASGFTFSASAMSWVRQAPGKGLEWVAYISGGGGDTYYSSSVKGRFTISRDNAKNSLYLQMNSLRAEDTAVYYCARHSNPNYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 54 - protein, 218 aa, synthetic construct; identical to SEQ ID NO: 52

SEQ ID NO: 55 - protein, 446 aa, synthetic construct
EVMLVESGGGLVQPGGSLRLSCTASGFTFSKSAMSWVRQAPGKGLEWVAYISGGGGDTYYSSSVKGRFTISRDNAKNSLYLQMNSLRAEDTAVYYCARHSNVNYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 56 - protein, 218 aa, synthetic construct
EIVLTQSPATLSLSPGERATMSCRASENIDVSGISFMNWYQQKPGQAPKLLIYVASNQGSGIPARFSGSGSGTDFTLTISRLEPEDFAVYYCQQSKEVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 446 aa, synthetic construct; identical to SEQ ID NO: 55

SEQ ID NO: 58 - protein, 218 aa, synthetic construct; identical to SEQ ID NO: 56

SEQ ID NO: 59 - protein, 446 aa, synthetic construct; identical to SEQ ID NO: 55

SEQ ID NO: 60 - protein, 218 aa, synthetic construct; identical to SEQ ID NO: 56

SEQ ID NO: 61 - protein, 125 aa, synthetic construct
QVQLVESGGGLVQPGGSLRLSCAASGREFTSVSMAWFRQRPGKEREFVAFSARISETTEYADFVKGRFTIWRDNANAKVTVYLQMNNLKPEDTGVYYCAASEGPATIRQNTYPDWGQGTQVTVSS

SEQ ID NO: 62 - protein, 114 aa, synthetic construct
EVQLVESGGGLVQPGGSLRLSCAASGFTFSSYYMYWIRQAPGKGLEWVSTIKPNGSGIYGNSVAGRFTISRDNAKNMLYLQMNMLRPEDTALYYCARDVRGTVRGQGTQVTVSS

SEQ ID NO: 63 - protein, 114 aa, synthetic construct
QVQLVESGGGLVQPGGSLRLSCAASGNQLSLYNMGWYRQAPGKQRELVASISRAGRSSYGDSVKGRFTISRDNAKNTVYLQMSSLKPEDTAVYYCKASFLDDYWGQGTQVTVSS

SEQ ID NO: 64 - protein, 115 aa, synthetic construct
EVQLVESGGGLVQPGGSLRLSCAASGFSFSSSWMFWVRQPPGKGLEWVSTISPSGDYSRYADSVQGRFTISRDNTKNTLSLQMNSLKPEDTALYYCARDLRGTMRGQGTQVTVSS

SEQ ID NO: 65 - protein, 116 aa, synthetic construct
QVQLVESGGGLVQPGGSLRLSCAASGFTFSNFVMKWYHQAPGKERDLVASIDTTHFTNYADSVKGRFTISRDNSKNTVYLQMNSLKSEDTAVYYCKVRRRDYEDYWGQGTQVTVSS

SEQ ID NO: 66 - protein, 124 aa, synthetic construct
EVQLVESGGGLVQAGGSLRLSCAASGRTFSRYNMGWFRQAPGKEREFVAAIIWSGGVINYADSVKGRFTISRDNAKNTVYLQMNSLKPEDTARYYCAADDKYYDRIVRGTADYWGQGTQVTVSS

SEQ ID NO: 67 - protein, 120 aa, synthetic construct
EVQLVESGGGLVQPGGSLRLSCVASGSIGSVVSWGWYRQAPGLERELVASDASGGRPNYQDSVKGRFTISRDSAKNTVYLQMNSLKPEDTAVYYCNLRGLQLDMGLYDSWGQGTQVTVSS

SEQ ID NO: 68 - protein, 123 aa, synthetic construct
EVQLVESGGGLVQAGDSLKLSCATSGRPSRDYAMGWFRQAPGKKRDFVAGISSGGGFTNYADSVKARFTISKDNAKNTVYLQMNSLKPEDTAVYYCAAQSGTNYISRTSPPYWGQGTQVTVSS

SEQ ID NO: 69 - protein, 119 aa, synthetic construct
EVQLVESGGGLVHPGGSLRLSCVASGFRFTPYTMGWYRQAPGKQRELVASISSVYSTNYADSVKGRFTVSRDNVKGTVSLQMNSLKPEDTAVYYCNAPGLLHEEGTDYWGQGTQVTVSS

SEQ ID NO: 70 - protein, 125 aa, synthetic construct
QVQLVESGGGSVQVGGSLRLSCVGSGRTLNMYNMGWFRQAPGKEREFVAAISGKGLISDYRDSVKGRFTISRDNARNTMYLQMNSLKPEDTAVYHCAAGQWSAGPFTRERSYEYWGQGTQVTVSS

SEQ ID NO: 71 - protein, 125 aa, synthetic construct
EVQLVESGGGLVQAGGSLRLSCAASGRTFSRWTMAWFRQAPGKERDFVAAVGWWNDSTYYADSVKGRFTISRDNNENTLYLQMNSLKPEDTAVYICASTEKYGLGQPNPRRYDYWGQGTQVTVSS

SEQ ID NO: 72 - protein, 124 aa, synthetic construct
QVQLVESGGGLVQPGGSLRLSCAASGRTLSSYTMAWFRQAPGKEREFVATISPVGFIMDYADSVKGRFTISRDNTKNTVYLQMNSLKHEDTALYYCATDLGRKLGTQSREYGYWGQGTQVTVSS

SEQ ID NO: 73 - protein, 123 aa, synthetic construct
EVQLVESGGGLVQAGGSLRLSCAASGRTFSTYAVGWFRQAPGKEREFVAAISWGGGTVRYADSVKGRSTISRDDAKNTVYLQMNSLKPEDTAIYYCAARVLHIATKAVDFGSWGQGTQVTVSS

SEQ ID NO: 74 - protein, 125 aa, synthetic construct
EVQLVESGGGSVQVGGSLRLSCVASGRTFNMYVMGWFRQAPGKEREFVAAISGEGLISDYRDSVKGRFTISRDNAKNTMYLQMNSLKPEDTAVYHCAAGQWTNGPFTRERTYEYWGQGTQVTVSS

SEQ ID NO: 75 - protein, 116 aa, synthetic construct
EVQLVESGGGLVQPGGSLRLSCAASGRDFDRSTMAWYRQAPGKQRELVASIPSDIGTKYADSVKGRFFISRVYAKNTNTVYLQMNSLKPEDTAVYYCYAHIDSDYWGQGTQVTVSS

SEQ ID NO: 76 - protein, 123 aa, synthetic construct
EVQLVESGGGLVQPGGSLRLSCAASGRTFSSRAMGWFRQAPGKEREFVAAISFGGGTIRYADSVKGRFTISRDDAKNTVYLQMNSLKPEDTAVYYCAARRLHIATLAADFDSWGQGTQVTVSS

SEQ ID NO: 77 - protein, 127 aa, synthetic construct
EVQLVESGGGLVQAGGSLRLSCSASGRSLNDYIVGWFRQAPGKERELVAAISSGGYIQHYIDSVKGRFTISRDNAKNTVYLQMNGLKPEDTAVYYCAANQLNGVARKKIESDDYDYWGQGTQVTVSS

SEQ ID NO: 78 - protein, 126 aa, synthetic construct
EVQLVESGGGLVEAGGSLRVSCAASGGTFSEYAMGWIRQAPGKEREFVAGISRGAGRTVYADSVKGRFTISRDNHKNTVYLQMNSLKPEDTAVYYCGADDVSYNRVTTAPGEYDYWGQGIQVTVSS

SEQ ID NO: 79 - protein, 125 aa, synthetic construct
EVQLVESGGGAVQAGGALKLSCAYSGRAFSRSLMGWFRQAPGKEREFVAAISWVSVTPDYGDSVKGRFTISRDNAKSTVTLQMNSLKPEDTAVYYCAASERYGTPRRRPNDYDYWGQGTQVTVSS

SEQ ID NO: 80 - protein, 120 aa, synthetic construct
EVQLVESGGGLVQPGGSLRLSCVASGSIGSITSMAWYRQGTGKQRELVASISSGGRPSYQDSVKGRFTISRDNAENTVYLQMNSLKPEDTAVYHCNVRGLHLDTGLYESWGQGTQVTVSS

SEQ ID NO: 81 - protein, 116 aa, synthetic construct
EVQLVESGGGLVQPGGSLRLSCASPGSISTLYVMGWYRQAPGKQRDLVARITRGGSTSYANSVKGRFTISRDNVNNTINLQMNSLKPEDTAVYYCYAQTAVGPDYWGQGTQVTVSS

SEQ ID NO: 82 - protein, 113 aa, synthetic construct
QVQLVESGGGLVQAGGSLRLSCAASGSIFSTLNAIGWYRQAPGKQAELVARITHDGRIVYGDSVKGRFTISRDNAKNTAYLQMNSLNPEDTAVYFCVAPGMVRGQGTQVTVSS

SEQ ID NO: 83 - protein, 127 aa, synthetic construct
QVQLVESGGGLVQAGDSLRLSCTASGRTLTLSMVTVGWFRQGSGKEREFVAAISWRGGRSYVADDVKGRFTISRDNARNTVYLQMNSVKPEDTAVYYCAAREAIQDLAWTANDFTYWGQGTQVTVSS

SEQ ID NO: 84 - protein, 123 aa, synthetic construct
QVQLVESGGGSVQAGGSLRLSCAASGRTFNTKAIGWFRQAPGKEREFVAAISWGGGTIRYADSVKGRVTISRDDAKNTVYLQMNSLKPEDTAVYYFATRQLHIATLAADFDSRRQGTQVTVSS

SEQ ID NO: 85 - protein, 124 aa, synthetic construct
EVQLVESGGGLVQPGGSLRLSCAASGRTLSSYTMAWFRQAPGKEREFVATISPVGFIMDYADSVKGRFTISRDNTKNTVYLQMNSLKHEDTALYYCATDLGRKLGTQSCEYGYWGQGTQVTVSS

SEQ ID NO: 86 - protein, 120 aa, synthetic construct
EVQLVESGGGLVQPGGSLRLSCVASGSTGSITSMAWYRQAPGKQRELVASINSGGRPNYQESVKGRFTISRDNAENTLYLQMNSLSPEDTAVYLCNLRGLRLDTGLYESWGQGTQVTVSS

SEQ ID NO: 87 - protein, 123 aa, synthetic construct
QVQLVESGGGSVQAGGSLRLSCAASGRTFSSRAMGWFRQAPGKEREFVAAISFGGGTIRYADSVKGRFTISRDDAKNTVYLQMNSLKPEDTAVYYCAARRLHIATLAADFDSWGQGTQVTVSS

SEQ ID NO: 88 - protein, 124 aa, synthetic construct
EVQLVESGGGLVQTGGSLRLSCAASGLTNGYVMAWFRQAPGKEREFVSGIGWGSSRTYYADSVKGRFTISRDNAINTVALQMNSLKPEDTAVYYCAAQGRISPIYTRANEYPYWGQGTQVTVSS

SEQ ID NO: 89 - protein, 114 aa, synthetic construct
QVQLVESGGGLVQPGGSLRLSCAASGFTFSSYGMSWYRQAPGKEHELVAGISAGSTKYADSVKGRFTISRDNAKKTVYLQMNSLKPEDTAVYYCSRWPRLFEDWGQGTQVTVSS

SEQ ID NO: 90 - protein, 120 aa, synthetic construct
EVQLVESGGGLVQPGGSLRLSCVASGDIGSMTSTGWYRQAPGKQRDLVASINSGGRPNYQDSVKGRFTISRDSAKNTVYLQMNSLKPEDTAVYYCNLRGLVLSTGLYESWGQGTQVTVSS

SEQ ID NO: 91 - protein, 116 aa, synthetic construct
QVQLVESGGGLVQPGGSLRLSCAAPGSIATLYVMGWYRQAPGKQRELVARITRGGSTSYANAVKGRFTISRDNAKNTVNLQMNSLKPEDTAIYYCNAQTAVGPDYWGQGTQVTVSS

SEQ ID NO: 92 - protein, 124 aa, synthetic construct
EVQLVESGGGLVQAGGSLRPSCAASGRTFSNYNMGWFRQAPGKERESVATISRSGVITDYADSVKGRFTISRDNAKNTVYLQMDSLKPEDTAVYYCAAARSPVWGRGPDEYDTWGQGTQVTVSS

SEQ ID NO: 93 - protein, 124 aa, synthetic construct
EVQLVESGGGLVQAGGSLRLSCAASGRTFSAYVMGWFRQTPGKGREFVAAISTGGQISDYANSVKGRFTISRDNAKNTAYLQMNNLKPEDTAVYYCAANRENFLNRGAGDYEYWGQGTQVTVSS

SEQ ID NO: 94 - protein, 120 aa, synthetic construct
QVQLVESGGGLVQPGGSLRLSCVASGSTGSITSMAWYRQAPGKQRELVASINSGGRPNYQESVKGRFTISRDNAENTLYLQMNSLSPEDTAVYLCNLRGLRLDTGLYESWGQGTQVTVSS

SEQ ID NO: 95 - protein, 117 aa, synthetic construct
QVQLVESGGGLVQPGGSLRLSCAASGSIAEIYVMGWYRQAPGKQREIVATTPSSGRTNIADSVKGRFIISRDFVKNTVALQMNSLKPEDTAVYYCYARLTPTSVASWGPGTQVTVSS

SEQ ID NO: 96 - protein, 123 aa, synthetic construct
EVQLVESGGGSVQAGGSLRLSCATSGRGFSTYAMGWFRQAPGKEREFVAAISFGGGTVRYVDSVKGRFTISRDDAKGTVYLQMNSLKPEDTAVYYCAARRLHIATLAADFGSWGQGTQVTVSS

SEQ ID NO: 97 - protein, 126 aa, synthetic construct
EVQLVESGGGLVQAGDSLRLSCAASGRTFSTYVMGWFRQAPGKEREFVAYISTGGLISDHADSVKGRFTISRDNAKNTVYLQMNSLRPEDTAVYYCAAASRTRRPIATIKDEYDYWGQGTQVTVSS

SEQ ID NO: 98 - protein, 127 aa, synthetic construct
EVQLVESGGGLVQAGDSLRLSCTASGRTLTLSMVTVGWFRQGSGKEREFVAAISWRGGRSSVADDVKGRFTISRDNARNTVYLQMNSVKPEDTAVYYCAARTAIQDLAWTANDFTYWGQGTQVTVSS

SEQ ID NO: 99 - protein, 124 aa, synthetic construct
QVQLVESGGGLVQAGGSLRLSCAASGRTFETIAMGWFRQVPGKEREFVAVIRGSGVATYYPDSVKGRFTISKDSAKNTVYLQMNNLKPEDTAVYYCAATTNRFKNTDYTTYDYWGQGTQVTVSS

SEQ ID NO: 100 - protein, 125 aa, synthetic construct
EVQLVESGGGSVQVGGFLRLSCVGSGRTLNMYNMGWFRQAPGKEREFVAAISGKGLISDYRDSVKGRFTISRDNARNTMYLQMNSLKPEDTAVYHCAAGQWSAGPFTRERSYEYWGQGTQVTVSS

SEQ ID NO: 101 - protein, 124 aa, synthetic construct
EVQLVESGGGLVQAGGSLRLSCASSGRTFSNYNMGWFRQAPGKERESVATISRSGVITDYADYVKGRFTISRDNAKNTVYLQMDSLKPEDTAVYYCAAARSPVWGRGPDEYDTWGQGTQVTVSS

SEQ ID NO: 102 - protein, 123 aa, synthetic construct
EVQLVESGGGSVQAGGSLRLSCAASGRTFNTKAIGWFRQAPGKEREFVAAISWGGGTIRYADSVKGRVTISRDDAKNTVYLQMNSLKPEDTAVYYCAARQLHIATLAADFDSWGQGTQVTVSS

SEQ ID NO: 103 - protein, 126 aa, synthetic construct
EVQLVESGGGLVQAGGSLRLSCAASGRTYSTVAMGWFRQAPGKEREFVGAITWSVGNTAVADSVKGRFAISRDSAKNTVYLQMNSLKVEDTAVYYCASRTNIGAFNLFRENHYNYWGQGTQVTVSS

SEQ ID NO: 104 - protein, 120 aa, synthetic construct
EVQLVESGGGLVQPGGSLRLSCKASGSIGSVTSMGWYRQAPGKQRDLVASADSNGRTTFQDFVQGRFTISRDSAKNTWYLQMNSLKPEDTAVYYCHLRGLQLTMGLYESWGQGTQVTVSS

SEQ ID NO: 105 - protein, 125 aa, synthetic construct
EVQLVESGGGAVQAGGALNVSCAASGRAFSRTLMGWFRQAPGKEREFVAGISWVSVTPDYGNSVKGRFTISRDNAKSTVYLQMNSLKPEDTAVYYCAASQRYGTPRRRPNDYEYWGQGIQVTVSS

SEQ ID NO: 106 - protein, 127 aa, synthetic construct
EVQLVESGGGSVQTGGSLRLSCKVSEGSFMRYNMGWFRQAPGKERDFVAAMSGALALIRYADSVKGRFTISRDNSKNTVYLDMNSLKPEDTAVYYCAADLEPQYWTKAGQRDTYDVWGQGTQVTVSS

SEQ ID NO: 107 - protein, 124 aa, synthetic construct
EVQLVESGGGLVQAGGSLRLSCAASGSISSNIMGWFRQAPGKEREFVAVISRRGLILDYGDSVKGRFTMSRDNAKKAVYLQMNSLKPEDTAVYYCAVGKTTDRFSEIPSDYDYWGQGTQVTVSS

SEQ ID NO: 108 - protein, 124 aa, synthetic construct
EVQLVESGGGLVQPGGSLRLSCAASGRTFETIAMGWFRQAPGKGLEFVAVIRGSGVATYYAESVKGRFTISKDSSKNTVYLQMNSLRAEDTAVYYCAATTNRFKNTDYTTYDYWGQGTQVTVSS

SEQ ID NO: 109 - protein, 124 aa, synthetic construct
EVQLVESGGGLVQPGGSLRLSCAASGRTFETIAMGWFRQAPGKGLEFVAVIRGSGVATYYAESVKGRFTISKDSSKNTVYLQMNSLRAEDTAVYYCAATTNRFKNREYTTYDYWGQGTQVTVSS

SEQ ID NO: 110 - protein, 10 aa, synthetic construct
GREFTSVSMA

SEQ ID NO: 111 - protein, 6 aa, synthetic construct
ARISET

SEQ ID NO: 112 - protein, 10 aa, synthetic construct
GFTFSSYYMY

SEQ ID NO: 113 - protein, 5 aa, synthetic construct
KPNGS

SEQ ID NO: 114 - protein, 10 aa, synthetic construct
GNQLSLYNMG

SEQ ID NO: 115 - protein, 5 aa, synthetic construct
SRAGR

SEQ ID NO: 116 - protein, 10 aa, synthetic construct
GFSFSSSWMF

SEQ ID NO: 117 - protein, 6 aa, synthetic construct
SPSGDY

SEQ ID NO: 118 - protein, 10 aa, synthetic construct
GFTFSNFVMK

SEQ ID NO: 119 - protein, 5 aa, synthetic construct
DTTHF

SEQ ID NO: 120 - protein, 10 aa, synthetic construct
GRTFSRYNMG

SEQ ID NO: 121 - protein, 6 aa, synthetic construct
IWSGGV

SEQ ID NO: 122 - protein, 10 aa, synthetic construct
GSIGSVVSWG

SEQ ID NO: 123 - protein, 5 aa, synthetic construct
ASGGR

SEQ ID NO: 124 - protein, 10 aa, synthetic construct
GRPSRDYAMG

SEQ ID NO: 125 - protein, 6 aa, synthetic construct
SSGGGF

SEQ ID NO: 126 - protein, 10 aa, synthetic construct
GFRFTPYTMG

SEQ ID NO: 127 - protein, 5 aa, synthetic construct
SSVYS

SEQ ID NO: 128 - protein, 10 aa, synthetic construct
GRTLNMYNMG

SEQ ID NO: 129 - protein, 6 aa, synthetic construct
SGKGLI

SEQ ID NO: 130 - protein, 10 aa, synthetic construct
GRTFSRWTMA

SEQ ID NO: 131 - protein, 6 aa, synthetic construct
GWWNDS

SEQ ID NO: 132 - protein, 10 aa, synthetic construct
GRTLSSYTMA

SEQ ID NO: 133 - protein, 6 aa, synthetic construct
SPVGFI

SEQ ID NO: 134 - protein, 10 aa, synthetic construct
GRTFSTYAVG

SEQ ID NO: 135 - protein, 6 aa, synthetic construct
SWGGGT

SEQ ID NO: 136 - protein, 10 aa, synthetic construct
GRTFNMYVMG

SEQ ID NO: 137 - protein, 6 aa, synthetic construct
SGEGLI

SEQ ID NO: 138 - protein, 10 aa, synthetic construct
GRDFDRSTMA

SEQ ID NO: 139 - protein, 5 aa, synthetic construct
PSDIG

SEQ ID NO: 140 - protein, 10 aa, synthetic construct
GRTFSSRAMG

SEQ ID NO: 141 - protein, 6 aa, synthetic construct
SFGGGT

SEQ ID NO: 142 - protein, 10 aa, synthetic construct
GRSLNDYIVG

SEQ ID NO: 143 - protein, 6 aa, synthetic construct
SSGGYI

SEQ ID NO: 144 - protein, 10 aa, synthetic construct
GGTFSEYAMG

SEQ ID NO: 145 - protein, 6 aa, synthetic construct
SRGAGR

SEQ ID NO: 146 - protein, 10 aa, synthetic construct
GRAFSRSLMG

SEQ ID NO: 147 - protein, 6 aa, synthetic construct
SWVSVT

SEQ ID NO: 148 - protein, 10 aa, synthetic construct
GSIGSITSMA

SEQ ID NO: 149 - protein, 5 aa, synthetic construct
SSGGR

SEQ ID NO: 150 - protein, 10 aa, synthetic construct
GSISTLYVMG

SEQ ID NO: 151 - protein, 5 aa, synthetic construct
TRGGS

SEQ ID NO: 152 - protein, 11 aa, synthetic construct
GSIFSTLNAIG

SEQ ID NO: 153 - protein, 5 aa, synthetic construct
THDGR

SEQ ID NO: 154 - protein, 12 aa, synthetic construct
GRTLTLSMVTVG

SEQ ID NO: 155 - protein, 6 aa, synthetic construct
SWRGGR

SEQ ID NO: 156 - protein, 10 aa, synthetic construct
GRTFNTKAIG

SEQ ID NO: 157 - protein, 6 aa, synthetic construct
SWGGGT

SEQ ID NO: 158 - protein, 10 aa, synthetic construct
GRTLSSYTMA

SEQ ID NO: 159 - protein, 6 aa, synthetic construct
SPVGFI

SEQ ID NO: 160 - protein, 10 aa, synthetic construct
GSTGSITSMA

SEQ ID NO: 161 - protein, 5 aa, synthetic construct
NSGGR

SEQ ID NO: 162 - protein, 10 aa, synthetic construct
GRTFSSRAMG

SEQ ID NO: 163 - protein, 6 aa, synthetic construct
SFGGGT

SEQ ID NO: 164 - protein, 9 aa, synthetic construct
GLTNGYVMA

SEQ ID NO: 165 - protein, 6 aa, synthetic construct
GWGSSR

SEQ ID NO: 166 - protein, 10 aa, synthetic construct
GFTFSSYGMS

SEQ ID NO: 167 - protein, 4 aa, synthetic construct
SAGS

SEQ ID NO: 168 - protein, 10 aa, synthetic construct
GDIGSMTSTG

SEQ ID NO: 169 - protein, 5 aa, synthetic construct
NSGGR

SEQ ID NO: 170 - protein, 10 aa, synthetic construct
GSIATLYVMG

SEQ ID NO: 171 - protein, 5 aa, synthetic construct
TRGGS

SEQ ID NO: 172 - protein, 10 aa, synthetic construct
GRTFSNYNMG

SEQ ID NO: 173 - protein, 6 aa, synthetic construct
SRSGVI

SEQ ID NO: 174 - protein, 10 aa, synthetic construct
GRTFSAYVMG

SEQ ID NO: 175 - protein, 6 aa, synthetic construct
STGGQI

SEQ ID NO: 176 - protein, 10 aa, synthetic construct
GSTGSITSMA

SEQ ID NO: 177 - protein, 5 aa, synthetic construct
NSGGR

SEQ ID NO: 178 - protein, 10 aa, synthetic construct
GSIAEIYVMG

SEQ ID NO: 179 - protein, 5 aa, synthetic construct
PSSGR

SEQ ID NO: 180 - protein, 10 aa, synthetic construct
GRGFSTYAMG

SEQ ID NO: 181 - protein, 6 aa, synthetic construct
SFGGGT

SEQ ID NO: 182 - protein, 10 aa, synthetic construct
GRTFSTYVMG

SEQ ID NO: 183 - protein, 6 aa, synthetic construct
STGGLI

SEQ ID NO: 184 - protein, 12 aa, synthetic construct
GRTLTLSMVTVG

SEQ ID NO: 185 - protein, 6 aa, synthetic construct
SWRGGR

SEQ ID NO: 186 - protein, 10 aa, synthetic construct
GRTFETIAMG

SEQ ID NO: 187 - protein, 6 aa, synthetic construct
RGSGVA

SEQ ID NO: 188 - protein, 10 aa, synthetic construct
GRTLNMYNMG

SEQ ID NO: 189 - protein, 6 aa, synthetic construct
SGKGLI

SEQ ID NO: 190 - protein, 10 aa, synthetic construct
GRTFSNYNMG

SEQ ID NO: 191 - protein, 6 aa, synthetic construct
SRSGVI

SEQ ID NO: 192 - protein, 10 aa, synthetic construct
GRTFNTKAIG

SEQ ID NO: 193 - protein, 6 aa, synthetic construct
SWGGGT

SEQ ID NO: 194 - protein, 10 aa, synthetic construct
GRTYSTVAMG

SEQ ID NO: 195 - protein, 6 aa, synthetic construct
TWSVGN

SEQ ID NO: 196 - protein, 10 aa, synthetic construct
GSIGSVTSMG

SEQ ID NO: 197 - protein, 5 aa, synthetic construct
DSNGR

SEQ ID NO: 198 - protein, 10 aa, synthetic construct
GRAFSRTLMG

SEQ ID NO: 199 - protein, 6 aa, synthetic construct
SWVSVT

SEQ ID NO: 200 - protein, 10 aa, synthetic construct
EGSFMRYNMG

SEQ ID NO: 201 - protein, 6 aa, synthetic construct
SGALAL

SEQ ID NO: 202 - protein, 9 aa, synthetic construct
GSISSNIMG

SEQ ID NO: 203 - protein, 6 aa, synthetic construct
SRRGLI

SEQ ID NO: 204 - protein, 10 aa, synthetic construct
GRTFETIAMG

SEQ ID NO: 205 - protein, 6 aa, synthetic construct
RGSGVA

SEQ ID NO: 206 - protein, 10 aa, synthetic construct
GRTFETIAMG

SEQ ID NO: 207 - protein, 6 aa, synthetic construct
RGSGVA

SEQ ID NO: 208 - protein, 777 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGGPLGVRGLRELHLDNNGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 209 - protein, 381 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGGGGGSGGGGSGGGGSGGGGSGGGGSEVQLVESGGGLVQPGGSLRLSCAASGRTFETIAMGWFRQAPGKGLEFVAVIRGSGVATYYAESVKGRFTISKDSSKNTVYLQMNSLRAEDTAVYYCAATTNRFKNTDYTTYDYWGQGTQVTVSSA

SEQ ID NO: 210 - protein, 783 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGGPLGVRGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGGLRELHLDNN

SEQ ID NO: 211 - protein, 381 aa, synthetic construct; identical to SEQ ID NO: 209

SEQ ID NO: 212 - protein, 782 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGGPLGVRGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSLRELHLDNNGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 213 - protein, 381 aa, synthetic construct; identical to SEQ ID NO: 209

SEQ ID NO: 214 - protein, 780 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGGPLGVRGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGGGGWSHW

SEQ ID NO: 215 - protein, 381 aa, synthetic construct; identical to SEQ ID NO: 209

SEQ ID NO: 216 - protein, 783 aa, synthetic construct; identical to SEQ ID NO: 210

SEQ ID NO: 217 - protein, 381 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGGGGGSGGGGSGGGGSGGGGSGGGGSEVQLVESGGGLVQPGGSLRLSCAASGRTFETIAMGWFRQAPGKGLEFVAVIRGSGVATYYAESVKGRFTISKDSSKNTVYLQMNSLRAEDTAVYYCAATTNRFKNREYTTYDYWGQGTQVTVSSA

SEQ ID NO: 218 - protein, 782 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGGPLGVRGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGGLRELHLDN

SEQ ID NO: 219 - protein, 381 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGGGGGSGGGGSGGGGSGGGGSGGGGSEVQLVESGGGLVQPGGSLRLSCAASGRTFETIAMGWFRQAPGKGLEFVAVIRGSGVATYYAESVKGRFTISKDSSKNTVYLQMNSLRAEDTAVYYCAATTNRFKNTDYTTYDYWGQGTQVTVSSA

SEQ ID NO: 220 - protein, 773 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGGPLGVRGLRELHLDNNGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 221 - protein, 381 aa, synthetic construct; identical to SEQ ID NO: 209

SEQ ID NO: 222 - protein, 779 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGGPLGVRGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGSGGLRELHLDNN

SEQ ID NO: 223 - protein, 381 aa, synthetic construct; identical to SEQ ID NO: 209

SEQ ID NO: 224 - protein, 778 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGGPLGVRGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSLRELHLDNNGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 225 - protein, 381 aa, synthetic construct; identical to SEQ ID NO: 209

SEQ ID NO: 226 - protein, 776 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGGPLGVRGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGSGGGGWSHW

SEQ ID NO: 227 - protein, 381 aa, synthetic construct; identical to SEQ ID NO: 209

SEQ ID NO: 228 - protein, 779 aa, synthetic construct; identical to SEQ ID NO: 222

SEQ ID NO: 229 - protein, 381 aa, synthetic construct; identical to SEQ ID NO: 217

SEQ ID NO: 230 - protein, 779 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGGPLGVRGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGSGGLRELHLDNN

SEQ ID NO: 231 - protein, 381 aa, synthetic construct; identical to SEQ ID NO: 219

SEQ ID NO: 232 - protein, 7 aa, synthetic construct
GPLGVRG

SEQ ID NO: 233 - protein, 7 aa, synthetic construct
GPLGLRG

SEQ ID NO: 234 - protein, 7 aa, synthetic construct
GPLGLAR

SEQ ID NO: 235 - protein, 8 aa, synthetic construct
GPAALVGA

SEQ ID NO: 236 - protein, 8 aa, synthetic construct
GPAALIGG

SEQ ID NO: 237 - protein, 8 aa, synthetic construct
GPLNLVGR

SEQ ID NO: 238 - protein, 8 aa, synthetic construct
GPAGLVAD

SEQ ID NO: 239 - protein, 8 aa, synthetic construct
GPANLVAP

SEQ ID NO: 240 - protein, 8 aa, synthetic construct
VPLSLYSG

SEQ ID NO: 241 - protein, 10 aa, synthetic construct
SGESPAYYTA

SEQ ID NO: 242 - protein, 770 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGGPLGVRGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 243 - protein, 382 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGGGGGSGGGGSGGGGSGGGGSGGGGSQVQLVESGGGLVQAGGSLRLSCAASGRTFETIAMGWFRQVPGKEREFVAVIRGSGVATYYPDSVKGRFTISKDSAKNTVYLQMNNLKPEDTAVYYCAATTNRFKNTDYTTYDYWGQGTQVTVSSSG

SEQ ID NO: 244 - protein, 124 aa, synthetic construct
EVQLVESGGGLVQPGGSLRLSCAASGRTFETIAMGWFRQAPGKEREFVAVIRGSGVATYYPDSVKGRFTISKDSSKNTVYLQMNSLRAEDTAVYYCAATTNRFKNTDYTTYDYWGQGTQVTVSS

SEQ ID NO: 245 - protein, 788 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGGPLGVRGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGSEGKSSGSGSESKSTGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGGLRELHLDNN

SEQ ID NO: 246 - protein, 381 aa, synthetic construct; identical to SEQ ID NO: 209

SEQ ID NO: 247 - protein, 788 aa, synthetic construct; identical to SEQ ID NO: 245

SEQ ID NO: 248 - protein, 381 aa, synthetic construct; identical to SEQ ID NO: 217

SEQ ID NO: 249 - protein, 782 aa, synthetic construct; identical to SEQ ID NO: 212

SEQ ID NO: 250 - protein, 381 aa, synthetic construct; identical to SEQ ID NO: 217

SEQ ID NO: 251 - protein, 787 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGGPLGVRGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGSEGKSSGSGSESKSTGGSLRELHLDNNGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 252 - protein, 381 aa, synthetic construct; identical to SEQ ID NO: 209

SEQ ID NO: 253 - protein, 787 aa, synthetic construct; identical to SEQ ID NO: 251

SEQ ID NO: 254 - protein, 381 aa, synthetic construct; identical to SEQ ID NO: 217

SEQ ID NO: 255 - protein, 777 aa, synthetic construct; identical to SEQ ID NO: 208

SEQ ID NO: 256 - protein, 381 aa, synthetic construct; identical to SEQ ID NO: 217

SEQ ID NO: 257 - protein, 782 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGGPLGVRGLRELHLDNNGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGSEGKSSGSGSESKSTGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 258 - protein, 381 aa, synthetic construct; identical to SEQ ID NO: 209

SEQ ID NO: 259 - protein, 782 aa, synthetic construct; identical to SEQ ID NO: 257

SEQ ID NO: 260 - protein, 381 aa, synthetic construct; identical to SEQ ID NO: 217

SEQ ID NO: 261 - protein, 780 aa, synthetic construct; identical to SEQ ID NO: 214

SEQ ID NO: 262 - protein, 381 aa, synthetic construct; identical to SEQ ID NO: 217

SEQ ID NO: 263 - protein, 785 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGGPLGVRGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGSEGKSSGSGSESKSTGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGGGGWSHW

SEQ ID NO: 264 - protein, 381 aa, synthetic construct; identical to SEQ ID NO: 209

SEQ ID NO: 265 - protein, 785 aa, synthetic construct; identical to SEQ ID NO: 263

SEQ ID NO: 266 - protein, 381 aa, synthetic construct; identical to SEQ ID NO: 217

SEQ ID NO: 267 - protein, 780 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGGPLGVRGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSSGGGGSGGGGSGGGGSGGWSHWGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 268 - protein, 381 aa, synthetic construct; identical to SEQ ID NO: 209

SEQ ID NO: 269 - protein, 780 aa, synthetic construct; identical to SEQ ID NO: 267

SEQ ID NO: 270 - protein, 381 aa, synthetic construct; identical to SEQ ID NO: 217

SEQ ID NO: 271 - protein, 784 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGGPLGVRGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGSEGKSSGSGSESKSTGGSGGWSHWGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 272 - protein, 381 aa, synthetic construct; identical to SEQ ID NO: 209

SEQ ID NO: 273 - protein, 784 aa, synthetic construct; identical to SEQ ID NO: 271

SEQ ID NO: 274 - protein, 381 aa, synthetic construct; identical to SEQ ID NO: 217

SEQ ID NO: 275 - protein, 777 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGGPLGVRGGGSGGWSHWGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 276 - protein, 381 aa, synthetic construct; identical to SEQ ID NO: 209

SEQ ID NO: 277 - protein, 777 aa, synthetic construct; identical to SEQ ID NO: 275

SEQ ID NO: 278 - protein, 381 aa, synthetic construct; identical to SEQ ID NO: 217

SEQ ID NO: 279 - protein, 782 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGGPLGVRGGGSGGWSHWGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGSEGKSSGSGSESKSTGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 280 - protein, 381 aa, synthetic construct; identical to SEQ ID NO: 209

SEQ ID NO: 281 - protein, 782 aa, synthetic construct; identical to SEQ ID NO: 279

SEQ ID NO: 282 - protein, 381 aa, synthetic construct; identical to SEQ ID NO: 217

SEQ ID NO: 283 - protein, 783 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGGPLGVRGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGGLRELHLDNN

SEQ ID NO: 284 - protein, 381 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGGGGGSGGGGSGGGGSGGGGSGGGGSEVQLVESGGGLVQPGGSLRLSCAASGRTFETIAMGWFRQAPGKGLEFVAVIRGSGVATYYAESVKGRFTISKDSSKNTVYLQMNSLRAEDTAVYYCAATTNRFKNREYTTYDYWGQGTQVTVSSA

SEQ ID NO: 285 - protein, 788 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGGPLGVRGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGSEGKSSGSGSESKSTGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGGLRELHLDNN

SEQ ID NO: 286 - protein, 381 aa, synthetic construct; identical to SEQ ID NO: 219

SEQ ID NO: 287 - protein, 788 aa, synthetic construct; identical to SEQ ID NO: 285

SEQ ID NO: 288 - protein, 381 aa, synthetic construct; identical to SEQ ID NO: 284

SEQ ID NO: 289 - protein, 782 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGGPLGVRGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSLRELHLDNNGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 290 - protein, 381 aa, synthetic construct; identical to SEQ ID NO: 219

SEQ ID NO: 291 - protein, 782 aa, synthetic construct; identical to SEQ ID NO: 289

SEQ ID NO: 292 - protein, 381 aa, synthetic construct; identical to SEQ ID NO: 284

SEQ ID NO: 293 - protein, 787 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGGPLGVRGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGSEGKSSGSGSESKSTGGSLRELHLDNNGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 294 - protein, 381 aa, synthetic construct; identical to SEQ ID NO: 219

SEQ ID NO: 295 - protein, 787 aa, synthetic construct; identical to SEQ ID NO: 293

SEQ ID NO: 296 - protein, 381 aa, synthetic construct; identical to SEQ ID NO: 284

SEQ ID NO: 297 - protein, 777 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGGPLGVRGLRELHLDNNGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 298 - protein, 381 aa, synthetic construct; identical to SEQ ID NO: 219

SEQ ID NO: 299 - protein, 777 aa, synthetic construct; identical to SEQ ID NO: 297

SEQ ID NO: 300 - protein, 381 aa, synthetic construct; identical to SEQ ID NO: 284

SEQ ID NO: 301 - protein, 782 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGGPLGVRGLRELHLDNNGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGSEGKSSGSGSESKSTGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 302 - protein, 381 aa, synthetic construct; identical to SEQ ID NO: 219

SEQ ID NO: 303 - protein, 782 aa, synthetic construct; identical to SEQ ID NO: 301

SEQ ID NO: 304 - protein, 381 aa, synthetic construct; identical to SEQ ID NO: 284

SEQ ID NO: 305 - protein, 780 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGGPLGVRGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGGGGWSHW

SEQ ID NO: 306 - protein, 381 aa, synthetic construct; identical to SEQ ID NO: 219

SEQ ID NO: 307 - protein, 780 aa, synthetic construct; identical to SEQ ID NO: 305

SEQ ID NO: 308 - protein, 381 aa, synthetic construct; identical to SEQ ID NO: 284

SEQ ID NO: 309 - protein, 785 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGGPLGVRGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGSEGKSSGSGSESKSTGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGGGGWSHW

SEQ ID NO: 310 - protein, 381 aa, synthetic construct; identical to SEQ ID NO: 219

SEQ ID NO: 311 - protein, 785 aa, synthetic construct; identical to SEQ ID NO: 309

SEQ ID NO: 312 - protein, 381 aa, synthetic construct; identical to SEQ ID NO: 284

SEQ ID NO: 313 - protein, 780 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGGPLGVRGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSSGGGGSGGGGSGGGGSGGWSHWGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 314 - protein, 381 aa, synthetic construct; identical to SEQ ID NO: 219

SEQ ID NO: 315 - protein, 780 aa, synthetic construct; identical to SEQ ID NO: 313

SEQ ID NO: 316 - protein, 381 aa, synthetic construct; identical to SEQ ID NO: 284

SEQ ID NO: 317 - protein, 784 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGGPLGVRGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGSEGKSSGSGSESKSTGGSGGWSHWGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 318 - protein, 381 aa, synthetic construct; identical to SEQ ID NO: 219

SEQ ID NO: 319 - protein, 784 aa, synthetic construct; identical to SEQ ID NO: 317

SEQ ID NO: 320 - protein, 381 aa, synthetic construct; identical to SEQ ID NO: 284

SEQ ID NO: 321 - protein, 777 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGGPLGVRGGGSGGWSHWGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 322 - protein, 381 aa, synthetic construct; identical to SEQ ID NO: 219

SEQ ID NO: 323 - protein, 777 aa, synthetic construct; identical to SEQ ID NO: 321

SEQ ID NO: 324 - protein, 381 aa, synthetic construct; identical to SEQ ID NO: 284

SEQ ID NO: 325 - protein, 782 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGGPLGVRGGGSGGWSHWGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGSEGKSSGSGSESKSTGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 326 - protein, 381 aa, synthetic construct; identical to SEQ ID NO: 219

SEQ ID NO: 327 - protein, 782 aa, synthetic construct; identical to SEQ ID NO: 325

SEQ ID NO: 328 - protein, 381 aa, synthetic construct; identical to SEQ ID NO: 284

SEQ ID NO: 329 - protein, 770 aa, synthetic construct; identical to SEQ ID NO: 242

SEQ ID NO: 330 - protein, 381 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGGGGGSGGGGSGGGGSGGGGSGGGGSQVQLVESGGGLVQAGGSLRLSCAASGRTFETIAMGWFRQVPGKEREFVAVIRGSGVATYYPDSVKGRFTISKDSAKNTVYLQMNNLKPEDTAVYYCAATTNRFKNTDYTTYDYWGQGTQVTVSSA

SEQ ID NO: 331 - protein, 766 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGGPLGVRGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 332 - protein, 381 aa, synthetic construct; identical to SEQ ID NO: 209

SEQ ID NO: 333 - protein, 5 aa, synthetic construct
SVSMA

SEQ ID NO: 334 - protein, 17 aa, synthetic construct
FSARISETTEYADFVKG

SEQ ID NO: 335 - protein, 14 aa, synthetic construct
SEGPATIRQNTYPD

SEQ ID NO: 336 - protein, 5 aa, synthetic construct
SYYMY

SEQ ID NO: 337 - protein, 16 aa, synthetic construct
TIKPNGSGIYGNSVAG

SEQ ID NO: 338 - protein, 6 aa, synthetic construct
DVRGTV

SEQ ID NO: 339 - protein, 5 aa, synthetic construct
LYNMG

SEQ ID NO: 340 - protein, 16 aa, synthetic construct
SISRAGRSSYGDSVKG

SEQ ID NO: 341 - protein, 6 aa, synthetic construct
SFLDDY

SEQ ID NO: 342 - protein, 5 aa, synthetic construct
SSWMF

SEQ ID NO: 343 - protein, 17 aa, synthetic construct
TISPSGDYSRYADSVQG

SEQ ID NO: 344 - protein, 6 aa, synthetic construct
DLRGTM

SEQ ID NO: 345 - protein, 5 aa, synthetic construct
NFVMK

SEQ ID NO: 346 - protein, 16 aa, synthetic construct
SIDTTHFTNYADSVKG

SEQ ID NO: 347 - protein, 8 aa, synthetic construct
RRRDYEDY

SEQ ID NO: 348 - protein, 5 aa, synthetic construct
RYNMG

SEQ ID NO: 349 - protein, 17 aa, synthetic construct
AIIWSGGVINYADSVKG

SEQ ID NO: 350 - protein, 15 aa, synthetic construct
DDKYYDRIVRGTADY

SEQ ID NO: 351 - protein, 5 aa, synthetic construct
VVSWG

SEQ ID NO: 352 - protein, 16 aa, synthetic construct
SDASGGRPNYQDSVKG

SEQ ID NO: 353 - protein, 12 aa, synthetic construct
RGLQLDMGLYDS

SEQ ID NO: 354 - protein, 5 aa, synthetic construct
DYAMG

SEQ ID NO: 355 - protein, 17 aa, synthetic construct
GISSGGGFTNYADSVKA

SEQ ID NO: 356 - protein, 14 aa, synthetic construct
QSGTNYISRTSPPY

SEQ ID NO: 357 - protein, 5 aa, synthetic construct
PYTMG

SEQ ID NO: 358 - protein, 16 aa, synthetic construct
SISSVYSTNYADSVKG

SEQ ID NO: 359 - protein, 11 aa, synthetic construct
PGLLHEEGTDY

SEQ ID NO: 360 - protein, 5 aa, synthetic construct
MYNMG

SEQ ID NO: 361 - protein, 17 aa, synthetic construct
AISGKGLISDYRDSVKG

SEQ ID NO: 362 - protein, 16 aa, synthetic construct
GQWSAGPFTRERSYEY

SEQ ID NO: 363 - protein, 5 aa, synthetic construct
RWTMA

SEQ ID NO: 364 - protein, 17 aa, synthetic construct
AVGWWNDSTYYADSVKG

SEQ ID NO: 365 - protein, 16 aa, synthetic construct
TEKYGLGQPNPRRYDY

SEQ ID NO: 366 - protein, 5 aa, synthetic construct
SYTMA

SEQ ID NO: 367 - protein, 17 aa, synthetic construct
TISPVGFIMDYADSVKG

SEQ ID NO: 368 - protein, 15 aa, synthetic construct
DLGRKLGTQSREYGY

SEQ ID NO: 369 - protein, 5 aa, synthetic construct
TYAVG

SEQ ID NO: 370 - protein, 17 aa, synthetic construct
AISWGGGTVRYADSVKG

SEQ ID NO: 371 - protein, 14 aa, synthetic construct
RVLHIATKAVDFGS

SEQ ID NO: 372 - protein, 5 aa, synthetic construct
MYVMG

SEQ ID NO: 373 - protein, 17 aa, synthetic construct
AISGEGLISDYRDSVKG

SEQ ID NO: 374 - protein, 16 aa, synthetic construct
GQWTNGPFTRERTYEY

SEQ ID NO: 375 - protein, 5 aa, synthetic construct
RSTMA

SEQ ID NO: 376 - protein, 16 aa, synthetic construct
SIPSDIGTKYADSVKG

SEQ ID NO: 377 - protein, 6 aa, synthetic construct
HIDSDY

SEQ ID NO: 378 - protein, 5 aa, synthetic construct
SRAMG

SEQ ID NO: 379 - protein, 17 aa, synthetic construct
AISFGGGTIRYADSVKG

SEQ ID NO: 380 - protein, 14 aa, synthetic construct
RRLHIATLAADFDS

SEQ ID NO: 381 - protein, 5 aa, synthetic construct
DYIVG

SEQ ID NO: 382 - protein, 17 aa, synthetic construct
AISSGGYIQHYIDSVKG

SEQ ID NO: 383 - protein, 18 aa, synthetic construct
NQLNGVARKKIESDDYDY

SEQ ID NO: 384 - protein, 5 aa, synthetic construct
EYAMG

SEQ ID NO: 385 - protein, 17 aa, synthetic construct
GISRGAGRTVYADSVKG

SEQ ID NO: 386 - protein, 17 aa, synthetic construct
DDVSYNRVTTAPGEYDY

SEQ ID NO: 387 - protein, 5 aa, synthetic construct
RSLMG

SEQ ID NO: 388 - protein, 17 aa, synthetic construct
AISWVSVTPDYGDSVKG

SEQ ID NO: 389 - protein, 16 aa, synthetic construct
SERYGTPRRRPNDYDY

SEQ ID NO: 390 - protein, 5 aa, synthetic construct
ITSMA

SEQ ID NO: 391 - protein, 16 aa, synthetic construct
SISSGGRPSYQDSVKG

SEQ ID NO: 392 - protein, 12 aa, synthetic construct
RGLHLDTGLYES

SEQ ID NO: 393 - protein, 5 aa, synthetic construct
LYVMG

SEQ ID NO: 394 - protein, 16 aa, synthetic construct
RITRGGSTSYANSVKG

SEQ ID NO: 395 - protein, 8 aa, synthetic construct
QTAVGPDY

SEQ ID NO: 396 - protein, 6 aa, synthetic construct
TLNAIG

SEQ ID NO: 397 - protein, 16 aa, synthetic construct
RITHDGRIVYGDSVKG

SEQ ID NO: 398 - protein, 4 aa, synthetic construct
PGMV

SEQ ID NO: 399 - protein, 7 aa, synthetic construct
LSMVTVG

SEQ ID NO: 400 - protein, 17 aa, synthetic construct
AISWRGGRSYVADDVKG

SEQ ID NO: 401 - protein, 16 aa, synthetic construct
REAIQDLAWTANDFTY

SEQ ID NO: 402 - protein, 5 aa, synthetic construct
TKAIG

SEQ ID NO: 403 - protein, 17 aa, synthetic construct
AISWGGGTIRYADSVKG

SEQ ID NO: 404 - protein, 14 aa, synthetic construct
RQLHIATLAADFDS

SEQ ID NO: 405 - protein, 5 aa, synthetic construct
SYTMA

SEQ ID NO: 406 - protein, 17 aa, synthetic construct
TISPVGFIMDYADSVKG

SEQ ID NO: 407 - protein, 15 aa, synthetic construct
DLGRKLGTQSCEYGY

SEQ ID NO: 408 - protein, 5 aa, synthetic construct
ITSMA

SEQ ID NO: 409 - protein, 16 aa, synthetic construct
SINSGGRPNYQESVKG

SEQ ID NO: 410 - protein, 12 aa, synthetic construct
RGLRLDTGLYES

SEQ ID NO: 411 - protein, 5 aa, synthetic construct
SRAMG

SEQ ID NO: 412 - protein, 17 aa, synthetic construct
AISFGGGTIRYADSVKG

SEQ ID NO: 413 - protein, 14 aa, synthetic construct
RRLHIATLAADFDS

SEQ ID NO: 414 - protein, 4 aa, synthetic construct
YVMA

SEQ ID NO: 415 - protein, 17 aa, synthetic construct
GIGWGSSRTYYADSVKG

SEQ ID NO: 416 - protein, 16 aa, synthetic construct
QGRISPIYTRANEYPY

SEQ ID NO: 417 - protein, 5 aa, synthetic construct
SYGMS

SEQ ID NO: 418 - protein, 15 aa, synthetic construct
GISAGSTKYADSVKG

SEQ ID NO: 419 - protein, 7 aa, synthetic construct
WPRLFED

SEQ ID NO: 420 - protein, 5 aa, synthetic construct
MTSTG

SEQ ID NO: 421 - protein, 16 aa, synthetic construct
SINSGGRPNYQDSVKG

SEQ ID NO: 422 - protein, 12 aa, synthetic construct
RGLVLSTGLYES

SEQ ID NO: 423 - protein, 5 aa, synthetic construct
LYVMG

SEQ ID NO: 424 - protein, 16 aa, synthetic construct
RITRGGSTSYANAVKG

SEQ ID NO: 425 - protein, 8 aa, synthetic construct
QTAVGPDY

SEQ ID NO: 426 - protein, 5 aa, synthetic construct
NYNMG

SEQ ID NO: 427 - protein, 17 aa, synthetic construct
TISRSGVITDYADSVKG

SEQ ID NO: 428 - protein, 15 aa, synthetic construct
ARSPVWGRGPDEYDT

SEQ ID NO: 429 - protein, 5 aa, synthetic construct
AYVMG

SEQ ID NO: 430 - protein, 17 aa, synthetic construct
AISTGGQISDYANSVKG

SEQ ID NO: 431 - protein, 15 aa, synthetic construct
NRENFLNRGAGDYEY

SEQ ID NO: 432 - protein, 5 aa, synthetic construct
ITSMA

SEQ ID NO: 433 - protein, 16 aa, synthetic construct
SINSGGRPNYQESVKG

SEQ ID NO: 434 - protein, 12 aa, synthetic construct
RGLRLDTGLYES

SEQ ID NO: 435 - protein, 5 aa, synthetic construct
IYVMG

SEQ ID NO: 436 - protein, 16 aa, synthetic construct
TTPSSGRTNIADSVKG

SEQ ID NO: 437 - protein, 9 aa, synthetic construct
RLTPTSVAS

SEQ ID NO: 438 - protein, 5 aa, synthetic construct
TYAMG

SEQ ID NO: 439 - protein, 17 aa, synthetic construct
AISFGGGTVRYVDSVKG

SEQ ID NO: 440 - protein, 14 aa, synthetic construct
RRLHIATLAADFGS

SEQ ID NO: 441 - protein, 5 aa, synthetic construct
TYVMG

SEQ ID NO: 442 - protein, 17 aa, synthetic construct
YISTGGLISDHADSVKG

SEQ ID NO: 443 - protein, 17 aa, synthetic construct
ASRTRRPIATIKDEYDY

SEQ ID NO: 444 - protein, 7 aa, synthetic construct
LSMVTVG

SEQ ID NO: 445 - protein, 17 aa, synthetic construct
AISWRGGRSSVADDVKG

SEQ ID NO: 446 - protein, 16 aa, synthetic construct
RTAIQDLAWTANDFTY

SEQ ID NO: 447 - protein, 5 aa, synthetic construct
TIAMG

SEQ ID NO: 448 - protein, 17 aa, synthetic construct
VIRGSGVATYYPDSVKG

SEQ ID NO: 449 - protein, 15 aa, synthetic construct
TTNRFKNTDYTTYDY

SEQ ID NO: 450 - protein, 5 aa, synthetic construct
MYNMG

SEQ ID NO: 451 - protein, 17 aa, synthetic construct
AISGKGLISDYRDSVKG

SEQ ID NO: 452 - protein, 16 aa, synthetic construct
GQWSAGPFTRERSYEY

SEQ ID NO: 453 - protein, 5 aa, synthetic construct
NYNMG

SEQ ID NO: 454 - protein, 17 aa, synthetic construct
TISRSGVITDYADYVKG

SEQ ID NO: 455 - protein, 15 aa, synthetic construct
ARSPVWGRGPDEYDT

SEQ ID NO: 456 - protein, 5 aa, synthetic construct
TKAIG

SEQ ID NO: 457 - protein, 17 aa, synthetic construct
AISWGGGTIRYADSVKG

SEQ ID NO: 458 - protein, 14 aa, synthetic construct
RQLHIATLAADFDS

SEQ ID NO: 459 - protein, 5 aa, synthetic construct
TVAMG

SEQ ID NO: 460 - protein, 17 aa, synthetic construct
AITWSVGNTAVADSVKG

SEQ ID NO: 461 - protein, 17 aa, synthetic construct
RTNIGAFNLFRENHYNY

SEQ ID NO: 462 - protein, 5 aa, synthetic construct
VTSMG

SEQ ID NO: 463 - protein, 16 aa, synthetic construct
SADSNGRTTFQDFVQG

SEQ ID NO: 464 - protein, 12 aa, synthetic construct
RGLQLTMGLYES

SEQ ID NO: 465 - protein, 5 aa, synthetic construct
RTLMG

SEQ ID NO: 466 - protein, 17 aa, synthetic construct
GISWVSVTPDYGNSVKG

SEQ ID NO: 467 - protein, 16 aa, synthetic construct
SQRYGTPRRRPNDYEY

SEQ ID NO: 468 - protein, 5 aa, synthetic construct
RYNMG

SEQ ID NO: 469 - protein, 17 aa, synthetic construct
AMSGALALIRYADSVKG

SEQ ID NO: 470 - protein, 18 aa, synthetic construct
DLEPQYWTKAGQRDTYDV

SEQ ID NO: 471 - protein, 4 aa, synthetic construct
NIMG

SEQ ID NO: 472 - protein, 17 aa, synthetic construct
VISRRGLILDYGDSVKG

SEQ ID NO: 473 - protein, 16 aa, synthetic construct
GKTTDRFSEIPSDYDY

SEQ ID NO: 474 - protein, 5 aa, synthetic construct
TIAMG

SEQ ID NO: 475 - protein, 17 aa, synthetic construct
VIRGSGVATYYAESVKG

SEQ ID NO: 476 - protein, 15 aa, synthetic construct
TTNRFKNTDYTTYDY

SEQ ID NO: 477 - protein, 5 aa, synthetic construct
TIAMG

SEQ ID NO: 478 - protein, 17 aa, synthetic construct
VIRGSGVATYYAESVKG

SEQ ID NO: 479 - protein, 15 aa, synthetic construct
TTNRFKNREYTTYDY

SEQ ID NO: 480 - protein, 770 aa, synthetic construct; identical to SEQ ID NO: 242

SEQ ID NO: 481 - protein, 382 aa, synthetic construct; identical to SEQ ID NO: 243

SEQ ID NO: 482 - protein, 785 aa, synthetic construct
MGWSCIILFLVATATGVHSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNRFTQKSLSLSPGGGGGSGGGGGVRLGPGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 483 - protein, 401 aa, synthetic construct
MGWSCIILFLVATATGVHSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSGGGGSEVQLVESGGGLVQPGGSLRLSCAASGRTFETIAMGWFRQAPGKGLEFVAVIRGSGVATYYAESVKGRFTISKDSSKNTVYLQMNSLRAEDTAVYYCAATTNRFKNTDYTTYDYWGQGTQVTVSSAA

SEQ ID NO: 484 - protein, 798 aa, synthetic construct
MGWSCIILFLVATATGVHSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNRFTQKSLSLSPGGGGGSGGGGGVRLGPGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGSGGLRELHLDNN

SEQ ID NO: 485 - protein, 401 aa, synthetic construct; identical to SEQ ID NO: 483